(19) United States
(12) Patent Application Publication
(10) Pub. No.: US 2024/0016941 A1
(43) Pub. Date: Jan. 18, 2024

(54) COMBINATION THERAPY

(71) Applicant: Antabio SAS, Labege (FR)

(72) Inventors: Martin Everett, Labege (FR); Simon Leiris, Labege (FR); David Thomas Davies, Labege (FR); Nicolas Sprynski, Labege (FR); Lilha Beyria, Labege (FR); Thomas David Pallin, Margate (GB); Andrew Peter Cridland, Margate (GB); Toby Jonathan Blench, Margate (GB); Richard Leonard Elliott, Margate (GB); David Edward Clark, Margate (GB)

(73) Assignee: Antabio SAS, Labege (FR)

(21) Appl. No.: 17/912,275

(22) PCT Filed: Mar. 23, 2021
(86) PCT No.: PCT/EP2021/057497

§ 371 Date: Sep. 16, 2022

Related U.S. Application Data
(60) Provisional application No. 62/993,842, filed on Mar. 24, 2020.

(51) Int. Cl. A61K 47/54; A61P 31/04; A61K 31/443; A61K 45/06

(52) U.S. Cl. CPC A61K 47/545; A61K 45/06; A61K 31/443; A61P 31/04

(57) ABSTRACT

The invention provides a combinations and pharmaceutical compositions comprising (i) a compound which is an indane according to Formula (I) or a pharmaceutically acceptable salt thereof; and (ii) one or more CFTR modulator; wherein R¹, R², R³, R⁴, n, Lk, (A), G and m are as defined herein. Also provided are therapeutic uses of such combinations and compositions in the treatment of conditions such as cystic fibrosis.

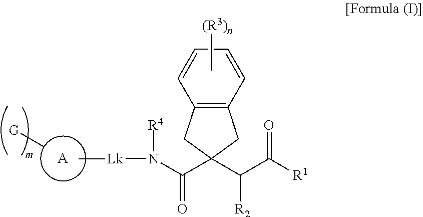

### FIELD OF THE INVENTION

The present invention relates to combinations of compounds which are useful in treating conditions such as cystic fibrosis. The invention also relates to the use of compounds included in the provided combinations together with other therapies such as genetic therapies for treating conditions such as cystic fibrosis. The compounds used in such combinations and associated therapies are indanes. The compounds act as inhibitors of the enzyme LasB. The invention provides combinations of said indanes with CFTR modulators, and pharmaceutical compositions comprising said indanes and one or more CFTR modulator. Also provided are medical uses of the indane compounds, combinations and compositions, and associated methods of treating conditions such as cystic fibrosis.

### BACKGROUND

Cystic fibrosis (CF) is a life-threatening disease affecting approximately 70,000 sufferers worldwide. CF is the most common lethal, hereditary disease in Caucasian populations, resulting from mutations in the cystic fibrosis transmembrane conductance regulator (CFTR) gene. The prevalence of CF in Europe is 1 in every 2,000-3,000 live births, and in North America is about 1 in every 3,500 births. In the UK there are approximately 9,800 people with CF.

Cystic Fibrosis is an inherited recessive genetic disease caused by mutations in both copies of the gene coding for the cystic fibrosis transmembrane conductance regulator (CFTR) protein. Most common mutations in this protein (i) impair protein synthesis, (ii) prevent the protein from migrating to the surface of the cell and/or (iii) lead to synthesis of an ion channel that fails to open correctly. The consequence of a defective ion channel is that chloride ions cannot flow in and out of the cell as they should do, leading to an imbalance of ionic concentrations resulting in an increase of mucus thickness and its accumulation in several organs such as lung, pancreas and gut. In lungs, mucus accumulation results in a reduction of lung function, chronic inflammation and persistent infections. These chronic infections worsen both the general pathology and the lung function by increasing the inflammation and degrading lung tissue integrity.

Individuals with CF typically have to contend with infections caused by bacteria including Staphylococcus aureus, Haemophilus influenza, Pseudomonas aeruginosa and Burkholderia cepacia. Pseudomonas aeruginosa (PA) is the most common cause of chronic lung infection in individuals with CF, and chronic infection with PA is found in 9% of pre-school children, 32% of 10-15 year olds and the majority (between 59% and 80%) of adults with CF, leading to progressive lung damage and early death.

As the lung of the individual with CF is colonised by PA, the growth pattern of the bacteria changes and its capacity for survival improves. In chronic infection, PA bacteria on mucosal and epithelial surfaces, or in sputum, form biofilms as well as producing large quantities of alginate (the so-called mucoid phenotype) which reduce the effectiveness of phagocytosis and antibiotic therapy. This leads to chronic colonisation of the lung by PA that is not cleared by conventional antibiotic therapy. Patients who are colonised with PA show a more rapid decline in lung function, faster decline in chest radiograph score, poor weight gain, increased hospitalisation rates and an increased need for antibiotic therapy. Median survival is reduced and mortality is significantly increased. Most disease-related morbidity and mortality in CF is caused by progressive lung disease as a result of bacterial infection and airway inflammation, primarily associated with the effects of chronic PA lung infection and the persistence of PA biofilms.

Considerable efforts have been made to identify effective treatments for CF. To date, many treatments have sought to manage the symptoms of CF including the bacterial infection common to CF patients. However, despite intensive antibiotic treatment, adaptive mechanisms such as biofilm formation allow PA to resist both immune and antibiotic pressures, leading to recurrent exacerbations and respiratory failure. In a different approach, treatments have been recently explored for addressing the defect in the CFTR protein which leads to the CF symptoms.

Recently a number of drugs (CFTR modulators) have been introduced into the clinic either as standalone drugs or in combination, and which can address certain mutant CFTR proteins. The broad class of CFTR modulators includes CFTR potentiators, CFTR correctors and CFTR amplifiers. CFTR potentiators preferentially stabilise the CFTR channel in the open form in order to facilitate ion channel movement through the CFTR protein. CFTR correctors typically promote wild-type-like folding of the mutant CFTR protein thereby promoting cellular transport to the cell membrane. CFTR amplifiers typically increase cellular levels of CFTR mRNA leading to increased protein expression.

Another early stage approach to treat the disease of CF is gene therapy. In one aspect, plasmid DNA encoding for a non-mutant form of the CTFR gene can be directly administered to a CF patient, causing the increased expression of active protein. Such plasmid DNA can be administered in a variety of ways, e.g. by encapsulation in a cationic liposome, which can for instance be administered to a subject in need by inhalation. Early data is encouraging for the benefits of this approach. A related approach is to administer the mRNA that can be translated into the CFTR protein directly to the patient, for example via inhalation of lipid nanoparticles containing the mRNA to deliver the mRNA to the lungs of the individual.

Although as explained above CFTR mutations are responsible for disease in individuals with CF and lead to chronic infections by bacteria such as Pseudomonas aeruginosa, CFTR deficiencies are also implicated in other clinical indications. For example, CFTR expression has been reported as being downregulated in epithelial cells treated with cigarette smoke, and CFTR-sufficient smokers have been described as exhibiting a decrease in CFTR function. Decreased CFTR protein expression has also been associated with non-CF pulmonary conditions such as COPD. Accordingly, strategies for improving therapies for CF patients may also prove useful in treating other conditions associated with CFTR downregulation or decreased CFTR function, including COPD, particularly in subjects also suffering from bacterial infections.

Despite the advantages that therapies such as CFTR modulators offer in treating CF, challenges remain. One key issue is that even successful CFTR correction therapies (CFTR modulators, genetic therapies etc) can sometimes still be insufficient to counter the negative effects of bacterial infection. Not only is such infection dangerous in a primary sense (i.e. leading to inflammation, etc), but secondary effects of such infection include a reduced efficacy of CFTR correction therapies, e.g. based on the in vitro activity of the therapies and their in vivo activity in otherwise healthy organisms. There is thus a pressing need for new strategies for improving the efficacy of existing treatments for CF and other conditions associated with CFTR downregulation or decreased CFTR function.

### SUMMARY OF THE INVENTION

The effect which bacteria may have on CFTR function is highly complex. For example, Laselva et al (Biomolecules 2020, 10, 334) describes how expression of mature CFTR has been found to decrease upon infection with P. aeruginosa, and Trinh et al (Eur Respir J 2015, 45, 1590-1602) describes how PA exoproducts may reduce CFTR protein synthesis. Other studies have indicated that LasB, a metalloprotease present in the P. aeruginosa secretome and a virulence factor in CF secretions downregulates interleukin (IL)-6, a antimicrobial-lung repair pathway involved in upregulating expression of the antimicrobial molecule trappin-21 infection, and may also be implicated in CFTR degradation.

The present inventors have recognized that in view of such activity, CFTR correction therapies, e.g. the use of CFTR modulators, will be hampered by LasB expressed by the bacteria typically colonizing subjects suffering from CF.

One possible approach to address such issues could perhaps be to administer CFTR modulators together with antibiotics in order to combat the bacterial infection. A large number of antibiotic compounds are known and have been shown to exhibit antibacterial activity against a wide range of LasB expressing bacteria. However, currently available antibiotics are incapable of controlling some bacterial infections. In some cases, this is because the target bacteria have acquired antibiotic resistance, for example via horizontal gene transfer. For example, Laselva et al (Biomolecules 2020, 10, 334) describes how a combination therapy involving CFTR modulators and the antibiotic compound tobramycin was incapable of restoring CFTR function in cells infected by tobramycin-resistance PA bacteria. In other cases, antibiotic treatment is unsuccessful because the target bacteria are found in a state in which the efficacy of antibiotics which would otherwise be highly active is reduced. One such state is a bacterial biofilm.

Bacteria in biofilms are enclosed in a self-produced extracellular biopolymer matrix, which may include polysaccharides, proteins and DNA. Bacteria in biofilms typically exhibit different properties from free-living bacteria of the same species. Such properties typically include increased resistance to antibiotics and detergents and increased lateral gene transfer. For example, bacteria in biofilms typically display up to 1,000-fold higher tolerance to antibiotic challenge than their single cell, planktonic (free-living) counterparts.

Pathogenic bacteria are typically the target of antibacterial treatments, but biofilms formed by such bacteria are typically extremely difficult to eradicate using conventional therapeutic regimes. In particular, antibiotic compounds are often incapable of effectively penetrating the biofilm matrix to target the bacteria. This limitation in the efficacy of antibacterial compounds is especially important for individuals who through immunodeficiency or other diseases or conditions cannot adequately combat bacterial infection. Such individuals include those suffering from cystic fibrosis.

Appreciating this difficulty, the present inventors have recognised that a new avenue of therapy for diseases associated with CFTR downregulation and/or decreased CFTR function lies in increasing the efficacy of CFTR correction therapies, e.g. the use of CFTR modulators, by targeting specifically the LasB protein expressed by such bacteria. In this context, the present inventors have developed a series of compounds which are highly active against LasB. Such compounds enhance the activity of CFTR correction therapies by reducing the LasB-induced degradation of the functional CFTR protein.

As described in more detail herein, the inventors have found that compounds of Formula (I) are potent inhibitors of the Pseudomonas aeruginosa-derived elastase enzyme LasB.

Accordingly, the invention provides a combination comprising (i) a compound which is an indane according to Formula (I) or a pharmaceutically acceptable salt thereof, and (ii) one or more CFTR modulator;

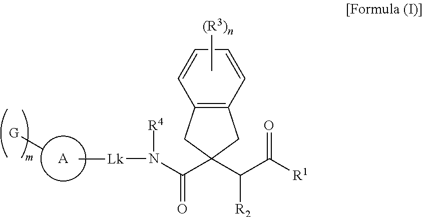

wherein:
R¹ is selected from:
   —NHOH, —OH, —OR^{1a} and —OCH₂OC(O)R^{1a}, wherein R^{1a} is selected from an unsubstituted C₁ to C₄ alkyl group and phenyl; and
   wherein when the compound of Formula (I) contains a positively charged nitrogen atom, R¹ may be O⁻, such that the compound forms a zwitterion;
R² is selected from H and unsubstituted C₁ to C₂ alkyl;
each R³ group is independently selected from halogen, —OH, —NH₂, methyl and —CF₃;
n is an integer from 0 to 4;
R⁴ is selected from H and unsubstituted C₁ to C₂ alkyl;
Lk is a linking group;
\textcircled{A} is a cyclic group selected from C₆ to C₁₀ aryl, 5- to 14-membered heteroaryl, and 4- to 14-membered carbocyclic and heterocyclic groups; wherein when \textcircled{A} is a heterocyclic or heteroaryl group comprising at least one nitrogen atom, said nitrogen atom(s) are independently selected from secondary, tertiary and quaternary nitrogen atom(s);
m is an integer from 0 to 3; and
each G group is selected from:
   a 4- to 10-membered nitrogen-containing heterocyclic group which is unsubstituted or is substituted; wherein the nitrogen atom(s) in said heterocyclic group are independently selected from secondary, tertiary and quaternary nitrogen atom(s);
   C₂ to C₄ alkoxy; C₁ to C₄ alkyl; C₂ to C₄ alkenyl; C₂ to C₄ alkynyl; and
   —NR^{Y}—C₁ to C₄ alkyl each of which is unsubstituted or is substituted; wherein R^{Y} is H or unsubstituted C₁ to C₃ alkyl;
   methoxy which is unsubstituted or is substituted by one, two or three halogen substituents; halogen; —OH; —NR²⁰R²¹ and —N⁺R²⁰R²¹R²², wherein R²⁰ and R²¹ are each independently selected from H and optionally substituted C₁ to C₃ alkyl;
   C₃ to C₆ carbocyclyl; —O—C₃ to C₆ carbocyclyl; and —NR^{Y}—C₃ to C₆ carbocyclyl; wherein R^{Y} is H or unsubstituted C₁ to C₃ alkyl; and
   R⁶, wherein each R⁶ group is independently selected from:
      —R^{6a}R^{A}, —O—R^{6a}R^{A}, —NR²⁰—R^{6a}R^{A}, —R^{6b}R^{B}, —O—R^{6b}R^{B}, and —NR²⁰—R^{6b}R^{B};
      R^{X}R^{R}, —O—R^{X}R^{R}, —O—R^{X}—C(O)—R^{R}, —R^{X}—C(O)—R^{R}, —NR²⁰—R^{X}R^{R}, and —NR²⁰—R^{X}—C(O)—R^{R}; and
      CN; —C(O)NR²⁰R²¹; —C(O)NR²¹—R^{X}R^{B}; —C(O)NR⁴⁰R⁴¹; —SO₂R²⁰; —SO₂—R^{X}R^{B}; —SO₂NR²⁰R²¹; —SO₂—NR²⁰—R^{X}R^{B}; and —SO₂NR⁴⁰R⁴¹;
      wherein:
      each R^{X} is independently selected from R^{6a} and R^{6b};
      each R^{6a} is independently selected from C₁ to C₄ alkylene, C₂ to C₄ alkenylene and C₂ to C₄ alkynylene; and each R^{6a} is independently unsubstituted or is substituted;
      each R^{6b} is independently selected from [C₁ to C₃ alkylene]-[5-6-membered carbocyclyl or heterocyclyl], [C₂ to C₃ alkenylene]-[5-6-membered carbocyclyl or heterocyclyl] and [C₂ to C₃ alkynylene]-[5-6-membered carbocyclyl or heterocyclyl];
      R^{A} is selected from —NR²⁰R³⁰; —N⁺R²⁰R²¹R³⁰; —NR²⁰NR²¹R²²; —NR²⁰NR²¹R²²R²³; —N⁺R²⁰R²¹NR²²R²³; —NR²⁰C(NR²¹)NR²²R³⁰; —NR²⁰C(N⁺R²¹R²²)NR²³R³⁰; —C(NR²⁰)NR²¹R²²; and —C(N⁺R²⁰R²¹)NR²²R²³;
      R^{B} is selected from —NR²⁰R²¹; —N⁺R²⁰R²¹R²²; —NR²⁰NR²¹R²²; —NR²⁰N⁺R²¹R²²R²³; —N⁺R²⁰R²¹NR²²R²³; —NR²⁰C(NR²¹)NR²²R²³; —NR²⁰C(N⁺R²¹R²²)NR²³R²⁴; —C(NR²⁰)NR²¹R²²; and —C(N⁺R²⁰R²¹)NR²²R²³;
      R⁴⁰ and R⁴¹, together with the nitrogen atom to which they are attached, form an optionally substituted 4- to 6-membered heterocyclic group, wherein any nitrogen atom in the ring is independently selected from secondary, tertiary and quaternary nitrogen atoms;
      each R^{R} is independently an optionally substituted 4- to 10-membered heteroaryl or heterocyclic group comprising at least one nitrogen atom, and said nitrogen atom(s) are independently selected from secondary, tertiary and quaternary nitrogen atom(s);
      R²⁰, R²¹, R²², R²³ and R²⁴ are each independently selected from H and optionally substituted C₁ to C₃ alkyl; and
      each R³⁰ is independently selected from optionally substituted C₂ to C₃ alkyl.

Also provided is a pharmaceutical composition comprising (i) a compound which is an indane according to Formula (I) as defined herein or a pharmaceutically acceptable salt thereof; (ii) one or more CFTR modulator and (iii) one or more pharmaceutically acceptable excipient, carrier or diluent.

In the combination or pharmaceutical composition as provided herein, said CFTR modulator is typically selected from CFTR potentiators, CFTR correctors and CFTR amplifiers. Exemplary CFTR modulators include ivacaftor, lumacaftor, tezacaftor, elexacaftor, VX659, VX152 and VX-440 and combinations thereof.

Also provided is a combination or pharmaceutical composition as defined herein, further comprising an antibiotic agent. The antibiotic agent is preferably selected from tobramycin, neomycin, streptomycin, gentamycin, ceftazidime, ticarcillin, piperacillin, tazobactam, imipenem, meropenem, rifampicin, ciprofloxacin, amikacin, colistin, aztreonam, azithromycin and levofloxacin.

Also provided is a combination or pharmaceutical composition as defined herein for use in treating a disease associated with CFTR downregulation or decreased CFTR function in a subject.

The invention further provides a compound which is an indane according to Formula (I) or a pharmaceutically acceptable salt thereof as defined herein for use in treating a disease associated with CFTR downregulation or decreased CFTR function in a subject, said use comprising administering said compound in combination with a CFTR modulator to said subject. Further provided is a CFTR modulator for use in treating a disease associated with CFTR downregulation or decreased CFTR function in a subject, said use comprising administering said CFTR modulator in combination with a compound which is an indane according to Formula (I) or a pharmaceutically acceptable salt thereof as defined herein. Preferably, the CFTR modulator is as described herein. Preferably, said disease is cystic fibrosis (CF) or chronic obstructive pulmonary disease.

Also provided is the use of a combination, pharmaceutical composition or compound according to Formula (I) as defined herein, in treating bacterial infection in a subject. Preferably, said subject suffers from cystic fibrosis.

Still further provided is a compound which is an indane according to Formula (I) or a pharmaceutically acceptable salt thereof as defined herein for use in treating a disease associated with CFTR downregulation or decreased CFTR function in a subject receiving a genetic therapy for said disease. Preferably said genetic therapy is selected from an integrating gene therapy; a non-integrating gene therapy; and an RNA therapy.

### BRIEF DESCRIPTION OF THE FIGURES

FIG. 1 shows incidences of mortality vs survival and chronic colonization vs bacterial clearance in a mouse model of lung infection, 7 days post-infection with wt and ΔlasB mutant PA strains. Results are discussed in Example 57.

** p<0.01.

FIG. 2 shows quantification of active IL-1β in the lung following infection by wild-type and ΔlasB mutant PAO1, with and without treatment with compounds of Formula (I) in murine lungs at 24 hours post infection. Results are discussed in Example 59. **p<0.001, ****p<0.0001.

RU=relative light units, proportional to the levels of IL-1β in this experiment.

FIG. 3 shows total colony forming units of wild-type and ΔlasB mutant PAO1, with and without treatment with compounds of Formula (I) in murine lungs at 24 hours post infection. Results are discussed in Example 59.

**p<0.01, ***p<0.001

FIG. 4 shows quantification of active IL-1β in the lung following infection by wild-type and ΔlasB mutant PAO1, with and without treatment with compounds of Formula (I) in murine lungs at 24 hours post infection. Results are discussed in Example 59.

**p<0.001, ****p<0.0001.

RU=relative light units, proportional to the levels of IL-1β in this experiment.

FIG. 5 shows total colony forming units of wild-type and ΔlasB mutant PAO1, with and without treatment with compounds of Formula (I) in murine lungs at 24 hours post infection. Results are discussed in Example 59.

**p<0.01, ***p<0.001

FIGS. 6A and 6B show the ability of a LasB inhibitor as described herein (the compound of example 23) to counteract the reduction in CFTR expression in LasB-exposed cells in a dose-dependent manner, returning the CFTR level to a similar level as observed in the non-LasB exposed cells. Results are discussed in Example 61.

### DETAILED DESCRIPTION OF THE INVENTION

As used herein, a C₁ to C₄ alkyl group is a linear or branched alkyl group containing from 1 to 4 carbon atoms. A C₁ to C₄ alkyl group is often a C₁ to C₃ alkyl group. Examples of C₁ to C₄ alkyl groups include methyl, ethyl, n-propyl, iso-propyl, n-butyl, sec-butyl, and tert-butyl. A C₁ to C₃ alkyl group is typically a C₁ to C₂ alkyl group. A C₁ to C₂ alkyl group is methyl or ethyl, typically methyl. For the avoidance of doubt, where two alkyl groups are present, the alkyl groups may be the same or different.

As used herein, an alkoxy group is typically a said alkyl group attached to an oxygen atom. Thus, a C₂ to C₄ alkoxy group is a C₂ to C₄ alkyl group attached to an oxygen atom. A C₁ to C₃ alkoxy group is a C₁ to C₃ alkyl group attached to an oxygen atom. Examples of C₂ to C₄ alkoxy groups include ethoxy, n-propyoxy, iso-propoxy, n-butoxy, sec-butoxy, and tert-butoxy. Examples of C₁ to C₃ alkoxy groups include methoxy, ethoxy n-propyoxy and iso-propoxy. Typically, a C₁ to C₃ alkoxy group is a C₁ to C₂ alkoxy group such as a methoxy or ethoxy group. For the avoidance of doubt, where two alkoxy groups are present, the alkoxy groups may be the same or different.

As used herein, a C₂ to C₄ alkenyl group is a linear or branched alkenyl group containing from 2 to 4 carbon atoms and having one or more, e.g. one or two, typically one double bonds. Typically a C₂ to C₄ alkenyl group is a C₂ to C₃ alkenyl group. Examples of C₂ to C₄ alkenyl groups include ethenyl, propenyl and butenyl. For the avoidance of doubt, where two alkenyl groups are present, the alkenyl groups may be the same or different.

As used herein, a C₂ to C₄ alkynyl group is a linear or branched alkynyl group containing from 2 to 4 carbon atoms and having one or more, e.g. one or two, typically one triple bonds. Typically a C₂ to C₄ alkynyl group is a C₂ to C₃ alkynyl group. Examples of C₂ to C₄ alkynyl groups include ethynyl, propynyl and butynyl. For the avoidance of doubt, where two alkynyl groups are present, the alkynyl groups may be the same or different.

Unless otherwise stated, an alkyl, alkoxy, alkenyl or alkynyl group as defined herein may be unsubstituted or substituted as provided herein. The substituents on a substituted alkyl, alkenyl, alkynyl or alkoxy group are typically themselves unsubstituted. Where more than one substituent is present, these may be the same or different.

As used herein, a halogen is typically chlorine, fluorine, bromine or iodine and is preferably chlorine, bromine or fluorine, especially chorine or fluorine.

A 3- to 14-membered carbocyclic group is a cyclic hydrocarbon containing from 3 to 14 carbon atoms. A 4- to 10-membered carbocyclic group is a cyclic hydrocarbon containing from 4 to 10 carbon atoms. A carbocyclic group may be saturated or partially unsaturated, but is typically saturated. A 4- to 10-membered carbocyclic group may be a fused bicyclic group or a spiro bicyclic group, as defined herein. A 4- to 10-membered carbocyclic group may be a saturated 4- to 6-membered, preferably 5- or 6-membered carbocyclic group. Examples of 4- to 6-membered saturated carbocyclic groups include cyclobutyl, cyclopentyl and cyclohexyl groups.

A 3- to 14-membered heterocyclic group is a cyclic group containing from 3 to 14 atoms selected from C, O, N and S in the ring, including at least one heteroatom, and typically one or two heteroatoms. The heteroatom or heteroatoms are typically selected from O, N, and S, most typically from S and N, especially N. A 4- to 10-membered heterocyclic group is a cyclic group containing from 4 to 10 atoms selected from C, O, N and S in the ring, including at least one heteroatom, and typically one or two heteroatoms. The heteroatom or heteroatoms are typically selected from O, N, and S, most typically from O and N, especially N. A heterocyclic group may be saturated or partially unsaturated, but is typically saturated. A 4- to 10-membered heterocyclic group may be a fused bicyclic group or a spiro bicyclic group, as defined herein. A 4- to 10-membered heterocyclic group may be a saturated 4- to 6-membered, preferably 5- or 6-membered heterocyclic group. References herein to heterocyclic group(s) include quaternised derivatives thereof, as defined herein. Preferred nitrogen-containing heterocyclic groups include azetidine, morpholine, 1,4-oxazepane, octahydropyrrolo[3,4-c]pyrrole, piperazine, piperidine, and pyrrolidine, including quaternised derivatives thereof, as defined herein.

As used herein, a C₆ to C₁₀ aryl group is a substituted or unsubstituted, monocyclic or fused polycyclic aromatic group containing from 6 to 10 carbon atoms in the ring portion. Examples include monocyclic groups such as phenyl and fused bicyclic groups such as naphthyl and indenyl. Phenyl (benzene) is preferred.

As used herein, a 5- to 10-membered heteroaryl group is a substituted or unsubstituted monocyclic or fused polycyclic aromatic group containing from 5 to 10 atoms in the ring portion, including at least one heteroatom, for example 1, 2 or 3 heteroatoms, typically selected from O, S and N. A heteroaryl group is typically a 5- or 6-membered heteroaryl group or a 9- or 10-membered heteroaryl group. Preferably, the heteroaryl group comprises 1, 2 or 3, preferably 1 or 2 nitrogen atoms. References herein to heteroaryl group(s) include quaternised derivatives thereof, as defined herein. Preferred nitrogen-containing heteroaryl groups include imidazole, pyridine, pyrimidine and pyrazine, including quaternised derivatives thereof, as defined herein.

As used herein, a fused bicyclic group is a group comprising two cyclic moieties sharing a common bond between two atoms. A spiro bicyclic group is a group comprising two cyclic moieties sharing a common atom.

A carbocyclic, heterocyclic, aryl or heteroaryl group may be unsubstituted or substituted as described herein. The substituents on a substituted carbocyclic, heterocyclic, aryl or heteroaryl group are typically themselves unsubstituted, unless otherwise stated.

A number of the compounds described herein comprise heterocyclic or heteroaryl groups comprising at least one nitrogen atom. In such compounds, said nitrogen atom(s) are independently selected from secondary, tertiary and quaternary nitrogen atom(s). A quaternary nitrogen atom is present when the compound comprises a quaternised derivative of one or more monocyclic groups or fused bicyclic groups. As used herein, a quaternised derivative of a moiety such as a cyclic moiety is formed by bonding an additional alkyl group to a nitrogen atom in the moiety such that the valency of the said nitrogen atom increases from 3 to 4 and the nitrogen atom is positively charged.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as oxalic, citric, fumaric, maleic, malic, ascorbic, succinic, tartaric, palmitic, benzoic, acetic, triphenylacetic, methanesulphonic, ethanesulphonic, 1-hydroxy-2-naphthenoic, isethionic, benzenesulphonic orp-toluenesulphonic acid. Pharmaceutically acceptable bases include alkali metal (e.g. sodium or potassium), alkali earth metal (e.g. calcium or magnesium) and zinc bases, for example hydroxides, carbonates, and bicarbonates, and organic bases such as alkyl amines, aralkyl (i.e. aryl-substituted alkyl; e.g. benzyl) amines and heterocyclic amines.

Where the compound of Formula (I) contains a positively charged nitrogen atom, the compound may exist as a zwitterion, where R¹ is O⁻, thus leaving a COO⁻ group. Such compounds may also be provided in the form of a pharmaceutically acceptable salt. Suitable salts include those formed with pharmaceutically acceptable acids, which provide a proton to the COO⁻ group, and a counter-ion to balance the positive charge on the quaternary nitrogen atom. Suitable pharmaceutically acceptable acids include hydrochloric acid, sulphonic acids including methanesulphonic acid and toluene sulphonic acid, ascorbic acid and citric acid. Hydrochloric acid and sulphonic acids are preferred, in particular hydrochloric acid. Alternatively, zwitterions can be combined with pharmaceutically acceptable bases as mentioned above, for example, alkali metal (e.g. sodium or potassium) and alkali earth metal (e.g. calcium or magnesium) hydroxides. The compounds of Formula (I) can also be provided as so-called triple salts. Typically a triple salt of a compound of Formula (I) comprises both a zwitterion pair within the Formula (I) compound and a further charged group which is associated with a counterion in order to form a salt. For example, the compound of Formula (I) may comprise two negatively charged groups and one positively charged group, such that a zwitterion pair forms between one of the negatively charged groups and the positively charged group, and the remaining negatively charged group forms a salt. Alternatively, the compound of Formula (I) may comprise two positively charged groups and one negatively charged group, such that a zwitterion pair forms between one of the positively charged groups and the negatively charged group, and the remaining positively charged group forms a salt.

In Formula (I), the stereochemistry is not limited. In particular, compounds of Formula (I) containing one or more stereocentre (e.g. one or more chiral centre) may be used in enantiomerically or diastereoisomerically pure form, or in the form of a mixture of isomers. Further, for the avoidance of doubt, the compounds of the invention may be used in any tautomeric form. Typically, the agent or substance described herein contains at least 50%, preferably at least 60, 75%, 90% or 95% of a compound according to Formula (I) which is enantiomerically or diasteriomerically pure. Thus, the compound is preferably substantially optically pure.

For the avoidance of doubt, the terms ‘indane’, ‘indanyl derivative’ and ‘indane derivative’ may be used interchangeably and unless otherwise indicated refer to compounds of the invention, such as compounds of Formula (I).

Compounds of Formula (I)

Usually, in Formula (I),
R¹ is selected from:
   —NHOH, —OH, —OR^{1a} and —OCH₂OC(O)R^{1a}, wherein R^{1a} is selected from an unsubstituted C₁ to C₄ alkyl group and phenyl; and
   wherein when the compound of Formula (I) contains a positively charged nitrogen atom, R¹ may be O⁻, such that the compound forms a zwitterion;
R² is selected from H and unsubstituted C₁ to C₂ alkyl;
each R³ group is independently selected from halogen, —OH, —NH₂, methyl and —CF₃;
n is an integer from 0 to 4;
R⁴ is selected from H and unsubstituted C₁ to C₂ alkyl;
Lk is a linking group;
\textcircled{A} is a cyclic group selected from C₆ to C₁₀ aryl, 5- to 14-membered heteroaryl, and 4- to 14-membered carbocyclic and heterocyclic groups; wherein when \textcircled{A} is a heterocyclic or heteroaryl group comprising at least one nitrogen atom, said nitrogen atom(s) are independently selected from secondary, tertiary and quaternary nitrogen atom(s);
m is an integer from 0 to 3; and
each G group is selected from:

A:
i) a 4- to 10-membered nitrogen-containing heterocyclic group which is unsubstituted or is substituted by one or two substituents independently selected from —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; —C(NR¹¹)R¹²; halogen, —OH; and C₁ to C₄ alkoxy; C1 to C₄ alkyl; C₂ to C₄ alkenyl; C₂ to C₄ alkynyl; and —NR^{Y}—C₁ to C₄ alkyl; wherein each alkyl, alkenyl, alkoxy and alkynyl group is independently unsubstituted or is substituted with one, two or three groups independently selected from —OH, halogen; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²; wherein the nitrogen atom(s) in said heterocyclic group are independently selected from secondary, tertiary and quaternary nitrogen atom(s);
ii) C₂ to C₄ alkoxy; C1 to C₄ alkyl; C₂ to C₄ alkenyl; C₂ to C₄ alkynyl; and —NR^{Y}—C₁ to C₄ alkyl each of which is unsubstituted or is substituted with one, two or three groups independently selected from —OH, halogen; —NR¹¹R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²; and methoxy which is substituted by one, two or three halogen substituents;
iii) halogen, —OH and unsubstituted methoxy; and
iv) C₃ to C₆ carbocyclyl; —O—C₃ to C₆ carbocyclyl; and —NR^{Y}—C₃ to C₆ carbocyclyl; wherein each carbocyclyl group is unsubstituted or is substituted with one or two groups independently selected from —OH, halogen; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; —C(NR¹¹)R¹²; C₁ to C₄ alkoxy; C₁ to C₄ alkyl; C₂ to C₄ alkenyl; C₂ to C₄ alkynyl; and —NR^{Y}—C₁ to C₄ alkyl; wherein each alkyl, alkenyl, alkoxy and alkynyl group is independently unsubstituted or is substituted with one, two or three groups independently selected from —OH, halogen; methoxy; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²;
wherein each R¹⁰, R¹¹, R¹², R¹³ and R¹⁴ is independently H or methyl; and R^{Y} is H or unsubstituted C₁ to C₃ alkyl;

or

B:
—OMe, —OH, halogen, —NR¹⁰R¹¹; —N⁺R¹⁰R¹¹R¹², and —CF₃;
C₂ to C₄ alkoxy which is unsubstituted or is substituted with a group selected from —OH; —NR¹⁰R¹¹; —N⁺R¹⁰R¹¹R¹²; —OR^{6c} and —NR¹⁰R^{6c}, wherein R^{6c} is a C₁ to C₃ alkyl group which is unsubstituted or substituted with a group selected from OH;
—NR¹⁰R¹¹; —N⁺R¹⁰R¹¹R¹²; —NR¹⁰NR¹¹R¹²; —NR¹⁰N⁺R¹¹R¹²R¹³; —N⁺R¹⁰R¹¹NR¹²R¹³; —NR¹⁰C(NR¹¹)NR¹²R¹³; —NR¹⁰C(N⁺R¹¹R¹²)NR¹³R¹⁴; —C(NR¹⁰)NR¹¹R¹²; and —C(N⁺R¹⁰R¹¹)NR¹²R¹³;
wherein each R¹⁰, R¹¹, R¹², R¹³ and R¹⁴ is independently H or methyl-NR¹⁰R¹¹; —N⁺R¹⁰R¹¹R¹², and —CF₃;

or

C:
—OMe, —OH, halogen, —NR¹⁰R¹¹; —N⁺R¹⁰R¹¹R¹², and —CF₃;
R⁶, wherein each R⁶ group is independently selected from:
   —R^{6a}R^{A}, —O—R^{6a}R^{A}, —NR²⁰—R^{6a}R^{A}, —R^{6b}R^{B}, —O—R^{6b}R^{B}, and —NR²⁰—R^{6b}R^{B};
   —R^{X}R^{R}, —O—R^{X}R^{R}, —O—R^{X}—C(O)—R^{R}, —R^{X}—C(O)—R^{R}, —NR²⁰—R^{X}R^{R}, and —NR²⁰—R^{X}—C(O)—R^{R}; and
   —CN; —C(O)NR²⁰R²¹; —C(O)NR²¹—R^{X}R^{B}; —C(O)NR⁴⁰R⁴¹; —SO₂R²⁰; —SO₂—R^{X}R^{B}; —SO₂NR²⁰R²¹; —SO₂—NR²⁰—R^{X}R^{B}; and —SO₂NR⁴⁰R⁴¹;
wherein:
   each R^{X} is independently selected from R^{6a} and R^{6b};
   each R^{6a} is independently selected from C₁ to C₄ alkylene, C₂ to C₄ alkenylene and C₂ to C₄ alkynylene; and each R^{6a} is independently unsubstituted or is substituted by one group selected from —OH, halogen; —NR²⁰R²¹; —N⁺R²⁰R²¹R²²; —NR²⁰C(NR²¹)NR²²R²³; —NR²⁰C(N⁺R²¹R²²)NR²³R²⁴; —NR²⁰C(NR²¹)R²²; —NR²⁰C(N⁺R²¹R²²)R²³; —C(NR²⁰)NR²¹R²²; —C(N⁺R²⁰R²¹)NR²²R²³; —C(NR²⁰)R²¹; and —C(N⁺R²⁰R²¹)R²²; —C(O)NR²⁰R²¹; —C(O)N⁺R²⁰R²¹R²²; —C(O)—R²⁰, and methoxy which is unsubstituted or is substituted by one, two or three halogen substituents;
   each R^{b} is independently selected from [C₁ to C₃ alkylene]-C(R^{z})₂, [C₂ to C₃ alkenylene]—C(R^{z})₂ and [C₂ to C₃ alkynylene]—C(R^{z})₂; wherein the two R^{Z} groups are attached together to form, together with the atom to which they are attached, a 5- or 6-membered carbocyclic or heterocyclic group;
   R^{A} is selected from —NR²⁰R³⁰; —N⁺R²⁰R²¹R³⁰; —NR²⁰NR²¹R²²; —NR²⁰N⁺R²¹R²²R²³; —N⁺R²⁰R²¹NR²²R²³; —NR²⁰C(NR²¹)NR²²R²³; —NR²⁰C(N⁺R²¹R²²)NR²³R³⁰; —C(NR²⁰)NR²¹R²²; and —C(N⁺R²⁰R²¹)NR²²R²³;
   R^{B} is selected from —NR²⁰R²¹; —N⁺R²⁰R²¹R²²; —NR²⁰NR²¹R²²; —NR²⁰N⁺R²¹R²²R²³; —N⁺R²⁰R²¹NR²²R²³; —NR²⁰C(NR²¹)NR²²R²³; —NR²⁰C(N⁺R²¹R²²)NR²³R²⁴; —C(NR²⁰)NR²¹R²²; and —C(N⁺R²⁰R²¹)NR²²R²³;
   R⁴⁰ and R⁴¹, together with the nitrogen atom to which they are attached, form a 4- to 6-membered heterocyclic group, wherein any nitrogen atom in the ring is independently selected from secondary, tertiary and quaternary nitrogen atoms;
   each R^{R} is independently a 4- to 10-membered heteroaryl or heterocyclic group comprising at least one nitrogen atom, and said nitrogen atom(s) are independently selected from secondary, tertiary and quaternary nitrogen atom(s);
      wherein each R^{R}, and each ring formed by —NR⁴⁰R⁴¹, is independently unsubstituted or is substituted with one, two or three groups independently selected from
   i) halogen, —CN;
   ii) oxo, providing that said R^{R} group is a heterocyclic group;
   iii) —R²⁰, —R⁷—OR²⁰; —R⁷—NR²⁰R²¹; —R⁷—N⁺R²⁰R²¹R²²; —R⁷—NR²⁰C(NR²¹)NR²²R²³; —R⁷—NR²⁰C(N⁺R²¹R²²)NR²³R²⁴; —R⁷—NR²⁰C(NR²¹)R²²; —R⁷—NR²⁰C(N⁺R²¹R²²)R²³; —R⁷—C(NR²⁰)NR²¹R²²; —R⁷—C(N⁺R²⁰R²¹)NR²²R²³; —R⁷—C(NR²⁰)R²¹; and —R⁷—C(N⁺R²⁰R²¹)R²²;
   each R⁷ is independently selected from a bond and unsubstituted C₁ to C₃ alkylene;
   R²⁰, R²¹, R²², R²³ and R²⁴ are each independently selected from H and C₁ to C₃ alkyl which is unsubstituted or is substituted with one —OH or —OMe group or with one, two or three halogen groups; and
   each R³⁰ is independently selected from C₂ to C₃ alkyl which is unsubstituted or is substituted with one —OH or —OMe group or with one, two or three halogen groups.

Typically, in Formula (I), R¹ is selected from OH, NHOH and OR^{1a}, or where the compound of Formula (I) contains a positively charged nitrogen atom, R¹ may be O⁻, such that the compound forms a zwitterion. R^{1a} is typically an unsubstituted C₁ to C₄ alkyl group, such as an unsubstituted C₁ to C₂ alkyl group. More preferably, R^{1a} is methyl or t-butyl.

More preferably, R¹ is OH or NHOH, or where the compound of Formula (I) contains a positively charged nitrogen atom, R¹ may be O⁻, such that the compound forms a zwitterion. Still more preferably, R¹ is OH, or where the compound of Formula (I) contains a positively charged nitrogen atom, R¹ may be O⁻, such that the compound forms a zwitterion.

Preferably, R² is selected from H and unsubstituted C₁ to C₂ alkyl; preferably R² is selected from H and methyl. More preferably, R² is H. Preferably, R⁴ is selected from H and methyl. More preferably, R⁴ is H. Still more preferably, R² and R⁴ are independently H or methyl, most preferably they are both H.

Preferably, therefore, R¹ is selected from OH, NHOH and OR^{1a}; or where the compound of Formula (I) contains a positively charged nitrogen atom, R¹ may be O⁻, such that the compound forms a zwitterion; R² is selected from H and unsubstituted C₁ to C₂ alkyl; and R⁴ is H.

Each R³ group is typically independently selected from halogen; and —OH; and —NH₂. More preferably, each R³ group is independently selected from halogen (e.g. fluorine or chlorine) and —OH. Yet more preferably each R³ group is halogen (e.g. fluorine or chlorine), most preferably fluorine. Typically, n is an integer from 0 to 2; more preferably n is 0 or 2; most preferably n is 0. Preferably, where more than one R³ group is present, each R³ is the same.

Preferably, therefore, in Formula (I):
R¹ is OH or NHOH, or where the compound of Formula (I) contains a positively charged nitrogen atom, R¹ may be O⁻, such that the compound forms a zwitterion;
R² is selected from H and methyl;
each R³ group is independently selected from halogen (e.g. fluorine or chlorine) and —OH;
n is an integer from 0 to 2; and
R⁴ is selected from H and methyl.

Preferably, in Formula (I), Lk is selected from -L- and —(CH₂)_{d}-L′-(CH₂)ₑ—; wherein:
i) L is selected from a bond and a C₁ to C₃ alkylene group which is unsubstituted or is substituted by one group selected from halogen, —OH, —OMe, —NR²⁰R²¹;
   —N⁺R²⁰R²¹R²², and —CF₃; wherein R²⁰, R²¹ and R²² are each independently selected from H and C₁ to C₃ alkyl which is unsubstituted or is substituted with one —OH or —OMe group or with one, two or three halogen groups;
   and
ii) d is 0 or 1; e is 0 or 1; and L′ is selected from the moieties:

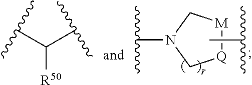

wherein
   R⁵⁰ is selected from —R⁶⁰, —C(O)OR⁶⁰; —C(O)NR¹⁰R⁶⁰; and —C(O)R⁶⁰;
   R⁶⁰ is selected from
   i) H;
   ii) a C₁ to C₄ alkyl group which is unsubstituted or is substituted with one, two or three groups independently selected from —OH; —NR¹⁰R¹¹; —N⁺R¹⁰R¹¹R¹²; and halogen; and
   iii) a cyclic group selected from 3- to 10-membered carbocyclic and heterocyclic groups, 5- to 10-membered heteroaromatic groups and 6- to 10-membered aromatic groups; which cyclic group is unsubstituted or is substituted by one or two substituents independently selected from —OH; —NR¹⁰R¹¹; —N⁺R¹⁰R¹¹R¹²; halogen; and C₁ to C₄ alkyl groups which are themselves each independently unsubstituted or substituted with one, two or three groups independently selected from —OH; —NR¹⁰R¹¹; —N⁺R¹⁰R¹¹R¹²; and halogen;
      wherein when said cyclic group is a heterocyclic group comprising at least one nitrogen atom, said nitrogen atom(s) are independently selected from secondary, tertiary and quaternary nitrogen atom(s);
   the moiety -M-Q- is selected from —CH₂—CH₂—; —CH₂—NH—; and —CH₂—O—;
      wherein a hydrogen atom from one of M and Q is replaced with the bond to the moiety —(CH₂)ₑ—NR⁴—; with the proviso that when e is 0, the moiety -M-Q- is bonded to the —NR⁴— moiety of Formula (I) via a ring carbon atom;
   r is 1 or 2;
   each R¹⁰, R¹¹ and R¹² is independently H or methyl.

When Lk is L, L is preferably selected from a bond and an unsubstituted C₁ to C₂ alkylene group. More preferably, when Lk is L, L is an unsubstituted C₁ alkylene (i.e. methylene) group.

Typically, when Lk is —(CH₂)_{d}-L′-(CH₂)ₑ—, e is 0. Often, d is 0. Preferably, e is 0 and d is 0 or 1. More preferably, e is 0 and d is 0.

In one embodiment, L′ is the moiety

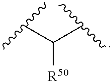

Typically, R⁵⁰ is selected from —R⁶⁰, —C(O)OR⁶⁰; and —C(O)R⁶⁰. Alternatively, R⁵⁰ can be selected from —R⁶⁰, —C(O)OR⁶⁰; and —C(O)NR¹⁰R⁶⁰, where R¹⁰ is H or methyl, typically H. Preferably, R⁵⁰ is selected from —R⁶⁰ and —C(O)OR⁶⁰. More preferably, R⁵⁰ is —R⁶⁰.

R⁶⁰ is preferably selected from:
(i) H;
(ii) a C₁ to C₄ alkyl group which is unsubstituted or is substituted with one, two or three groups independently selected from —OH; —NR¹⁰R¹¹; —N⁺R¹⁰R¹¹R¹²; and halogen; and
(iii) a cyclic group selected from 3- to 10-membered carbocyclic and heterocyclic groups; which cyclic group is unsubstituted or is substituted by one or two substituents independently selected from —OH; —NR¹⁰R¹¹; —N⁺R¹⁰R¹¹R¹²; halogen; and C₁ to C₄ alkyl groups which are each independently unsubstituted or substituted with one, two or three groups independently selected from —OH; —NR¹⁰R¹¹; —N⁺R¹⁰R¹¹R¹²; and halogen.

When R⁶⁰ is according to option (ii) above, R⁶⁰ is typically a C₁ to C₄ alkyl group which is unsubstituted or is substituted with one or two groups, preferably one group, independently selected from —OH; —NR¹⁰R¹¹; and —N⁺R¹⁰R¹¹R¹²; more preferably R⁶⁰ is a t-butyl group or is a C₁ to C₂ alkyl group which is unsubstituted or is substituted with one group selected from —OH; —NR¹⁰R¹¹; and —N⁺R¹⁰R¹¹R¹²; still more preferably R^{6c} is a C₁ to C₂ alkyl group which is unsubstituted or is substituted with one group selected from —NR¹⁰R¹¹ and —N⁺R¹⁰R¹¹R¹², and most preferably R⁶⁰ is methyl or ethyl, preferably methyl.

When R⁶⁰ is according to option (iii) above, R⁶⁰ is a preferably a 3- to 10-membered carbocyclic or heterocyclic group or a 5- to 10-membered heteroaromatic group; more preferably a 3- to 10-membered heterocyclic group or a 5- to 10-membered heteroaromatic group; still more preferably a 5- to 6-membered heterocyclic group or a 5- to 6-membered heteroaromatic group. When R⁶⁰ is according to option (iii) above, R⁶⁰ is more typically a 3- to 10-membered heterocyclic group; preferably a 5- to 6-membered heterocyclic group. When R⁶⁰ is a heteroaromatic group, it is preferably a nitrogen-containing heteroaromatic group, more preferably an oxadiazole group e.g. 1,3,4-oxadiazole. When R⁶⁰ is a heterocyclic group, the heterocyclic group is preferably saturated. When R⁶⁰ is a heterocyclic group, it is preferably a nitrogen-containing heterocyclic group. More preferably, when R⁶⁰ is a heterocyclic group, R⁶⁰ is piperidine or piperazine, most preferably piperidine.

When R⁶⁰ is according to option (iii) above, R⁶⁰ is typically unsubstituted or is substituted by one or two substituents independently selected from C₁ to C₄ alkyl groups which are each independently unsubstituted or substituted with one, two or three groups independently selected from —OH; —NR¹⁰R¹¹; —N⁺R¹⁰R¹¹R¹²; and halogen; more preferably R⁶⁰ is unsubstituted or is substituted by one or two substituents independently selected from C₁ to C₂ alkyl groups which are each independently unsubstituted or substituted with one group selected from —OH; —NR¹⁰R¹¹; —N⁺R¹⁰R¹¹R¹²; and still more preferably R⁶⁰ is unsubstituted or is substituted by one or two methyl substituents.

Preferably, R⁶⁰ is according to option (i) above or option (ii) above. More preferably, R⁶⁰ is H (i.e., R⁶⁰ is according to option (i) above).

In another embodiment, L′ is the moiety

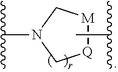

### Preferably, r is 1.

The moiety -M-Q- is selected from —CH₂—CH₂—; —CH₂—NH—; and —CH₂—O—; wherein a hydrogen atom from one of M and Q is replaced with the bond to the moiety —(CH₂)ₑ—NR⁴— of Formula (I). In other words, the moiety -M-Q- is selected from —CH( [symbol] )—CH₂—; —CH₂—CH( [symbol] )-; —CH( [symbol] )—NH—; —CH₂—N( [symbol] )-; and —CH( [symbol] )—O—; wherein [symbol] indicates the point of attachment to the moiety —(CH₂)ₑ—NR⁴— of Formula (I).

When e is 0, the moiety —(CH₂)ₑ—NR⁴— of Formula (I) is —NR⁴—, and the moiety -M-Q- is bonded to the —NR⁴— moiety of Formula (I) via a ring carbon atom. In other words, when e is 0, -M-Q- is selected from —CH( [symbol] )—CH₂—; —CH₂—CH( [symbol] )-; —CH( [symbol] )—NH—; and —CH( [symbol] )—O—. Preferably, -M-Q- is selected from —CH₂—CH₂— and —CH₂—NH—, more preferably —CH₂—CH₂—. Still more preferably, -M-Q- is selected from —CH₂—CH( [symbol] )- and —CH₂—N( [symbol] )-, more preferably —CH₂—CH( [symbol] )-. Most preferably, the moiety

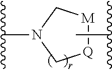

is piperidinylene or pyrrolidinylene, preferably pyrrolidinylene.

Preferably, in Formula (I), L′ is the moiety

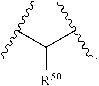

Preferably, therefore, in Formula (I):
e is 0;
d is 0 or 1;
L′ is selected from the moieties

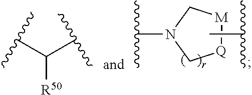

R⁵⁰ is selected from —R⁶⁰, —C(O)OR⁶⁰; —C(O)NR¹⁰R⁶⁰; and —C(O)R⁶⁰, preferably from —R⁶⁰ and —C(O)OR⁶⁰;
R⁶⁰ is selected from:
(i) H;
(ii) a C₁ to C₄ alkyl group which is unsubstituted or is substituted with one or two groups, preferably one group, independently selected from —OH; —NR¹⁰R¹¹; and —N⁺R¹⁰R¹¹R¹².
and
(iii) a 5- to 6-membered heterocyclic group which is unsubstituted or is substituted by one or two substituents independently selected from C₁ to C₂ alkyl groups which are each independently unsubstituted or substituted with one group selected from —OH; —NR¹⁰R¹¹; —N⁺R¹⁰R¹¹R¹²; and
the moiety

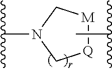

is selected from piperidinylene or pyrrolidinylene.

More preferably, in Formula (I):
e is 0;
d is 0;
L′ is the moiety

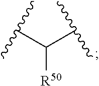

R⁵⁰ is —R⁶⁰; and
R⁶⁰ is selected from:
(i) H;
(ii) a C₁ to C₂ alkyl group which is unsubstituted or is substituted with one group selected from —NR¹⁰R¹¹ and —N⁺R¹⁰R¹¹R¹²;
and
(iii) piperidine and piperazine, each of which is unsubstituted or is substituted by one or two methyl substituents.

In a particularly preferred embodiment, d=e=0 and L′ represents a group —CH₂—.

Therefore, in Formula (I), most preferably Lk is —CH₂—.

In Formula (I), \textcircled{A} is preferably selected from benzothiazole, thiazole, pyrazole, benzene, benzofuran, benzimidazole, benzothiophene, benzoxazole, indole, isoquinoline, 2,3-dihydrobenzofuran, 2,3-dihydrobenzo[b][1,4]dioxine, and 4,5,6,7-tetrahydrothiazolo[5,4-c]pyridine. More preferably, \textcircled{A} is selected from benzene, benzothiazole, thiazole, benzofuran, 2,3-dihydrobenzofuran, benzimidazole, benzothiophene, indole, and 2,3-dihydrobenzo[b][1,4]dioxine. Still more preferably, \textcircled{A} is selected from benzene, benzothiazole, thiazole, benzofuran, and indole. Sometimes, \textcircled{A} is not benzene. Even more preferably, \textcircled{A} is selected from benzothiazole, thiazole, benzofuran and indole, e.g. \textcircled{A} is benzothiazole or thiazole. Most preferably, \textcircled{A} is benzothiazole.

In Formula (I), preferably m is 1 or 2. More preferably, m is 2.

In some preferred compounds, \textcircled{A} is benzothiazole and, preferably, m is 2.

Preferably, therefore, in Formula (I):
R¹ is selected from —OH and —NHOH, or where the compound of Formula (I) contains a positively charged nitrogen atom, R¹ may be O⁻, such that the compound forms a zwitterion;
R² is H;
R⁴ is H;
n is 0; or n is 2 and each R³ group is halogen, preferably fluorine.
Lk is —CH₂—;
\textcircled{A} is benzothiazole; and
m is 2.

In a first preferred embodiment of Formula (I), each G group is independently selected from:
i) a 4- to 10-membered nitrogen-containing heterocyclic group which is unsubstituted or is substituted by one or two substituents independently selected from —NR¹¹R¹¹; —N⁺R¹⁰R¹¹R¹²; —NR¹⁰C(NR¹¹)NR¹²R¹³; —NR¹⁰C(N⁺R¹¹R¹²)NR¹³R¹⁴;
   —NR¹⁰C(NR¹¹)R¹²; —C(NR¹¹)R¹²; halogen, —OH; and C₁ to C₄ alkoxy; C₁ to C₄ alkyl; C₂ to C₄ alkenyl; C₂ to C₄ alkynyl; and —NR^{Y}—C₁ to C₄ alkyl; wherein each alkyl, alkenyl, alkoxy and alkynyl group is independently unsubstituted or is substituted with one, two or three groups independently selected from —OH, halogen; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²; wherein the nitrogen atom(s) in said heterocyclic group are independently selected from secondary, tertiary and quaternary nitrogen atom(s);
ii) C₂ to C₄ alkoxy; C₁ to C₄ alkyl; C₂ to C₄ alkenyl; C₂ to C₄ alkynyl; and —NR^{Y}—C₁ to C₄ alkyl each of which is unsubstituted or is substituted with one, two or three groups independently selected from —OH, halogen; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²; and methoxy which is substituted by one, two or three halogen substituents;
iii) halogen, —OH and unsubstituted methoxy; and
iv) C₃ to C₆ carbocyclyl; —O—C₃ to C₆ carbocyclyl; and —NR^{Y}—C₃ to C₆ carbocyclyl; wherein each carbocyclyl group is unsubstituted or is substituted with one or two groups independently selected from —OH, halogen; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; —C(NR¹¹)R¹²; C₁ to C₄ alkoxy; C₁ to C₄ alkyl; C₂ to C₄ alkenyl; C₂ to C₄ alkynyl; and —NR^{Y}—C₁ to C₄ alkyl; wherein each alkyl, alkenyl, alkoxy and alkynyl group is independently unsubstituted or is substituted with one, two or three groups independently selected from —OH, halogen; methoxy; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²;
   wherein each R¹⁰, R¹¹, R¹², R¹³ and R¹⁴ is independently H or methyl; and R^{Y} is H or unsubstituted C₁ to C₃ alkyl.

Preferably, in this embodiment, each G is independently selected from:
(i) a 4- to 6-membered nitrogen-containing heterocyclic group which is unsubstituted or is substituted by one or two substituents selected from C₁ to C₂ alkyl; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; NR¹⁰C(NR¹¹)R¹²; and —C(NR¹¹)R¹²;
(ii) C₂ to C₄ alkoxy; C₁ to C₄ alkyl; C₂ to C₄ alkenyl; C₂ to C₄ alkynyl; and —NR^{Y}—C₁ to C₄ alkyl; each of which is unsubstituted or is substituted with one or two groups independently selected from —OH, halogen; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; NR¹⁰C(NR¹¹)R¹²; and —C(NR¹¹)R¹²;
and
(iii) halogen, —OH and methoxy;
G is preferably a basic group; i.e. a species having a lone pair of electrons that is capable of binding to a proton, or such a species in its protonated form. Alkylated analogues are also typically suitable.

In embodiments when G is a 4- to 10-membered nitrogen-containing heterocyclic group according to option (i) above, G is preferably a 4- to 7-membered nitrogen-containing heterocyclic group. More preferably G is a 4- to 6-membered nitrogen-containing heterocyclic group or a spiro 7-membered nitrogen-containing heterocyclic group, for example a 4- to 6-membered nitrogen-containing heterocyclic group. Preferably, when G is a nitrogen-containing heterocyclic group according to option (i), G is unsubstituted or is substituted by one or two substituents independently selected from —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; —C(NR¹¹)R¹²; and unsubstituted or substituted C₁ to C₂ alkyl; wherein each substituted alkyl group is substituted with one, two or three groups independently selected from —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²; preferably by one or two groups independently selected from —NR¹⁰R¹¹ and —N⁺R¹⁰R¹¹R¹². More preferably, G is unsubstituted or is substituted by one or two substituents selected from C₁ to C₂ alkyl; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; NR¹⁰C(NR¹¹)R¹²; and —C(NR¹¹)R¹².

Accordingly, when G is according to option (i) above, G is preferably a 4- to 7-membered nitrogen-containing heterocyclic group which is unsubstituted or is substituted by one or two substituents independently selected from —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; —C(NR¹¹)R¹²; and unsubstituted or substituted C₁ to C₂ alkyl; wherein each substituted alkyl group is substituted with one, two or three groups independently selected from —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²; preferably by one or two groups independently selected from —NR¹⁰R¹¹ and —N⁺R¹⁰R¹¹R¹². When G is a 4- to 7-membered nitrogen-containing heterocyclic group, G is preferably selected from piperazine, piperidine, pyrrolidine and azetidine and 2,6-diazaspiro[3.3]heptane. More preferably, when G is according to option (i) above, G is preferably a 4- to 7- or 4- to 6-membered nitrogen-containing heterocyclic group which is unsubstituted or is substituted by one or two substituents independently selected from —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; —C(NR¹¹)R¹²; and C₁ to C₂ alkyl which is unsubstituted or is substituted with one or two groups independently selected from —NR¹⁰R¹¹ and —N⁺R¹⁰R¹¹R¹². More preferably, when G is according to option (i), G is a 4- to 6-membered nitrogen-containing heterocyclic group or a spiro 7-membered nitrogen-containing heterocyclic group, for example a 4- to 6-membered nitrogen-containing heterocyclic group which is unsubstituted or is substituted by one or two substituents selected from C₁ to C₂ alkyl; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; NR¹⁰C(NR¹¹)R¹²; and —C(NR¹¹)R¹², more preferably by —NR¹⁰R¹¹ or —N⁺R¹⁰R¹¹R¹².

Still more preferably, when G is according to option (i), G is a 4- to 6-membered nitrogen-containing heterocyclic group which is unsubstituted or is substituted by one or two substituents selected from C₁ to C₂ alkyl; —NR¹⁰R¹¹; and —N⁺R¹⁰R¹¹R¹². When G is according to option (i), G is most preferably substituted by one substituent. When G is a 4- to 6-membered nitrogen-containing heterocyclic group, G is preferably selected from piperazine, piperidine, pyrrolidine and azetidine; more preferably from piperazine, piperidine and pyrrolidine; most preferably piperazine; each of which is unsubstituted or is substituted by one or two substituents selected from C₁ to C₂ alkyl; —NR¹⁰R¹¹; and —N⁺R¹⁰R¹¹R¹²; more preferably by one substituent selected from methyl, —NH₂ and —N⁺Me₃. For the avoidance of doubt, a substituted nitrogen-containing heterocyclic group G may be substituted at a ring nitrogen atom (for example a piperazine ring may be substituted by one or two methyl groups so that G is 1-methylpiperazine or 1,1-dimethylpiperazin-1-ium etc.) or at a ring carbon atom (for example a pyrrolidine ring may be substituted by an —NR¹⁰R¹¹ or —N⁺R¹⁰R¹¹R¹² group so that G is pyrrolidin-3-amine, N,N-dimethylpyrrolidin-3-amine; or N,N,N-trimethylpyrrolidin-3-aminium etc.)

When G is according to option (ii), G is preferably selected from C₂ to C₄ alkoxy; C₁ to C₄ alkyl; C₂ to C₄ alkenyl; C₂ to C₄ alkynyl; and —NR^{Y}—C₁ to C₄ alkyl each of which is unsubstituted or is substituted with one, two or three groups independently selected from —OH, halogen; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²; or G is methoxy which is substituted by one, two or three fluorine substituents; e.g. G may be —OCF₃. More preferably, when G is according to option (ii), G is selected from C₂ to C₄ alkoxy; C₁ to C₄ alkyl; C₂ to C₄ alkenyl; C₂ to C₄ alkynyl; and —NR^{Y}—C₁ to C₄ alkyl; each of which is unsubstituted or is substituted with one or two groups independently selected from —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; NR¹⁰C(NR¹¹)R¹²; and —C(NR¹¹)R¹². More preferably, when G is according to option (ii), G is selected from C₂ to C₄ alkoxy; C₁ to C₄ alkyl; C₂ to C₄ alkenyl; C₂ to C₄ alkynyl; and —NR^{Y}—C₁ to C₄ alkyl; each of which is unsubstituted or is substituted with one or two groups independently selected from —NR¹⁰R¹¹ and —N⁺R¹⁰R¹¹R¹². Still more preferably, when Y is according to option (ii), Y is selected from C₂ to C₃ alkoxy; C₁ to C₃ alkyl; C₂ to C₃ alkenyl; C₂ to C₃ alkynyl; and —NR^{Y}—C₁ to C₃ alkyl; each of which is unsubstituted or is substituted with one group selected from —NR¹⁰R¹¹ and —N⁺R¹⁰R¹¹R¹². In one embodiment, G is not —OMe. Most preferably, when G is according to option (ii), G is selected from C₁ to C₃ alkyl; C₂ to C₄ alkenyl; and C₂ to C₃ alkynyl, each of which is substituted with one group selected from —NR¹⁰R¹¹ and —N⁺R¹⁰R¹¹R¹².

R^{Y} is typically H or C₁ to C₂ alkyl; more preferably H or methyl, most preferably H.

When G is according to option (iii), G is preferably chlorine, bromine, —OH, or methoxy; preferably methoxy or —OH, more preferably —OH.

When G is according to option (iv), G is preferably selected from C₃ to C₆ carbocyclyl; and —O—C₃ to C₆ carbocyclyl; wherein each carbocyclyl group is unsubstituted or is substituted with one or two groups independently selected from —NR¹⁰R¹¹; —N⁺R¹⁰R¹¹R¹²; and C₁ to C₄ alkyl which is unsubstituted or is substituted with one or two groups independently selected from —NR¹⁰R¹¹; and —N⁺R¹⁰R¹¹R¹². More preferably, when G is according to option (iv), G is selected from C₃ to C₄ carbocyclyl; and —O—C₃ to C₄ carbocyclyl; wherein each carbocyclyl group is unsubstituted or is substituted with one or two groups independently selected from —NR¹⁰R¹¹; —N⁺R¹⁰R¹¹R¹²; and C₁ to C₂ alkyl which is unsubstituted or is substituted with one or two groups independently selected from —NR¹⁰R¹¹; and —N⁺R¹⁰R¹¹R¹². Most preferably, when G is according to option (iv), G is selected from C₄ carbocyclyl and —O—C₄ carbocyclyl and is unsubstituted or is substituted with one group selected from —NR¹⁰R¹¹; —N⁺R¹⁰R¹¹R¹² and C₁ to C₂ alkyl which is unsubstituted or is substituted with one or two groups independently selected from —NR¹⁰R¹¹; and —N⁺R¹⁰R¹¹R¹². Preferably, G is according to option (i), (ii) or (iii) above. More preferably, G is according to option (i) or (ii) above, or G is methoxy. More preferably, G is according to option (ii) or is methoxy, most preferably G is according to option (ii).

Still more preferably, therefore each G is independently selected from:
(i) a 4- to 6-membered nitrogen-containing heterocyclic group which is unsubstituted or is substituted by one or two substituents selected from C₁ to C₂ alkyl; —NR¹⁰R¹¹; and —N⁺R¹⁰R¹¹R¹².
(ii) C₂ to C₄ alkoxy; C₁ to C₄ alkyl; C₂ to C₄ alkenyl; C₂ to C₄ alkynyl; and —NR^{Y}—C₁ to C₄ alkyl; each of which is unsubstituted or is substituted with one or two groups independently selected from —NR¹⁰R¹¹ and —N⁺R¹⁰R¹¹R¹²;
and
(iii) chlorine, bromine, —OH or methoxy;

When G is bonded to a nitrogen atom, G can also usefully be a protecting group such as Boc [^{t}Bu-OC(O)—]. Such compounds are useful as intermediates in the preparation of the compounds of Formula (I).

Preferred compounds of Formula (I) according to this embodiment include:
2-(2-{[(5-chloro-1H-1,3-benzodiazol-2-yl)methyl]carbamoyl}-2,3-dihydro-1H-inden-2-yl)acetic acid;
2-[2-(3-isoquinolylmethylcarbamoyl)indan-2-yl]acetic acid;
2-[2-[(1-methylpyrazol-4-yl)methylcarbamoyl]indan-2-yl]acetic acid;
2-[2-[(5-methoxy-1H-benzimidazol-2-yl)methylcarbamoyl]indan-2-yl]acetic acid;
2-(2-{[(1-benzofuran-2-yl)methyl]carbamoyl}-2,3-dihydro-1H-inden-2-yl)acetic acid;
2-(2-{[(5-methyl-1-benzofuran-2-yl)methyl]carbamoyl}-2,3-dihydro-1H-inden-2-yl)acetic acid;
2-[2-[(5-methyl-2,3-dihydrobenzofuran-2-yl)methylcarbamoyl]indan-2-yl]acetic acid;
2-[2-[[1-(3-chlorophenyl)pyrrolidin-3-yl]carbamoyl]indan-2-yl]acetic acid;
2-[2-[(1-methylindol-2-yl)methylcarbamoyl]indan-2-yl]acetic acid;
2-(2-{[(1,3-benzothiazol-2-yl)methyl]carbamoyl}-2,3-dihydro-1H-inden-2-yl)acetic acid;
2-(2-{[2-(3-methyl-1-benzofuran-2-yl)methyl]carbamoyl}-2,3-dihydro-1H-inden-2-yl)acetic acid;
2-[2-[2-(1H-benzimidazol-2-yl)ethylcarbamoyl]indan-2-yl]acetic acid;
2-[2-[2-(4-hydroxyphenyl)ethylcarbamoyl]indan-2-yl]acetic acid;
2-[2-[2-(1H-indol-3-yl)ethylcarbamoyl]indan-2-yl]acetic acid;
2-[2-[(4-hydroxyphenyl)methylcarbamoyl]indan-2-yl]acetic acid;
2-[2-(benzylcarbamoyl)indan-2-yl]acetic acid;
2-[2-(2,3-dihydro-1,4-benzodioxin-3-ylmethylcarbamoyl)indan-2-yl]acetic acid;
2-[2-[(5-bromobenzofuran-2-yl)methylcarbamoyl]indan-2-yl]acetic acid;
2-[2-[2-(benzofuran-2-yl)ethylcarbamoyl]indan-2-yl]acetic acid;
2-[2-[(2-methylbenzofuran-3-yl)methylcarbamoyl]indan-2-yl]acetic acid;
2-[2-[(1-methylimidazol-4-yl)methylcarbamoyl]indan-2-yl]acetic acid;
2-[2-[2-(1,3-benzothiazol-2-yl)ethylcarbamoyl]indan-2-yl]acetic acid;
2-[2-(benzothiophen-2-ylmethylcarbamoyl)indan-2-yl]acetic acid;
2-[2-[(5-methoxy-1,3-benzothiazol-2-yl)methylcarbamoyl]indan-2-yl]acetic acid;
2-[2-[(5-chloro-1,3-benzothiazol-2-yl)methylcarbamoyl]indan-2-yl]acetic acid;
2-[2-(1H-indol-3-ylmethylcarbamoyl)indan-2-yl]acetic acid;
2-[2-[2-(1H-indol-2-yl)ethylcarbamoyl]indan-2-yl]acetic acid;
2-[2-(benzofuran-3-ylmethylcarbamoyl)indan-2-yl]acetic acid;
2-[2-(1H-indol-2-ylmethylcarbamoyl)indan-2-yl]acetic acid;
2-[2-[2-(benzothiophen-2-yl)ethylcarbamoyl]indan-2-yl]acetic acid;
2-[2-[2-(1,3-benzoxazol-2-yl)ethylcarbamoyl]indan-2-yl]acetic acid;
2-[2-[[6-(3-aminopropyl)-1,3-benzothiazol-2-yl]methylcarbamoyl]indan-2-yl]acetic acid;
2-[2-[(6-methoxy-1,3-benzothiazol-2-yl)methylcarbamoyl]indan-2-yl]acetic acid;
2-[2-(4,5,6,7-tetrahydrothiazolo[5,4-c]pyridin-2-ylmethylcarbamoyl)indan-2-yl]acetic acid;
(2-{[(2S)-1-hydroxy-3-(1H-indol-3-yl)propan-2-yl]carbamoyl}-1,3-dihydroinden-2-yl)acetic acid;
(2-{[(1S)-1-{[2-(dimethylamino)ethyl]carbamoyl}-2-(1H-indol-3-yl)ethyl]carbamoyl}-1,3-dihydroinden-2-yl)acetic acid;
2-(2-{[(2S)-3-(1H-indol-3-yl)-1-[(1-methylpiperidin-4-yl)oxy]-1-oxopropan-2-yl]carbamoyl}-2,3-dihydro-1H-inden-2-yl)acetic acid;
(2-{[(1S)-2-(1H-indol-3-yl)-1-{[2-(trimethylammonio)ethyl]carbamoyl}ethyl]carbamoyl}-1,3-dihydroinden-2-yl)acetate
2-[2-[[6-[3-(dimethylamino)prop-1-ynyl]-1,3-benzothiazol-2-yl]methylcarbamoyl]indan-2-yl]acetic acid;
2-[2-[[6-[3-(dimethylamino)propyl]-1,3-benzothiazol-2-yl]methylcarbamoyl]indan-2-yl]acetic acid;
3-[2-[[[2-(carboxymethyl)indane-2-carbonyl]amino]methyl]-1,3-benzothiazol-6-yl]propyl-trimethyl-ammonium;
2-[2-[[6-[(E)-3-aminoprop-1-enyl]-1,3-benzothiazol-2-yl]methylcarbamoyl]indan-2-yl]acetic acid;
2-[2-[[5-(3-aminopropyl)-1,3-benzothiazol-2-yl]methylcarbamoyl]indan-2-yl]acetic acid;
2-[2-[(6-piperazin-1-yl-1,3-benzothiazol-2-yl)methylcarbamoyl]indan-2-yl]acetic acid;
2-[2-[[6-(4-methylpiperazin-1-yl)-1,3-benzothiazol-2-yl]methylcarbamoyl]indan-2-yl]acetic acid;
2-[2-[[6-(4,4-dimethylpiperazin-4-ium-1-yl)-1,3-benzothiazol-2-yl]methylcarbamoyl]indan-2-yl]acetic acid;
(S)-2-(2-((1-(tert-butoxy)-3-(1H-indol-3-yl)-1-oxopropan-2-yl)carbamoyl)-2,3-dihydro-1H-inden-2-yl)acetic acid;
(S)-2-(2-((1-amino-3-(4-hydroxyphenyl)-1-oxopropan-2-yl)carbamoyl)-2,3-dihydro-1H-inden-2-yl)acetic acid;
(2-(carboxymethyl)-2,3-dihydro-1H-indene-2-carbonyl)-L-tyrosine;
(2-(carboxymethyl)-2,3-dihydro-1H-indene-2-carbonyl)-L-tryptophan;
2-(2-(((1H-benzo[d]imidazol-2-yl)methyl)carbamoyl)-2,3-dihydro-1H-inden-2-yl)acetic acid;
(S)-2-(2-((1-amino-3-(1H-indol-3-yl)-1-oxopropan-2-yl)carbamoyl)-2,3-dihydro-1H-inden-2-yl)acetic acid;
(S)-2-(2-((3-(1H-indol-3-yl)-1-methoxy-1-oxopropan-2-yl)carbamoyl)-2,3-dihydro-1H-inden-2-yl)acetic acid;
2-(2-((thiazol-2-ylmethyl)carbamoyl)-2,3-dihydro-1H-inden-2-yl)acetic acid;
2-(2-((quinolin-2-ylmethyl)carbamoyl)-2,3-dihydro-1H-inden-2-yl)acetic acid;
2-(2-(((1-methyl-1H-benzo[d]imidazol-2-yl)methyl)carbamoyl)-2,3-dihydro-1H-inden-2-yl)acetic acid;
2-[2-(benzofuran-3-ylmethylcarbamoyl)indan-2-yl]acetic acid;
2-[2-[[(1R*)-1-(benzofuran-2-yl)ethyl]carbamoyl]indan-2-yl]acetic acid;
2-[2-[[(1S*)-1-(benzofuran-2-yl)ethyl]carbamoyl]indan-2-yl]acetic acid;
2-(2-(((4-fluorobenzo[d]thiazol-2-yl)methyl)carbamoyl)-2,3-dihydro-1H-inden-2-yl)acetic acid;
2-(2-(((4-bromobenzo[d]thiazol-2-yl)methyl)carbamoyl)-2,3-dihydro-1H-inden-2-yl)acetic acid;
2-(2-(((4-methoxybenzo[d]thiazol-2-yl)methyl)carbamoyl)-2,3-dihydro-1H-inden-2-yl)acetic acid;
2-(2-(((4-iodobenzo[d]thiazol-2-yl)methyl)carbamoyl)-2,3-dihydro-1H-inden-2-yl)acetic acid;
2-(2-(((4-ethoxybenzo[d]thiazol-2-yl)methyl)carbamoyl)-2,3-dihydro-1H-inden-2-yl)acetic acid;
2-(2-(((4-methylbenzo[d]thiazol-2-yl)methyl)carbamoyl)-2,3-dihydro-1H-inden-2-yl)acetic acid;
2-(2-(((4-morpholinobenzo[d]thiazol-2-yl)methyl)carbamoyl)-2,3-dihydro-1H-inden-2-yl)acetic acid;
2-(2-(((6-(3-(dimethylamino)azetidin-1-yl)benzo[d]thiazol-2-yl)methyl)carbamoyl)-2,3-dihydro-1H-inden-2-yl)acetic acid;
2-[2-[[6-[3-(trimethylammonio)azetidin-1-yl]-1,3-benzothiazol-2-yl]methylcarbamoyl]indan-2-yl]acetate;
2-[2-[[6-(2-aminoethylamino)-1,3-benzothiazol-2-yl]methylcarbamoyl]indan-2-yl]acetic acid;
2-[2-[[6-[3-(2-aminoethyl)azetidin-1-yl]-1,3-benzothiazol-2-yl]methylcarbamoyl]indan-2-yl]acetic acid;
2-[2-[[6-(6-methyl-2,6-diazaspiro[3.3]heptan-2-yl)-1,3-benzothiazol-2-yl]methylcarbamoyl]indan-2-yl]acetic acid;
2-[2-[[5-(2-aminoethylamino)-1,3-benzothiazol-2-yl]methylcarbamoyl]indan-2-yl]acetic acid;
2-[2-[[5-(3-aminoazetidin-1-yl)-1,3-benzothiazol-2-yl]methylcarbamoyl]indan-2-yl]acetic acid;
2-[2-[[5-[3-(dimethylamino)azetidin-1-yl]-1,3-benzothiazol-2-yl]methylcarbamoyl]indan-2-yl]acetic acid;
2-[2-[[5-[3-(trimethylammonio)azetidin-1-yl]-1,3-benzothiazol-2-yl]methylcarbamoyl]indan-2-yl]acetate;
2-[2-[[5-[2-(dimethylamino)ethylamino]-1,3-benzothiazol-2-yl]methylcarbamoyl]indan-2-yl]acetic acid;
2-[2-[[5-[2-(trimethylammonio)ethylamino]-1,3-benzothiazol-2-yl]methylcarbamoyl]indan-2-yl]acetate;
2-[2-[[6-(3-aminocyclobutoxy)-1,3-benzothiazol-2-yl]methylcarbamoyl]indan-2-yl]acetic acid;
2-[2-[[6-[3-(dimethylamino)cyclobutoxy]-1,3-benzothiazol-2-yl]methylcarbamoyl]indan-2-yl]acetic acid;
2-[2-[[6-(2-aminoethyl)-1,3-benzothiazol-2-yl]methylcarbamoyl]indan-2-yl]acetic acid;
2-[2-[[6-[(dimethylamino)methyl]-1,3-benzothiazol-2-yl]methylcarbamoyl]indan-2-yl]acetic acid;
2-[2-[[6-[(trimethylammonio)methyl]-1,3-benzothiazol-2-yl]methylcarbamoyl]indan-2-yl]acetate;
2-[2-[[5-(2-aminoethyl)-1,3-benzothiazol-2-yl]methylcarbamoyl]indan-2-yl]acetic acid;
2-[2-[[5-[(dimethylamino)methyl]-1,3-benzothiazol-2-yl]methylcarbamoyl]indan-2-yl]acetic acid;
2-[2-[[5-[(trimethylammonio)methyl]-1,3-benzothiazol-2-yl]methylcarbamoyl]indan-2-yl]acetate;
2-[2-[[6-[3-(trimethylammonio)prop-1-ynyl]-1,3-benzothiazol-2-yl]methylcarbamoyl]indan-2-yl]acetate;
2-[2-[[5-[(E)-3-aminoprop-1-enyl]-1,3-benzothiazol-2-yl]methylcarbamoyl]indan-2-yl]acetic acid;
2-[2-[[5-[3-(dimethylamino)prop-1-ynyl]-1,3-benzothiazol-2-yl]methylcarbamoyl]indan-2-yl]acetic acid;
2-[2-[[5-[3-(dimethylamino)propyl]-1,3-benzothiazol-2-yl]methylcarbamoyl]indan-2-yl]acetic acid;
2-[2-[[5-[3-(trimethylammonio)propyl]-1,3-benzothiazol-2-yl]methylcarbamoyl]indan-2-yl]acetate;
2-(2-(((6-((1r,3r)-3-((dimethylamino)methyl)cyclobutyl)benzo[d]thiazol-2-yl)methyl)carbamoyl)-2,3-dihydro-1H-inden-2-yl)acetic acid;
2-[2-[[6-[(1r,3r)-3-[(trimethylammonio)methyl]cyclobutyl]-1,3-benzothiazol-2-yl]methylcarbamoyl]indan-2-yl]acetate;
2-(2-(((6-((1s,3s)-3-((dimethylamino)methyl)cyclobutyl)benzo[d]thiazol-2-yl)methyl)carbamoyl)-2,3-dihydro-1H-inden-2-yl)acetic acid;
2-[2-[[6-[(1s,3s)-3-[(trimethylammonio)methyl]cyclobutyl]-1,3-benzothiazol-2-yl]methylcarbamoyl]indan-2-yl]acetate;
2-[2-[[6-[2-(dimethylamino)ethyl]-1,3-benzothiazol-2-yl]methylcarbamoyl]indan-2-yl]acetic acid;
2-[2-[[6-[2-(trimethylammonio)ethyl]-1,3-benzothiazol-2-yl]methylcarbamoyl]indan-2-yl]acetate;
2-[2-[[5-[2-(dimethylamino)ethyl]-1,3-benzothiazol-2-yl]methylcarbamoyl]indan-2-yl]acetic acid;
2-[2-[[5-[2-(trimethylammonio)ethyl]-1,3-benzothiazol-2-yl]methylcarbamoyl]indan-2-yl]acetate;
2-[2-[[6-[3-(aminomethyl)azetidin-1-yl]-1,3-benzothiazol-2-yl]methylcarbamoyl]indan-2-yl]acetic acid;
2-[2-[[6-(3-aminoazetidin-1-yl)-1,3-benzothiazol-2-yl]methylcarbamoyl]indan-2-yl]acetic acid;
2-[2-[[6-[(3S)-3-aminopyrrolidin-1-yl]-1,3-benzothiazol-2-yl]methylcarbamoyl]indan-2-yl]acetic acid;
2-[2-[[6-[(3R)-3-aminopyrrolidin-1-yl]-1,3-benzothiazol-2-yl]methylcarbamoyl]indan-2-yl]acetic acid;
2-[2-[[5-[(3S)-3-aminopyrrolidin-1-yl]-1,3-benzothiazol-2-yl]methylcarbamoyl]indan-2-yl]acetic acid;
2-[2-[[5-[(3R)-3-aminopyrrolidin-1-yl]-1,3-benzothiazol-2-yl]methylcarbamoyl]indan-2-yl]acetic acid;
2-[2-[[6-[2-(dimethylamino)ethylamino]-1,3-benzothiazol-2-yl]methylcarbamoyl]indan-2-yl]acetic acid;
2-[2-[[5-(4-methylpiperazin-1-yl)-1,3-benzothiazol-2-yl]methylcarbamoyl]indan-2-yl]acetic acid;
2-[2-[(6-hydroxy-1,3-benzothiazol-2-yl)methylcarbamoyl]indan-2-yl]acetic acid;
2-[2-[(6-ethoxy-1,3-benzothiazol-2-yl)methylcarbamoyl]indan-2-yl]acetic acid;
2-[2-[[6-(2-hydroxyethoxy)-1,3-benzothiazol-2-yl]methylcarbamoyl]indan-2-yl]acetic acid;
2-[2-[[6-[2-(dimethylamino)ethoxy]-1,3-benzothiazol-2-yl]methylcarbamoyl]indan-2-yl]acetic acid;
2-[2-[[6-[2-(trimethylammonio)ethoxy]-1,3-benzothiazol-2-yl]methylcarbamoyl]indan-2-yl]acetate;
2-[2-[[5-[2-(dimethylamino)ethoxy]-1,3-benzothiazol-2-yl]methylcarbamoyl]indan-2-yl]acetic acid;
2-[2-[[5-[2-(trimethylammonio)ethoxy]-1,3-benzothiazol-2-yl]methylcarbamoyl]indan-2-yl]acetate;
2-[2-[(5,6-dimethoxy-1,3-benzothiazol-2-yl)methylcarbamoyl]indan-2-yl]acetic acid;
2-[2-([1,3]dioxolo[4,5-f][1,3]benzothiazol-6-ylmethylcarbamoyl)indan-2-yl]acetic acid;
(S)-2-(2-((1-((1,1-dimethylpiperidin-1-ium-4-yl)oxy)-3-(1H-indol-3-yl)-1-oxopropan-2-yl)carbamoyl)-2,3-dihydro-1H-inden-2-yl)acetate;
(S)-2-(2-((2-(1H-indol-3-yl)-1-(5-methyl-1,3,4-oxadiazol-2-yl)ethyl)carbamoyl)-2,3-dihydro-1H-inden-2-yl)acetic acid;
(S)-2-(2-((1-(5-(aminomethyl)-1,3,4-oxadiazol-2-yl)-2-(1H-indol-3-yl)ethyl)carbamoyl)-2,3-dihydro-1H-inden-2-yl)acetic acid;
(S)-2-(2-((1-(5-(2-aminopropan-2-yl)-1,3,4-oxadiazol-2-yl)-2-(1H-indol-3-yl)ethyl)carbamoyl)-2,3-dihydro-1H-inden-2-yl)acetic acid;
2-(2-((benzo[d]thiazol-2-ylmethyl)carbamoyl)-5,6-difluoro-2,3-dihydro-1H-inden-2-yl)acetic acid;
2-(2-((benzo[d]thiazol-2-ylmethyl)carbamoyl)-5,6-dichloro-2,3-dihydro-1H-inden-2-yl)acetic acid;
2-(2-(((5-(3-(dimethylamino)azetidin-1-yl)-6-methoxybenzo[d]thiazol-2-yl)methyl)carbamoyl)-2,3-dihydro-1H-inden-2-yl)acetic acid;
2-(2-(((6-methoxy-5-(3-(trimethylammonio)azetidin-1-yl)benzo[d]thiazol-2-yl)methyl)carbamoyl)-2,3-dihydro-1H-inden-2-yl)acetate;
2-(2-(((5-(3-(dimethylamino)propoxy)-6-methoxybenzo[d]thiazol-2-yl)methyl)carbamoyl)-5,6-difluoro-2,3-dihydro-1H-inden-2-yl)acetic acid;
2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate;
2-(2-(((5-(2-(dimethylamino)ethoxy)-6-methoxybenzo[d]thiazol-2-yl)methyl)carbamoyl)-2,3-dihydro-1H-inden-2-yl)acetic acid;
2-[2-(thiazolo[4,5-c]pyridin-2-ylmethylcarbamoyl)indan-2-yl]acetic acid;
2-[2-[[6-(trifluoromethoxy)-1,3-benzothiazol-2-yl]methylcarbamoyl]indan-2-yl]acetic acid;
2-[2-[(5-methyl-6,7-dihydro-4H-thiazolo[5,4-c]pyridin-2-yl)methylcarbamoyl]indan-2-yl]acetic acid; and
2-[2-[(5-hydroxy-1,3-benzothiazol-2-yl)methylcarbamoyl]indan-2-yl]acetic acid;
   and pharmaceutically acceptable salts thereof.

More particularly preferred compounds of Formula (I) according to this embodiment are:
2-(2-{[(5-chloro-1H-1,3-benzodiazol-2-yl)methyl]carbamoyl}-2,3-dihydro-1H-inden-2-yl)acetic acid;
2-[2-(3-isoquinolylmethylcarbamoyl)indan-2-yl]acetic acid;
2-[2-[(1-methylpyrazol-4-yl)methylcarbamoyl]indan-2-yl]acetic acid;
2-[2-[(5-methoxy-1H-benzimidazol-2-yl)methylcarbamoyl]indan-2-yl]acetic acid;
2-(2-{[(1-benzofuran-2-yl)methyl]carbamoyl}-2,3-dihydro-1H-inden-2-yl)acetic acid;
2-(2-{[(5-methyl-1-benzofuran-2-yl)methyl]carbamoyl}-2,3-dihydro-1H-inden-2-yl)acetic acid;
2-[2-[(5-methyl-2,3-dihydrobenzofuran-2-yl)methylcarbamoyl]indan-2-yl]acetic acid;
2-[2-[[1-(3-chlorophenyl)pyrrolidin-3-yl]carbamoyl]indan-2-yl]acetic acid;
2-[2-[(1-methylindol-2-yl)methylcarbamoyl]indan-2-yl]acetic acid;
2-(2-{[(1,3-benzothiazol-2-yl)methyl]carbamoyl}-2,3-dihydro-1H-inden-2-yl)acetic acid;
2-(2-{[2-(3-methyl-1-benzofuran-2-yl)methyl]carbamoyl}-2,3-dihydro-1H-inden-2-yl)acetic acid;
2-[2-[2-(1H-benzimidazol-2-yl)ethylcarbamoyl]indan-2-yl]acetic acid;
2-[2-[2-(4-hydroxyphenyl)ethylcarbamoyl]indan-2-yl]acetic acid;
2-[2-[2-(1H-indol-3-yl)ethylcarbamoyl]indan-2-yl]acetic acid;
2-[2-[(4-hydroxyphenyl)methylcarbamoyl]indan-2-yl]acetic acid;
2-[2-(benzylcarbamoyl)indan-2-yl]acetic acid;
2-[2-(2,3-dihydro-1,4-benzodioxin-3-ylmethylcarbamoyl)indan-2-yl]acetic acid;
2-[2-[(5-bromobenzofuran-2-yl)methylcarbamoyl]indan-2-yl]acetic acid;
2-[2-[2-(benzofuran-2-yl)ethylcarbamoyl]indan-2-yl]acetic acid;
2-[2-[(2-methylbenzofuran-3-yl)methylcarbamoyl]indan-2-yl]acetic acid;
2-[2-[(1-methylimidazol-4-yl)methylcarbamoyl]indan-2-yl]acetic acid;
2-[2-[2-(1,3-benzothiazol-2-yl)ethylcarbamoyl]indan-2-yl]acetic acid;
2-[2-(benzothiophen-2-ylmethylcarbamoyl)indan-2-yl]acetic acid;
2-[2-[(5-methoxy-1,3-benzothiazol-2-yl)methylcarbamoyl]indan-2-yl]acetic acid;
2-[2-[(5-chloro-1,3-benzothiazol-2-yl)methylcarbamoyl]indan-2-yl]acetic acid;
2-[2-(1H-indol-3-ylmethylcarbamoyl)indan-2-yl]acetic acid;
2-[2-[2-(1H-indol-2-yl)ethylcarbamoyl]indan-2-yl]acetic acid;
2-[2-(benzofuran-3-ylmethylcarbamoyl)indan-2-yl]acetic acid;
2-[2-(1H-indol-2-ylmethylcarbamoyl)indan-2-yl]acetic acid;
2-[2-[2-(benzothiophen-2-yl)ethylcarbamoyl]indan-2-yl]acetic acid;
2-[2-[2-(1,3-benzoxazol-2-yl)ethylcarbamoyl]indan-2-yl]acetic acid;
2-[2-[[6-(3-aminopropyl)-1,3-benzothiazol-2-yl]methylcarbamoyl]indan-2-yl]acetic acid;
2-[2-[(6-methoxy-1,3-benzothiazol-2-yl)methylcarbamoyl]indan-2-yl]acetic acid;
2-[2-(4,5,6,7-tetrahydrothiazolo[5,4-c]pyridin-2-ylmethylcarbamoyl)indan-2-yl]acetic acid;
(2-{[(2S)-1-hydroxy-3-(1H-indol-3-yl)propan-2-yl]carbamoyl}-1,3-dihydroinden-2-yl)acetic acid;
(2-{[(1S)-1-{[2-(dimethylamino)ethyl]carbamoyl}-2-(1H-indol-3-yl)ethyl]carbamoyl}-1,3-dihydroinden-2-yl)acetic acid;
2-(2-{[(2S)-3-(1H-indol-3-yl)-1-[(1-methylpiperidin-4-yl)oxy]-1-oxopropan-2-yl]carbamoyl}-2,3-dihydro-1H-inden-2-yl)acetic acid;
(2-{[(1S)-2-(1H-indol-3-yl)-1-{[2-(trimethylammonio)ethyl]carbamoyl}ethyl]carbamoyl}-1,3-dihydroinden-2-yl)acetate
2-[2-[[6-[3-(dimethylamino)prop-1-ynyl]-1,3-benzothiazol-2-yl]methylcarbamoyl]indan-2-yl]acetic acid;
2-[2-[[6-[3-(dimethylamino)propyl]-1,3-benzothiazol-2-yl]methylcarbamoyl]indan-2-yl]acetic acid;
3-[2-[[[2-(carboxymethyl)indane-2-carbonyl]amino]methyl]-1,3-benzothiazol-6-yl]propyl-trimethyl-ammonium;
2-[2-[[6-[(E)-3-aminoprop-1-enyl]-1,3-benzothiazol-2-yl]methylcarbamoyl]indan-2-yl]acetic acid;
2-[2-[[5-(3-aminopropyl)-1,3-benzothiazol-2-yl]methylcarbamoyl]indan-2-yl]acetic acid;
2-[2-[(6-piperazin-1-yl-1,3-benzothiazol-2-yl)methylcarbamoyl]indan-2-yl]acetic acid;
2-[2-[[6-(4-methylpiperazin-1-yl)-1,3-benzothiazol-2-yl]methylcarbamoyl]indan-2-yl]acetic acid; and
2-[2-[[6-(4,4-dimethylpiperazin-4-ium-1-yl)-1,3-benzothiazol-2-yl]methylcarbamoyl]indan-2-yl]acetic acid
2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate;
   and pharmaceutically acceptable salts thereof.

Further preferred compounds of Formula (I) according to this embodiment are:
2-[2-[2-(1-methyl-4-piperidyl)ethylcarbamoyl]indan-2-yl]acetic acid;
2-[2-[2-(1,1-dimethylpiperidin-1-ium-4-yl)ethylcarbamoyl]indan-2-yl]acetate;
2-[2-[(1-benzylpyrrolidin-3-yl)carbamoyl]indan-2-yl]acetic acid;
2-[2-[(1,3-dimethylbenzimidazol-3-ium-2-yl)methylcarbamoyl]indan-2-yl]acetate;
2-[2-[(2-methylisoquinolin-2-ium-3-yl)methylcarbamoyl]indan-2-yl]acetate;
2-[2-[(1-methyl-4-piperidyl)methylcarbamoyl]indan-2-yl]acetic acid;
2-[2-[(1,1-dimethylpiperidin-1-ium-4-yl)methylcarbamoyl]indan-2-yl]acetate;
2-[2-[2-(1-methylimidazol-4-yl)ethylcarbamoyl]indan-2-yl]acetic acid;
2-[2-[(5,5-dimethyl-6,7-dihydro-4H-thiazolo[5,4-c]pyridin-5-ium-2-yl)methylcarbamoyl]indan-2-yl]acetate;
2-[2-[[(3R)-1-phenylpyrrolidin-3-yl]carbamoyl]indan-2-yl]acetic acid;
2-[2-[[(3S)-1-phenylpyrrolidin-3-yl]carbamoyl]indan-2-yl]acetic acid;
2-[2-(imidazo[1,2-a]pyridin-2-ylmethylcarbamoyl)indan-2-yl]acetic acid;
2-[2-(1,3-benzoxazol-2-ylmethylcarbamoyl)indan-2-yl]acetic acid;
2-[2-[(3-hydroxyphenyl)methylcarbamoyl]indan-2-yl]acetic acid;
2-[2-[(5-methylthiazolo[4,5-c]pyridin-5-ium-2-yl)methylcarbamoyl]indan-2-yl]acetate;
2-[2-[(5-hydroxy-2-pyridyl)methylcarbamoyl]indan-2-yl]acetic acid; and
2-[2-[1,3-benzothiazol-2-ylmethyl(methyl)carbamoyl]indan-2-yl]acetic acid;
2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate;
   and pharmaceutically acceptable salts thereof.

In a second preferred embodiment of Formula (I), the moiety \textcircled{A}-(G)ₘ is

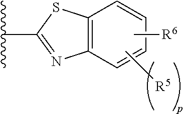

wherein:
p is 0 or 1;
R⁵ is selected from —OMe, —OH, halogen, —NR²⁰R²¹; —N⁺R²⁰R²¹R²², —CF₃, and R⁶; and
each R⁶ is independently selected from:
   C₂ to C₄ alkoxy which is unsubstituted or is substituted with a group selected from —OH; —NR¹⁰R¹¹; —N⁺R¹⁰R¹¹R¹²; —OR^{6c} and —NR¹⁰R^{6c}, wherein R^{6c} is a C₁ to C₃ alkyl group which is unsubstituted or substituted with a group selected from OH; —NR¹⁰R¹¹; —N⁺R¹⁰R¹¹R¹²; —NR¹⁰NR¹¹R¹²; —NR¹⁰N⁺R¹¹R¹²R¹³; —N⁺R¹⁰R¹¹NR¹²R¹³; —NR¹⁰C(NR¹¹)NR¹²R¹³; —NR¹⁰C(N⁺R¹¹R¹²)NR¹³R¹⁴; —C(NR¹⁰)NR¹¹R¹²; and —C(N⁺R¹⁰R¹¹)NR¹²R¹³; and each R¹⁰, R¹¹, R¹², R¹³ and R¹⁴ is independently H or methyl;
   —R^{6a}R^{A}, —O—R^{6a}R^{A}, —NR²⁰—R^{6a}R^{A}, —R^{6b}R^{B}, —O—R^{6b}R^{B}, and —NR²⁰—R^{6b}R^{B};
   —R^{X}R^{R}, —O—R^{X}R^{R}, —O—R^{X}—C(O)—R^{R}, —R^{X}—C(O)—R^{R}, —NR²⁰—R^{X}R^{R}, and —NR²⁰—R^{X}—C(O)—R^{R}; and
   —CN; —C(O)NR²⁰R²¹; —C(O)NR²¹—R^{X}R^{B}; —C(O)NR⁴⁰R⁴¹; —SO₂R²⁰; —SO₂—R^{X}R^{B}; —SO₂NR²⁰R²¹; —SO₂—NR²⁰—R^{X}R^{B}; and —SO₂NR⁴⁰R⁴¹;
wherein:
   each R^{X} is independently selected from R^{6a} and R^{6b};
   each R^{6a} is independently selected from C₁ to C₄ alkylene, C₂ to C₄ alkenylene and C₂ to C₄ alkynylene; and each R^{6a} is independently unsubstituted or is substituted by one group selected from —OH, halogen; —NR²⁰R²¹; —N⁺R²⁰R²¹R²²; —NR²⁰C(NR²¹)NR²²R²³; —NR²⁰C(N⁺R²¹R²²)NR²³R²⁴; —NR²⁰C(NR²¹)R²²; —NR²⁰C(N⁺R²¹R²²)R²³; —C(NR²⁰)NR²¹R²²; —C(N⁺R²⁰R²¹)NR²²R²³; —C(NR²⁰)R²¹; and —C(N⁺R²⁰R²¹)R²²; —C(O)NR²⁰R²¹; —C(O)N⁺R²⁰R²¹R²²; —C(O)—R²⁰, and methoxy which is unsubstituted or is substituted by one, two or three halogen substituents;
   each R^{b} is independently selected from [C₁ to C₃ alkylene]-C(R^{z})₂, [C₂ to C₃ alkenylene]—C(R^{z})₂ and [C₂ to C₃ alkynylene]—C(R^{z})₂; wherein the two R^{z} groups are attached together to form, together with the atom to which they are attached, a 5- or 6-membered carbocyclic or heterocyclic group;
   R^{A} is selected from —NR²⁰R³⁰; —N⁺R²⁰R²¹R³⁰; —NR²⁰NR²¹R²²; —NR²⁰N⁺R²¹R²²R²³; —N⁺R²⁰R²¹NR²²R²³; —NR²⁰C(NR²¹)NR²²R³⁰; —NR²⁰C(N⁺R²¹R²²)NR²³R³⁰; —C(NR²⁰)NR²¹R²²; and —C(N⁺R²⁰R²¹)NR²²R²³;
   R^{B} is selected from —NR²⁰R²¹; —N⁺R²⁰R²¹R²²; —NR²⁰NR²¹R²²; —NR²⁰N⁺R²¹R²²R²³; —N⁺R²⁰R²¹NR²²R²³; —NR²⁰C(NR²¹)NR²²R²³; —NR²⁰C(N⁺R²¹R²²)NR²³R²⁴; —C(NR²⁰)NR²¹R²²; and —C(N⁺R²⁰R²¹)NR²²R²³;
   R⁴⁰ and R⁴¹, together with the nitrogen atom to which they are attached, form a 4- to 6-membered heterocyclic group, wherein any nitrogen atom in the ring is independently selected from secondary, tertiary and quaternary nitrogen atoms;
   each R^{R} is independently a 4- to 10-membered heteroaryl or heterocyclic group comprising at least one nitrogen atom, and said nitrogen atom(s) are independently selected from secondary, tertiary and quaternary nitrogen atom(s);
      wherein each R^{R}, and each ring formed by —NR⁴⁰R⁴¹, is independently unsubstituted or is substituted with one, two or three groups independently selected from
      i) halogen, —CN;
      ii) oxo, providing that said R^{R} group is a heterocyclic group;
      iii) —R²⁰, —R⁷—OR²⁰; —R⁷—NR²⁰R²¹; —R⁷—N⁺R²⁰R²¹R²²; —R⁷—NR²⁰C(NR²¹)NR²²R²³; —R⁷—NR²⁰C(N⁺R²¹R²²)NR²³R²⁴; —R⁷—NR²⁰C(NR²¹)R²²; —R⁷—NR²⁰C(N⁺R²¹R²²)R²³; —R⁷—C(NR²⁰)NR²¹R²²; —R⁷—C(N⁺R²⁰R²¹)NR²²R²³; —R⁷—C(NR²⁰)R²¹; and —R⁷—C(N⁺R²⁰R²¹)R²²;
each R⁷ is independently selected from a bond and unsubstituted C₁ to C₃ alkylene;
R²⁰, R²¹, R²², R²³ and R²⁴ are each independently selected from H and C₁ to C₃ alkyl which is unsubstituted or is substituted with one —OH or —OMe group or with one, two or three halogen groups;
each R³⁰ is independently selected from C₂ to C₃ alkyl which is unsubstituted or is substituted with one —OH or —OMe group or with one, two or three halogen groups.

Preferably, in this embodiment, the compound of Formula (I) is other than:
2-(2-(((4-ethoxybenzo[d]thiazol-2-yl)methyl)carbamoyl)-2,3-dihydro-1H-inden-2-yl)acetic acid;
2-[2-[(6-ethoxy-1,3-benzothiazol-2-yl)methylcarbamoyl]indan-2-yl]acetic acid;
2-[2-[[6-(2-hydroxyethoxy)-1,3-benzothiazol-2-yl]methylcarbamoyl]indan-2-yl]acetic acid;
2-[2-[[6-[2-(dimethylamino)ethoxy]-1,3-benzothiazol-2-yl]methylcarbamoyl]indan-2-yl]acetic acid;
2-[2-[[6-[2-(trimethylammonio)ethoxy]-1,3-benzothiazol-2-yl]methylcarbamoyl]indan-2-yl]acetate;
2-[2-[[5-[2-(dimethylamino)ethoxy]-1,3-benzothiazol-2-yl]methylcarbamoyl]indan-2-yl]acetic acid;
2-[2-[[5-[2-(trimethylammonio)ethoxy]-1,3-benzothiazol-2-yl]methylcarbamoyl]indan-2-yl]acetate;
2-(2-(((5-(3-(dimethylamino)propoxy)-6-methoxybenzo[d]thiazol-2-yl)methyl)carbamoyl)-5,6-difluoro-2,3-dihydro-1H-inden-2-yl)acetic acid;
2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate; and
2-(2-(((5-(2-(dimethylamino)ethoxy)-6-methoxybenzo[d]thiazol-2-yl)methyl)carbamoyl)-2,3-dihydro-1H-inden-2-yl)acetic acid.

In a first preferred aspect of this embodiment:
Lk is —CH₂—;
R⁵ is selected from —OMe, —OH, halogen, —NR¹⁰R¹¹; —N⁺R¹⁰R¹¹R¹², and —CF₃;
R⁶ is C₂ to C₄ alkoxy which is unsubstituted or is substituted with a group selected from —OH; —NR¹⁰R¹¹; —N⁺R¹⁰R¹¹R¹²; —OR^{6c} and —NR¹⁰R^{6c}, wherein R^{6c} is a C₁ to C₃ alkyl group which is unsubstituted or substituted with a group selected from OH;
—NR¹⁰R¹¹; —N⁺R¹⁰R¹¹R¹²; —NR¹⁰NR¹¹R¹²; —NR¹⁰N⁺R¹¹R¹²R¹³; —N⁺R¹⁰R¹¹NR¹²R¹³; —NR¹⁰C(NR¹¹)NR¹²R¹³; —NR¹⁰C(N⁺R¹¹R¹²)NR¹³R¹⁴; —C(NR¹⁰)NR¹¹R¹²; and —C(N⁺R¹⁰R¹¹)NR¹²R¹³;
and
each R¹⁰, R¹¹, R¹², R³ and R¹⁴ is independently H or methyl;
with the proviso that the compound is other than:
2-(2-(((4-ethoxybenzo[d]thiazol-2-yl)methyl)carbamoyl)-2,3-dihydro-1H-inden-2-yl)acetic acid;
2-[2-[(6-ethoxy-1,3-benzothiazol-2-yl)methylcarbamoyl]indan-2-yl]acetic acid;
2-[2-[[6-(2-hydroxyethoxy)-1,3-benzothiazol-2-yl]methylcarbamoyl]indan-2-yl]acetic acid;
2-[2-[[6-[2-(dimethylamino)ethoxy]-1,3-benzothiazol-2-yl]methylcarbamoyl]indan-2-yl]acetic acid;
2-[2-[[6-[2-(trimethylammonio)ethoxy]-1,3-benzothiazol-2-yl]methylcarbamoyl]indan-2-yl]acetate;
2-[2-[[5-[2-(dimethylamino)ethoxy]-1,3-benzothiazol-2-yl]methylcarbamoyl]indan-2-yl]acetic acid;
2-[2-[[5-[2-(trimethylammonio)ethoxy]-1,3-benzothiazol-2-yl]methylcarbamoyl]indan-2-yl]acetate;
2-(2-(((5-(3-(dimethylamino)propoxy)-6-methoxybenzo[d]thiazol-2-yl)methyl)carbamoyl)-5,6-difluoro-2,3-dihydro-1H-inden-2-yl)acetic acid;
2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate; and
2-(2-(((5-(2-(dimethylamino)ethoxy)-6-methoxybenzo[d]thiazol-2-yl)methyl)carbamoyl)-2,3-dihydro-1H-inden-2-yl)acetic acid.

Typically, in this aspect, R⁵ if present is methoxy.

Typically R⁶ is C₂ to C₄ alkoxy, for example ethoxy, n-propoxy or n-butoxy, preferably ethoxy or n-propoxy, each of which may be unsubstituted or substituted.

Typically, R⁶ is unsubstituted or is substituted with a group selected from —OH; —NR¹⁰R¹¹; —N⁺R¹⁰R¹¹R¹²; and —OR^{6c}. In one embodiment, R⁶ is C₂ to C₄ alkoxy which is substituted with a group selected from —OH; —NR¹⁰R¹¹; —N⁺R¹⁰R¹¹R¹²; —OR^{6c} and —NR¹⁰R^{6c}. Preferably, R⁶ is C₂ to C₄ alkoxy which is substituted with a group selected from —OH; —NR¹⁰R¹¹; —N⁺R¹⁰R¹¹R¹²; and —OR^{6c}. More preferably, R⁶ is C₂ to C₄ alkoxy which is substituted with a group selected from —NR¹⁰R¹¹; —N⁺R¹⁰R¹¹R¹²; and —OR^{6c}. Most preferably, R⁶ is C₂ to C₄ alkoxy which is substituted with a group —OR^{6c}.

In some preferred compounds, R^{6c} is a C₁ alkyl group which is unsubstituted or substituted with a group selected from OH; —NR¹⁰R¹¹; —N⁺R¹⁰R¹¹R¹²; —NR¹⁰NR¹¹R¹²; —NR¹⁰N⁺R¹¹R¹²R¹³; —N⁺R¹⁰R¹¹NR¹²R¹³; —NR¹⁰C(NR¹¹)NR¹²R¹³; —NR¹⁰C(N⁺R¹¹R¹²)NR¹³R¹⁴; —C(NR¹⁰)NR¹¹R¹²; and —C(N⁺R¹⁰R¹¹)NR¹²R¹³; or is a C₂ to C₃ alkyl group which is substituted with a group selected from —NR¹⁰R¹¹; —N⁺R¹⁰R¹¹R¹²; —NR¹⁰NR¹¹R¹²; —NR¹⁰N⁺R¹¹R¹²R¹³; —N⁺R¹⁰R¹¹NR¹²R¹³; —NR¹⁰C(NR¹¹)NR¹²R¹³; —NR¹⁰C(N⁺R¹¹R¹²)NR¹³R¹⁴; —C(NR¹⁰)NR¹¹R¹²; and —C(N⁺R¹⁰R¹¹)NR¹²R¹³. In other preferred compounds R^{6c} is a C₁ to C₃ alkyl group which is substituted with a group selected from —NR¹⁰R¹¹; —N⁺R¹⁰R¹¹R¹²; —NR¹⁰NR¹¹R¹²; —NR¹⁰N⁺R¹¹R¹²R¹³; —N⁺R¹⁰R¹¹NR¹²R¹³; —NR¹⁰C(NR¹¹)NR¹²R¹³; —NR¹⁰C(N⁺R¹¹R¹²)NR¹³R¹⁴; —C(NR¹⁰)NR¹¹R¹²; and —C(N⁺R¹⁰R¹¹)NR¹²R¹³.

R⁶ is typically a C₁ to C₃ alkyl group which is unsubstituted or substituted with a group selected from OH; —NR¹⁰R¹¹; and —N⁺R¹⁰R¹¹R¹². Preferably, R^{6c} is a C₁ to C₃ alkyl group which is unsubstituted or substituted with a group selected from OH; —NMe₂; and —N⁺Me₃. More preferably, R^{6c} is a C₁ to C₂ alkyl group which is unsubstituted or substituted with a group selected from OH; —NMe₂; and —N⁺Me₃.

Thus, R⁶ is preferably C₂ to C₄ alkoxy which is unsubstituted or is substituted with a group selected from —OH; —NR¹⁰R¹¹; —N⁺R¹⁰R¹¹R¹²; and —OR^{6c}, wherein R^{6c} is a C₁ to C₃ alkyl group which is unsubstituted or substituted with a group selected from OH; —NR¹⁰R¹¹; and —N⁺R¹⁰R¹¹R¹². More preferably, R⁶ is C₂ to C₄ alkoxy which is unsubstituted or is substituted with a group selected from —OH; —NMe₂; —N⁺Me₃; and —OR^{6c}, wherein R^{6c} is a C₁ to C₃ alkyl group which is unsubstituted or substituted with a group selected from —NMe₂; and —N⁺(Me)₃. Preferably, R⁶ is C₂ to C₄ alkoxy which is unsubstituted or is substituted with a group selected from —OH; —NMe₂; —N⁺(Me)₃; and —OR^{6c}, wherein R^{6c} is a C₁ to C₂alkyl group which is substituted with a group selected from —NMe₂; and —N⁺(Me)₃. Most preferably, R⁶ is C₂ to C₄ alkoxy which is unsubstituted or is substituted with a group selected from —OH; —NMe₂; —N⁺(Me)₃; —O(CH₂)—NMe₂; and —O(CH₂)—N⁺(Me)₃.

Typically, in this aspect, therefore,
Lk is —CH₂—;
R⁵ is selected from —OMe and —OH; and
R⁶ is C₂ to C₄ alkoxy which is substituted with a group selected from —NR¹⁰R¹¹; —N⁺R¹⁰R¹¹R¹²; and —OR^{6c}, wherein R^{6c} is a C₁ to C₃ alkyl group which is unsubstituted or substituted with a group selected from —NR¹⁰R¹¹; and —N⁺R¹⁰R¹¹R¹².

More preferably, therefore, in this aspect:
R¹ is selected from —OH and —NHOH, or where the compound of Formula (I) contains a positively charged nitrogen atom, R¹ may be O⁻, such that the compound forms a zwitterion;
R² is H;
R⁴ is H;
n is 0; or n is 2 and each R³ group is fluorine;
Lk is —CH₂—;
The moiety \textcircled{A}-(G)ₘ is

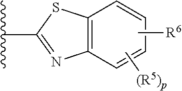

p is 1;
R⁵ is selected from —OMe and —OH; and
R⁶ is C₂ to C₄ alkoxy which is substituted with a group selected from —NR¹⁰R¹¹; —N⁺R¹⁰R¹¹R¹²; and —OR^{6c}, wherein R^{6c} is a C₁ to C₃ alkyl group which is unsubstituted or substituted with a group selected from —NR¹⁰R¹¹; and —N⁺R¹⁰R¹¹R¹².

Preferably, in this embodiment, the compound of Formula (I) is other than:
2-(2-(((5-(3-(dimethylamino)propoxy)-6-methoxybenzo[d]thiazol-2-yl)methyl)carbamoyl)-5,6-difluoro-2,3-dihydro-1H-inden-2-yl)acetic acid;
2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate; and
2-(2-(((5-(2-(dimethylamino)ethoxy)-6-methoxybenzo[d]thiazol-2-yl)methyl)carbamoyl)-2,3-dihydro-1H-inden-2-yl)acetic acid.

Typically, R⁶ is bonded at the ring position marked as 1 below. If a group R is present, this is typically present at the position marked as 2 below.

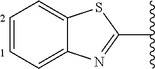 [SMILES: *c1nc2ccccc2s1]

Preferred compounds of Formula (I) according to this aspect include:
2-[2-[[6-(3-hydroxypropoxy)-1,3-benzothiazol-2-yl]methylcarbamoyl]indan-2-yl]acetic acid;
2-[2-[(6-propoxy-1,3-benzothiazol-2-yl)methylcarbamoyl]indan-2-yl]acetic acid;
2-[2-[[5-[3-(dimethylamino)propoxy]-6-methoxy-1,3-benzothiazol-2-yl]methylcarbamoyl]indan-2-yl]acetic acid;
2-[2-[[6-methoxy-5-[3-(trimethylammonio)propoxy]-1,3-benzothiazol-2-yl]methylcarbamoyl]indan-2-yl]acetate;
2-[2-[[6-methoxy-5-[2-[2-(trimethylammonio)ethoxy]ethoxy]-1,3-benzothiazol-2-yl]methylcarbamoyl]indan-2-yl]acetate;
2-[5,6-difluoro-2-[[6-methoxy-5-[2-[2-(trimethylammonio)ethoxy]ethoxy]-1,3-benzothiazol-2-yl]methylcarbamoyl]indan-2-yl]acetate;
2-[5,6-difluoro-2-[[6-methoxy-5-[2-(trimethylammonio)ethoxy]-1,3-benzothiazol-2-yl]methylcarbamoyl]indan-2-yl]acetate;
2-(2-(((5-(4-(dimethylamino)butoxy)-6-methoxybenzo[d]thiazol-2-yl)methyl)carbamoyl)-2,3-dihydro-1H-inden-2-yl)acetic acid; and
2-(2-(((6-methoxy-5-(4-(trimethylammonio)butoxy)benzo[d]thiazol-2-yl)methyl)carbamoyl)-2,3-dihydro-1H-inden-2-yl)acetate;
   and pharmaceutically acceptable salts thereof.

More preferred compounds according to this aspect are:
2-[2-[[6-(3-hydroxypropoxy)-1,3-benzothiazol-2-yl]methylcarbamoyl]indan-2-yl]acetic acid;
2-[2-[(6-propoxy-1,3-benzothiazol-2-yl)methylcarbamoyl]indan-2-yl]acetic acid;
2-[2-[[5-[3-(dimethylamino)propoxy]-6-methoxy-1,3-benzothiazol-2-yl]methylcarbamoyl]indan-2-yl]acetic acid;
2-[2-[[6-methoxy-5-[3-(trimethylammonio)propoxy]-1,3-benzothiazol-2-yl]methylcarbamoyl]indan-2-yl]acetate;
2-[2-[[6-methoxy-5-[2-[2-(trimethylammonio)ethoxy]ethoxy]-1,3-benzothiazol-2-yl]methylcarbamoyl]indan-2-yl]acetate;
2-[5,6-difluoro-2-[[6-methoxy-5-[2-[2-(trimethylammonio)ethoxy]ethoxy]-1,3-benzothiazol-2-yl]methylcarbamoyl]indan-2-yl]acetate; and
2-[5,6-difluoro-2-[[6-methoxy-5-[2-(trimethylammonio)ethoxy]-1,3-benzothiazol-2-yl]methylcarbamoyl]indan-2-yl]acetate;
   and pharmaceutically acceptable salts thereof.

Most preferred compounds of this aspect are 2-[5,6-difluoro-2-[[6-methoxy-5-[2-[2-(trimethylammonio)ethoxy]ethoxy]-1,3-benzothiazol-2-yl]methylcarbamoyl]indan-2-yl]acetate; 2-[2-[[6-methoxy-5-[3-(trimethylammonio)propoxy]-1,3-benzothiazol-2-yl]methylcarbamoyl]indan-2-yl]acetate; 2-[2-[[6-methoxy-5-[2-[2-(trimethylammonio)ethoxy]ethoxy]-1,3-benzothiazol-2-yl]methylcarbamoyl]indan-2-yl]acetate and pharmaceutically acceptable salts of these compounds.

In a second preferred aspect of this embodiment,
Lk is selected from a bond and a C₁ to C₃ alkylene group which is unsubstituted or is substituted by one group selected from halogen, —OH, —OMe, —NR²⁰R²¹; —N⁺R²⁰R²¹R²², and —CF₃; wherein R²⁰, R²¹ and R²² are each independently selected from H and C₁ to C₃ alkyl which is unsubstituted or is substituted with one —OH or —OMe group or with one, two or three halogen groups;
R⁵ is selected from —OMe, —OH, halogen, —NR²⁰R²¹; —N⁺R²⁰R²¹R²², —CF₃, and R⁶;
each R⁶ is independently selected from:
   —R^{6a}R^{A}, —O—R^{6a}R^{A}, —NR²⁰—R^{6a}R^{A}, —R^{6b}R^{B}, —O—R^{6b}R^{B}, and —NR²⁰—R^{6b}R^{B};
   —R^{X}R^{R}, —O—R^{X}R^{R}, —O—R^{X}—C(O)—R^{R}, —R^{X}—C(O)—R^{R}, —NR²⁰—R^{X}R^{R}, and —NR²⁰—R^{X}—C(O)—R^{R}; and
   —CN; —C(O)NR²⁰R²¹; —C(O)NR²¹—R^{X}R^{B}; —C(O)NR⁴⁰R⁴¹; —SO₂R²⁰; —SO₂—R^{X}R^{B}; —SO₂NR²⁰R²¹; —SO₂—NR²⁰—R^{X}R^{B}; and —SO₂NR⁴⁰R⁴¹;
wherein:
   each R^{X} is independently selected from R^{6a} and R^{6b};
   each R^{6a} is independently selected from C₁ to C₄ alkylene, C₂ to C₄ alkenylene and C₂ to C₄ alkynylene; and each R^{6a} is independently unsubstituted or is substituted by one group selected from —OH, halogen; —NR²⁰R²¹; —NR²⁰R²¹R²²; —NR²⁰C(NR²¹)NR²²R²³; —NR²⁰C(N⁺R²¹R²²)NR²³R²⁴; —NR²⁰C(NR²¹)R²²; —NR²⁰C(N⁺R²¹R²²)R²³; —C(NR²⁰)NR²¹R²²; —C(N⁺R²⁰R²¹)NR²²R²³; —C(NR²⁰)R²¹; and —C(N⁺R²⁰R²¹)R²²; —C(O)NR²⁰R²¹; —C(O)N⁺R²⁰R²¹R²²; —C(O)—R²⁰, and methoxy which is unsubstituted or is substituted by one, two or three halogen substituents;
   each R^{6b} is independently selected from [C₁ to C₃ alkylene]-C(R^{z})₂, [C₂ to C₃ alkenylene]—C(R^{z})₂ and [C₂ to C₃ alkynylene]—C(R^{z})₂; wherein the two R^{z} groups are attached together to form, together with the atom to which they are attached, a 5- or 6-membered carbocyclic or heterocyclic group;
   R^{A} is selected from —NR²⁰R³⁰; —N⁺R²⁰R²¹R³⁰; —NR²⁰NR²¹R²²; —NR²⁰N⁺R²¹R²²R²³; —N⁺R²⁰R²¹NR²²R²³; —NR²⁰C(NR²¹)NR²²R²³; —NR²⁰C(N⁺R²¹R²²)NR²³R³⁰; —C(NR²⁰)NR²¹R²²; and —C(N⁺R²⁰R²¹)NR²²R²³;
   R^{B} is selected from —NR²⁰R²¹; —N⁺R²⁰R²¹R²²; —NR²⁰NR²¹R²²; —NR²⁰N⁺R²¹R²²R²³; —N⁺R²⁰R²¹NR²²R²³; —NR²⁰C(NR²¹)NR²²R²³; —NR²⁰C(N⁺R²¹R²²)NR²³R²⁴; —C(NR²⁰)NR²¹R²²; and —C(N⁺R²⁰R²¹)NR²²R²³;
   R⁴⁰ and R⁴¹, together with the nitrogen atom to which they are attached, form a 4- to 6-membered heterocyclic group, wherein any nitrogen atom in the ring is independently selected from secondary, tertiary and quaternary nitrogen atoms;
   each R^{R} is independently a 4- to 10-membered heteroaryl or heterocyclic group comprising at least one nitrogen atom, and said nitrogen atom(s) are independently selected from secondary, tertiary and quaternary nitrogen atom(s);
      wherein each R^{R}, and each ring formed by —NR⁴⁰R⁴¹, is independently unsubstituted or is substituted with one, two or three groups independently selected from
      i) halogen, —CN;
      ii) oxo, providing that said R^{R} group is a heterocyclic group;
      iii) —R²⁰, —R⁷—OR²⁰; —R⁷—NR²⁰R²¹; —R⁷—N⁺R²⁰R²¹R²²; —R⁷—NR²⁰C(NR²¹)NR²²R²³; —R⁷—NR²⁰C(N⁺R²¹R²²)NR²³R²⁴; —R⁷—NR²⁰C(NR²¹)R²²; —R⁷—NR²⁰C(N⁺R²¹R²²)R²³; —R⁷—C(NR²⁰)NR²¹R²²; —R⁷—C(N⁺R²⁰R²¹)NR²²R²³; —R⁷—C(NR²⁰)R²¹; and —R⁷—C(N⁺R²⁰R²¹)R²².
each R⁷ is independently selected from a bond and unsubstituted C₁ to C₃ alkylene;
R²⁰, R²¹, R²², R²³ and R²⁴ are each independently selected from H and C₁ to C₃ alkyl which is unsubstituted or is substituted with one —OH or —OMe group or with one, two or three halogen groups;
each R³⁰ is independently selected from C₂ to C₃ alkyl which is unsubstituted or is substituted with one —OH or —OMe group or with one, two or three halogen groups.

In a first form of this aspect, each R⁶ is preferably independently selected from: —R^{6a}R^{A}, —O—R^{6a}R^{A}, —NR²⁰—R^{6a}R^{A}, —R^{6b}R^{B}, —O—R^{6b}R^{B}, —NR²⁰—R^{6b}R^{B}, —R^{X}R^{R}, —O—R^{X}R^{R}, —O—R^{X}—C(O)—R^{R}, and —R^{X}—C(O)—R^{R}. More preferably, each R⁶ is independently selected from: —O—R^{6a}R^{A}, —NR²⁰—R^{6a}R^{A}, —O—R^{6b}R^{B}, —NR²⁰—R^{6b}R^{B}, —O—R^{X}R^{R}, and —O—R^{X}—C(O)—R^{R}. Most preferably, each R⁶ is independently selected from: —O—R^{6a}R^{A}, —O—R^{6b}R^{B}, —O—R^{X}R^{R}, and —O—R^{X}—C(O)—R^{R}.

In this form, each R^{X} is preferably an R^{6a} group. Each R^{6a} is preferably independently a C₁ to C₄ alkylene group and is independently unsubstituted or is substituted by one group selected from —OH, halogen; —NR²⁰R²¹; —N⁺R²⁰R²¹R²²; and unsubstituted methoxy. Most preferably, each R^{6a} is independently an unsubstituted C₁ to C₄ alkylene group; preferably an unsubstituted C₁ to C₃ alkylene group.

In this form, each R^{6b} is preferably independently a [C₁ to C₃ alkylene]-C(R^{z})₂R^{b} group; wherein the two R^{Z} groups are attached together to form, together with the atom to which they are attached, a 5- or 6-membered carbocyclic or heterocyclic group. More preferably, the two R^{Z} groups are attached together to form, together with the atom to which they are attached, a 5- or 6-membered heterocyclic group, most preferably a piperidine or an oxane group, preferably an oxane group. The carbocyclic or heterocyclic group formed by the two R^{Z} groups is preferably unsubstituted or is substituted by one substituted selected from —CH₃, —OH and —OCH₃. Most preferably the carbocyclic or heterocyclic group formed by the two R^{Z} groups is unsubstituted.

In this form, R^{A} is preferably selected from —NR²⁰R³⁰; —N⁺R²⁰R²¹R³⁰; —NR²⁰NR²¹R²²; and —NR²⁰N⁺R²¹R²²R²³. More preferably, R^{A} is selected from —NR²⁰R³⁰; and —N⁺R²⁰R²¹R³⁰.

In this form, R^{B} is preferably selected from —NR²⁰R²¹; —N⁺R²⁰R²¹R²²; —NR²⁰NR²¹R²²; and —NR²⁰N⁺R²¹R²²R²³. More preferably, R^{B} is selected from —NR²⁰R²¹; and —N⁺R²⁰R²¹R²².

In this form, each R^{R} is preferably independently a 5- to 6-membered heteroaryl or 4- to 6-membered heterocyclic group comprising at least one nitrogen atom, and said nitrogen atom(s) are independently selected from secondary, tertiary and quaternary nitrogen atom(s). More preferably, each R^{R} is independently a 4- to 6-membered heterocyclic group, e.g. a 5- or 6-membered heterocyclic group, and comprises at least one nitrogen atom, and said nitrogen atom(s) are independently selected from secondary, tertiary and quaternary nitrogen atom(s). Most preferably, each R^{R} is independently selected from azetidine, morpholine, piperazine, piperidine, pyrrolidine and triazole. For avoidance of doubt, the nitrogen atom(s) in said groups may be quaternized as defined herein.

Preferably, each R^{R} is independently unsubstituted or is substituted with one, two or three groups independently selected from —R²⁰, —R⁷—OR²⁰; —R⁷—NR²⁰R²¹; and —R⁷—N⁺R²⁰R²¹R²². More preferably each R^{R} is independently unsubstituted or is substituted with one or two groups independently selected from —R²⁰; —R⁷—NR²⁰R²¹; and —R⁷—N⁺R²⁰R²¹R²². Yet more preferably each R^{R} is independently unsubstituted or is substituted with one or two —R²⁰ groups.

Accordingly, therefore, in this form, each R⁶ is preferably independently selected from: —R^{6a}R^{A}, —O—R^{6a}R^{A}, —NR²⁰—R^{6a}R^{A}, —R^{6b}R^{B}, —O—R^{6b}R^{B}, —NR²⁰—R^{6b}R^{B}, —R^{X}R^{R},

—O—R^{X}R^{R}, —O—R^{X}—C(O)—R^{R}, and —R^{X}—C(O)—R^{R}, wherein:
each R^{X} is an R^{6a} group;
each R^{6a} is independently a C₁ to C₄ alkylene group and each R^{6a} is independently unsubstituted or is substituted by one group selected from —OH, halogen; —NR²⁰R²¹; —N⁺R²⁰R²¹R²²; and unsubstituted methoxy;
each R^{6b} is independently a [C₁ to C₃ alkylene]-C(R^{z})₂R^{b} group; wherein the two R^{z} groups are attached together to form, together with the atom to which they are attached, a 5- or 6-membered carbocyclic or heterocyclic group;
R^{A} is selected from —NR²⁰R³⁰; —N⁺R²⁰R²¹R³⁰; —NR²⁰NR²¹R²²; and —NR²⁰N⁺R²¹R²²R²³;
R^{B} is selected from —NR²⁰R²¹; —N⁺R²⁰R²¹R²²; —NR²⁰NR²¹R²²; and —NR²⁰N⁺R²¹R²²R²³;
each R^{R} is independently a 5- to 6-membered heteroaryl or 4- to 6-membered heterocyclic group comprising at least one nitrogen atom, and said nitrogen atom(s) are independently selected from secondary, tertiary and quaternary nitrogen atom(s);
   wherein each R^{R} is independently unsubstituted or is substituted with one, two or three groups independently selected from —R²⁰, —R⁷—OR²⁰; —R⁷—NR²⁰R²¹; and —R⁷—N⁺R²⁰R²¹R²².

More preferably, in this form, each R⁶ is independently selected from: —O—R^{6a}R^{A}, —O—R^{6b}R^{B}, —O—R^{X}R^{R}, and —O—R^{X}—C(O)—R^{R}, wherein:
each R^{X} is an R^{6a} group;
each R^{6a} is independently an unsubstituted C₁ to C₄ alkylene group;
each R^{b} is independently a [C₁ to C₃ alkylene]-C(R^{z})₂R^{b} group; wherein the two R^{z} groups are attached together to form, together with the atom to which they are attached, a 5- or 6-membered heterocyclic group, preferably an oxane group;
R^{A} is selected from —NR²⁰R³⁰; —N⁺R²⁰R²¹R³⁰; —NR²⁰NR²¹R²²; and —NR²⁰N⁺R²¹R²²R²³;
R^{B} is selected from —NR²⁰R²¹; —N⁺R²⁰R²¹R²²; —NR²⁰NR²¹R²²; and —NR²⁰N⁺R²¹R²²R²³;
each R^{R} is independently a 5- to 6-membered heteroaryl or 4- to 6-membered heterocyclic group comprising at least one nitrogen atom, and said nitrogen atom(s) are independently selected from secondary, tertiary and quaternary nitrogen atom(s);
   wherein each R^{R} is independently unsubstituted or is substituted with one or two groups independently selected from —R²⁰; —R⁷—NR²⁰R²¹; and —R⁷—N⁺R²⁰R²¹R²².

In a second form of this aspect, each R⁶ is preferably independently selected from: —CN; —C(O)NR²⁰R²¹; —C(O)NR²¹—R^{X}R^{B}; —C(O)NR⁴⁰R⁴¹; —SO₂R²⁰; —SO₂NR²⁰R²¹; and —SO₂NR⁴⁰R⁴¹. More preferably, each R⁶ is independently selected from: —C(O)NR²⁰R²¹; —C(O)NR²¹—R^{X}R^{B}; —C(O)NR⁴⁰R⁴¹; and —SO₂NR⁴⁰R⁴¹. Yet more preferably each R⁶ is independently selected from —SO₂NR⁴⁰R⁴¹ and —C(O)NR⁴⁰R⁴¹. Most preferably, each R⁶ is independently a C(O)NR²⁰R²¹ group.

In this form, each R^{X} is preferably an R^{6a} group. Each R^{6a} is preferably independently a C₁ to C₄ alkylene group and is independently unsubstituted or is substituted by one group selected from —OH, halogen; —NR²⁰R²¹; —N⁺R²⁰R²¹R²²; and unsubstituted methoxy. Most preferably, each R^{6a} is independently an unsubstituted C₁ to C₄ alkylene group; preferably an unsubstituted C₁ to C₃ alkylene group.

In this form, R^{B} is preferably selected from —NR²⁰R²¹; —N⁺R²⁰R²¹R²²; —NR²⁰NR²¹R²²; and —NR²⁰N⁺R²¹R²²R²³. More preferably, R^{B} is selected from —NR²⁰R²¹; and —N⁺R²⁰R²¹R²².

In this form, each R⁴⁰ and R⁴¹ together with the nitrogen atom to which they are attached, preferably independently form a 4- to 6-membered heterocyclic group, e.g. a 4- or 6-membered heterocyclic group, wherein any nitrogen atom in the ring is independently selected from secondary, tertiary and quaternary nitrogen atoms. Most preferably, each ring formed by —NR⁴⁰R⁴¹ if present is independently selected from azetidine, morpholine, piperazine, piperidine, pyrrolidine and triazole. For avoidance of doubt, the nitrogen atom(s) in said groups may be quaternized as defined herein.

Preferably, each ring formed by —NR⁴⁰R⁴¹ is independently unsubstituted or is substituted with one, two or three groups independently selected from —R²⁰, —R⁷—OR²⁰; —R⁷—NR²⁰R²¹; and —R⁷—N⁺R²⁰R²¹R²². More preferably, each ring formed by NR⁴⁰R⁴¹ is independently unsubstituted or is substituted with one or two groups independently selected from —R²⁰; —R⁷—NR²⁰R²¹; and —R⁷—N⁺R²⁰R²¹R²². Most preferably, each ring formed by NR⁴⁰R⁴¹ is independently unsubstituted or is substituted with one or two groups independently selected from —R²⁰ and —R⁷—NR²⁰R²¹.

Accordingly, therefore, in this form, each R⁶ is preferably independently selected from: —CN; —C(O)NR²⁰R²¹; —C(O)NR²¹—R^{X}R^{B}; —C(O)NR⁴⁰R⁴¹; —SO₂R²⁰; —SO₂NR²⁰R²¹; and —SO₂NR⁴⁰R⁴¹; wherein:
each R^{X} is a R^{6a} group;
each R^{6a} is independently a C₁ to C₄ alkylene group; and each R^{6a} is independently unsubstituted or is substituted by one group selected from —OH, halogen; —NR²⁰R²¹; —N⁺R²⁰R²¹R²²; and unsubstituted methoxy;
R^{B} is selected from —NR²⁰R²¹; —N⁺R²⁰R²¹R²²; —NR²⁰NR²¹R²²; and —NR²⁰N⁺R²¹R²²R²³.
each R⁴⁰ and R⁴¹ together with the nitrogen atom to which they are attached, independently form a 4- to 6-membered heterocyclic group, wherein any nitrogen atom in the ring is independently selected from secondary, tertiary and quaternary nitrogen atoms;
   wherein each ring formed by —NR⁴⁰R⁴¹ is independently unsubstituted or is substituted with one, two or three groups independently selected from —R²⁰, —R⁷—OR²⁰; —R⁷—NR²⁰R²¹; and —R⁷—N⁺R²⁰R²¹R²².

More preferably, in this form, each R⁶ is independently selected from: —CN; —C(O)NR²⁰R²¹; —C(O)NR²¹—R^{X}R^{B}; —C(O)NR⁴⁰R⁴¹; —SO₂R²⁰; —SO₂NR²⁰R²¹; and —SO₂NR⁴⁰R⁴¹; wherein:
each R^{X} is a R^{6a} group;
each R^{6a} is independently an unsubstituted C₁ to C₄ alkylene group;
R^{B} is selected from —NR²⁰R²¹ and —N⁺R²⁰R²¹R²²;
each R⁴⁰ and R⁴¹ together with the nitrogen atom to which they are attached, independently form a 4- to 6-membered heterocyclic group, wherein any nitrogen atom in the ring is independently selected from secondary, tertiary and quaternary nitrogen atoms;
wherein each ring formed by NR⁴⁰R⁴¹ is independently unsubstituted or is substituted with one or two groups independently selected from —R²⁰; —R⁷—NR²⁰R²¹; and —R⁷—N⁺R²⁰R²¹R²².

In this aspect, preferred compounds of Formula (I) are thus those in which
Lk is —CH₂—;
p is 0; or p is 1 and R⁵ is —OMe; preferably p is 0;
each R⁶ is preferably independently selected from:
A:
   —R^{6a}R^{A}, —O—R^{6a}R^{A}, —NR²⁰—R^{6a}R^{A}, —R^{6b}R^{B}, —O—R^{6b}R^{B}, —NR²⁰—R^{6b}R^{B}, —R^{X}R^{R}, —O—R^{X}R^{R}, —O—R^{X}—C(O)—R^{R}, and —R^{X}—C(O)—R^{R}, wherein:
      each R^{X} is an R^{6a} group;
      each R^{6a} is independently a C₁ to C₄ alkylene group and each R^{6a} is independently unsubstituted or is substituted by one group selected from —OH, halogen; —NR²⁰R²¹.
      —N⁺R²⁰R²¹R²²; and unsubstituted methoxy;
      each R^{6b} is independently a [C₁ to C₃ alkylene]-C(R^{z})₂R^{b} group; wherein the two R^{Z} groups are attached together to form, together with the atom to which they are attached, a 5- or 6-membered carbocyclic or heterocyclic group;
      R^{A} is selected from —NR²⁰R³⁰; —NR²⁰R²¹R³⁰; —NR²⁰NR²¹R²²; and —NR²⁰N⁺R²¹R²²R²³;
      R^{B} is selected from —NR²⁰R²¹; —N⁺R²⁰R²¹R²²; —NR²⁰NR²¹R²²; and —NR²⁰N⁺R²¹R²²R²³;
      each R^{R} is independently a 5- to 6-membered heteroaryl or 4- to 6-membered heterocyclic group comprising at least one nitrogen atom, and said nitrogen atom(s) are independently selected from secondary, tertiary and quaternary nitrogen atom(s);
         wherein each R^{R} is independently unsubstituted or is substituted with one, two or three groups independently selected from —R²⁰, —R⁷—OR²⁰; —R⁷—NR²⁰R²¹; and —R⁷—N⁺R²⁰R²¹R²²; and
B:
   —CN; —C(O)NR²⁰R²¹; —C(O)NR²¹—R^{X}R^{B}; —C(O)NR⁴⁰R⁴¹; —SO₂R²⁰; —SO₂NR²⁰R²¹; and —SO₂NR⁴⁰R⁴¹; wherein:
      each R^{X} is a R^{6a} group;
      each R^{6a} is independently a C₁ to C₄ alkylene group; and each R^{6a} is independently unsubstituted or is substituted by one group selected from —OH, halogen; —NR²⁰R²¹;
      —N⁺R²⁰R²¹R²²; and unsubstituted methoxy;
      R^{B} is selected from —NR²⁰R²¹; —N⁺R²⁰R²¹R²²; —NR²⁰NR²¹R²²; and —NR²⁰N⁺R²¹R²²R²³;
      each R⁴⁰ and R⁴¹ together with the nitrogen atom to which they are attached, independently form a 4- to 6-membered heterocyclic group, wherein any nitrogen atom in the ring is independently selected from secondary, tertiary and quaternary nitrogen atoms;
         wherein each ring formed by —NR⁴⁰R⁴¹ is independently unsubstituted or is substituted with one, two or three groups independently selected from —R²⁰, —R⁷—OR²⁰; —R⁷—NR²⁰R²¹; and —R⁷—N⁺R²⁰R²¹R²².

In still more particularly compounds of this aspect:
Lk is —CH₂—;
p is 0; or p is 1 and R⁵ is —OMe; preferably p is 1 and R⁵ is —OMe;
each R⁶ is preferably independently selected from:
A:
   —O—R^{6a}R^{A}, —O—R^{6b}R^{B}, —O—R^{X}R^{R}, and —O—R^{X}—C(O)—R^{R}, wherein:
      each R^{X} is an R^{6a} group;
      each R^{6a} is independently an unsubstituted C₁ to C₄ alkylene group;
      each R^{6b} is independently a [C₁ to C₃ alkylene]-C(R^{z})₂R^{b} group; wherein the two R^{Z} groups are attached together to form, together with the atom to which they are attached, a 5- or 6-membered heterocyclic group, preferably an oxane group;
      R^{A} is selected from —NR²⁰R³⁰; —N⁺R²⁰R²¹R³⁰; —NR²⁰NR²¹R²²; and —NR²⁰N⁺R²¹R²²R²³;
      R^{B} is selected from —NR²⁰R²¹; —N⁺R²⁰R²¹R²²; —NR²⁰NR²¹R²²; and —NR²⁰N⁺R²¹R²²R²³;
      each R^{R} is independently a 5- to 6-membered heteroaryl or 4- to 6-membered heterocyclic group comprising at least one nitrogen atom, and said nitrogen atom(s) are independently selected from secondary, tertiary and quaternary nitrogen atom(s);
         wherein each R^{R} is independently unsubstituted or is substituted with one or two groups independently selected from —R²⁰; —R⁷—NR²⁰R²¹; and —R⁷—N⁺R²⁰R²¹R²²; and
B:
   —CN; —C(O)NR²⁰R²¹; —C(O)NR²¹—R^{X}R^{B}; —C(O)NR⁴⁰R⁴¹; —SO₂R²⁰; —SO₂NR²⁰R²¹; and —SO₂NR⁴⁰R⁴¹; wherein:
      each R^{X} is a R^{6a} group;
      each R^{6a} is independently an unsubstituted C₁ to C₄ alkylene group;
      R^{B} is selected from —NR²⁰R²¹ and —N⁺R²⁰R²¹R²²;
      each R⁴⁰ and R⁴¹ together with the nitrogen atom to which they are attached, independently form a 4- to 6-membered heterocyclic group, wherein any nitrogen atom in the ring is independently selected from secondary, tertiary and quaternary nitrogen atoms;
      wherein each ring formed by NR⁴⁰R⁴¹ is independently unsubstituted or is substituted with one or two groups independently selected from —R²⁰; —R⁷—NR²⁰R²¹; and —R⁷—N⁺R²⁰R²¹R²².

Preferably, therefore, in this aspect:
Lk is —CH₂—;
R⁵ is selected from —OMe and —OH; preferably —OMe; and
R⁶ is selected from: —O—R^{6a}R^{A}, —O—R^{6b}R^{B}, —O—R^{X}R^{R}, and —O—R^{X}—C(O)—R^{R}, wherein:
   each R^{X} is an R^{6a} group;
   each R^{6a} is independently an unsubstituted C₁ to C₄ alkylene group;
   each R^{6b} is independently a [C₁ to C₃ alkylene]-C(R^{z})₂ group; wherein the two R^{z} groups are attached together to form, together with the atom to which they are attached, a 5- or 6-membered heterocyclic group, preferably an oxane group;
   R^{A} is selected from —NR²⁰R³⁰; —N⁺R²⁰R²¹R³⁰; —NR²⁰NR²¹R²²; and —NR²⁰N⁺R²¹R²²R²³;
   R^{B} is selected from —NR²⁰R²¹; —N⁺R²⁰R²¹R²²; —NR²⁰NR²¹R²²; and —NR²⁰N⁺R²¹R²²R²³;
   each R^{R} is independently a 5- to 6-membered heteroaryl or 4- to 6-membered heterocyclic group comprising at least one nitrogen atom, and said nitrogen atom(s) are independently selected from secondary, tertiary and quaternary nitrogen atom(s);
   wherein each R^{R} is independently unsubstituted or is substituted with one or two groups independently selected from —R²⁰; —R⁷—NR²⁰R²¹; and —R⁷—N⁺R²⁰R²¹R²².

Preferably, in this aspect, R⁷ is selected from a bond and unsubstituted C₁ alkylene; more preferably R⁷ is a bond. R²⁰, R²¹, R²², R²³ and R²⁴ are preferably each independently selected from H and C₁ to C₂ alkyl which is unsubstituted or is substituted with one OMe group. More preferably, R²⁰, R²¹, R²², R²³ and R²⁴ are each independently selected from H and unsubstituted C₁ to C₂ alkyl; most preferably R²⁰, R²¹, R²², R²³ and R²⁴ are each independently selected from H and methyl. Each R³⁰ is preferably independently C₂ or C₃ alkyl which is unsubstituted or is substituted with one OMe group. More preferably, each R³⁰ is independently C₂ alkyl which is unsubstituted or is substituted with one OMe group. Most preferably, each R³⁰ is independently unsubstituted C₂ alkyl.

More preferably, therefore, in this aspect:
R¹ is selected from —OH and —NHOH, or where the compound of Formula (I) contains a positively charged nitrogen atom, R¹ may be O⁻, such that the compound forms a zwitterion;
R² is H;
R⁴ is H;
n is 0; or n is 2 and each R³ group is fluorine;
Lk is —CH₂—;

The moiety \textcircled{A}-(G)ₘ is

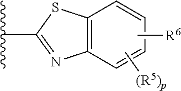

p is 1;
R⁵ is selected from —OMe and —OH; preferably —OMe; and
R⁶ is selected from: —O—R^{6a}R^{A}, —O—R^{6b}R^{B}, —O—R^{X}R^{R}, and —O—R^{X}—C(O)—R^{R}, wherein:
   each R^{X} is an R^{6a} group;
   each R^{6a} is independently an unsubstituted C₁ to C₄ alkylene group;
   each R^{6b} is independently a [C₁ to C₃ alkylene]-C(R^{z})₂ group; wherein the two R^{Z} groups are attached together to form, together with the atom to which they are attached, a 5- or 6-membered heterocyclic group, preferably an oxane group;
   R^{A} is selected from —NR²⁰R³⁰; —N⁺R²⁰R²¹R³⁰; —NR²⁰NR²¹R²²; and —NR²⁰N⁺R²¹R²²R²³;
   R^{B} is selected from —NR²⁰R²¹; —N⁺R²⁰R²¹R²²; —NR²⁰NR²¹R²²; and —NR²⁰N⁺R²¹R²²R²³.
   each R^{R} is independently a 5- to 6-membered heteroaryl or 4- to 6-membered heterocyclic group comprising at least one nitrogen atom, and said nitrogen atom(s) are independently selected from secondary, tertiary and quaternary nitrogen atom(s);
   wherein each R^{R} is independently unsubstituted or is substituted with one or two groups independently selected from —R²⁰; —R⁷—NR²⁰R²¹; and —R⁷—N⁺R²⁰R²¹R²².

Preferred compounds of this aspect include: 2-[2-[[6-methoxy-5-[2-(4-methylpiperazin-1-yl)-2-oxo-ethoxy]-1,3-benzothiazol-2-yl]methylcarbamoyl]indan-2-yl]acetic acid; 2-[2-[[6-methoxy-5-(2-morpholinoethoxy)-1,3-benzothiazol-2-yl]methylcarbamoyl]indan-2-yl]acetic acid; 2-[5,6-difluoro-2-[[6-methoxy-5-(2-morpholinoethoxy)-1,3-benzothiazol-2-yl]methylcarbamoyl]indan-2-yl]acetic acid; 2-[5,6-difluoro-2-[[6-methoxy-5-[(1-methyl-4-piperidyl)methoxy]-1,3-benzothiazol-2-yl]methylcarbamoyl]indan-2-yl]acetic acid; 2-[5,6-difluoro-2-[[6-methoxy-5-[2-(4-methylpiperazin-1-yl)-2-oxo-ethoxy]-1,3-benzothiazol-2-yl]methylcarbamoyl]indan-2-yl]acetic acid; 2-[5,6-difluoro-2-[[6-methoxy-5-[2-(4-methylmorpholin-4-ium-4-yl)ethoxy]-1,3-benzothiazol-2-yl]methylcarbamoyl]indan-2-yl]acetate; 2-[2-[[5-[2-(4,4-dimethylpiperazin-4-ium-1-yl)-2-oxo-ethoxy]-6-methoxy-1,3-benzothiazol-2-yl]methylcarbamoyl]-5,6-difluoro-indan-2-yl]acetate; 2-[5,6-difluoro-2-[[6-methoxy-5-[3-(4-methylmorpholin-4-ium-4-yl)propoxy]-1,3-benzothiazol-2-yl]methylcarbamoyl]indan-2-yl]acetate; 2-[2-[[6-methoxy-5-[3-(4-methylmorpholin-4-ium-4-yl)propoxy]-1,3-benzothiazol-2-yl]methylcarbamoyl]indan-2-yl]acetate; 2-[2-[[5-[(1,1-dimethylpiperidin-1-ium-4-yl)methoxy]-6-methoxy-1,3-benzothiazol-2-yl]methylcarbamoyl]indan-2-yl]acetate; 2-[2-[[5-[3-[diethyl(methyl)ammonio]propoxy]-6-methoxy-1,3-benzothiazol-2-yl]methylcarbamoyl]indan-2-yl]acetate; 2-[5,6-difluoro-2-[[6-methoxy-5-[3-(1-methylpyrrolidin-1-ium-1-yl)propoxy]-1,3-benzothiazol-2-yl]methylcarbamoyl]indan-2-yl]acetate; 2-[2-[[5-[3-[2-hydroxyethyl(dimethyl)ammonio]propoxy]-6-methoxy-1,3-benzothiazol-2-yl]methylcarbamoyl]indan-2-yl]acetate; 2-[5,6-difluoro-2-[[5-[3-[2-hydroxyethyl(dimethyl)ammonio]propoxy]-6-methoxy-1,3-benzothiazol-2-yl]methylcarbamoyl]indan-2-yl]acetate; 2-[2-[[5-[3-[bis(2-hydroxyethyl)-methyl-ammonio]propoxy]-6-methoxy-1,3-benzothiazol-2-yl]methylcarbamoyl]indan-2-yl]acetate; 2-[2-[[5-[3-[bis(2-hydroxyethyl)-methyl-ammonio]propoxy]-6-methoxy-1,3-benzothiazol-2-yl]methylcarbamoyl]-5,6-difluoro-indan-2-yl]acetate; 2-[2-[[6-methoxy-5-(4-methylpiperazine-1-carbonyl)-1,3-benzothiazol-2-yl]methylcarbamoyl]indan-2-yl]acetic acid; 2-[5,6-difluoro-2-[[6-methoxy-5-(4-methylpiperazine-1-carbonyl)-1,3-benzothiazol-2-yl]methylcarbamoyl]indan-2-yl]acetic acid; 2-[2-[[5-(4,4-dimethylpiperazin-4-ium-1-carbonyl)-6-methoxy-1,3-benzothiazol-2-yl]methylcarbamoyl]-5,6-difluoro-indan-2-yl]acetate; 2-[2-[[5-[3-(dimethylamino)azetidine-1-carbonyl]-6-methoxy-1,3-benzothiazol-2-yl]methylcarbamoyl]indan-2-yl]acetic acid; 2-[2-[[5-[3-(dimethylamino)azetidine-1-carbonyl]-6-methoxy-1,3-benzothiazol-2-yl]methylcarbamoyl]-5,6-difluoro-indan-2-yl]acetic acid; 2-[2-[[5-[4-(dimethylamino)piperidine-1-carbonyl]-6-methoxy-1,3-benzothiazol-2-yl]methylcarbamoyl]indan-2-yl]acetic acid; 2-[2-[[5-[2-[(dimethylamino)methyl]morpholine-4-carbonyl]-6-methoxy-1,3-benzothiazol-2-yl]methylcarbamoyl]indan-2-yl]acetic acid; 2-[2-[[6-methoxy-5-[2-[(trimethylammonio)methyl]morpholine-4-carbonyl]-1,3-benzothiazol-2-yl]methylcarbamoyl]indan-2-yl]acetate; 2-[5,6-difluoro-2-[[6-methoxy-5-[3-(trimethylammonio)azetidine-1-carbonyl]-1,3-benzothiazol-2-yl]methylcarbamoyl]indan-2-yl]acetate; 2-[2-[[5-[(1,1-dimethylpiperidin-1-ium-4-yl)methoxy]-6-methoxy-1,3-benzothiazol-2-yl]methylcarbamoyl]-5,6-difluoro-indan-2-yl]acetate; 2-[2-[[6-methoxy-5-(4-methylpiperazin-1-yl)sulfonyl-1,3-benzothiazol-2-yl]methylcarbamoyl]indan-2-yl]acetic acid; 2-[2-[[5-[[4-(dimethylamino)-1-piperidyl]sulfonyl]-6-methoxy-1,3-benzothiazol-2-yl]methylcarbamoyl]indan-2-yl]acetic acid; and 2-[2-[[6-methoxy-5-[[4-(trimethylammonio)-1-piperidyl]sulfonyl]-1,3-benzothiazol-2-yl]methylcarbamoyl]indan-2-yl]acetate; and pharmaceutically acceptable salts thereof.

More preferred compounds of this aspect are selected from: 2-[2-[[6-methoxy-5-(2-morpholinoethoxy)-1,3-benzothiazol-2-yl]methylcarbamoyl]indan-2-yl]acetic acid; 2-[5,6-difluoro-2-[[6-methoxy-5-(2-morpholinoethoxy)-1,3-benzothiazol-2-yl]methylcarbamoyl]indan-2-yl]acetic acid; 2-[5,6-difluoro-2-[[6-methoxy-5-[(1-methyl-4-piperidyl)methoxy]-1,3-benzothiazol-2-yl]methylcarbamoyl]indan-2-yl]acetic acid; 2-[2-[[6-methoxy-5-[3-(4-methylmorpholin-4-ium-4-yl)propoxy]-1,3-benzothiazol-2-yl]methylcarbamoyl]indan-2-yl]acetate; 2-[2-[[5-[3-[diethyl(methyl)ammonio]propoxy]-6-methoxy-1,3-benzothiazol-2-yl]methylcarbamoyl]indan-2-yl]acetate; 2-[5,6-difluoro-2-[[6-methoxy-5-[3-(1-methylpyrrolidin-1-ium-1-yl)propoxy]-1,3-benzothiazol-2-yl]methylcarbamoyl]indan-2-yl]acetate; 2-[2-[[5-[3-[2-hydroxyethyl(dimethyl)ammonio]propoxy]-6-methoxy-1,3-benzothiazol-2-yl]methylcarbamoyl]indan-2-yl]acetate; 2-[5,6-difluoro-2-[[5-[3-[2-hydroxyethyl(dimethyl)ammonio]propoxy]-6-methoxy-1,3-benzothiazol-2-yl]methylcarbamoyl]indan-2-yl]acetate; 2-[2-[[5-[3-[bis(2-hydroxyethyl)-methyl-ammonio]propoxy]-6-methoxy-1,3-benzothiazol-2-yl]methylcarbamoyl]indan-2-yl]acetate; 2-[2-[[5-[3-[bis(2-hydroxyethyl)-methyl-ammonio]propoxy]-6-methoxy-1,3-benzothiazol-2-yl]methylcarbamoyl]-5,6-difluoro-indan-2-yl]acetate; 2-[2-[[6-methoxy-5-(4-methylpiperazine-1-carbonyl)-1,3-benzothiazol-2-yl]methylcarbamoyl]indan-2-yl]acetic acid; 2-[5,6-difluoro-2-[[6-methoxy-5-(4-methylpiperazine-1-carbonyl)-1,3-benzothiazol-2-yl]methylcarbamoyl]indan-2-yl]acetic acid; 2-[2-[[5-[3-(dimethylamino)azetidine-1-carbonyl]-6-methoxy-1,3-benzothiazol-2-yl]methylcarbamoyl]indan-2-yl]acetic acid; 2-[2-[[5-[3-(dimethylamino)azetidine-1-carbonyl]-6-methoxy-1,3-benzothiazol-2-yl]methylcarbamoyl]-5,6-difluoro-indan-2-yl]acetic acid; 2-[2-[[5-[(1,1-dimethylpiperidin-1-ium-4-yl)methoxy]-6-methoxy-1,3-benzothiazol-2-yl]methylcarbamoyl]-5,6-difluoro-indan-2-yl]acetate; 2-[5,6-difluoro-2-[[6-methoxy-5-[3-(4-methylmorpholin-4-ium-4-yl)propoxy]-1,3-benzothiazol-2-yl]methylcarbamoyl]indan-2-yl]acetate; 2-[5,6-difluoro-2-[[6-methoxy-5-[3-(4-methylmorpholin-4-ium-4-yl)propoxy]-1,3-benzothiazol-2-yl]methylcarbamoyl]indan-2-yl]acetate and pharmaceutically acceptable salts thereof.

Still more preferred compounds according to this aspect are selected from: 2-[2-[[6-methoxy-5-(2-morpholinoethoxy)-1,3-benzothiazol-2-yl]methylcarbamoyl]indan-2-yl]acetic acid; 2-[5,6-difluoro-2-[[6-methoxy-5-(2-morpholinoethoxy)-1,3-benzothiazol-2-yl]methylcarbamoyl]indan-2-yl]acetic acid; 2-[5,6-difluoro-2-[[6-methoxy-5-[(1-methyl-4-piperidyl)methoxy]-1,3-benzothiazol-2-yl]methylcarbamoyl]indan-2-yl]acetic acid; 2-[5,6-difluoro-2-[[6-methoxy-5-[3-(1-methylpyrrolidin-1-ium-1-yl)propoxy]-1,3-benzothiazol-2-yl]methylcarbamoyl]indan-2-yl]acetate; 2-[5,6-difluoro-2-[[5-[3-[2-hydroxyethyl(dimethyl)ammonio]propoxy]-6-methoxy-1,3-benzothiazol-2-yl]methylcarbamoyl]indan-2-yl]acetate; 2-[2-[[5-[3-[bis(2-hydroxyethyl)-methyl-ammonio]propoxy]-6-methoxy-1,3-benzothiazol-2-yl]methylcarbamoyl]-5,6-difluoro-indan-2-yl]acetate; 2-[5,6-difluoro-2-[[6-methoxy-5-(4-methylpiperazine-1-carbonyl)-1,3-benzothiazol-2-yl]methylcarbamoyl]indan-2-yl]acetic acid; 2-[2-[[5-[3-(dimethylamino)azetidine-1-carbonyl]-6-methoxy-1,3-benzothiazol-2-yl]methylcarbamoyl]indan-2-yl]acetic acid; 2-[2-[[5-[3-(dimethylamino)azetidine-1-carbonyl]-6-methoxy-1,3-benzothiazol-2-yl]methylcarbamoyl]-5,6-difluoro-indan-2-yl]acetic acid; 2-[2-[[5-[(1,1-dimethylpiperidin-1-ium-4-yl)methoxy]-6-methoxy-1,3-benzothiazol-2-yl]methylcarbamoyl]-5,6-difluoro-indan-2-yl]acetate; 2-[5,6-difluoro-2-[[6-methoxy-5-[3-(4-methylmorpholin-4-ium-4-yl)propoxy]-1,3-benzothiazol-2-yl]methylcarbamoyl]indan-2-yl]acetate; 2-[5,6-difluoro-2-[[6-methoxy-5-[3-(4-methylmorpholin-4-ium-4-yl)propoxy]-1,3-benzothiazol-2-yl]methylcarbamoyl]indan-2-yl]acetate; 2-[2-[[5-[3-[2-hydroxyethyl(dimethyl)ammonio]propoxy]-6-methoxy-1,3-benzothiazol-2-yl]methylcarbamoyl]indan-2-yl]acetate; 2-[5,6-difluoro-2-[[5-[3-[2-hydroxyethyl(dimethyl)ammonio]propoxy]-6-methoxy-1,3-benzothiazol-2-yl]methylcarbamoyl]indan-2-yl]acetate; and pharmaceutically acceptable salts thereof.

In preferred compounds of Formula (I), therefore,
R¹ is selected from —OH and —NHOH, or where the compound of Formula (I) contains a positively charged nitrogen atom, R¹ may be O⁻, such that the compound forms a zwitterion;
R² is H;
R⁴ is H;
n is 0; or n is 2 and each R³ group is fluorine;
Lk is —CH₂—;
The moiety \textcircled{A}-(G)ₘ is

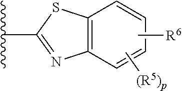

p is 1;
R⁵ is —OMe; and
R⁶ is selected from:
   —O—R^{6a}R^{A} and —O—R^{X}R^{R}; and
   C₂ to C₄ alkoxy which is substituted with a group selected from —NR¹⁰R¹¹; —N⁺R¹⁰R¹¹R¹²; and —OR^{6c}, wherein R^{6c} is a C₁ to C₃ alkyl group which is unsubstituted or substituted with a group selected from —NR¹⁰R¹¹; and —N⁺R¹⁰R¹¹R¹².
R^{X} is R^{6a};
R^{6a} is an unsubstituted C₁ to C₄ alkylene group;
R^{A} is selected from —NR²⁰R³⁰; —N⁺R²⁰R²¹R³⁰;
R^{R} is a 5- to 6-membered heterocyclic group comprising at least one nitrogen atom, and said nitrogen atom(s) are independently selected from secondary, tertiary and quaternary nitrogen atom(s); and R^{R} is unsubstituted or is substituted with one or two R²⁰ groups;
R¹⁰, R¹¹ and R¹² are each independently H or methyl;
R²⁰ and R²¹ are each independently selected from H and C₁ to C₃ alkyl which is unsubstituted or is substituted with one —OH or —OMe group;
each R³⁰ is independently selected from C₂ to C₃ alkyl which is unsubstituted or is substituted with one —OH or —OMe group.

Preferably, the compound of Formula (I) is other than:
2-(2-(((5-(3-(dimethylamino)propoxy)-6-methoxybenzo[d]thiazol-2-yl)methyl)carbamoyl)-5,6-difluoro-2,3-dihydro-1H-inden-2-yl)acetic acid;
2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate; and
2-(2-(((5-(2-(dimethylamino)ethoxy)-6-methoxybenzo[d]thiazol-2-yl)methyl)carbamoyl)-2,3-dihydro-1H-inden-2-yl)acetic acid.

Particularly preferred compounds of Formula (I) thus include:
2-[2-[[5-[3-[bis(2-hydroxyethyl)-methyl-ammonio]propoxy]-6-methoxy-1,3-benzothiazol-2-yl]methylcarbamoyl]-5,6-difluoro-indan-2-yl]acetate;
2-[2-[[6-methoxy-5-[3-(trimethylammonio)propoxy]-1,3-benzothiazol-2-yl]methylcarbamoyl]indan-2-yl]acetate;
2-[2-[[5-[(1,1-dimethylpiperidin-1-ium-4-yl)methoxy]-6-methoxy-1,3-benzothiazol-2-yl]methylcarbamoyl]-5,6-difluoro-indan-2-yl]acetate;
2-[5,6-difluoro-2-[[6-methoxy-5-[3-(1-methylpyrrolidin-1-ium-1-yl)propoxy]-1,3-benzothiazol-2-yl]methylcarbamoyl]indan-2-yl]acetate;
2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate;
2-[2-[[6-methoxy-5-[2-[2-(trimethylammonio)ethoxy]ethoxy]-1,3-benzothiazol-2-yl]methylcarbamoyl]indan-2-yl]acetate;
2-[5,6-difluoro-2-[[6-methoxy-5-[2-[2-(trimethylammonio)ethoxy]ethoxy]-1,3-benzothiazol-2-yl]methylcarbamoyl]indan-2-yl]acetate;
2-[5,6-difluoro-2-[[6-methoxy-5-[3-(4-methylmorpholin-4-ium-4-yl)propoxy]-1,3-benzothiazol-2-yl]methylcarbamoyl]indan-2-yl]acetate;
2-[2-[[5-[(1,1-dimethylpiperidin-1-ium-4-yl)methoxy]-6-methoxy-1,3-benzothiazol-2-yl]methylcarbamoyl]indan-2-yl]acetate;
2-[2-[[5-[3-[2-hydroxyethyl(dimethyl)ammonio]propoxy]-6-methoxy-1,3-benzothiazol-2-yl]methylcarbamoyl]indan-2-yl]acetate;
2-[5,6-difluoro-2-[[5-[3-[2-hydroxyethyl(dimethyl)ammonio]propoxy]-6-methoxy-1,3-benzothiazol-2-yl]methylcarbamoyl]indan-2-yl]acetate;
   and pharmaceutically acceptable salts thereof.

### Synthesis

Various synthetic routes can be used to product the compounds of Formula (I) described herein. Some exemplary routes are set out below:

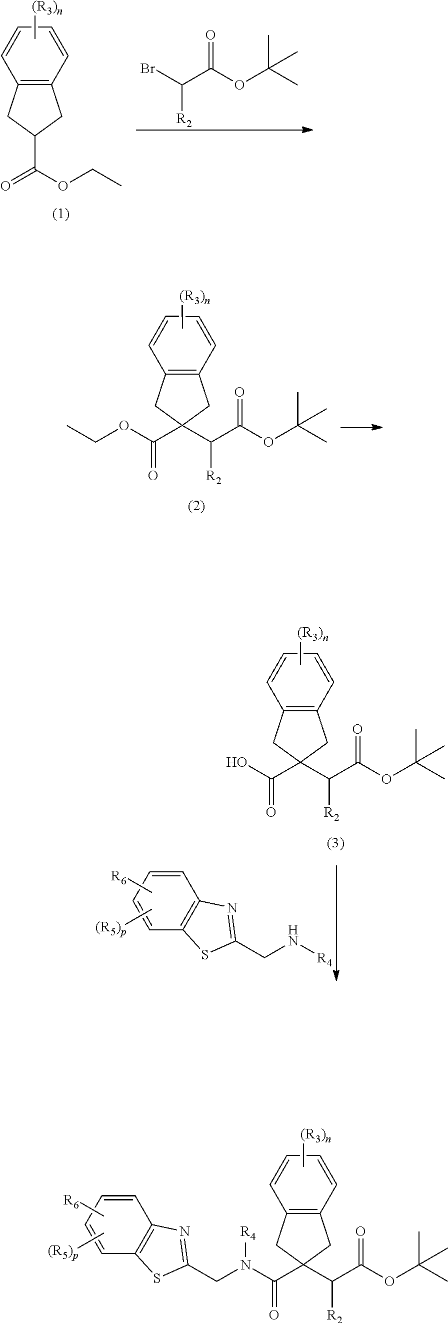

The compounds of Formula (I) can be prepared by any suitable method. For example, as described in more detail below, deprotonation of commercially available ethyl esters (1) with strong base (such as sodium hexamethyldisilazide) then alkylation of the anion with tert-butyl bromoacetates gives diester (2) (Bell, I. M. and Stump, C. A., WO2006/29153; Robinson, R. P. et al, Bioorganic and Medicinal Chemistry Letters, 1996, 1719). Basic hydrolysis of the ethyl ester in the presence of the tert-butyl ester gives (3). Amide formation with a suitable 2-aminomethyl benzothiazole followed by treatment with TFA to remove the tert-butyl ester then affords the desired acids. For compounds of Formula (I) in which ring (A) is other than benzothiazole, corresponding amino-functionalised reagents can be used. Examples of suitable protocols for formation of amino-methyl benzothiazoles (4) are provided below. For example, substituents G (specifically R₅ and R₆) can be introduced by derivatization of commercially available halo-substituted thiazoles (e.g. by halo displacement) or OH-substituted thiazoles (e.g. by alkylation at the hydroxy position). The acids can be converted to esters (R¹═OR^{1a}) or other prodrug forms (R¹═OCH₂OC(O)R^{1a}) by techniques known to the skilled person.

There are numerous ways of accessing hydroxamic acids (for a review see Ganeshpurkar, A., et al, Current Organic Syntheses, 2018, 15, 154-165) but a very reliable procedure is to couple acids with O-(oxan-2-yl)hydroxylamine using peptide coupling conditions to give protected hydroxamates then deprotect with TFA to generate the hydroxamic acids (see for example Ding, C., et al, Bioorg. Med. Chem. Lett, 2017, 25, 27-37).

Some methods that are specifically suitable for synthesising compounds of Formula (I) include the following.

### Method A. Non-Regioselective Ring Opening of Lactone (2)

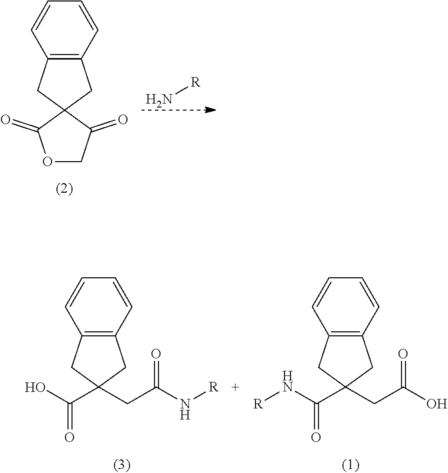

Lactone (2) is commercially available from suppliers such as Enamine and is also readily prepared from cyclisation of the corresponding commercially-available diacid with acetyl chloride (Bell, I. M. and Stump, C. A., WO 2006/29153). Reaction of (2) with amines gives a regioisomeric mixture of major isomer (3) and minor isomer (1) which are separable by standard column chromatography.

### Method B. Regioselective Synthesis of Key Intermediate (4)

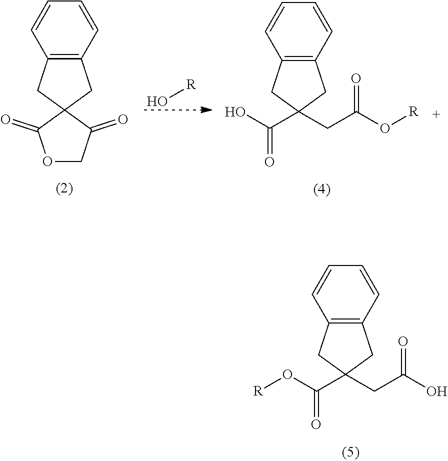

Lactone (2) can be reacted with alcohols ROH to give a regioisomeric mixture of major isomer (4) and minor isomer (5) which are separable by standard column chromatography or recrystallization. Suitable alcohols are benzyl alcohol or 3,4-dimethoxybenzyl alcohol.

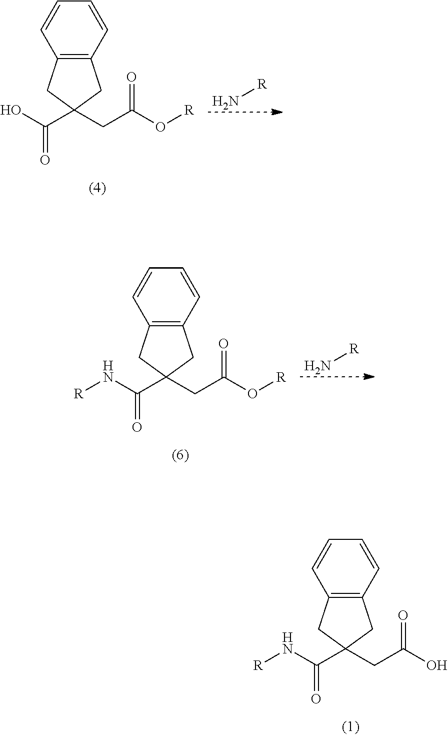

Amide formation can be performed on pure acid (4) by standard amide coupling methods to give (6) followed by ester removal for example by hydrogenation (for benzyl ester) or trifluoroacetic acid treatment (for 3,4-dimethoxybenzyl ester) then gives amide (1).

### Method C. Regiospecific Synthesis of Key Intermediate (4)

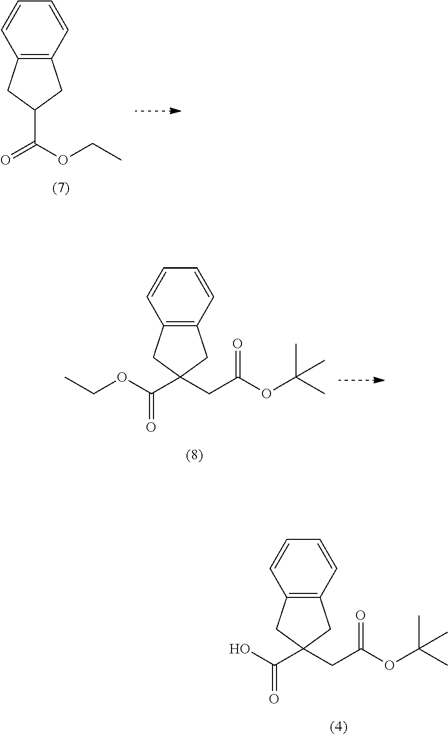 [SMILES: CCOC(=O)C1Cc2ccccc2C1 ; CCOC(=O)C1(CC(=O)OC(C)(C)C)Cc2ccccc2C1 ; CC(C)(C)OC(=O)CC1(C(=O)O)Cc2ccccc2C1]

Deprotonation of commercially available ethyl ester (7) with strong base (such as sodium hexamethyldisilazide) then alkylation of the anion with tert-butyl bromoacetate gives known diester (8) (Bell, I. M. and Stump, C. A., WO2006/29153; Robinson, R. P. et al, Bioorganic and Medicinal Chemistry Letters, 1996, 1719). Basic hydrolysis of the ethyl ester in the presence of the tert-butyl ester gives (4) where R=tert-butyl. Amide formation and treatment with TFA to remove the tert-butyl ester then affords the desired acid (1).

### Method D. Synthesis of 4-Substituted Benzothiazole (13)

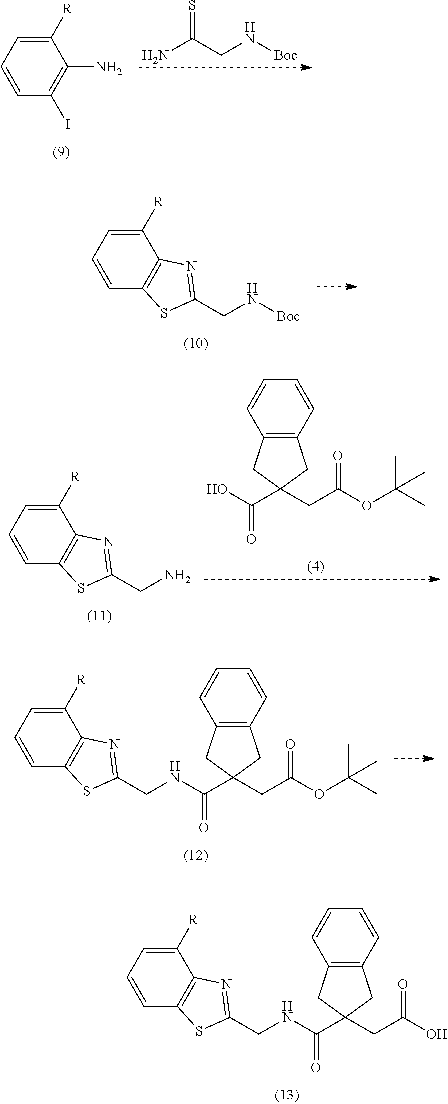

Palladium catalysed cyclisation of commercial aniline (9) with Boc-protected thioamide glycine derivative followed by Boc deprotection affords primary amine (11) Amide formation and treatment with TFA to remove the tert-butyl ester then affords the desired acid (13).

### Method E. Coupling Reactions to Access 5- or 6-Substituted Benzothiazole Derivatives

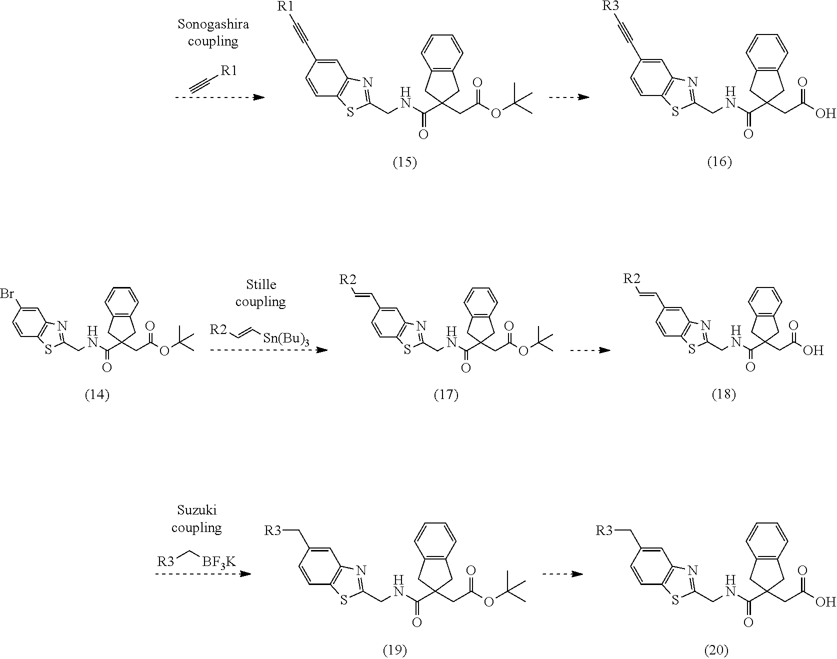

Note that this method has been used to prepare 5- and 6- substituted benzothiazole compounds.

Intermediate (14) is obtained following Method D starting with 5-bromo-2-iodo-aniline (or 4-bromo-2-iodo-aniline for 6-substituted benzothiazole). Sonogashira, Stille or Suzuki coupling reactions afford (15), (17) and (18) which after treatment with TFA and removal of the tert-butyl ester give the corresponding carboxylic acids (16), (18) and (20).

### Method F. Chan-Lam Coupling Reactions to Access 5- or 6-Substituted Benzothiazole Derivative (24)

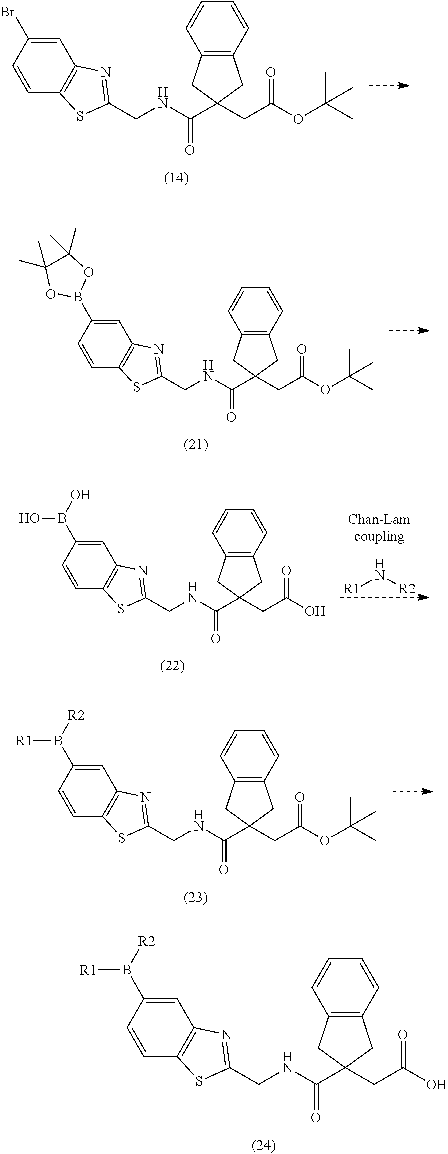

Note that this method has been used to prepare 5- and 6- substituted benzothiazole compounds. Boronic acid (22) is obtained through the formation of the boronate ester analogue of intermediate (14) followed by hydrolysis. Chan-Lam coupling with primary or secondary amine and treatment with TFA then affords the desired carboxylic acids (24).

### Method G. Coupling Reactions Described in Method E and F Before Peptide Coupling

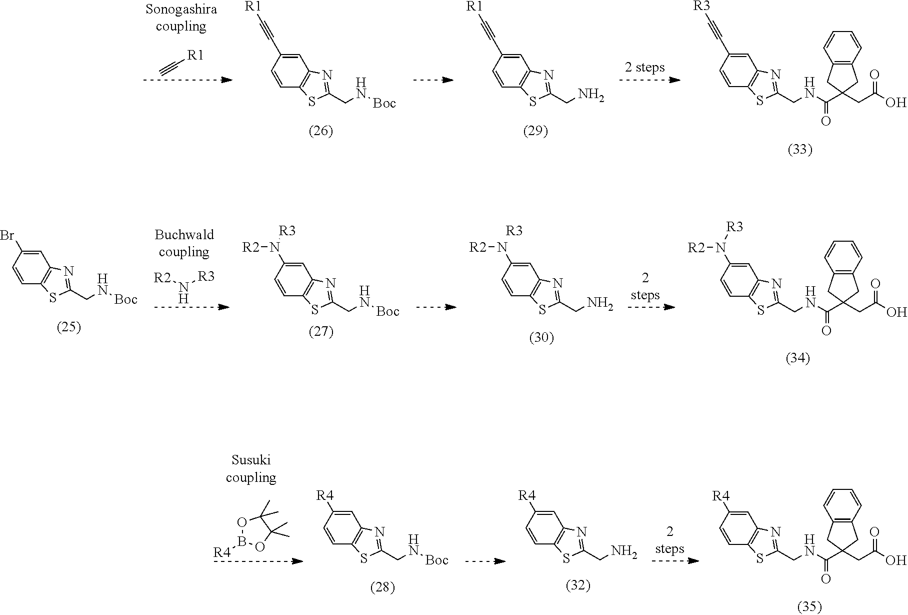

Note that this method has been used to prepare 5- and 6- substituted benzothiazole compounds. This alternative method is based on the introduction of substituents in 5- or 6- position of the benzothiazole before the peptide coupling step. Intermediate (25) is obtained following method D above starting with 5-bromo-2-iodo-aniline (or 4-bromo-2-iodo-aniline for 6-substituted benzothiazole). Sonogashira, Buchwald or Suzuki coupling reactions afford (26), (27) and (28) respectively. Removal of the Boc protection with TFA affords the primary amines (29), (30) and (31) which lead to carboxylic acids (33), (34) and (35) following method D.

### Method H. Buchwald Coupling on Carboxylic Acid Scaffold

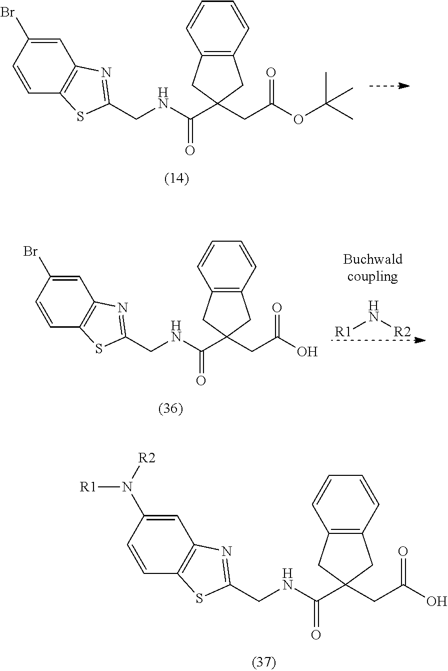

Note that this method has been used to prepare 5- and 6- substituted benzothiazole compounds. 5-substituted benzothiazoles (37) are obtained after Buchwald coupling on 5-bromo benzothiazole carboxylic acid intermediate (36) which is obtained after treatment with TFA and removal of the tert-butyl ester of (14).

### Combinations

As explained herein, the present invention provides a combination comprising (i) a compound which is an indane according to Formula (I) or a pharmaceutically acceptable salt thereof as defined herein; and (ii) one or more CFTR modulator. Typically, the CFTR used in the invention is selected from CFTR potentiators, CFTR correctors and CFTR amplifiers.

CFTR potentiators preferentially stabilise the CFTR channel in the open form in order to facilitate ion channel movement through the CFTR protein. Examples of CFTR potentiators suitable for use in the invention include ivacaftor (Vertex Pharmaceuticals); PTI-808 (dirocaftor, Proteostasis Therapeutics); QBW251 (Novartis Pharmaceuticals); VX-561/CTP-656 (deuterated ivacaftor, Vertex Pharmaceuticals); and GLPG1837, GLPG2451, GLPG3067 (all Galapogos NV/AbbVie). CFTR correctors typically promote wild-type-like folding of the mutant CFTR protein thereby promoting cellular transport to the cell membrane. Examples of CFTR correctors suitable for use in the invention include lumacaftor (Vertex Pharmaceuticals); tezacaftor (Vertex Pharmaceuticals); VX-445 (elexacaftor), VX-152, VX-121, VX-440 (all Vertex Pharmaceuticals); GLPL2222, GLPG (2737) (both Galapogos NV/AbbVie); FDL169 (Flatley Discovery Lab), and PTI-801 (Proteostasis Therapeutics). CFTR amplifiers typically increase cellular levels of CFTR mRNA leading to increased protein expression. An example of a CFTR amplifier suitable for use in the invention is PTI-428 (Proteostasis Therapeutics). Other CFTR modulators suitable for use in the invention include QR-010 (ProQR Therapeutics), MRT5005 (Translate Bio), and ELX-02 (Eloxx Pharmaceuticals).

Preferably, in the invention, the one or more CFTR modulator(s) are selected from ivacaftor, lumacaftor, tezacaftor, elexacaftor, VX659, VX152 and VX-440 and combinations thereof.

Combinations of the above CFTR modulators are within the scope of the invention. The combination of the invention may comprise a combination of any of the above CFTR modulators. For example, the combination may comprise one or more CFTR potentiator(s) and one or more CFTR corrector(s). Preferred combinations of CFTR modulators include elexacaftor/tezacaftor/ivacaftor (“Trikafta”), lumacaftor/ivacaftor (“Orkambi”) and tezacaftor/ivacaftor (“Symdeko”).

### Compositions

The invention also provides a pharmaceutical composition, the pharmaceutical composition comprising (i) a compound which is an indane according to Formula (I) or a pharmaceutically acceptable salt thereof as defined herein, (ii) one or more CFTR modulator as described herein; and (iii) one or more pharmaceutically acceptable excipient, carrier or diluent.

Typically, the composition provided herein contains up to 85 wt % of a compound of Formula (I). More typically, it contains up to 50 wt % of a compound of Formula (I). Sometimes, the composition provided herein contains a total of up to 85 wt % of the compound of Formula (I) and the one or more CFTR modulator(s). Sometimes, the composition provided herein contains a total of up to 50 wt % of the compound of Formula (I) and the one or more CFTR modulator(s).

Preferred pharmaceutical compositions are sterile and pyrogen free. Further, when the pharmaceutical compositions provided by the invention contain a compound of Formula (I) which is optically active, the compound of Formula (I) is typically a substantially pure optical isomer. For the avoidance of doubt, the agent may comprise a compound of Formula (I) in the form of a solvate.

As explained above, the compounds of Formula (I) are useful as inhibitors of LasB, in particular LasB of Pseudomonas aeruginosa (PA). The compounds of Formula (I) thus find use in both enhancing the activity of CFTR modulators when used in the treatment of diseases associated with CFTR downregulation or decreased CFTR function in a subject, such as cystic fibrosis and COPD, preferably cystic fibrosis; and also in treating or preventing bacterial infection. This dual action is an advantage of the present invention because, as explained above, individuals suffering from cystic fibrosis often also suffer from chronic bacterial infection, such as Pseudomonas (e.g. P. aeruginosa) infection.

Accordingly, a combination or pharmaceutical composition of the invention may therefore further comprise an antibiotic agent. This can be beneficial in fighting a bacterial infection thus reducing the extent of LasB expression and CFTR degradation. Preferably, the antibiotic agent is efficacious against Pseudomonas infection. When an antibiotic agent is present, the antibiotic agent is preferably selected from tobramycin, neomycin, streptomycin, gentamycin, ceftazidime, ticarcillin, piperacillin, tazobactam, imipenem, meropenem, rifampicin, ciprofloxacin, amikacin, colistin, aztreonam, azithromycin, levofloxacin and SPR206 (Spero Therapeutics). More preferably, the antibiotic is tobramycin, neomycin, streptomycin, gentamycin, ceftazidime, ticarcillin, piperacillin, tazobactam, imipenem, meropenem, rifampicin, ciprofloxacin, amikacin, colistin, aztreonam, levofloxacin or SPR206 (Spero Therapeutics). Meropenem is particularly preferred.

The invention also provides a product containing (i) a compound which is an indane of Formula (I) or a pharmaceutically acceptable salt thereof and (ii) one or more CFTR modulator, as a combined preparation for simultaneous, separate or sequential use in the treatment of a disease associated with CFTR downregulation or decreased CFTR function in a subject, such as cystic fibrosis. The product may further comprise an antibiotic agent, for example an antibiotic agent as defined herein.

The compound of Formula (I) and the one or more CFTR modulator(s), and the antibiotic agent if present, may be provided in a single formulation, or they may be separately formulated. For example, the compound of Formula (I) may be formulated with one or more CFTR modulators and an antibiotic agent if present may be present in a separate formulation. Alternatively, the compound of Formula (I) may be formulated with an antibiotic agent if present and the one or more CFTR modulators may be present in a separate formulation. Where separately formulated, the agents may be administered simultaneously or separately.

The combination or composition of the invention may be provided as a kit comprising instructions to enable the kit to be used in the methods described herein and/or details regarding which subjects the method may be used for. The kit may for example comprise separate containers comprising (i) a compound of Formula (I) and (ii) one or more CFTR modulators, respectively. If an antibiotic is present in the kit, it may be provided in a separate container, or in the same container as the compound of Formula (I) and/or the CFTR modulator(s).

The combination or composition provided herein may be administered in a variety of dosage forms.

Thus, they can be administered orally, for example as tablets, troches, lozenges, aqueous or oily suspensions, dispersible powders or granules. They may also be administered parenterally, whether subcutaneously, intravenously, intramuscularly, intrasternally, transdermally or by infusion techniques. The combination or composition may also be administered as a suppository.

Preferably, the combination or composition may be administered via inhaled (aerosolised) or intravenous administration, most preferably by inhaled (aerosolised) administration.

The combination or composition is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

The combination or composition may be formulated for inhaled (aerosolised) administration as a solution or suspension. The combination or composition may be administered by a metered dose inhaler (MDI) or a nebulizer such as an electronic or jet nebulizer. Alternatively, the combination or composition may be formulated for inhaled administration as a powdered drug, such formulations may be administered from a dry powder inhaler (DPI). When formulated for inhaled administration, the combination or composition may be delivered in the form of particles which have a mass median aerodynamic diameter (MMAD) of from 1 to 100 μm, preferably from 1 to 50 μm, more preferably from 1 to 20 μm such as from 3 to 10 μm, e.g. from 4 to 6 μm. When the combination or composition is delivered as a nebulized aerosol, the reference to particle diameters defines the MMAD of the droplets of the aerosol. The MMAD can be measured by any suitable technique such as laser diffraction.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections or inhalation may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used.

It will be appreciated that when the compound of Formula (I) is not formulated together with the one or more CFTR modulator(s), the compound of Formula (I) may be administered by a different administration route to the one or more CFTR modulator(s). When an antibiotic agent is also present, it may likewise be administered by the same or a different administration route to the one or more CFTR modulator(s) and/or the compound of Formula (I). Accordingly, the compound of Formula (I), the one or more CFTR modulator(s) and the antibiotic agent if present may be provided in the same or different dosage forms.

### Therapeutic Efficacy

The combinations and compositions provided herein are therapeutically useful. The present invention therefore provides combinations and compositions as described herein, for use in medicine. The present invention provides combinations and compositions as described herein, for use in treating the human or animal body.

The combinations and compositions provided herein are useful in treating diseases associated with CFTR downregulation or decreased CFTR function. Diseases suitable for treating with the combinations and compositions provided herein include cystic fibrosis and COPD, particularly cystic fibrosis.

In this regard, it has recently been shown by Saint Criq et al (Thorax 2018; 73:49-61, the entire contents of which are hereby incorporated by reference) that secretomes from Pseudomonas aeruginosa can degrade CFTR function, but that this can be ameliorated by trappin-2 and/or IL-6 overexpression. The wild-type (WT) Pseudomonas aeruginosa PAO1 secretome contains LasB.

When secretomes (SEC) from PAO1 WT and ΔLasB strains were used to treat CFTR-expressing cell lines, PAO1-SEC but not ΔLasB-SEC led to decreased measured CFTR. ΔLasB-SEC was also shown to induce IL-6 and IL-8 production compared to IL-1β alone, whereas WT-SEC abolished trappin-2 and IL-6 secretion without effect on IL-8. This downregulation of trappin-2 and IL-6 was prevented by phosphoramidon. IL-6 was separately shown to upregulate trappin-2 secretion. In vivo experiments showed that whilst LasB led to increased mortality in mice, induced overexpression of IL-6 had a protective effect, as did overexpression of trappin-2. The inventors have recognised in view of this activity that CFTR-correction therapies will be hampered by LasB, and have developed the combinations and compositions of the invention accordingly to counter this issue.

Accordingly, the invention provides combinations and compositions as described herein for use in treating a disease associated with CFTR downregulation or decreased CFTR function in a subject in need thereof. Also provided is a method of treating a disease associated with CFTR downregulation or decreased CFTR function in a subject in need thereof, which method comprises administering to said subject an effective amount of a combination or composition as described herein. Further provided is the use of a combination or composition as described herein in the manufacture of a medicament for use in treating a disease associated with CFTR downregulation or decreased CFTR function in a subject.

The invention also provides a compound which is an indane according to Formula (I) or a pharmaceutically acceptable salt thereof as defined herein for use in treating a disease associated with CFTR downregulation or decreased CFTR function in a subject, said use comprising administering said compound in combination with a CFTR modulator to said subject. The invention also provides the use of a compound which is an indane according to Formula (I) or a pharmaceutically acceptable salt thereof as defined herein in the manufacture of a medicament for treating a disease associated with CFTR downregulation or decreased CFTR function in a subject by co-administering the compound together with one or more CFTR modulator. Preferably, the one or more CFTR modulator is as described herein.

Further provided is a CFTR modulator, such as a CFTR modulator described herein, for use in treating a disease associated with CFTR downregulation or decreased CFTR function in a subject, said use comprising administering said CFTR modulator in combination with a compound which is an indane according to Formula (I) or a pharmaceutically acceptable salt thereof as defined herein. Also provided is the use of a CFTR modulator, such as a CFTR modulator described herein, in the manufacture of a medicament for treating a disease associated with CFTR downregulation or decreased CFTR function in a subject by co-administering the CFTR modulator with a compound which is an indane according to Formula (I) or a pharmaceutically acceptable salt thereof as defined herein.

Still further provided is a method of treating a disease associated with CFTR downregulation or decreased CFTR function in a subject in need thereof, which method comprises administering to said subject an effective amount of a compound which is an indane according to Formula (I) or a pharmaceutically acceptable salt thereof as defined herein and an effective amount of one or more CFTR modulators, preferably one or more CFTR modulators described herein.

The compounds, compositions and combinations described herein find particular therapeutic use in treating cystic fibrosis (CF) or chronic obstructive pulmonary disease, particularly cystic fibrosis. Any genotype of cystic fibrosis may be addressed with the therapies provided herein. A subject may have any of the six classes of CFTR mutation. Such classes include: class 1A (mutations which prevent CFTR mRNA from being synthesized, e.g. Dele2,3(21 kB) and 1717-1G→A; class 1B (mutations which prevent CFTR mRNA from forming CFTR protein, e.g. Gly542X and Trp1282X); class 2 (causing CFTR protein misfolding and failed transport to the cell membrane, e.g. F508del, N1303K and A561E); class 3 (gating defect mutations, e.g. G551D, S549R and G1349D); class 4 (decreased conductance mutations, e.g. R117H, R334W and A455E); class 5 (alternative splicing mutations, e.g. 3272-26A4G); and class 6 (instability mutations, such as c.120del123 and rPhe580del). Common CFTR mutations include F508del; G178R, G1244E, S549R, G551D, G1349D, S1251N, G551S, S549N, S1255P, A455E, E193K, R117C, A1067T, F1052V, R347H, D110E, F1074L, R352Q, D110H, G1069R, R1070Q, D579G, K1060T, R1070W, D1152H, L206W, S945L, D1270N, P67L, S977F, E56K, R74W, E831X and R117H.

Sometimes, the subject is a smoker. Sometimes, the subject has been exposed to cigarette smoke or other environmental pollution. Sometimes, the subject is a CFTR-sufficient smoker. Such subjects are particularly suited to treatment in accordance with the invention when the a disease associated with CFTR downregulation or decreased CFTR function is COPD.

Because the compounds of Formula (I) are inhibitors of LasB, in particular LasB of Pseudomonas aeruginosa (PA), the compositions and combinations provided herein may also be used in treating bacterial infection in a subject. Accordingly, the invention also provides a combination or composition as described herein for use in a method of treating or preventing bacterial infection, optionally by co-administration with an antibiotic agent. Also provided is a method for treating or preventing bacterial infection in a subject in need thereof, which method comprises administering to said subject an effective amount of a composition or combination described herein and optionally an antibiotic agent. Also provided is the use of a composition or combination as described herein in the manufacture of a medicament for use in treating or preventing bacterial infection, optionally by co-administration with an antibiotic agent.

The bacterium causing the infection may be any bacterium expressing LasB or an analogue thereof. Typically the bacterium causing the infection expresses LasB. The bacterium may, for instance, be any bacterium that can form a biofilm. In a preferred instance the bacterium is Gram-negative. The bacterium may in particular be a pathogenic bacterium. The bacterial infection may be caused by Pseudomonas or Actinetobacter. For example, the bacterium may be one selected from Pseudomonas aeruginosa and Actinetobater bauminii.

Preferably the bacterium is a Pseudomonas, particularly where the condition to be treated is pneumonia, and most preferably, the bacterium is Pseudomonas aeruginosa (PA).

As will be appreciated, when used to treat bacterial infection, the combinations and compositions provided herein are particularly useful when the subject suffers from cystic fibrosis.

As well as finding utility in treating cystic fibrosis, the compositions and combination provided herein may be used to treat or prevent infections and conditions caused by any one or a combination of the above-mentioned bacteria. In particular, the compound or combination of the invention may be used in the treatment or prevention of pneumonia. The compound or combination may also be used in the treatment of septic shock, urinary tract infection, and infections of the gastrointestinal tract, skin or soft tissue. They may also be used to treat or prevent inflammation in a subject. Without being bound by theory, such utility is believed to arise from the activity of the compounds to inhibit the activation of the pro-inflammatory cytokine interleukin-1-f (IL-1β), e.g. by inhibiting activity of LasB enzymes (such as PA LasB) to activate IL-1β by hydrolysis of pro-IL-1β at a distinct site from caspase-1. Accordingly, the compositions and combinations described herein are particularly suitable for treating inflammation caused by or associated with IL-1β activation in a subject. The compositions and combinations described herein are especially suitable in treating or preventing respiratory tract inflammation in a subject. The respiratory tract inflammation may be inflammation of any part of the respiratory tract, in particular the lower respiratory tract (e.g. inflammation of the trachea, bronchi or lungs). The combinations and compositions described herein are particularly suited to treating or preventing pulmonary inflammation in a subject. The respiratory tract inflammation (e.g. pulmonary inflammation) is typically caused by a bacterial infection, especially by an infection caused by bacteria which express one or more LasB enzymes or analogs thereof, as described above. In some aspects the respiratory tract inflammation (e.g. pulmonary inflammation) is caused by an infection caused by a bacterium of the family Pseudomonadaceae, such as a Pseudomonas aeruginosa (PA) infection.

Again, it will apparent that such treatments are particularly suited to subjects suffering from COPD or cystic fibrosis, particularly cystic fibrosis.

In one aspect, the subject is a mammal, in particular a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters. The subject can be any animal that is capable of suffering from diseases associated with CFTR downregulation or decreased CFTR function.

A compound, combination or composition provided herein can be administered to the subject in order to prevent the onset or reoccurrence of one or more symptoms of a condition as described herein. This is prophylaxis. When used to treat a subject with a disease associated with CFTR downregulation or decreased CFTR function the subject may be asymptomatic. A prophylactically effective amount of the agent or formulation is administered to such a subject. A prophylactically effective amount is an amount which prevents the onset of one or more symptoms of the disease.

A compound, combination or composition provided herein can be administered to the subject in order to treat one or more symptoms of the disease associated with CFTR downregulation or decreased CFTR function. In this embodiment, the subject is typically symptomatic. A therapeutically effective amount of the agent or formulation is administered to such a subject. A therapeutically effective amount is an amount effective to ameliorate one or more symptoms of the disorder.

A therapeutically or prophylactically effective amount of the compound, combination or composition provided herein is administered to a subject. The dose may be determined according to various parameters, especially according to the compound used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific agent, the age, weight and conditions of the subject to be treated, the type and severity of the disease and the frequency and route of administration. Preferably, daily dosage levels are from 5 mg to 2 g.

The dosage of the indane of Formula (I) or pharmaceutically acceptable salt thereof may be the same or different to the dosage of the one or more CFTR modulator. When two or more CFTR modulators are used, the dosage of the two or more CFTR modulators can be the same or different. Each active agent administered to the subject is typically administered in an independently determined amount of from about 0.01 to 100 mg per kg, preferably from about 0.1 mg/kg to 50 mg/kg, e.g. from about 1 to 10 mg/kg of body weight, according to the activity of the specific agent, the age, weight and conditions of the subject to be treated, the type and severity of the disease and the frequency and route of administration. Preferably, daily dosage levels are from 5 mg to 2 g.

### Genetic Therapies

As explained herein, the present inventors have found that inhibitors of LasB prevent the LasB-mediated degradation of CFTR. Accordingly, such inhibitors, e.g. compounds of Formula (I) and pharmaceutically acceptable salts thereof, can be used to improve the efficacy of therapies intended to restore CFTR function at the genetic level. At present, such therapies are limited not least by the fact that even restored CFTR protein remains susceptible to LasB-mediated degradation. Without being bound by theory, the inventors understand that by inhibiting the activity of LasB to degrade CFTR or to downregulate its expression, genetic therapies intended to restore a WT-like level of CFTR function in an individual can be improved.

Accordingly, the invention provides a compound which is an indane according to Formula (I) or a pharmaceutically acceptable salt thereof as defined herein for use in treating a disease associated with CFTR downregulation or decreased CFTR function in a subject receiving a genetic therapy for said disease. The invention also provides a method of treating a disease associated with CFTR downregulation or decreased CFTR function in a subject receiving a genetic therapy for said disease, said method comprising administering to said subject a therapeutically effective amount of a compound which is an indane according to Formula (I) or a pharmaceutically acceptable salt thereof. Further provided is the use of a compound which is an indane according to Formula (I) or a pharmaceutically acceptable salt thereof in the manufacture of a medicament for treating a disease associated with CFTR downregulation or decreased CFTR function in a subject receiving a genetic therapy for said disease.

Typically, the genetic therapy received by the subject is selected from an integrating gene therapy; a non-integrating gene therapy; and an RNA therapy.

Integrating gene therapies comprise delivering DNA for encoding WT CFTR (or WT-like) to a subject. The DNA becomes integrated in the individual's genome and thus leads to synthesis of the correct CFTR protein thus ameliorating the effect of the native mutant CFTR expression. Integrating genetic therapies for genetic conditions are known in the art, such as CAR-T therapies for leukemia and lymphoma. Non-integrating gene therapies comprise delivering DNA for encoding WT CFTR (or WT-like CFTR) to a subject, but such that the exogenous DNA does not become incorporated into the subject's genome. Non-integrating gene-therapies for CFTR have been described in, for example, Alton, Armstrong, Ashby, et. al. “Repeated nebulization of non-viral CFTR gene therapy in patients with cystic fibrosis: a randomized, double-blind, placebo-controlled, phase 2b trial”. Lancet. 3(9) P684-691 (2015). RNA therapies for CF comprise administering to individuals suffering from CF mRNA encoding WT (or WT-like) CFTR for translation into functional CFTR. RNA therapies typically do not comprise altering a patient's genome and are thus in some circumstances preferable. mRNA therapies for treating cystic fibrosis include MRT5005 administered in nebulised form as demonstrated in the RESTORE clinical trials described at https://clinicaltrials.gov/ct2/show/NCT03375047.

Typically, therefore, in the invention, said genetic therapy comprises:
i) administering DNA for encoding a CFTR protein to said patient; and/or
ii) administering to said patient a CFTR-encoding mRNA.

Preferably, when the genetic therapy comprises administering DNA to said patient, said DNA is administered as a plasmid containing a gene for expressing CFTR. Said plasmid is preferably attached to or encapsulated in one or more liposomes. For example, said plasmid may be attached to or encapsulated in one or more cationic liposomes.

Preferably, when the genetic therapy comprises administering to said patient a CFTR-encoding mRNA; said mRNA is attached to or encapsulated in one or more nanoparticles.

Preferably, the administration of the DNA and/or mRNA is via inhalation.

### EXAMPLES

The compounds of Formula (I) disclosed herein are synthetically accessible to those skilled in the art.

For example, the detailed synthesis and LasB inhibitory activity of compounds such as 2-(2-{1[(5-chloro-1H-1,3-benzodiazol-2-yl)methyl]carbamoyl}-2,3-dihydro-1H-inden-2-yl)acetic acid; 2-[2-(3-isoquinolylmethylcarbamoyl)indan-2-yl]acetic acid; 2-[2-[(1-methylpyrazol-4-yl)methylcarbamoyl]indan-2-yl]acetic acid; 2-[2-[(5-methoxy-1H-benzimidazol-2-yl)methylcarbamoyl]indan-2-yl]acetic acid; 2-(2-{[(1-benzofuran-2-yl)methyl]carbamoyl}-2,3-dihydro-1H-inden-2-yl)acetic acid; 2-(2-{[(5-methyl-1-benzofuran-2-yl)methyl]carbamoyl}-2,3-dihydro-1H-inden-2-yl)acetic acid; 2-[2-[(5-methyl-2,3-dihydrobenzofuran-2-yl)methylcarbamoyl]indan-2-yl]acetic acid; 2-[2-[[1-(3-chlorophenyl)pyrrolidin-3-yl]carbamoyl]indan-2-yl]acetic acid; 2-[2-[(1-methylindol-2-yl)methylcarbamoyl]indan-2-yl]acetic acid; 2-(2-{[(1,3-benzothiazol-2-yl)methyl]carbamoyl}-2,3-dihydro-1H-inden-2-yl)acetic acid; 2-(2-{[2-(3-methyl-1-benzofuran-2-yl)methyl]carbamoyl}-2,3-dihydro-1H-inden-2-yl)acetic acid; 2-[2-[2-(1H-benzimidazol-2-yl)ethylcarbamoyl]indan-2-yl]acetic acid; 2-[2-[2-(4-hydroxyphenyl)ethylcarbamoyl]indan-2-yl]acetic acid; 2-[2-[2-(1H-indol-3-yl)ethylcarbamoyl]indan-2-yl]acetic acid; 2-[2-[(4-hydroxyphenyl)methylcarbamoyl]indan-2-yl]acetic acid; 2-[2-(benzylcarbamoyl)indan-2-yl]acetic acid; 2-[2-(2,3-dihydro-1,4-benzodioxin-3-ylmethylcarbamoyl)indan-2-yl]acetic acid; 2-[2-[(5-bromobenzofuran-2-yl)methylcarbamoyl]indan-2-yl]acetic acid; 2-[2-[2-(benzofuran-2-yl)ethylcarbamoyl]indan-2-yl]acetic acid; 2-[2-[(2-methylbenzofuran-3-yl)methylcarbamoyl]indan-2-yl]acetic acid; 2-[2-[(1-methylimidazol-4-yl)methylcarbamoyl]indan-2-yl]acetic acid; 2-[2-[2-(1,3-benzothiazol-2-yl)ethylcarbamoyl]indan-2-yl]acetic acid; 2-[2-(benzothiophen-2-ylmethylcarbamoyl)indan-2-yl]acetic acid; 2-[2-[(5-methoxy-1,3-benzothiazol-2-yl)methylcarbamoyl]indan-2-yl]acetic acid; 2-[2-[(5-chloro-1,3-benzothiazol-2-yl)methylcarbamoyl]indan-2-yl]acetic acid; 2-[2-(1H-indol-3-ylmethylcarbamoyl)indan-2-yl]acetic acid; 2-[2-[2-(1H-indol-2-yl)ethylcarbamoyl]indan-2-yl]acetic acid; 2-[2-(benzofuran-3-ylmethylcarbamoyl)indan-2-yl]acetic acid; 2-[2-(1H-indol-2-ylmethylcarbamoyl)indan-2-yl]acetic acid; 2-[2-[2-(benzothiophen-2-yl)ethylcarbamoyl]indan-2-yl]acetic acid; 2-[2-[2-(1,3-benzoxazol-2-yl)ethylcarbamoyl]indan-2-yl]acetic acid; 2-[2-[[6-(3-aminopropyl)-1,3-benzothiazol-2-yl]methylcarbamoyl]indan-2-yl]acetic acid; 2-[2-[(6-methoxy-1,3-benzothiazol-2-yl)methylcarbamoyl]indan-2-yl]acetic acid; 2-[2-(4,5,6,7-tetrahydrothiazolo[5,4-c]pyridin-2-ylmethylcarbamoyl)indan-2-yl]acetic acid; (2-{[(2S)-1-hydroxy-3-(1H-indol-3-yl)propan-2-yl]carbamoyl}-1,3-dihydroinden-2-yl)acetic acid; (2-{[(1S)-1-{[2-(dimethylamino)ethyl]carbamoyl}-2-(1H-indol-3-yl)ethyl]carbamoyl}-1,3-dihydroinden-2-yl)acetic acid; 2-(2-{[(2S)-3-(1H-indol-3-yl)-1-[(1-methylpiperidin-4-yl)oxy]-1-oxopropan-2-yl]carbamoyl}-2,3-dihydro-1H-inden-2-yl)acetic acid; (2-{[(1S)-2-(1H-indol-3-yl)-1-{[2-(trimethylammonio)ethyl]carbamoyl}ethyl]carbamoyl}-1,3-dihydroinden-2-yl)acetate 2-[2-[[6-[3-(dimethylamino)prop-1-ynyl]-1,3-benzothiazol-2-yl]methylcarbamoyl]indan-2-yl]acetic acid; 2-[2-[[6-[3-(dimethylamino)propyl]-1,3-benzothiazol-2-yl]methylcarbamoyl]indan-2-yl]acetic acid; 3-[2-[[[2-(carboxymethyl)indane-2-carbonyl]amino]methyl]-1,3-benzothiazol-6-yl]propyl-trimethyl-ammonium; 2-[2-[[6-[(E)-3-aminoprop-1-enyl]-1,3-benzothiazol-2-yl]methylcarbamoyl]indan-2-yl]acetic acid; 2-[2-[[5-(3-aminopropyl)-1,3-benzothiazol-2-yl]methylcarbamoyl]indan-2-yl]acetic acid; 2-[2-[(6-piperazin-1-yl-1,3-benzothiazol-2-yl)methylcarbamoyl]indan-2-yl]acetic acid; 2-[2-[[6-(4-methylpiperazin-1-yl)-1,3-benzothiazol-2-yl]methylcarbamoyl]indan-2-yl]acetic acid; 2-[2-[[6-(4,4-dimethylpiperazin-4-ium-1-yl)-1,3-benzothiazol-2-yl]methylcarbamoyl]indan-2-yl]acetic acid; (S)-2-(2-((1-(tert-butoxy)-3-(1H-indol-3-yl)-1-oxopropan-2-yl)carbamoyl)-2,3-dihydro-1H-inden-2-yl)acetic acid; (S)-2-(2-((1-amino-3-(4-hydroxyphenyl)-1-oxopropan-2-yl)carbamoyl)-2,3-dihydro-1H-inden-2-yl)acetic acid; (2-(carboxymethyl)-2,3-dihydro-1H-indene-2-carbonyl)-L-tyrosine; (2-(carboxymethyl)-2,3-dihydro-1H-indene-2-carbonyl)-L-tryptophan; 2-(2-(((1H-benzo[d]imidazol-2-yl)methyl)carbamoyl)-2,3-dihydro-1H-inden-2-yl)acetic acid; (S)-2-(2-((1-amino-3-(1H-indol-3-yl)-1-oxopropan-2-yl)carbamoyl)-2,3-dihydro-1H-inden-2-yl)acetic acid; (S)-2-(2-((3-(1H-indol-3-yl)-1-methoxy-1-oxopropan-2-yl)carbamoyl)-2,3-dihydro-1H-inden-2-yl)acetic acid; 2-(2-((thiazol-2-ylmethyl)carbamoyl)-2,3-dihydro-1H-inden-2-yl)acetic acid; 2-(2-((quinolin-2-ylmethyl)carbamoyl)-2,3-dihydro-1H-inden-2-yl)acetic acid; 2-(2-(((1-methyl-1H-benzo[d]imidazol-2-yl)methyl)carbamoyl)-2,3-dihydro-1H-inden-2-yl)acetic acid; 2-[2-(benzofuran-3-ylmethylcarbamoyl)indan-2-yl]acetic acid; 2-[2-[[(1R*)-1-(benzofuran-2-yl)ethyl]carbamoyl]indan-2-yl]acetic acid; 2-[2-[[(1S*)-1-(benzofuran-2-yl)ethyl]carbamoyl]indan-2-yl]acetic acid; 2-(2-(((4-fluorobenzo[d]thiazol-2-yl)methyl)carbamoyl)-2,3-dihydro-1H-inden-2-yl)acetic acid; 2-(2-(((4-bromobenzo[d]thiazol-2-yl)methyl)carbamoyl)-2,3-dihydro-1H-inden-2-yl)acetic acid; 2-(2-(((4-methoxybenzo[d]thiazol-2-yl)methyl)carbamoyl)-2,3-dihydro-1H-inden-2-yl)acetic acid; 2-(2-(((4-iodobenzo[d]thiazol-2-yl)methyl)carbamoyl)-2,3-dihydro-1H-inden-2-yl)acetic acid; 2-(2-(((4-ethoxybenzo[d]thiazol-2-yl)methyl)carbamoyl)-2,3-dihydro-1H-inden-2-yl)acetic acid; 2-(2-(((4-methylbenzo[d]thiazol-2-yl)methyl)carbamoyl)-2,3-dihydro-1H-inden-2-yl)acetic acid; 2-(2-(((4-morpholinobenzo[d]thiazol-2-yl)methyl)carbamoyl)-2,3-dihydro-1H-inden-2-yl)acetic acid; 2-(2-(((6-(3-(dimethylamino)azetidin-1-yl)benzo[d]thiazol-2-yl)methyl)carbamoyl)-2,3-dihydro-1H-inden-2-yl)acetic acid; 2-[2-[[6-[3-(trimethylammonio)azetidin-1-yl]-1,3-benzothiazol-2-yl]methylcarbamoyl]indan-2-yl]acetate; 2-[2-[[6-(2-aminoethylamino)-1,3-benzothiazol-2-yl]methylcarbamoyl]indan-2-yl]acetic acid; 2-[2-[[6-[3-(2-aminoethyl)azetidin-1-yl]-1,3-benzothiazol-2-yl]methylcarbamoyl]indan-2-yl]acetic acid; 2-[2-[[6-(6-methyl-2,6-diazaspiro[3.3]heptan-2-yl)-1,3-benzothiazol-2-yl]methylcarbamoyl]indan-2-yl]acetic acid; 2-[2-[[5-(2-aminoethylamino)-1,3-benzothiazol-2-yl]methylcarbamoyl]indan-2-yl]acetic acid; 2-[2-[[5-(3-aminoazetidin-1-yl)-1,3-benzothiazol-2-yl]methylcarbamoyl]indan-2-yl]acetic acid; 2-[2-[[5-[3-(dimethylamino)azetidin-1-yl]-1,3-benzothiazol-2-yl]methylcarbamoyl]indan-2-yl]acetic acid; 2-[2-[[5-[3-(trimethylammonio)azetidin-1-yl]-1,3-benzothiazol-2-yl]methylcarbamoyl]indan-2-yl]acetate; 2-[2-[[5-[2-(dimethylamino)ethylamino]-1,3-benzothiazol-2-yl]methylcarbamoyl]indan-2-yl]acetic acid; 2-[2-[[5-[2-(trimethylammonio)ethylamino]-1,3-benzothiazol-2-yl]methylcarbamoyl]indan-2-yl]acetate; 2-[2-[[6-(3-aminocyclobutoxy)-1,3-benzothiazol-2-yl]methylcarbamoyl]indan-2-yl]acetic acid; 2-[2-[[6-[3-(dimethylamino)cyclobutoxy]-1,3-benzothiazol-2-yl]methylcarbamoyl]indan-2-yl]acetic acid; 2-[2-[[6-(2-aminoethyl)-1,3-benzothiazol-2-yl]methylcarbamoyl]indan-2-yl]acetic acid; 2-[2-[[6-[(dimethylamino)methyl]-1,3-benzothiazol-2-yl]methylcarbamoyl]indan-2-yl]acetic acid; 2-[2-[[6-[(trimethylammonio)methyl]-1,3-benzothiazol-2-yl]methylcarbamoyl]indan-2-yl]acetate; 2-[2-[[5-(2-aminoethyl)-1,3-benzothiazol-2-yl]methylcarbamoyl]indan-2-yl]acetic acid; 2-[2-[[5-[(dimethylamino)methyl]-1,3-benzothiazol-2-yl]methylcarbamoyl]indan-2-yl]acetic acid; 2-[2-[[5-[(trimethylammonio)methyl]-1,3-benzothiazol-2-yl]methylcarbamoyl]indan-2-yl]acetate; 2-[2-[[6-[3-(trimethylammonio)prop-1-ynyl]-1,3-benzothiazol-2-yl]methylcarbamoyl]indan-2-yl]acetate; 2-[2-[[5-[(E)-3-aminoprop-1-enyl]-1,3-benzothiazol-2-yl]methylcarbamoyl]indan-2-yl]acetic acid; 2-[2-[[5-[3-(dimethylamino)prop-1-ynyl]-1,3-benzothiazol-2-yl]methylcarbamoyl]indan-2-yl]acetic acid; 2-[2-[[5-[3-(dimethylamino)propyl]-1,3-benzothiazol-2-yl]methylcarbamoyl]indan-2-yl]acetic acid; 2-[2-[[5-[3-(trimethylammonio)propyl]-1,3-benzothiazol-2-yl]methylcarbamoyl]indan-2-yl]acetate; 2-(2-(((6-((1r,3r)-3-((dimethylamino)methyl)cyclobutyl)benzo[d]thiazol-2-yl)methyl)carbamoyl)-2,3-dihydro-1H-inden-2-yl)acetic acid; 2-[2-[[6-[(1r,3r)-3-[(trimethylammonio)methyl]cyclobutyl]-1,3-benzothiazol-2-yl]methylcarbamoyl]indan-2-yl]acetate; 2-(2-(((6-((1s,3s)-3-((dimethylamino)methyl)cyclobutyl)benzo[d]thiazol-2-yl)methyl)carbamoyl)-2,3-dihydro-1H-inden-2-yl)acetic acid; 2-[2-[[6-[(1s,3s)-3-[(trimethylammonio)methyl]cyclobutyl]-1,3-benzothiazol-2-yl]methylcarbamoyl]indan-2-yl]acetate; 2-[2-[[6-[2-(dimethylamino)ethyl]-1,3-benzothiazol-2-yl]methylcarbamoyl]indan-2-yl]acetic acid; 2-[2-[[6-[2-(trimethylammonio)ethyl]-1,3-benzothiazol-2-yl]methylcarbamoyl]indan-2-yl]acetate; 2-[2-[[5-[2-(dimethylamino)ethyl]-1,3-benzothiazol-2-yl]methylcarbamoyl]indan-2-yl]acetic acid; 2-[2-[[5-[2-(trimethylammonio)ethyl]-1,3-benzothiazol-2-yl]methylcarbamoyl]indan-2-yl]acetate; 2-[2-[[6-[3-(aminomethyl)azetidin-1-yl]-1,3-benzothiazol-2-yl]methylcarbamoyl]indan-2-yl]acetic acid; 2-[2-[[6-(3-aminoazetidin-1-yl)-1,3-benzothiazol-2-yl]methylcarbamoyl]indan-2-yl]acetic acid; 2-[2-[[6-[(3S)-3-aminopyrrolidin-1-yl]-1,3-benzothiazol-2-yl]methylcarbamoyl]indan-2-yl]acetic acid; 2-[2-[[6-[(3R)-3-aminopyrrolidin-1-yl]-1,3-benzothiazol-2-yl]methylcarbamoyl]indan-2-yl]acetic acid; 2-[2-[[5-[(3S)-3-aminopyrrolidin-1-yl]-1,3-benzothiazol-2-yl]methylcarbamoyl]indan-2-yl]acetic acid; 2-[2-[[5-[(3R)-3-aminopyrrolidin-1-yl]-1,3-benzothiazol-2-yl]methylcarbamoyl]indan-2-yl]acetic acid; 2-[2-[[6-[2-(dimethylamino)ethylamino]-1,3-benzothiazol-2-yl]methylcarbamoyl]indan-2-yl]acetic acid; 2-[2-[[5-(4-methylpiperazin-1-yl)-1,3-benzothiazol-2-yl]methylcarbamoyl]indan-2-yl]acetic acid; 2-[2-[(6-hydroxy-1,3-benzothiazol-2-yl)methylcarbamoyl]indan-2-yl]acetic acid; 2-[2-[(6-ethoxy-1,3-benzothiazol-2-yl)methylcarbamoyl]indan-2-yl]acetic acid; 2-[2-[[6-(2-hydroxyethoxy)-1,3-benzothiazol-2-yl]methylcarbamoyl]indan-2-yl]acetic acid; 2-[2-[[6-[2-(dimethylamino)ethoxy]-1,3-benzothiazol-2-yl]methylcarbamoyl]indan-2-yl]acetic acid; 2-[2-[[6-[2-(trimethylammonio)ethoxy]-1,3-benzothiazol-2-yl]methylcarbamoyl]indan-2-yl]acetate; 2-[2-[[5-[2-(dimethylamino)ethoxy]-1,3-benzothiazol-2-yl]methylcarbamoyl]indan-2-yl]acetic acid; 2-[2-[[5-[2-(trimethylammonio)ethoxy]-1,3-benzothiazol-2-yl]methylcarbamoyl]indan-2-yl]acetate; 2-[2-[(5,6-dimethoxy-1,3-benzothiazol-2-yl)methylcarbamoyl]indan-2-yl]acetic acid; 2-[2-([1,3]dioxolo[4,5-f][1,3]benzothiazol-6-ylmethylcarbamoyl)indan-2-yl]acetic acid; (S)-2-(2-((1-((1,1-dimethylpiperidin-1-ium-4-yl)oxy)-3-(1H-indol-3-yl)-1-oxopropan-2-yl)carbamoyl)-2,3-dihydro-1H-inden-2-yl)acetate; (S)-2-(2-((2-(1H-indol-3-yl)-1-(5-methyl-1,3,4-oxadiazol-2-yl)ethyl)carbamoyl)-2,3-dihydro-1H-inden-2-yl)acetic acid; (S)-2-(2-((1-(5-(aminomethyl)-1,3,4-oxadiazol-2-yl)-2-(1H-indol-3-yl)ethyl)carbamoyl)-2,3-dihydro-1H-inden-2-yl)acetic acid; (S)-2-(2-((1-(5-(2-aminopropan-2-yl)-1,3,4-oxadiazol-2-yl)-2-(1H-indol-3-yl)ethyl)carbamoyl)-2,3-dihydro-1H-inden-2-yl)acetic acid; 2-(2-((benzo[d]thiazol-2-ylmethyl)carbamoyl)-5,6-difluoro-2,3-dihydro-1H-inden-2-yl)acetic acid; 2-(2-((benzo[d]thiazol-2-ylmethyl)carbamoyl)-5,6-dichloro-2,3-dihydro-1H-inden-2-yl)acetic acid; 2-(2-(((5-(3-(dimethylamino)azetidin-1-yl)-6-methoxybenzo[d]thiazol-2-yl)methyl)carbamoyl)-2,3-dihydro-1H-inden-2-yl)acetic acid; 2-(2-(((6-methoxy-5-(3-(trimethylammonio)azetidin-1-yl)benzo[d]thiazol-2-yl)methyl)carbamoyl)-2,3-dihydro-1H-inden-2-yl)acetate; 2-(2-(((5-(3-(dimethylamino)propoxy)-6-methoxybenzo[d]thiazol-2-yl)methyl)carbamoyl)-5,6-difluoro-2,3-dihydro-1H-inden-2-yl)acetic acid; 2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate; 2-(2-(((5-(2-(dimethylamino)ethoxy)-6-methoxybenzo[d]thiazol-2-yl)methyl)carbamoyl)-2,3-dihydro-1H-inden-2-yl)acetic acid; 2-[2-(thiazolo[4,5-c]pyridin-2-ylmethylcarbamoyl)indan-2-yl]acetic acid; 2-[2-[[6-(trifluoromethoxy)-1,3-benzothiazol-2-yl]methylcarbamoyl]indan-2-yl]acetic acid; 2-[2-[(5-methyl-6,7-dihydro-4H-thiazolo[5,4-c]pyridin-2-yl)methylcarbamoyl]indan-2-yl]acetic acid; 2-[2-[(5-hydroxy-1,3-benzothiazol-2-yl)methylcarbamoyl]indan-2-yl]acetic acid; 2-[2-[2-(1-methyl-4-piperidyl)ethylcarbamoyl]indan-2-yl]acetic acid; 2-[2-[2-(1,1-dimethylpiperidin-1-ium-4-yl)ethylcarbamoyl]indan-2-yl]acetate; 2-[2-[(1-benzylpyrrolidin-3-yl)carbamoyl]indan-2-yl]acetic acid; 2-[2-[(1,3-dimethylbenzimidazol-3-ium-2-yl)methylcarbamoyl]indan-2-yl]acetate; 2-[2-[(2-methylisoquinolin-2-ium-3-yl)methylcarbamoyl]indan-2-yl]acetate; 2-[2-[(1-methyl-4-piperidyl)methylcarbamoyl]indan-2-yl]acetic acid; 2-[2-[(1,1-dimethylpiperidin-1-ium-4-yl)methylcarbamoyl]indan-2-yl]acetate; 2-[2-[2-(1-methylimidazol-4-yl)ethylcarbamoyl]indan-2-yl]acetic acid; 2-[2-[(5,5-dimethyl-6,7-dihydro-4H-thiazolo[5,4-c]pyridin-5-ium-2-yl)methylcarbamoyl]indan-2-yl]acetate; 2-[2-[[(3R)-1-phenylpyrrolidin-3-yl]carbamoyl]indan-2-yl]acetic acid; 2-[2-[[(3S)-1-phenylpyrrolidin-3-yl]carbamoyl]indan-2-yl]acetic acid; 2-[2-(imidazo[1,2-a]pyridin-2-ylmethylcarbamoyl)indan-2-yl]acetic acid; 2-[2-(1,3-benzoxazol-2-ylmethylcarbamoyl)indan-2-yl]acetic acid; 2-[2-[(3-hydroxyphenyl)methylcarbamoyl]indan-2-yl]acetic acid; 2-[2-[(5-methylthiazolo[4,5-c]pyridin-5-ium-2-yl)methylcarbamoyl]indan-2-yl]acetate; 2-[2-[(5-hydroxy-2-pyridyl)methylcarbamoyl]indan-2-yl]acetic acid; and 2-[2-[1,3-benzothiazol-2-ylmethyl(methyl)carbamoyl]indan-2-yl]acetic acid is described in WO 2018/172423, the entire contents of which are hereby incorporated by reference.

The synthesis of further compounds of Formula (I) is as described below.

### General Synthetic Methodology

As described below, there are two synthetic methodologies to synthesize the compounds of the invention.

### Method A. Regiospecific Synthesis of Key Intermediate (3)

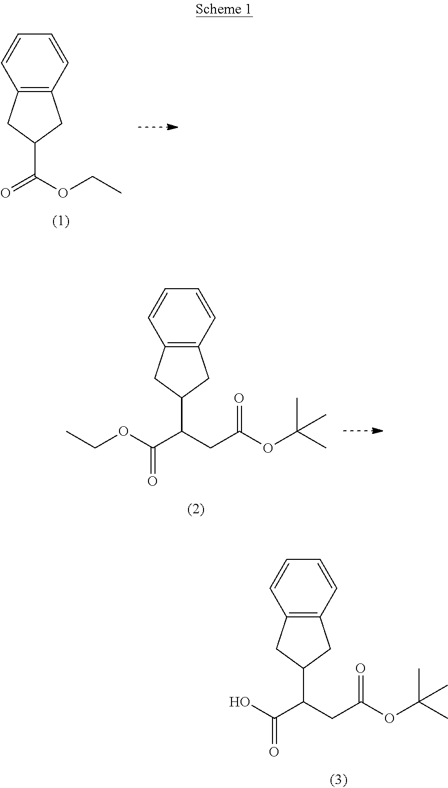 [SMILES: CCOC(=O)C1Cc2ccccc2C1 ; CCOC(=O)C(CC(=O)OC(C)(C)C)C1Cc2ccccc2C1 ; CC(C)(C)OC(=O)CC(C(=O)O)C1Cc2ccccc2C1]

Deprotonation of commercially available ethyl ester (1) with strong base (such as sodium hexamethyldisilazide) then alkylation of the anion with tert-butyl bromoacetate gives known diester (2) (Bell, I. M. and Stump, C. A., WO2006/29153; Robinson, R. P. et al, Bioorganic and Medicinal Chemistry Letters, 1996, 1719). Basic hydrolysis of the ethyl ester in the presence of the tert-butyl ester gives (3) where R=tert-butyl. Amide formation with a suitable 2-aminomethyl benzothiazole followed by treatment with TFA to remove the tert-butyl ester then affords the desired acids. The acids can be converted to esters (R¹═R^{1a}) or other produrug forms (R^{t}═CH₂OC(O)R^{1a}) by techniques known to the skilled person.

This methodology can be adapted to substituents on the indane ring.

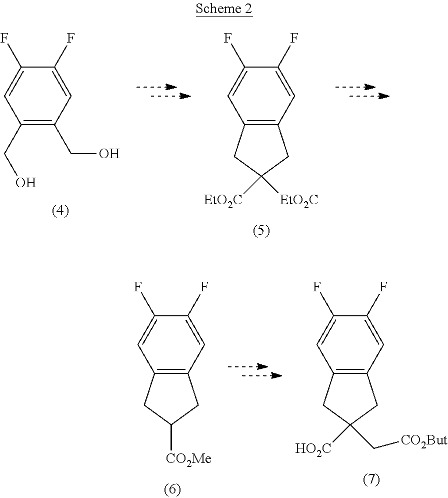

For example commercially available diol [4,5-difluoro-2-(hydroxymethyl)phenyl]methanol (4) can be converted into the bis bromomethyl analogue with either HBr (WO2008/151211) or phosphorus tribromide (US2006/223830) which can further be reacted with diethyl malonate to give indane (5). Standard hydrolysis of both esters followed by mono decarboxylation affords the mono acid (WO2006/125511) which can be esterified to give (6), the difluoro analogue of (1). Using the same methodology as applied to (1) then affords key acid (7), the difluoro analogue of intermediate (3). Similar chemistry can be applied to the corresponding analogues having different substituents on the indane ring.

There are numerous ways of accessing hydroxamic acids (for a review see Ganeshpurkar, A., et al, Current Organic Syntheses, 2018, 15, 154-165) but a very reliable procedure is to couple acids (64) with O-(oxan-2-yl)hydroxylamine using peptide coupling conditions to give protected hydroxamates (65) then deprotect with TFA to generate the hydroxamic acids (66), (see for example Ding, C., et al, Bioorg. Med. Chem. Lett, 2017, 25, 27-37).

### Method B. Synthesis of Protected 2-Aminomethyl Benzothiazoles

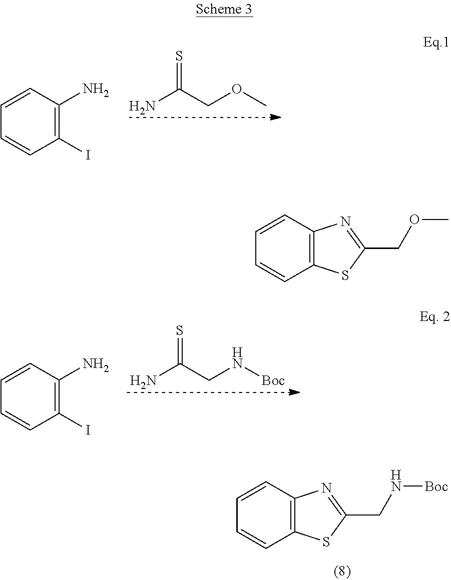

There are many ways of constructing benzothiazoles (for a review, see Seth, S; “A Comprehensive Review on Recent advances in Synthesis & Pharmacotherapeutic potential of Benzothiazoles”, Anti-Inflammatory & Anti-Allergy Agents in Medicinal Chemistry, 2015, 14, 98-112). However, most methods afford alkyl substitution at the C2-position necessitating further functional group manipulation to access the desired aminomethyl substituent required in this invention. In the 1980's the pioneering work of Takagi and colleagues led to a palladium-catalysed method of directly producing functionalised methyl groups (see Eq. 1, Scheme 2; Takagi, K. et al, Chemistry Letters, 1987, 16, 839-840). This chemistry was recently rediscovered by Mutabilis scientists who adapted the methodology to introduce a protected aminomethyl group into the benzothiazole core (8), (see Eq. 2, Scheme 2; Desroy, N., et al, Journal of Medicinal Chemistry, 2013, 56, 1418-1430). Application of this methodology accesses the protected 2-aminomethyl benzothiazoles of this invention.

### Method C. Functional Group Manipulation after Protected Aminomethylbenzothiazole

In many cases the desired substituent pattern on the phenyl ring can be established prior to benzothiazole formation using standard functional group transformations. In certain cases it is preferred to perform functional group transformations after benzothiazole formation.

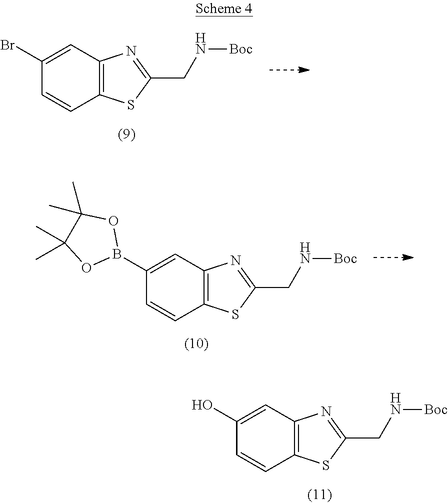 [SMILES: CC(C)(C)OC(=O)NCc1nc2cc(Br)ccc2s1 ; CC(C)(C)OC(=O)NCc1nc2cc(B3OC(C)(C)C(C)(C)O3)ccc2s1 ; CC(C)(C)OC(=O)NCc1nc2cc(O)ccc2s1]

For instance, in order to access a phenolic intermediate on the benzothiazole, one method (Scheme 4) is to construct the benzothiazole with a bromo substituent (9) then displace the bromide using bis(pinacolato)diboron and catalytic Pd(dppf)Cl₂·CH₂Cl₂, affording the boronic ester (10) after aqueous workup (for a related example see Malinger, A. et al, Journal of Medicinal Chemistry, 2016, 59, 1078-1101). Oxidation of the boronic ester to the phenol (11) can be accomplished with hydrogen peroxide (see Liu, J. et al, Tetrahedron Letters, 2017, 58, 1470-1473.) Further derivatisation of the phenol group can be achieved by standard alkylation reactions familiar to those skilled in the art.

### Method D. Functional Group Manipulation after Amide Coupling of Aminomethylbenzothiazole and Indanyl Moieties

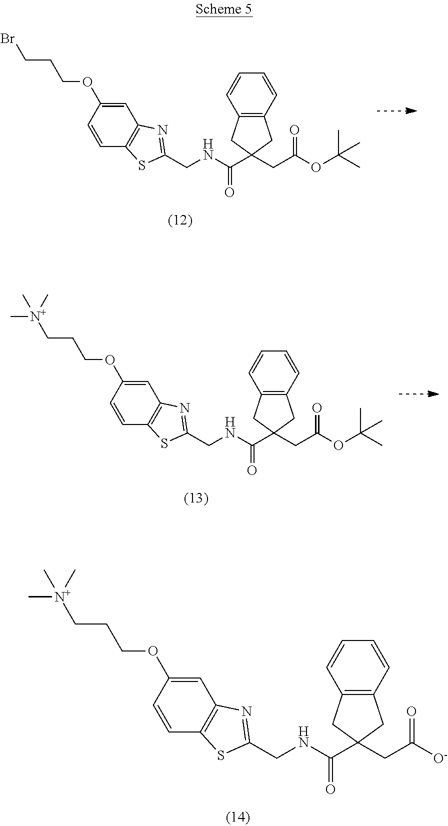

As an example of this approach, alkylation of phenol (11) with 1,3-dibromopropane, removal of the tert-butoxycarbonyl protecting group and coupling with acid (3) can generate the bromopropyloxy intermediate (12). Reaction with a tertiary amine such as trimethylamine then generates the corresponding quaternary ammonium salt (13) and finally removal of the tert-butyl ester reveals the carboxylate acid, generating zwitterionic (14) containing both a positive and a negative charge.

### Method E. Synthesis of Amide Substituents on the Benzothiazole Ring

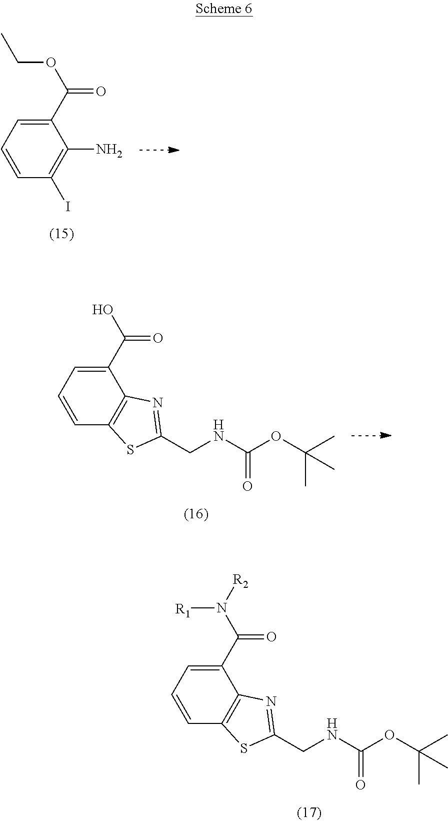

The ester (15) is subjected to the benzothiazole ring formation procedure during which hydrolysis of the ester also occurs, delivering benzothiazole acid (16). Standard amide formation with amines such as ammonia and pyrrolidine then accesses amides (17).

### Method F. Synthesis of Sulfonamide Substituents on the Benzothiazole Ring

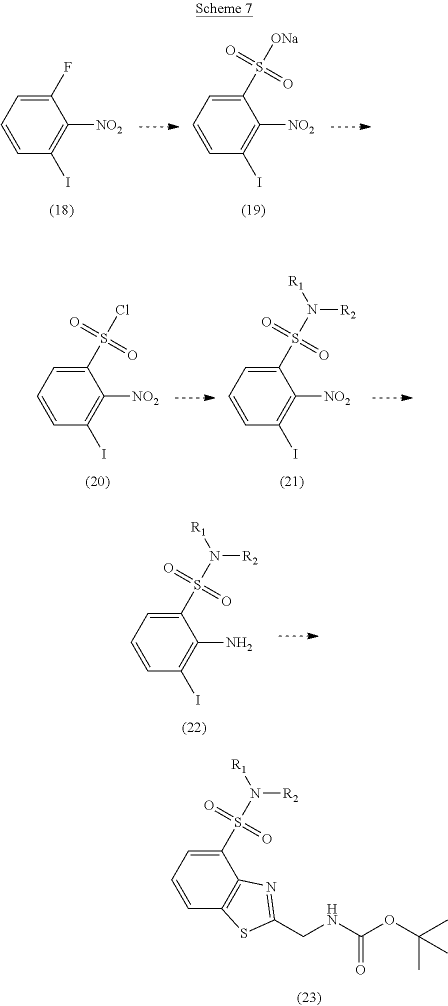

To access the analogous sulphonamides, different methodology is required. Reaction of o-fluoronitrobenzene (18) with sodium sulphite (see Sisodia, S., et al, Can. J. Chem., 1980, 58, 714-715) results in the sodium salt of the aryl sulfonic acid (19). This can be activated with standard activating agents (see Ashfaq, M., Mini-Reviews in Org. Chem., 2013, 10, 160-170) such as thionyl chloride or phosphoryl chloride to generate the arylsulfonyl chloride (20). Coupling with amines then afford sulphonamides (21). Reduction of the nitro to aniline (22) (for a recent review see Orlandi, M., et al, Organic Process Research and Development, 2018, 22, 430-445) then sets up the precursor for benzothiazole formation, accessing (23).

### Method G Final Stages to Synthesise the Examples

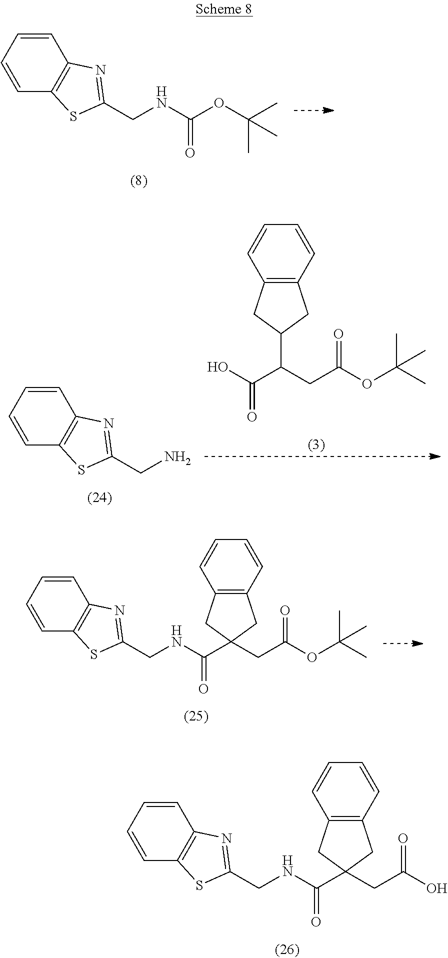 [SMILES: CC(C)(C)OC(=O)NCc1nc2ccccc2s1 ; NCc1nc2ccccc2s1 ; CC(C)(C)OC(=O)CC(C(=O)O)C1Cc2ccccc2C1 ; CC(C)(C)OC(=O)CC1(C(=O)NCc2nc3ccccc3s2)Cc2ccccc2C1 ; O=C(O)CC1(C(=O)NCc2nc3ccccc3s2)Cc2ccccc2C1]

The final stages of the syntheses generally involve acid-catalysed removal of the BOC group from (8) to reveal the free amines (24) followed by coupling with acids of type (3), usually with the standard peptide coupling reagent HATU (for a comprehensive review of the myriad available peptide coupling reagents, see Valeur, E. and Bradley, M, Chem. Soc. Rev., 2008, 28, 606-631). Finally further acid treatment with TFA removes the t-butyl ester to afford the Examples of the invention.

It is understood that these synthetic routes are not exclusive and functional group interconversion is possible at the phenyl precursor stage, the protected aminomethyl benzothiazole stage and the post-coupling amide stage.

### EXAMPLES

1H NMR spectra are reported at 300, 400 or 500 MHz in DMSO-d6 solutions (δ in ppm), using DMSO-d₅ as reference standard (2.50 ppm), or CDCl₃ solutions using chloroform as the reference standard (7.26 ppm). When peak multiplicities are reported, the following abbreviations are used: s (singlet), d (doublet), t (triplet), m (multiplet), bs (broadened singlet), bd (broadened doublet), dd (doublet of doublets), dt (doublet of triplets), q (quartet). Coupling constants, when given, are reported in hertz (Hz).

The term “purified by prep hplc (MDAP)” refers compound purification using a mass-directed auto purification system on an Agilent 1260 infinity machine with an XSelect CHS Prep C18 column, eluting with 0.1% FA in water/ACN and detection with a Quadrupole LC/MS.

### Abbreviations

ACN Acetonitrile
AcOH Acetic acid
aq. Aqueous
Bpin Bis(pinacolato)diboron
CaCl₂ Calcium chloride
Cs₂CO₃ Cesium carbonate
cfu Colony forming unit
Conc Concentrated
Cu(OAc)₂ Copper(II) acetate
CuO Copper oxide
DCM Dichloromethane
DEA Diethylamine
DIPEA N,N-Diisopropylethylamine
DMAP 4-dimethylaminopyridine
DMF N,N-Dimethylformamide
DMSO Dimethyl sulfoxide
dppf 1,1′-Bis(diphenylphosphino)ferrocene
EDC·HCl N-(3-Dimethylaminopropyl)-N′-ethylcarbodiimide hydrochloride
Et₂O Diethyl ether
EtOAc Ethyl acetate
EtOH Ethanol
Et₃N Triethylamine
Ex Excitation
FA Formic acid
FCC Flash column chromatography purification on silica
h Hour(s)
HATU 1-[Bis(dimethylamino)methylene]-1H-1,2,3-triazolo[4,5-b]pyridinium 3-oxid hexafluorophosphate
HCl Hydrochloric acid/hydrochloride salt
HOBt Hydroxybenzotriazole
H₂SO₄ Sulfuric Acid
Km Michaelis constant
KOAc Potassium acetate
KOH Potassium hydroxide
MeCN Acetonitrie
MeOH Methanol
Mel Methyl iodide
min Minute(s)
MgSO₄ Magnesium sulfate
N₂ Nitrogen
NBS N-bromo succinimide
Na₂CO₃ Sodium carbonate
NaHCO₃ Sodium bicarbonate
NaHMDS Sodium bis(trimethylsilyl)amide
Na₂SO₄ Sodium sulfate
Pd₂(dba)₃ Tris(dibenzylideneacetone)dipalladium(O)
PdCl₂(dppf) [1,1′-Bis(diphenylphosphino)ferrocene]dichloropalladium(II)
RT Room temperature
SCX-2 Strong cation exchange resin (silica-propyl sulfonic acid)
T&B Time, % solvent B
TES Triethylsilane
TFA Trifluoroacetic acid
THF Tetrahydrofuran
T3P Propylphosphinic anhydride

### Example 1 2-[2-[(4-carbamoyl-1,3-benzothiazol-2-yl)methylcarbamoyl]indan-2-yl]acetic acid

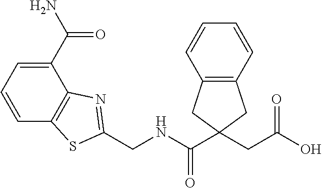 [SMILES: NC(=O)c1cccc2sc(CNC(=O)C3(CC(=O)O)Cc4ccccc4C3)nc12]

### a. Ethyl 2-amino-3-iodobenzoate

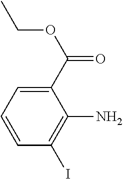

A solution of ethyl 2-aminobenzoate (1.1 g, 3.0 mmol) in toluene (75 mL) was treated with acetic acid (0.34 mL, 3.0 mmol) and N-iodosuccinimide (0.68 g, 3.0 mmol). After 70h the mixture was washed with saturated aqueous sodium bicarbonate solution, dried (Na₂SO₄) and evaporated. The residue was chromatographed on silica eluting 0-10% ethyl acetate in toluene affording a red oil that solidified on standing (0.26 g, 29%). M/z 292.5 (M+H)⁺.

### b. 2-({[(Tert-butoxy)carbonyl]amino}methyl)-1,3-benzothiazole-4-carboxylic acid

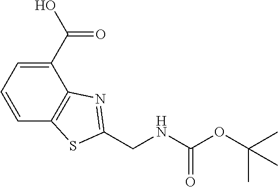 [SMILES: CC(C)(C)OC(=O)NCc1nc2c(C(=O)O)cccc2s1]

A solution of ethyl 2-amino-3-iodobenzoate (147 mg, 0.5 mmol) in ACN (2 mL) was treated with tert-butyl (2-amino-2-thioxoethyl)carbamate (115 mg, 0.61 mmol), calcium oxide (42 mg, 0.76 mmol), tris(dibenzylideneacetone)dipalladium(0) (92 mg, 0.1 mmol) and dppf (224 mg, 0.4 mmol). The flask was evacuated and refilled with nitrogen twice. The mixture was heated at 60° C. in a sealed vial for 1.5h then cooled and partitioned between ethyl acetate and 10% aqueous citric acid solution. The aqueous phase was further extracted with ethyl acetate and the combined extracts were dried (Na₂SO₄) and evaporated. The residue was chromatographed on silica eluting with 0-15% methanol in DCM affording an oil (151 mg, 97%). M/z 331.4 (M+Na)⁺.

### c. Tert-butyl N-[(4-carbamoyl-1,3-benzothiazol-2-yl)methyl]carbamate

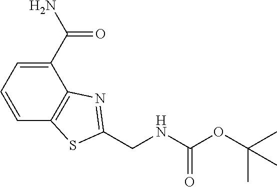 [SMILES: CC(C)(C)OC(=O)NCc1nc2c(C(N)=O)cccc2s1]

A solution of the above 2-({[(tert-butoxy)carbonyl]amino}methyl)-1,3-benzothiazole-4-carboxylic acid (170 mg) in DMF (2 mL) was treated with ammonium chloride (54 mg, 1 mmol), DIPEA (0.35 mL, 2 mmol) and HATU (0.29 g, 2 mmol). After 0.5h the mixture was partitioned between ethyl acetate and water. The aqueous phase was further extracted with ethyl acetate and the combined extracts were dried (Na2SO4) and evaporated. The residue was chromatographed on silica eluting with 30-100%% ethyl acetate in hexane affording a brown oil (151 mg, 100%). M/z 330.5 (M+Na)⁺.

### d. 2-(Aminomethyl)-1,3-benzothiazole-4-carboxamide

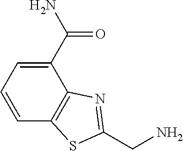 [SMILES: NCc1nc2c(C(N)=O)cccc2s1]

A solution of tert-butyl N-[(4-carbamoyl-1,3-benzothiazol-2-yl)methyl]carbamate (76 mg, 0.25 mmol) in DCM (3 mL) was treated with TFA (0.8 mL). After 1.25h toluene was added and the mixture evaporated. The residue was treated with a further portion of toluene and evaporated. The residue was added to an SCX cartridge, eluting with methanol then 2M ammonia in methanol affording a pale brown solid (18 mg, 33%). M/z 230.5 (M+Na)⁺.

### e. 2,3-dihydro-1H-indene-2-carboxylate

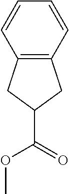

To a stirred solution of 2,3-dihydro-1H-indene-2-carboxylic acid (20 g, 123 mmol) in methanol (200 mL) was added con. H₂SO₄ (10 mL, 185 mmol) drop wise at room temperature and stirred at 80° C. for 16 h. The reaction mixture was evaporated to get residue. The residue was dissolved in water (100 mL) and extracted with EtOAc (2×100 mL). The organic layer was washed with sat. sodium bicarbonate, brine and evaporated affording a light brown liquid (20 g, 92%). M/z 177.1 (M+H)⁺.

### f. Methyl 2-(2-(tert-butoxy)-2-oxoethyl)-2,3-dihydro-1H-indene-2-carboxylate

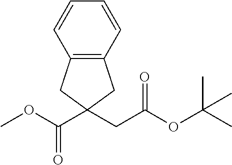 [SMILES: COC(=O)C1(CC(=O)OC(C)(C)C)Cc2ccccc2C1]

To a solution of methyl 2,3-dihydro-1H-indene-2-carboxylate (5 g, 28.3 mmol) in THF (100 mL) was added NaHMDS (21 mL, 42.5 mmol, 2M in THF) at −78° C. under argon and stirred at −78° C. for 1 h. Then tert-butyl 2-bromoacetate solution (6.4 mL, 42.5 mmol) in THF (30 mL) was added drop wise for 15 minutes at −78° C. and stirred at same temperature for 2 h. The reaction mixture was quenched with sat. ammonium chloride solution (50 mL) at −78° C. and allowed to stir at room temperature for 30 minutes. The organic layer was separated, aqueous layer was extracted with EtOAc (2×100 mL), and the combined organic layer was evaporated to get crude compound. The crude compound was triturated with n-pentane (50 mL) at −78° C. and stirred at same temperature for 15 minutes. The resulting solid was filtered and dried under vacuum affording an off white (3.7 g, 45%). M/z=313.0 (M+Na)⁺.

### g. 2-(2-(tert-butoxy)-2-oxoethyl)-2,3-dihydro-1H-indene-2-carboxylic acid

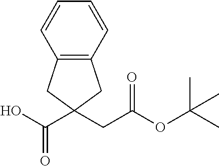 [SMILES: CC(C)(C)OC(=O)CC1(C(=O)O)Cc2ccccc2C1]

To a stirred solution of methyl 2-(2-(tert-butoxy)-2-oxoethyl)-2,3-dihydro-1H-indene-2-carboxylate (430 g, 1.48 mol) in THF (2.15 L) and ethanol (2.15 L) was added 0.5 M LiOH·H₂O (6.8 L, 2.96 mol) drop wise at room temperature and stirred at same temperature for 2 h. The reaction mixture was evaporated to get the residue and the residue was diluted with H₂O (1 L) and extracted with diethyl ether. The aqueous layer was acidified with 1N HCl to pH 3-4. The resulting precipitate was filtered, washed with water, n-pentane and dried under vacuum affording a white solid (254.5 g, 62%). M/z 275.2 (M−H)⁻. ¹H NMR (300 MHz, DMSO-d₆): δ 12.4 (1H, bs), 7.18-7.10 (4H, m), 3.39 (2H, d, J=16.2 Hz), 2.92 (2H, d, J=16.2 Hz), 2.64 (2H, s), 1.37 (9H, s).

### h. Tert-butyl 2-[2-[(4-carbamoyl-1,3-benzothiazol-2-yl)methylcarbamoyl]indan-2-yl]acetate

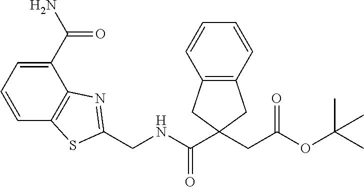 [SMILES: CC(C)(C)OC(=O)CC1(C(=O)NCc2nc3c(C(N)=O)cccc3s2)Cc2ccccc2C1]

A solution of 2-(aminomethyl)-1,3-benzothiazole-4-carboxamide (18 mg, 0.09 mmol), 2-(2-(tert-butoxy)-2-oxoethyl)-2,3-dihydro-1H-indene-2-carboxylic acid (26 mg, 0.1 mmol) and DIPEA (34 mg, 0.26 mmol) in DMF (0.5 mL) was treated with HATU (50 mg, 0.1 mmol). After 0.33 h the mixture was partitioned between ethyl acetate and 10% aqueous citric acid solution. The aqueous phase was further extracted with ethyl acetate and the combined extracts were washed with saturated aqueous sodium chloride solution, dried (Na₂SO₄) and evaporated. The residue was chromatographed on silica eluting with 50-100%% ethyl acetate in hexane affording a brown oil (36 mg, 90%). M/z 488.2 (M+Na)⁺.

### i. 2-[2-[(4-carbamoyl-1,3-benzothiazol-2-yl)methylcarbamoyl]indan-2-yl]acetic acid

A solution of Tert-butyl 2-[2-[(4-carbamoyl-1,3-benzothiazol-2-yl)methylcarbamoyl]indan-2-yl]acetate (36 mg, 0.08 mmol) in DCM (2 mL) was treated with TFA (0.8 mL). After 1.5h toluene was added and the mixture evaporated. The residue was treated with a further portion of toluene and evaporated. The residue was chromatographed on silica eluting with 2-12% methanol in DCM to afford the title compound as a white solid (14 mg, 43%). M/z 410.4 (M+H)⁺. ¹H NMR (400 MHz, d₆-DMSO) δ 12.20 (1H, bs), 9.20 (1H, bs), 9.00 (1H, bs), 8.30 (1H, d), 8.15 (1H, d), 7.90 (1H, bs), 7.55 (1H, t), 7.25 (2H, m), 7.15 (2H, m), 4.75 (2H, d), 3.50 (2H, d), 3.00 (2H, d).

### Example 2 2-[2-[[4-(pyrrolidine-1-carbonyl)-1,3-benzothiazol-2-yl]methylcarbamoyl]indan-2-yl]acetic acid

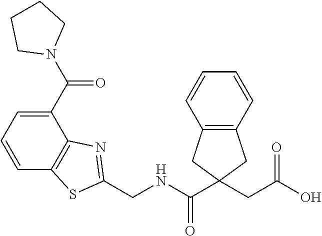 [SMILES: O=C(O)CC1(C(=O)NCc2nc3c(C(=O)N4CCCC4)cccc3s2)Cc2ccccc2C1]

This was prepared in a similar manner to Example 1 with the change that pyrrolidine was used in place of ammonium chloride, giving a white solid (5.0 mg). M/z 464.2 (M+H)⁺. ¹H NMR (400 MHz, d₆-DMSO) δ 12.20 (1H, bs), 9.00 (1H, bs), 8.10 (1H, d), 7.45 (2H, m), 7.20 (2H, m), 7.10 (2H, m), 4.70 (2H, d), 3.55 (2H, m), 3.50 (2H, d), 3.10 (2H, m), 3.00 (2H, d), 1.90 (2H, m), 1.80 (2H, m).

### Example 3 2-[2-[(4-pyrrolidin-1-ylsulfonyl-1,3-benzothiazol-2-yl)methylcarbamoyl]indan-2-yl]acetic acid

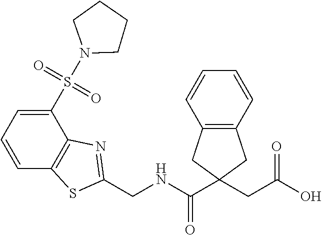 [SMILES: O=C(O)CC1(C(=O)NCc2nc3c(S(=O)(=O)N4CCCC4)cccc3s2)Cc2ccccc2C1]

### a. Sodium 3-iodo-2-nitrobenzene-1-sulfonate

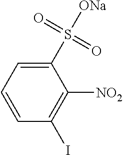

A solution of (commercially available) 1-fluoro-3-iodo-2-nitrobenzene (218 mg, 0.75 mmol) in ethanol (6 mL) was treated with a solution of sodium sulphite (236 mg, 1.9 mmol) in water (5 mL). The mixture was heated to reflux for 4 h. The cooled mixture was evaporated to dryness and chromatographed on reverse phase silica (C-18 cartridge) eluting with water then methanol affording a white solid (144 mg, 55%). M/z 328.2 (M−Na)⁻.

### b. 1-(3-Iodo-2-nitrobenzenesulfonyl)pyrrolidine

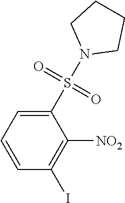 [SMILES: O=[N+]([O-])c1c(I)cccc1S(=O)(=O)N1CCCC1]

A suspension of sodium 3-iodo-2-nitrobenzene-1-sulfonate (133 mg, 0.38 mmol) in thionyl chloride (1 mL) was treated with DMF (1 drop) and the mixture was heated to reflux for 1.5 h then diluted with toluene and evaporated. The residue was re-dissolved in toluene and re-evaporated a further 3 times affording 3-iodo-2-nitrobenzene-1-sulfonyl chloride as an oil (124 mg, 94%). Half of this sample (62 mg, 0.18 mmol) was dissolved in toluene (0.5 mL) and added to a solution of pyrrolidine (213 mg, 3 mmol) in THF (2 mL) at 0° C. After the addition the mixture was stirred at room temperature for 0.5h then diluted with toluene and evaporated. The residue was chromatographed on silica eluting with 0-5% methanol in DCM affording a colourless solid (65 mg, 96%). M/z 383.3 (M+H)⁺.

### c. 2-Iodo-6-(pyrrolidine-1-sulfonyl)aniline

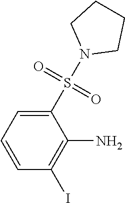 [SMILES: Nc1c(I)cccc1S(=O)(=O)N1CCCC1]

A solution of 1-(3-iodo-2-nitrobenzenesulfonyl)pyrrolidine (65 mg, 0.17 mmol) in ethanol (2 mL) was treated with iron powder (50 mg, 0.9 mmol) then acetic acid (200 mg, 3.4 mmol). The mixture was heated to 85° C. for 1.5 h then filtered through celite, washing with isopropanol. The filtrate was evaporated and the residue chromatographed on silica eluting with 0-15% ethyl acetate in toluene affording a colourless oil (46 mg, 73%). M/z 353.3 (M+H)⁺.

### d. Tert-butyl N-{[4-(pyrrolidine-1-sulfonyl)-1,3-benzothiazol-2-yl]methyl}carbamate

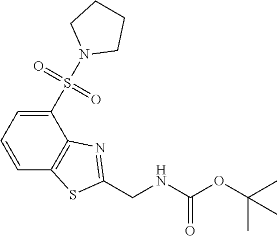 [SMILES: CC(C)(C)OC(=O)NCc1nc2c(S(=O)(=O)N3CCCC3)cccc2s1]

A solution of 2-iodo-6-(pyrrolidine-1-sulfonyl)aniline (46 mg, 0.13 mmol) in in ACN (1 mL) was treated with tert-butyl (2-amino-2-thioxoethyl)carbamate (30 mg, 0.16 mmol), calcium oxide (11 mg, 0.2 mmol), tris(dibenzylideneacetone)dipalladium(0) (24 mg, 0.03 mmol) and dppf (58 mg, 0.11 mmol). The mixture was heated at 60° C. in a sealed vial for 2 h then cooled, diluted with toluene and filtered through celite. The filtrate was added directly to a silica cartridge (10 g) and chromatographed eluting with 0-50% ethyl acetate in toluene affording an oil (33 mg, 64%). M/z 420.2 (M+Na)⁺.

### e. [4-(Pyrrolidine-1-sulfonyl)-1,3-benzothiazol-2-yl]methanamine

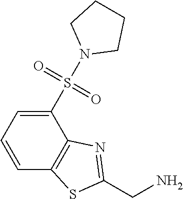 [SMILES: NCc1nc2c(S(=O)(=O)N3CCCC3)cccc2s1]

A solution of tert-butyl N-{[4-(pyrrolidine-1-sulfonyl)-1,3-benzothiazol-2-yl]methyl}carbamate (33 mg, 0.08 mmol) in DCM (2 mL) was treated with TFA (0.5 mL). After 2 h, toluene was added and the mixture evaporated. The residue was treated with a further portion of toluene and evaporated. The residue was dissolved in methanol:DCM (1:1) and loaded onto an SCX cartridge (10 g) and chromatographed eluting with 1M ammonia/methanol. Product-containing fractions were combined and evaporated and the residue further chromatographed on silica eluting with 0-6% 2M ammonia/methanol in DCM affording a brown oil (17 mg, 68%). M/z 298.4 (M+H)⁺.

### f. Tert-butyl 2-[2-[(4-pyrrolidin-1-ylsulfonyl-1,3-benzothiazol-2-yl)methylcarbamoyl]indan-2-yl]acetate

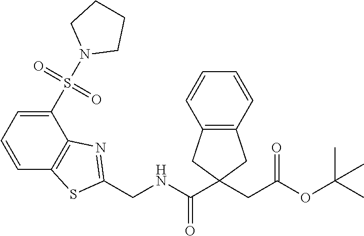 [SMILES: CC(C)(C)OC(=O)CC1(C(=O)NCc2nc3c(S(=O)(=O)N4CCCC4)cccc3s2)Cc2ccccc2C1]

A solution of [4-(pyrrolidine-1-sulfonyl)-1,3-benzothiazol-2-yl]methanamine (18 mg, 0.06 mmol), 2-(2-(tert-butoxy)-2-oxoethyl)-2,3-dihydro-1H-indene-2-carboxylic acid (18 mg, 0.07 mmol) and DIPEA (23 mg, 0.03 mmol) in DMF (0.5 mL) was treated with HATU (34 mg, 0.9 mmol). After 0.5 h the mixture was partitioned between ethyl acetate and 10% aqueous citric acid solution. The aqueous phase was further extracted with ethyl acetate and the combined extracts were washed with saturated aqueous sodium chloride solution, dried (Na₂SO₄) and evaporated. The residue was chromatographed on silica eluting with 30-60% ethyl acetate in hexane affording a brown oil (33 mg, 100%). M/z 578.3 (M+Na)⁺.

### g. 2-[2-[(4-pyrrolidin-1-ylsulfonyl-1,3-benzothiazol-2-yl)methylcarbamoyl]indan-2-yl]acetic acid

A solution of tert-butyl 2-[2-[(4-pyrrolidin-1-ylsulfonyl-1,3-benzothiazol-2-yl)methylcarbamoyl]indan-2-yl]acetate (33 mg, 0.06 mmol) in DCM (2 mL) was treated with TFA (0.8 mL). After 1.75 h toluene was added and the mixture evaporated. The residue was treated with a further portion of toluene and evaporated. The residue was chromatographed on silica eluting with 2-10% methanol in DCM to afford the title compound as a white solid (19 mg, 62%). M/z 500.1 (M+H)⁺. ¹H NMR (400 MHz, d₆-DMSO) δ 12.30 (1H, bs), 9.00 (1H, bs), 8.40 (1H, d), 7.95 (1H, d), 7.58 (1H, t), 7.23 (2H, m), 7.15 (2H, m), 4.70 (2H, d), 3.50 (2H, d), 3.40 (4H, m), 3.00 (2H, d), 1.7-1.6 (4H, m).

### Example 4 2-[2-[(4-sulfamoyl-1,3-benzothiazol-2-yl)methylcarbamoyl]indan-2-yl]acetic acid

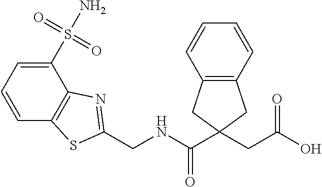 [SMILES: NS(=O)(=O)c1cccc2sc(CNC(=O)C3(CC(=O)O)Cc4ccccc4C3)nc12]

This was prepared by the same methodology as Example 3 with the exception that ammonium chloride (source of ammonia) was used instead of pyrrolidine in the sulphonamide formation step with 3-iodo-2-nitrobenzene-1-sulfonyl chloride. The title compound was isolated as a white solid (15 mg). M/z 446.1 (M+H)⁺. ¹H NMR (400 MHz, d₆-DMSO) δ 12.00 (1H, bs), 8.30 (1H, d), 7.92 (1H, d), 7.55 (1H, t), 7.30 (2H, s), 7.20 (2H, m), 7.10 (2H, m), 4.80 (2H, d), 3.45 (2H, d), 3.00 (2H, d).

### Example 5 2-[2-[(4-piperazin-1-ylsulfonyl-1,3-benzothiazol-2-yl)methylcarbamoyl]indan-2-yl]acetic acid

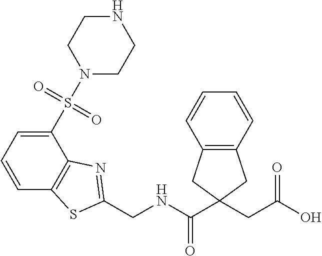 [SMILES: O=C(O)CC1(C(=O)NCc2nc3c(S(=O)(=O)N4CCNCC4)cccc3s2)Cc2ccccc2C1]

This was prepared by the same methodology as Example 3 with the exception that benzyl piperazine-1-carboxylate was used instead of pyrrolidine in the sulphonamide formation step with 3-iodo-2-nitrobenzene-1-sulfonyl chloride and removal of the benzyl carbamate protecting group necessitated a reaction time of 55 h at room temperature in neat TFA. The title compound was isolated after purification as a white solid (9 mg). M/z 515.3 (M+H)⁺. ¹H NMR (400 MHz, d₆-DMSO) δ 10.00 (1H, bs), 8.40 (1H, d), 7.95 (1H, d), 7.60 (1H, t), 7.20 (2H, m), 7.10 (2H, m), 4.75 (2H, d), 3.50 (4H, m), 3.20 (2H, m), 3.00 (2H, d), 2.80 (2H, m), 2.60 (2H, m).

### Example 6 2-[2-[[4-(3-aminopyrrolidin-1-yl)sulfonyl-1,3-benzothiazol-2-yl]methylcarbamoyl]indan-2-yl]acetic acid

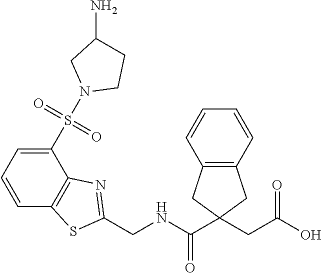 [SMILES: NC1CCN(S(=O)(=O)c2cccc3sc(CNC(=O)C4(CC(=O)O)Cc5ccccc5C4)nc23)C1]

This was prepared by the same methodology as Example 3 with the exception that (R, S)-benzyl N-(pyrrolidin-3-yl) carbamate was used instead of pyrrolidine in the sulphonamide formation step with 3-iodo-2-nitrobenzene-1-sulfonyl chloride and removal of the benzyl carbamate protecting group necessitated a reaction time of 48 h at room temperature in neat TFA. The title compound was isolated as a white solid (14 mg). M/z 515.4 (M+H)⁺. ¹H NMR (400 MHz, d₆-DMSO) δ 11.00 (1H, bs), 8.40 (1H, d), 8.00 (1H, d), 7.60 (1H, t), 7.20 (2H, m), 7.15 (2H, m), 4.75 (2H, m), 4.00 (1H, t), 3.60-3.20 (6H, m), 3.00-2.85 (2H, m), 1.95 (1H, m), 1.70 (1H, m).

### Example 7 2-[2-[(4-methylsulfonyl-1,3-benzothiazol-2-yl)methylcarbamoyl]indan-2-yl]acetic acid

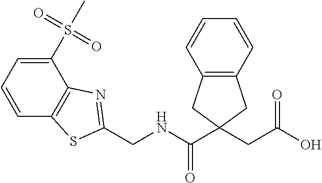 [SMILES: CS(=O)(=O)c1cccc2sc(CNC(=O)C3(CC(=O)O)Cc4ccccc4C3)nc12]

### a. 1-Iodo-3-methanesulfonyl-2-nitrobenzene

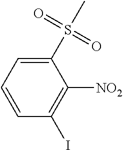

A solution of 2-fluoro-3-iodo-2-nitrobenzene (200 mg, 0.75 mmol) in THF (6 mL) was treated portionwise with sodium thiomethoxide then 15-crown-5 (1,4,7,10,13-pentaoxacyclopentadecane) (20 mg) was added. After 7 h, the mixture was diluted with DCM (6 mL) and 3-chloroperbenzoic acid (672 mg, 3 mmol) was added. After 16h the mixture was partitioned between ethyl acetate and 10% aqueous sodium metabisulfite solution. The aqueous phase was further extracted with ethyl acetate and the combined organic extracts washed with saturated aqueous sodium bicarbonate solution, saturated aqueous sodium chloride solution, dried (Na₂SO₄) and evaporated. The residue was chromatographed on silica eluting with 0-100% ethyl acetate in toluene affording a white solid (227 mg). This was further purified by chromatography on silica eluting with 0-2% methanol in chloroform affording a colourless solid (92 mg, 38%). M/z 246.3 (M+H)⁺.

### b. 2-[2-[(4-methylsulfonyl-1,3-benzothiazol-2-yl)methylcarbamoyl]indan-2-yl]acetic acid

This was prepared from 1-iodo-3-methanesulfonyl-2-nitrobenzene by the same reaction sequence as described for Example (3c) onwards, affording the title compound as a white solid (34 mg). M/z 445.4 (M+H)⁺. ¹H NMR (400 MHz, d-DMSO) δ 12.20 (1H, bs), 8.90 (1H, bs), 8.45 (1H, d), 8.00 (1H, d), 7.62 (1H, t), 7.22 (2H, m), 7.15 (2H, m), 4.75 (2H, d), 3.52 (3H, s), 3.50 (2H, d), 3.00 (2H, d).

### Example 8 2-[2-[[6-methoxy-5-[2-(4-methylpiperazin-1-yl)-2-oxo-ethoxy]-1,3-benzothiazol-2-yl]methylcarbamoyl]indan-2-yl]acetic acid

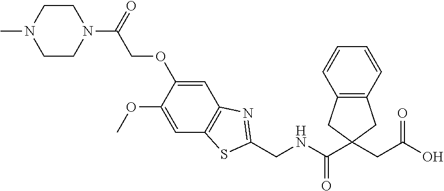 [SMILES: COc1cc2sc(CNC(=O)C3(CC(=O)O)Cc4ccccc4C3)nc2cc1OCC(=O)N1CCN(C)CC1]

### a. 4-bromo-5-methoxy-2-nitroaniline

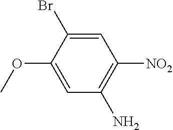

To a stirred solution of 5-methoxy-2-nitroaniline (100 g, 595 mmol) in acetonitrile (2.5 L) was added NBS (106 g, 595 mmol) portion wise at room temperature. The mixture was cooled to 0° C. and added TFA (46 mL, 595 mmol) drop wise for 30 minutes and allowed to stir at room temperature for 16 h. The reaction mixture was diluted with water (1 L) and adjusted the pH to ˜ 8 with 1N NaOH. The resulting precipitate was filtered, washed with water (500 mL) and dried under vacuum affording a yellow solid. (105 g, 72%). M/z 247 (M+H)⁺.

### b. 1-Bromo-4-iodo-2-methoxy-5-nitrobenzene

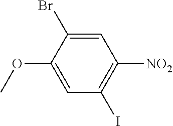

To a stirred solution of 4-bromo-5-methoxy-2-nitroaniline (50 g, 203 mmol) in acetonitrile (750 mL) was added concentrated H₂SO₄ (24 mL, 457 mmol) drop wise at −10° C. Then NaNO₂ (28 g, 406 mmol) in water (175 mL) was added drop wise at −10° C. for 15 minutes and stirred at same temperature for 30 min. After that KI solution (135 g, 813 mmol) in water (175 mL) was added drop wise at −10° C. for 20 minutes and stirred at same temperature for 30 min. The reaction mixture was quenched with sodium metabisulphite solution (309 g, 1.62 mmol) in water (1.6 L) at -10° C. to 0° C. for 1 h. Then water (1 L) was added and allowed to stir at room temperature for 30 minutes. The resulting precipitate was filtered, washed with water (1 L) and dried under vacuum affording a yellow solid. (60 g, 82%). M/z 357.8 (M+H)⁺.

### c. 5-Bromo-2-iodo-4-methoxyaniline

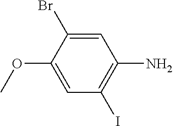

To a stirred solution of 1-bromo-4-iodo-2-methoxy-5-nitrobenzene (106 g, 296 mmol) in EtOH: H₂O (800 mL: 200 mL) was added Fe (49.7 g, 890 mmol), NH₄Cl (80 g, 1.48 mmol) at room temperature and stirred at 90° C. for 2 h. Then the reaction mixture was cooled to 60° C., added additional amount of Fe (33 g, 593 mmol), NH₄Cl (80 g 1.48 mmol) and stirred at 90° C. for 30 minutes. The reaction mixture was filtered through celite pad, washed the pad with methanol (1 L) and filtrate was concentrated to give residue. The residue was diluted with cold water (1 L) and adjusted the pH to ˜8 with 1N NaOH. The resulting precipitate was filtered and dried under vacuum affording a light brown solid (90 g, 92%). M/z 327.8 (M+H)⁺.

### d. Tert-butyl N-[(5-bromo-6-methoxy-1,3-benzothiazol-2-yl)methyl]carbamate

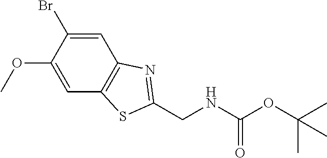 [SMILES: COc1cc2sc(CNC(=O)OC(C)(C)C)nc2cc1Br]

To a stirred solution of 5-bromo-2-iodo-4-methoxyaniline (50 g, 152 mmol) in acetonitrile (560 mL) was added tert-butyl (2-amino-2-thioxoethyl) carbamate (35 g, 183 mmol), CaO (17 g, 305 mmol) and degassed with argon for 20 minutes. Then Pd₂(dba)₃ (14 g, 15.2 mmol), dppf (25.4 g, 15.8 mmol) was added and purged with argon for further 5 minutes and the reaction mixture was stirred at 80° C. for 4 hour. The reaction mixture was filtered through celite pad and washed the pad with EtOAc (300 mL). The filtrate was washed with water and evaporated to get crude compound. The crude compound was dissolved in acetonitrile (200 mL), on standing for 1 h solid was precipitated out. The resulting solid was filtered, washed with acetonitrile (50 mL) and dried under vacuum affording an off white solid (34 g, 60%). M/z 372.9 (M+H)⁺.

### e. Tert-butyl N-[[6-methoxy-5-(4,4,5,5-tetramethyl-1,3,2-dioxaborolan-2-yl)-1,3-benzothiazol-2-yl]methyl]carbamate

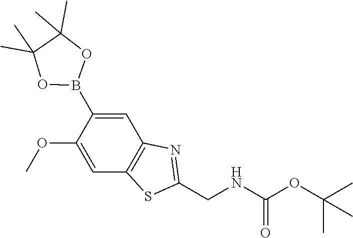 [SMILES: COc1cc2sc(CNC(=O)OC(C)(C)C)nc2cc1B1OC(C)(C)C(C)(C)O1]

To a stirred solution of tert-butyl ((5-bromo-6-methoxybenzo[d]thiazol-2-yl)methyl)carbamate (5 g, 13.44 mmol) in dioxane (100 mL) was added BPin (6.8 g, 26.8 mmol), KOAc (4.6 g, 47.0 mmol) and purged with argon for 15 minutes. Then Pd₂Cl₂(dppf). DCM (1.1 g, 1.34 mmol) was added and purged with argon for further 5 minutes. The reaction mixture was heated at 100° C. for 16 h. The reaction mixture was filtered through celite pad and washed the pad with EtOAc (50 mL). The filtrate was washed with water, brine and evaporated affording a white solid (12 g, crude). M/z 339 (M+H)⁺.

### f. Tert-butyl N-[(5-hydroxy-6-methoxy-1,3-benzothiazol-2-yl)methyl]carbamate

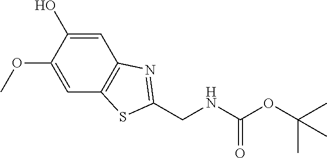 [SMILES: COc1cc2sc(CNC(=O)OC(C)(C)C)nc2cc1O]

To a stirred solution of tert-butyl N-[[6-methoxy-5-(4,4,5,5-tetramethyl-1,3,2-dioxaborolan-2-yl)-1,3-benzothiazol-2-yl]methyl]carbamate (12 g, 35.5 mmol) in THF (180 mL) was added 1N NaOH (35 mL, 35.5 mmol), 30% H₂O₂ (6.2 mL 81.6 mmol) at 0° C. and stirred at same temperature for 30 minutes. The reaction mixture was partitioned between water and EtOAc. The organic layer was separated washed with water, brine and evaporated to get crude compound. The crude compound was chromatographed on silica eluting with 30% EtOAc in petroleum ether affording an off white solid. (2.5 g 54%). M/z 311.0 (M+H)⁺. ¹H NMR (500 MHz, CDCl₃): δ 7.50 (1H, s), 7.25 (1H, s), 5.76 (1H, s), 5.30 (1H, s), 4.68 (2H, d, J=5.5 Hz), 3.97 (3H, s), 1.54 (9H, s). M/z 311.0 (M+H)⁺.

### g. Tert-butyl N-({6-methoxy-5-[2-(4-methylpiperazin-1-yl)-2-oxoethoxy]-1,3-benzothiazol-2-yl}methyl)carbamate

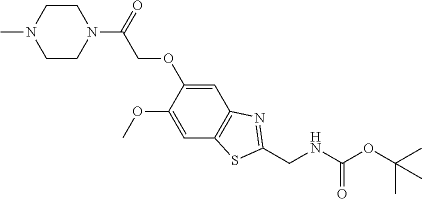 [SMILES: COc1cc2sc(CNC(=O)OC(C)(C)C)nc2cc1OCC(=O)N1CCN(C)CC1]

A mixture of tert-butyl N-[(5-hydroxy-6-methoxy-1,3-benzothiazol-2-yl)methyl]carbamate (310 mg, 1 mmol), (commercially-available) 1-(2-chloroacetyl)-4-methyl-piperazine hydrochloride (234 mg, 1.1 mmol) and caesium carbonate (980 mg, 3 mmol) in ACN (3 mL) was stirred for 18h then partitioned between DCM and saturated aqueous sodium chloride solution, and then the organic phase was dried (Na₂SO₄) and evaporated. The residue was chromatographed on silica eluting with 2-10% 7M ammonia/methanol in DCM affording a white solid (268 mg, 59%). M/z 451.6 (M+H)⁺.

### h. 2-{[2-(Aminomethyl)-6-methoxy-1,3-benzothiazol-5-yl]oxy}-1-(4-methylpiperazin-1-yl)ethan-1-one

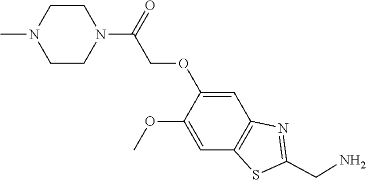 [SMILES: COc1cc2sc(CN)nc2cc1OCC(=O)N1CCN(C)CC1]

A solution of tert-butyl N-({6-methoxy-5-[2-(4-methylpiperazin-1-yl)-2-oxoethoxy]-1,3-benzothiazol-2-yl}methyl)carbamate (265 mg, 0.6 mmol) in DCM (3 mL) was treated with TFA (1.4 mL). After 1 h the mixture was added to an SCX-2 cartridge, pre-washed with methanol. This was washed with methanol then eluted to 7M ammonia/methanol. This latter fraction was evaporated to give an orange foam (195 mg, 95%). M/z 351.6 (M+H)⁺.

### i. Tert-butyl 2-[2-[[6-methoxy-5-[2-(4-methylpiperazin-1-yl)-2-oxo-ethoxy]-1,3-benzothiazol-2-yl]methylcarbamoyl]indan-2-yl]acetate

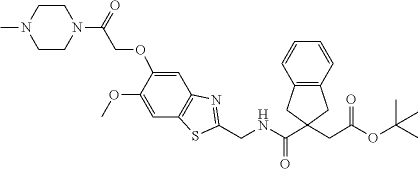 [SMILES: COc1cc2sc(CNC(=O)C3(CC(=O)OC(C)(C)C)Cc4ccccc4C3)nc2cc1OCC(=O)N1CCN(C)CC1]

A solution of 2-{[2-(aminomethyl)-6-methoxy-1,3-benzothiazol-5-yl]oxy}-1-(4-methylpiperazin-1-yl)ethan-1-one (151 mg, 0.55 mmol), 2-(2-(tert-butoxy)-2-oxoethyl)-2,3-dihydro-1H-indene-2-carboxylic acid (192 mg, 0.55 mmol) and triethylamine (0.23 mL, 166 mg, 0.66 mmol) in DCM (3 mL) was treated with HATU (250 mg, 0.66 mmol). After 2 h the mixture was partitioned between ethyl acetate and saturated aqueous sodium chloride solution. The organic phase was dried (Na₂SO₄) and evaporated. The residue was chromatographed on silica eluting with 2-10% 7M ammonia/methanol in DCM affording an off-white solid (209 mg, 63%). M/z 609.7 (M+H)⁺.

### j. 2-[2-[[6-methoxy-5-[2-(4-methylpiperazin-1-yl)-2-oxo-ethoxy]-1,3-benzothiazol-2-yl]methylcarbamoyl]indan-2-yl]acetic acid

A solution of tert-butyl 2-[2-[[6-methoxy-5-[2-(4-methylpiperazin-1-yl)-2-oxo-ethoxy]-1,3-benzothiazol-2-yl]methylcarbamoyl]indan-2-yl]acetate (206 mg, 0.33 mol) in DCM (3 mL) was treated with water (0.2 mL) then TFA (1.6 mL). After 2 h the mixture was evaporated. Toluene was added and the mixture re-evaporated. The residue was subjected to MDAP purification followed by freeze-drying of product-containing fractions to afford the title compound as white solid (108 mg, 58%). M/z 553.4 (M+H)⁺. ¹H NMR (400 MHz, d₆-DMSO) δ 8.80 (1H, bs), 7.60 (1H, s), 7.40 (1H, s), 7.20 (2H, m), 7.10 (2H, m) 4.90 (2H, s), 4.60 (2H, d), 3.80 (3H, s), 3.50 (4H, m), 3.00 (2H, d), 2.70 (2H, m), 2.40 (2H, m), 2.30 (2H, m), 2.20 (3H, s).

### Example 9 2-[2-[[6-methoxy-5-(2-morpholinoethoxy)-1,3-benzothiazol-2-yl]methylcarbamoyl]indan-2-yl]acetic acid

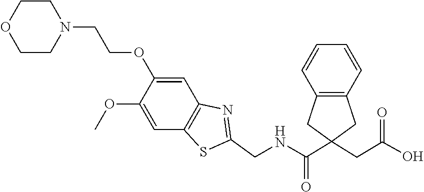 [SMILES: COc1cc2sc(CNC(=O)C3(CC(=O)O)Cc4ccccc4C3)nc2cc1OCCN1CCOCC1]

This was prepared as a white solid (101 mg) in an analogous manner to Example 8 with the change that commercially-available 4-(2-chloroethyl)morpholine hydrochloride was used in place of 1-(2-chloroacetyl)-4-methyl-piperazine hydrochloride. M/z 526.4 (M+H)⁺. ¹H NMR (400 MHz, d₆-DMSO) δ 12.00 (1H, bs), 8.80 (1H, bs), 7.60 (1H, s), 7.50 (1H, s), 7.20 (2H, m), 7.10 (2H, m), 4.60 (2H, d), 4.20 (2H, m), 3.80 (3H, s), 3.70 (2H, m), 3.50 (2H, d), 3.45-3.30 (10H, m), 3.00 (2H, d).

### Example 10 2-[5,6-difluoro-2-[[6-methoxy-5-(2-morpholinoethoxy)-1,3-benzothiazol-2-yl]methylcarbamoyl]indan-2-yl]acetic acid

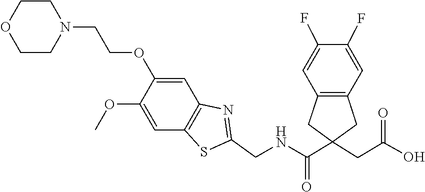 [SMILES: COc1cc2sc(CNC(=O)C3(CC(=O)O)Cc4cc(F)c(F)cc4C3)nc2cc1OCCN1CCOCC1]

### a. Dimethyl 4,5-difluorophthalate

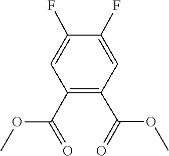

To an ice-cooled solution of 4,5-difluorophthalic acid (11.9 g, 58.9 mmol) in MeOH (250 mL) was added concentrated H₂SO₄ (40 mL, 0.75 mol) keeping the temperature <20° C. The mixture was stirred at 65° C. for 4 h. The cooled reaction mixture was concentrated in vacuo, then the residue was cautiously added to EtOAc and aq. NaHCO₃. The aq. phase was extracted with EtOAc and the combined organic extracts were washed with aq. NaHCO₃, then brine, dried (Na₂SO₄), filtered and concentrated in vacuo to yield the title compound as a colourless oil (12.98 g, 96%). ¹H NMR (CDCl₃) δ 7.56 (2H, t, J=8.7 Hz), 3.91 (6H, s).

### b. (4,5-Difluoro-1,2-phenylene)dimethanol

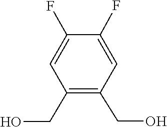 [SMILES: OCc1cc(F)c(F)cc1CO]

To an ice-cooled solution of lithium aluminium hydride (1M in THF, 226 mL, 0.226 mol) was added a solution of dimethyl 4,5-difluorophthalate (12.98 g, 56.4 mmol) in THF (100 mL) over 30 min keeping the temperature below 12° C. The mixture was stirred in the ice bath for 30 min, then at RT for 1 h. The reaction mixture was cooled to 0° C. then, cautiously, water (8.5 mL), 15% aq. NaOH (8.5 mL) and water (26 mL) were added successively, keeping the temperature below 15° C. Celite was added and the mixture stirred at RT for 1 h, then filtered through a celite pad, washing through with more THF. The filtrate was concentrated in vacuo to yield the title compound as a white solid (9.52 g, 97%). ¹H NMR (d6-DMSO) δ 7.36 (2H, t, J=10.1 Hz), 5.29 (2H, t, J=5.5 Hz), 4.47 (4H, d, J=5.4 Hz).

### c. 1,2-Bis(bromomethyl)-4,5-difluorobenzene

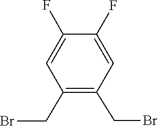 [SMILES: Fc1cc(CBr)c(CBr)cc1F]

A mixture of (4,5-difluoro-1,2-phenylene)dimethanol (9.52 g, 54.7 mmol) and 48% hydrobromic acid (68.5 mL) was stirred at 110° C. for 1 h. The cooled reaction mixture was diluted with water and then extracted with Et₂O. The aq. phase was extracted with Et₂O and the combined organic extracts were washed with water, then brine, dried (Na₂SO₄), filtered and concentrated in vacuo to leave a residue. FCC (1-10% EtOAc in hexane) to yield the title compound as a colourless oil (15.2 g, 93%). ¹H NMR (CDCl₃) δ 7.20 (2H, t, J=9.1 Hz), 4.55 (4H, s).

### d. Diethyl 5,6-difluoro-1,3-dihydro-2H-indene-2,2-dicarboxylate

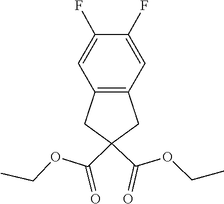 [SMILES: CCOC(=O)C1(C(=O)OCC)Cc2cc(F)c(F)cc2C1]

Sodium hydride (60% in oil, 4.46 g, 112 mmol) was added over 15 min to a mixture of 1,2-bis(bromomethyl)-4,5-difluorobenzene (15.2 g, 50.7 mmol) and diethyl malonate (9.74 g, 60.8 mmol) in THF (200 mL) keeping the temperature below 20° C. The mixture was stirred at RT for 4 h, then saturated ammonium chloride was added. The mixture was concentrated in vacuo and then extracted twice with EtOAc. The combined organic extracts were washed with brine, dried (Na₂SO₄), filtered and concentrated in vacuo to leave a residue. FCC (5-25% EtOAc in hexane) yielded the title compound as a colourless oil (9.95 g, 66%). ¹H NMR (CDCl₃) δ 6.97 (2H, t, J=8.7 Hz), 4.21 (4H, q, J=7.1 Hz), 3.52 (4H, s), 1.26 (6H, t, J=7.1 Hz).

### e. 5,6-Difluoro-2,3-dihydro-1H-indene-2-carboxylic acid

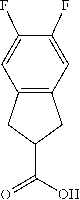 [SMILES: O=C(O)C1Cc2cc(F)c(F)cc2C1]

To a solution of diethyl 5,6-difluoro-1,3-dihydro-2H-indene-2,2-dicarboxylate (9.94 g, 33.3 mmol) in dioxane (130 mL) was added water (130 mL) and concentrated HCl (140 mL). The mixture was refluxed for 23 h. The cooled reaction mixture was diluted with water and extracted with Et₂O (×3). The combined organic extracts were washed with water, then brine, dried (Na₂SO₄), filtered and concentrated in vacuo to yield the title compound as a colourless solid (6.6 g, quant.). M/z 197 (M−H)⁻.

### f. Methyl 5,6-difluoro-2,3-dihydro-1H-indene-2-carboxylate

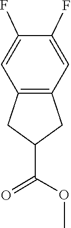

To an ice-cooled solution of 5,6-difluoro-2,3-dihydro-1H-indene-2-carboxylic acid (6.6 g, 33.3 mmol) in MeOH (200 mL) was added concentrated H₂SO₄ (40 mL, 0.75 mol) keeping the temperature <20° C. The mixture was stirred at 65° C. for 1 h. The cooled reaction mixture was concentrated in vacuo, then the residue was cautiously added to EtOAc and aq. NaHCO₃. The aq. phase was extracted with more EtOAc and the combined organic extracts were washed with brine, dried (Na₂SO₄), filtered and concentrated in vacuo to leave a residue. FCC (5-25% EtOAc in hexane) yielded the title compound as a pale yellow solid (5.97 g, 84%). ¹H NMR (CDCl₃) δ 6.98 (2H, t, J=8.8 Hz), 3.73 (3H, s), 3.39 (1H, m), 3.24-3.12 (4H, m).

### g. Methyl 2-(2-(tert-butoxy)-2-oxoethyl)-5,6-difluoro-2,3-dihydro-1H-indene-2-carboxylate

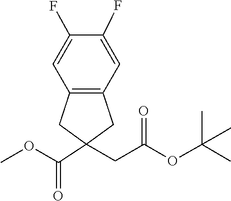 [SMILES: COC(=O)C1(CC(=O)OC(C)(C)C)Cc2cc(F)c(F)cc2C1]

To a solution of methyl 5,6-difluoro-2,3-dihydro-1H-indene-2-carboxylate (5.97 g, 28.2 mmol) in THF (120 mL), cooled to −78° C., was added sodium bis(trimethylsilyl)amide (1M in THF, 42.2 mL, 42.2 mol) over 15 min. The mixture was stirred at −78° C. for 45 min then a solution of tert-butyl bromoacetate (8.24 g, 42.2 mmol) in THF (15 mL) was added over 10 min. The reaction mixture was allowed to warm to −10° C. over 1 h. Saturated ammonium chloride was added, the mixture was concentrated under reduced pressure. The residue was extracted twice with EtOAc and the combined organic extracts were washed with brine, dried (Na₂SO₄), filtered and concentrated in vacuo to leave a residue. FCC (5-20% EtOAc in hexane) yielded the title compound as a pale yellow gum (8.78 g, 96%). ¹H NMR (CDCl₃) δ 6.96 (2H, t, J=8.9 Hz), 3.72 (3H, s), 3.47 (2H, d, J=16.2 Hz), 2.90 (2H, d, J=16.2 Hz), 2.71 (2H, s), 1.42 (9H, s).

### h. 2-[(tert-butoxy)carbonyl]-5,6-difluoro-2,3-dihydro-1H-indene-2-carboxylic acid

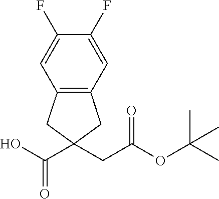 [SMILES: CC(C)(C)OC(=O)CC1(C(=O)O)Cc2cc(F)c(F)cc2C1]

To a solution of methyl 2-(2-(tert-butoxy)-2-oxoethyl)-5,6-difluoro-2,3-dihydro-1H-indene-2-carboxylate (0.834 g, 2.56 mmol) in THF (25 mL) and MeOH (10 mL) was added lithium hydroxide (0.5M in water, 10.2 mL, 5.1 mmol). The mixture was stirred at RT for 2.5 h, then concentrated in vacuo. The residual solution was layered with EtOAc and acidified by addition of 6M HCl. The aq. phase was extracted with more EtOAc and the combined organic extracts were washed with brine, dried (Na₂SO₄), filtered and concentrated in vacuo to leave a residue. FCC (2-6% MeOH in DCM) yielded the title compound as a cream solid (0.59 g, 74%). ¹H NMR (d6-DMSO) δ 12.47 (1H, bs), 7.26 (2H, t, J=9.2 Hz), 3.33 (2H, d, J=16.4 Hz), 2.91 (2H, d, J=16.4 Hz), 2.67 (2H, s), 1.37 (9H, s). M/z 311 (M−H)⁻.

### i. 2-[5,6-difluoro-2-[[6-methoxy-5-(2-morpholinoethoxy)-1,3-benzothiazol-2-yl]methylcarbamoyl]indan-2-yl]acetic acid

The title product was prepared as a white solid (18 mg) in an analogous manner to Example 9 with the change that 2-[(tert-butoxy)carbonyl]-5,6-difluoro-2,3-dihydro-1H-indene-2-carboxylic acid was used in place of 2-[(tert-butoxy)carbonyl]-2,3-dihydro-1H-indene-2-carboxylic acid. M/z 562.4 (M+H)⁺. ¹H NMR (400 MHz, d-DMSO) δ 12.00 (1H, bs), 8.80 (1H, bs), 7.60 (1H, s), 7.50 (1H, s), 7.30 (2H, t), 4.60 (2H, d), 4.20 (2H, t), 3.80 (3H, s), 3.60 (2H, t), 3.45 (2H, d), 3.30 (4H, m), 3.00 (2H, d), 2.75 (4H, m).

### Example 11 2-[5,6-difluoro-2-[[6-methoxy-5-[(1-methyl-4-piperidyl)methoxy]-1,3-benzothiazol-2-yl]methylcarbamoyl]indan-2-yl]acetic acid

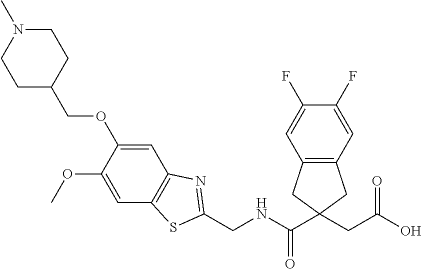 [SMILES: COc1cc2sc(CNC(=O)C3(CC(=O)O)Cc4cc(F)c(F)cc4C3)nc2cc1OCC1CCN(C)CC1]

This was prepared in a similar manner to Example 10 except that (1-methylpiperidin-4-yl)methanol hydrochloride was used in the alkylation step instead of 4-(2-chloroethyl)morpholine hydrochloride, affording the title compound as a white solid (58 mg). M/z 560.4 (M+H)⁺. ¹H NMR (400 MHz, d₆-DMSO) δ 9.00 (1H, bs), 7.60 (1H, s), 7.45 (1H, s), 7.20 (2H, t), 4.65 (2H, d), 3.95 (2H, m), 3.85 (3H, s), 3.45 (2H, d), 2.95 (2H, d), 2.80 (2H, m), 2.30 (3H, s), 2.00 (2H, m), 1.80 (3H, m), 1.40 (2H, m).

### Example 12 2-[5,6-difluoro-2-[[6-methoxy-5-[2-(4-methylpiperazin-1-yl)-2-oxo-ethoxy]-1,3-benzothiazol-2-yl]methylcarbamoyl]indan-2-yl]acetic acid

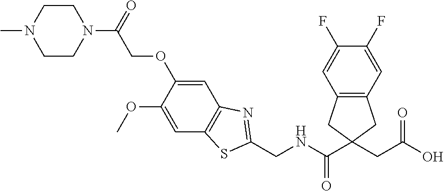 [SMILES: COc1cc2sc(CNC(=O)C3(CC(=O)O)Cc4cc(F)c(F)cc4C3)nc2cc1OCC(=O)N1CCN(C)CC1]

### a. Tert-butyl 2-[5,6-difluoro-2-[[6-methoxy-5-[2-(4-methylpiperazin-1-yl)-2-oxo-ethoxy]-1,3-benzothiazol-2-yl]methylcarbamoyl]indan-2-yl]acetate

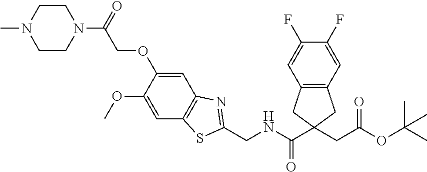 [SMILES: COc1cc2sc(CNC(=O)C3(CC(=O)OC(C)(C)C)Cc4cc(F)c(F)cc4C3)nc2cc1OCC(=O)N1CCN(C)CC1]

A solution of 2-{[2-(aminomethyl)-6-methoxy-1,3-benzothiazol-5-yl]oxy}-1-(4-methylpiperazin-1-yl)ethan-1-one (151 mg, 0.43 mmol), 2-[(tert-butoxy)carbonyl]-5,6-difluoro-2,3-dihydro-1H-indene-2-carboxylic acid (135 mg, 0.43 mmol) and triethylamine (0.18 mL, 131 mg, 1.3 mmol) in DCM (3 mL) was treated with HATU (197 mg, 0.52 mmol). After 2 h the mixture was partitioned between DCM and saturated aqueous sodium chloride solution. The organic phase was dried (Na₂SO₄) and evaporated. The residue was chromatographed on silica eluting with 1-10% 7M ammonia/methanol in DCM affording an off-white solid (169 mg, 61%). M/z 645.7 (M+H)⁺.

### b. 2-[5,6-difluoro-2-[[6-methoxy-5-[2-(4-methylpiperazin-1-yl)-2-oxo-ethoxy]-1,3-benzothiazol-2-yl]methylcarbamoyl]indan-2-yl]acetic acid

A solution of tert-butyl 2-[5,6-difluoro-2-[[6-methoxy-5-[2-(4-methylpiperazin-1-yl)-2-oxo-ethoxy]-1,3-benzothiazol-2-yl]methylcarbamoyl]indan-2-yl]acetate (65 mg, 0.1 mol) in DCM (2 mL) was treated with water (0.05 mL) then TFA (0.5 mL). After 2 h the mixture was evaporated. Toluene was added and the mixture re-evaporated. The residue was subjected to MDAP purification followed by freeze-drying of product-containing fractions to afford the title compound as white solid (11 mg, 19%). M/z 589.6 (M+H)⁺. ¹H NMR (400 MHz, d-DMSO) δ 12.20 (1H, bs), 9.80 (1H, bs), 7.60 (1H, s), 7.50 (1H, s), 7.25 (2H, t), 4.95 (2H, m), 4.65 (2H, m), 4.40 (1H, m), 4.10 (1H, m), 3.95 (2H, m), 3.80 (3H, s), 3.70-3.50 (3H, m), 3.45 (2H, d), 3.15 (1H, m), 3.00 (2H, d), 2.80 (3H, s).

### Example 13 2-[5,6-difluoro-2-[[6-methoxy-5-[2-(4-methylmorpholin-4-ium-4-yl)ethoxy]-1,3-benzothiazol-2-yl]methylcarbamoyl]indan-2-yl]acetate

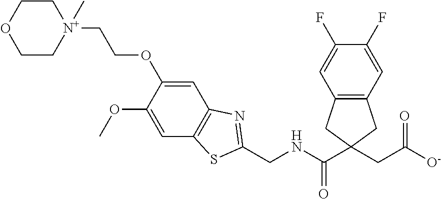 [SMILES: COc1cc2sc(CNC(=O)C3(CC(=O)[O-])Cc4cc(F)c(F)cc4C3)nc2cc1OCC[N+]1(C)CCOCC1]

### a. Tert-butyl 2-[5,6-difluoro-2-[[6-methoxy-5-[2-(4-methylmorpholin-4-ium-4-yl)ethoxy]-1,3-benzothiazol-2-yl]methylcarbamoyl]indan-2-yl]acetate iodide

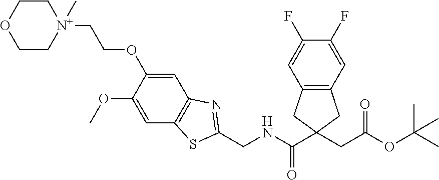 [SMILES: COc1cc2sc(CNC(=O)C3(CC(=O)OC(C)(C)C)Cc4cc(F)c(F)cc4C3)nc2cc1OCC[N+]1(C)CCOCC1]

A solution of tert-butyl 5,6-difluoro-2-[({6-methoxy-5-[2-(morpholin-4-yl)ethoxy]-1,3-benzothiazol-2-yl}methyl)carbamoyl]-2,3-dihydro-1H-indene-2-carboxylate (see Example 10) (140 mg, 0.23 mmol) in THF (2 mL) was treated with iodomethane (161 mg, 0.07 mL, 1.1 mmol) and stirred overnight. Evaporation gave an oil (0.2 g) which was used directly in the next step. M/z 632.6 (M)⁺

### b. 2-[5,6-difluoro-2-[[6-methoxy-5-[2-(4-methylmorpholin-4-ium-4-yl)ethoxy]-1,3-benzothiazol-2-yl]methylcarbamoyl]indan-2-yl]acetate

A solution of the above tert-butyl 2-[5,6-difluoro-2-[[6-methoxy-5-[2-(4-methylmorpholin-4-ium-4-yl)ethoxy]-1,3-benzothiazol-2-yl]methylcarbamoyl]indan-2-yl]acetate iodide (0.2 g, 0.23 mmol) in DCM (2 mL) was treated with water (0.1 mL) then TFA (1 mL). After 2 h the mixture was evaporated. Toluene was added and the mixture re-evaporated. The residue was subjected to MDAP purification followed by freeze-drying of product-containing fractions to afford the title compound as white solid (54 mg, 41%). M/z 576.4 (M+H)⁺. ¹H NMR (400 MHz, d₆-DMSO) δ 12.90 (1H, bs), 7.65 (1H, s), 7.60 (1H, s), 7.20 (2H, t), 4.65 (2H, m), 4.60 (2H, m), 4.10-3.90 (6H, m), 3.85 (3H, s), 3.70-3.50 (4H, m), 3.40 (2H, d), 3.30 (3H, s), 2.90 (2H, d).

### Example 14 2-[2-[[5-[2-(4,4-dimethylpiperazin-4-ium-1-yl)-2-oxo-ethoxy]-6-methoxy-1,3-benzothiazol-2-yl]methylcarbamoyl]-5,6-difluoro-indan-2-yl]acetate

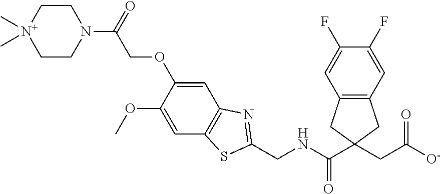 [SMILES: COc1cc2sc(CNC(=O)C3(CC(=O)[O-])Cc4cc(F)c(F)cc4C3)nc2cc1OCC(=O)N1CC[N+](C)(C)CC1]

This was prepared from tert-butyl 5,6-difluoro-2-[({6-methoxy-5-[2-(4-methylpiperazin-1-yl)-2-oxoethoxy]-1,3-benzothiazol-2-yl}methyl)carbamoyl]-2,3-dihydro-1H-indene-2-carboxylate, the precursor to Example 12, using the quaternisation with iodomethane followed by TFA deprotection protocol as described for Example 8 step-j to afford the title compound as a white solid (48 mg). M/z 603.3 (M+H)⁺. ¹H NMR (400 MHz, d₆-DMSO) δ 13.10 (1H, bs), 7.68 (1H, s), 7.50 (1H, s), 7.25 (2H, t), 4.95 (2H, m), 4.60 (2H, m), 4.00-3.90 (4H, m), 3.85 (3H, s), 3.50 (2H, m), 3.40 (2H, m), 3.20 (6H, s), 2.90 (2H, d), 2.40 (2H, d).

### Example 15 2-[5,6-difluoro-2-[[6-methoxy-5-[3-(4-methylmorpholin-4-ium-4-yl)propoxy]-1,3-benzothiazol-2-yl]methylcarbamoyl]indan-2-yl]acetate

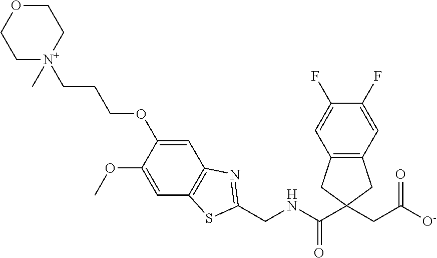 [SMILES: COc1cc2sc(CNC(=O)C3(CC(=O)[O-])Cc4cc(F)c(F)cc4C3)nc2cc1OCCC[N+]1(C)CCOCC1]

This was prepared from tert-butyl 5,6-difluoro-2-[({6-methoxy-5-[3-(morpholin-4-yl)propoxy]-1,3-benzothiazol-2-yl}methyl)carbamoyl]-2,3-dihydro-1H-indene-2-carboxylate, which was accessed using the same chemistry as for Example 10 with the change that 4-(2-chloropropyl)morpholine hydrochloride was used in the phenol alkylation step. Quaternisation of tert-butyl 5,6-difluoro-2-[({6-methoxy-5-[3-(morpholin-4-yl)propoxy]-1,3-benzothiazol-2-yl}methyl)carbamoyl]-2,3-dihydro-1H-indene-2-carboxylate with iodomethane followed by TFA deprotection protocol as described for Example 13 to afford the title compound as a white solid (80 mg). M/z 590.4 (M+H)⁺. ¹H NMR (400 MHz, d₆-DMSO). 11.80 (1H, bs), 7.60 (1H, s), 7.50 (1H, s), 7.25 (2H, t), 4.60 (2H, d), 4.20 (2H, t), 4.00 (2H, m), 3.87 (3H, s), 3.70 (2H, m), 3.50 (2H, m), 3.35 (2H, d), 3.20 (3H, s), 3.00 (2H, d), 2.80 (2H, m), 2.50 (2H, m), 2.25 (2H, m).

### Example 16 2-[2-[[6-methoxy-5-[3-(4-methylmorpholin-4-ium-4-yl)propoxy]-1,3-benzothiazol-2-yl]methylcarbamoyl]indan-2-yl]acetate

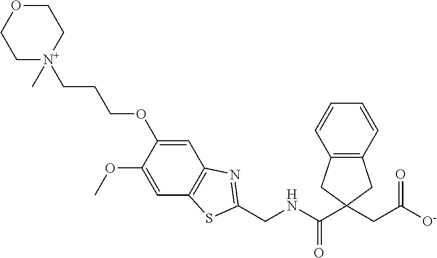 [SMILES: COc1cc2sc(CNC(=O)C3(CC(=O)[O-])Cc4ccccc4C3)nc2cc1OCCC[N+]1(C)CCOCC1]

This was prepared from tert-butyl 2-[({6-methoxy-5-[3-(morpholin-4-yl)propoxy]-1,3-benzothiazol-2-yl}methyl)carbamoyl]-2,3-dihydro-1H-indene-2-carboxylate, which was accessed using the same chemistry as for Example 8 with the change that 4-(2-chloropropyl)morpholine hydrochloride was used in the phenol alkylation step. Quaternisation of tert-butyl 2-[({6-methoxy-5-[3-(morpholin-4-yl)propoxy]-1,3-benzothiazol-2-yl}methyl)carbamoyl]-2,3-dihydro-1H-indene-2-carboxylate with iodomethane followed by TFA deprotection protocol as described for Example 13 to afford the title compound as a white solid. M/z 554.2 (M+H)⁺. ¹H NMR (500 MHz, DMSO-d₆): δ 11.80 (1H, bs), 7.63 (1H, s), 7.54 (1H, s), 7.18-7.16 (2H, m), 7.12-7.10 (2H, m), 4.61 (2H, d, J=5.5 Hz), 4.14 (2H, t, J=6.5 Hz), 3.94-3.92 (4H, m), 3.83 (3H, s), 3.66-3.63 (2H, m), 3.48-3.42 (4H, m), 3.39-3.35 (2H, bs), 3.17 (3H, s), 2.91 (2H, d, J=16.5 Hz), 2.46 (2H, s), 2.25-2.22 (2H, m).

### Example 17 2-[2-[[5-[(1,1-dimethylpiperidin-1-ium-4-yl)methoxy]-6-methoxy-1,3-benzothiazol-2-yl]methylcarbamoyl]indan-2-yl]acetate

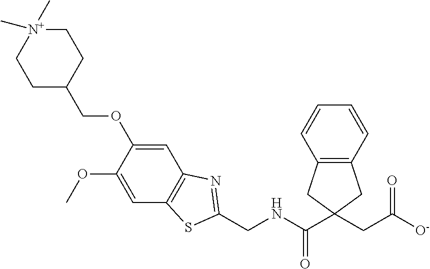 [SMILES: COc1cc2sc(CNC(=O)C3(CC(=O)[O-])Cc4ccccc4C3)nc2cc1OCC1CC[N+](C)(C)CC1]

This was prepared from tert-butyl 2-[2-[[6-methoxy-5-[(1-methyl-4-piperidyl)methoxy]-1,3-benzothiazol-2-yl]methylcarbamoyl]indan-2-yl]acetate, which was accessed using the same chemistry as for Example 8 with the change that 4-(chloromethyl)-1-methyl-piperidine was used in the phenol alkylation step. Quaternisation of tert-butyl 2-[2-[[6-methoxy-5-[(1-methyl-4-piperidyl)methoxy]-1,3-benzothiazol-2-yl]methylcarbamoyl]indan-2-yl]acetate with iodomethane followed by TFA deprotection protocol as described for Example 13 to afford the title compound as a white solid. M/z 538.1 (M+H)⁺. ¹H NMR (500 MHz, DMSO-d₆): δ 11.32 (1H, bs), 7.61 (1H, s), 7.50 (1H, s), 7.18-7.16 (2H, m), 7.12-7.10 (2H, m), 4.61 (2H, d, J=5.5 Hz), 4.02 (2H, d, J=6.5 Hz), 3.82 (3H, s), 3.45-3.33 (6H, bs), 3.10 (3H, s), 3.05 (3H, s), 2.91 (2H, d, J=16 Hz), 2.55-2.45 (2H, bs), 2.07-2.04 (1H, m), 1.94-1.92 (2H, m), 1.79-1.74 (2H, m).

### Example 18 2-[2-[[5-[3-[diethyl(methyl)ammonio]propoxy]-6-methoxy-1,3-benzothiazol-2-yl]methylcarbamoyl]indan-2-yl]acetate

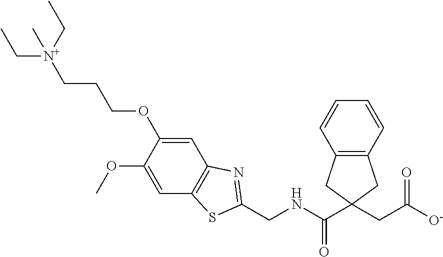 [SMILES: CC[N+](C)(CC)CCCOc1cc2nc(CNC(=O)C3(CC(=O)[O-])Cc4ccccc4C3)sc2cc1OC]

### a. Tert-butyl N-({5-[3-(diethylamino)propoxy]-6-methoxy-1,3-benzothiazol-2-yl}methyl)carbamate

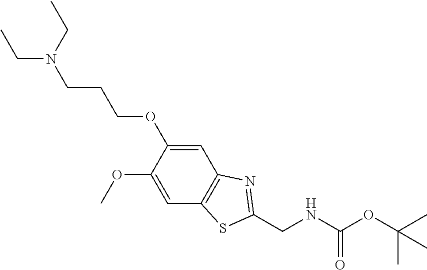 [SMILES: CCN(CC)CCCOc1cc2nc(CNC(=O)OC(C)(C)C)sc2cc1OC]

A solution of tert-butyl N-[(5-hydroxy-6-methoxy-1,3-benzothiazol-2-yl)methyl]carbamate (100 mg, 0.32 mmol), 3-diethylamino-1-propanol (50 mg, 0.38 mmol) and triphenylphosphine (100 mg, 0.38 mmol) in THF (2 mL) was treated with diethyl azodicarboxylate (67 mg, 0.38 mmol). After 2 h, 3-diethylamino-1-propanol (25 mg, 0.19 mmol), triphenylphosphine (50 mg, 0.19 mmol) and diethyl azodicarboxylate (34 mg, 0.19 mmol) were added. After 0.5 h, the mixture was diluted with toluene and evaporated. The residue was chromatographed on silica eluting with 2-12% 2M ammonia/methanol in DCM affording a pale-yellow oil (118 mg, 86%). M/z 424.4 (M+H)⁺.

### b. (3-{[2-({[(Tert-butoxy)carbonyl]amino}methyl)-6-methoxy-1,3-benzothiazol-5-yl]oxy}propyl)diethylmethylazanium iodide

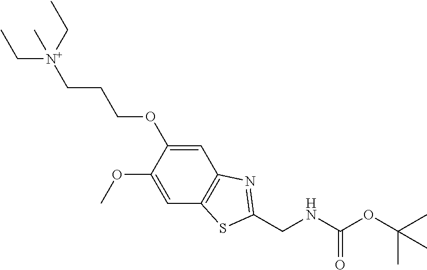 [SMILES: CC[N+](C)(CC)CCCOc1cc2nc(CNC(=O)OC(C)(C)C)sc2cc1OC]

A solution of tert-butyl N-({5-[3-(diethylamino)propoxy]-6-methoxy-1,3-benzothiazol-2-yl}methyl)carbamate (118 mg, 0.28 mmol) in ACN (3 mL) was treated with iodomethane (200 mg, 0.9 mmol). After 16 h the mixture was evaporated to dryness and the residue chromatographed on silica eluting with 3-20% 2M ammonia/methanol in DCM affording a colourless oil (95 mg, 61%). M/z 438.5 (M)⁺.

### c. (3-{[2-(Aminomethyl)-6-methoxy-1,3-benzothiazol-5-yl]oxy}propyl)diethylmethylazanium chloride, hydrochloride salt

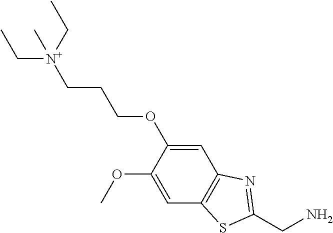 [SMILES: CC[N+](C)(CC)CCCOc1cc2nc(CN)sc2cc1OC]

A solution of (3-{[2-({1[(tert-butoxy)carbonyl]amino}methyl)-6-methoxy-1,3-benzothiazol-5-yl]oxy}propyl)diethylmethylazanium iodide (95 mg, 0.17 mmol) in methanol (1 mL) was treated with 4M hydrochloric acid in 1,4-dioxane (3 mL, 12 mmol). After 1.5 h, toluene was added and the mixture evaporated to give an oil (100 mg). M/z 338.4 (M)⁺.

### d. 3-[[2-[[[2-(2-tert-butoxy-2-oxo-ethyl)indane-2-carbonyl]amino]methyl]-6-methoxy-1,3-benzothiazol-5-yl]oxy]propyl-diethyl-methyl-ammonium chloride

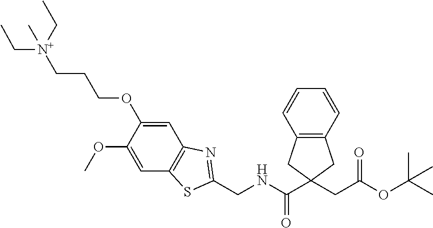 [SMILES: CC[N+](C)(CC)CCCOc1cc2nc(CNC(=O)C3(CC(=O)OC(C)(C)C)Cc4ccccc4C3)sc2cc1OC]

A solution of (3-{[2-(aminomethyl)-6-methoxy-1,3-benzothiazol-5-yl]oxy}propyl)diethylmethylazanium chloride, hydrochloride salt (100 mg), 2-(2-(tert-butoxy)-2-oxoethyl)-2,3-dihydro-1H-indene-2-carboxylic acid (51 mg, 0.18 mmol) and DIPEA (65 mg, 0.5 mmol) in DMF (1.5 mL) was treated with HATU (95 mg, 0.18 mmol). After 20 minutes the mixture was chromatographed on reverse phase C18 silica eluting with 20-50% 0.01M hydrochloric acid in ACN. Product-containing fractions were freeze-dried to afford a light brown solid (94 mg, 89% over the two stages). M/z 596.4 (M)⁺.

### e. 2-[2-[[5-[3-[diethyl(methyl)ammonio]propoxy]-6-methoxy-1,3-benzothiazol-2-yl]methylcarbamoyl]indan-2-yl]acetate

A solution of 3-[[2-[[[2-(2-tert-butoxy-2-oxo-ethyl)indane-2-carbonyl]amino]methyl]-6-methoxy-1,3-benzothiazol-5-yl]oxy]propyl-diethyl-methyl-ammonium chloride (94 mg, 0.15 mmol) in DCM (3 mL) was treated with TFA (1.5 mL). After 2 h toluene was added and the mixture evaporated. The residue was chromatographed on reverse phase C₁₈ silica eluting with 10-30% 2M ammonia/methanol in ACN. Product-containing fractions were freeze-dried to afford the title compound as a white solid (37 mg, 46%). M/z 540.3 (M+H)⁺. ¹H NMR (400 MHz, d₆-DMSO) δ 11.50 (1H, bs), 7.65 (1H, s), 7.52 (1H, s), 7.20 (2H, m), 7.10 (2H, m), 4.62 (2H, d), 4.15 (2H, t), 3.85 (3H, s), 3.50-3.20 (6H, m), 3.00 (3H, s), 2.90 (2H, d), 2.40 (2H, m), 2.20 (2H, m), 1.25 (6H, t).

### Example 19 2-[5,6-difluoro-2-[[6-methoxy-5-[3-(1-methylpyrrolidin-1-ium-1-yl)propoxy]-1,3-benzothiazol-2-yl]methylcarbamoyl]indan-2-yl]acetate

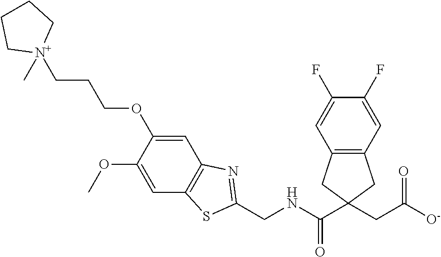 [SMILES: COc1cc2sc(CNC(=O)C3(CC(=O)[O-])Cc4cc(F)c(F)cc4C3)nc2cc1OCCC[N+]1(C)CCCC1]

This was prepared in an analogous manner to Example 18 with the changes that 3-(pyrrolidine-1-yl)propan-1-ol was used in place of 3-diethylamino-1-propanol and 2-[(tert-butoxy)carbonyl]-5,6-difluoro-2,3-dihydro-1H-indene-2-carboxylic acid was used in place of 2-[(tert-butoxy)carbonyl]-2,3-dihydro-1H-indene-2-carboxylic acid. The title compound was isolated as a white solid (45 mg). M/z 574.4 (M+H)⁺. ¹H NMR (400 MHz, d-DMSO) δ 7.68 (1H, s), 7.55 (1H, s), 7.22 (2H, t), 4.65 (2H, d), 4.15 (2H, t), 3.85 (3H, s), 3.50 (6H, m), 3.40 (2H, d), 3.05 (3H, s), 2.90 (2H, d), 2.30 (2H, m), 2.10 (4H, m).

### Example 20

### 2-[2-[[5-[3-[2-hydroxyethyl(dimethyl)ammonio]propoxy]-6-methoxy-1,3-benzothiazol-2-yl]methylcarbamoyl]indan-2-yl]acetate

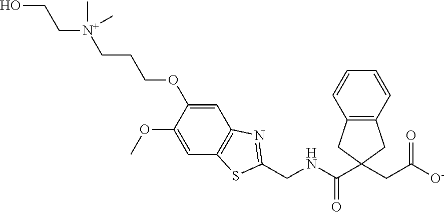 [SMILES: COc1cc2sc(CNC(=O)C3(CC(=O)[O-])Cc4ccccc4C3)nc2cc1OCCC[N+](C)(C)CCO]

### a. Tert-butyl N-{[6-methoxy-5-(3-{methyl[2-(oxan-2-yloxy)ethyl]amino}propoxy)-1,3-benzothiazol-2-yl]methyl}carbamate

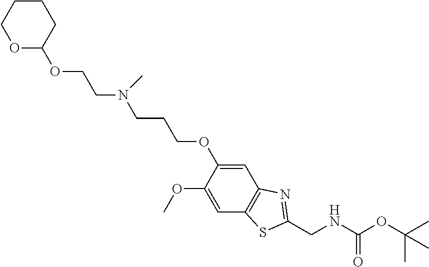 [SMILES: COc1cc2sc(CNC(=O)OC(C)(C)C)nc2cc1OCCCN(C)CCOC1CCCCO1]

This was prepared by the same procedure as for Example (18a) with the change that 3-(methyl(2-((tetrahydro-2H-pyran-2-yl)oxy)ethyl)amino)propan-1-ol was used in place of 3-diethylamino-1-propanol, affording a colourless oil (212 mg, 54%). M/z 510.4 (M+H)⁺.

### b. (3-{[2-({[(Tert-butoxy)carbonyl]amino}methyl)-6-methoxy-1,3-benzothiazol-5-yl]oxy}propyl)dimethyl[2-(oxan-2-yloxy)ethyl]azanium iodide

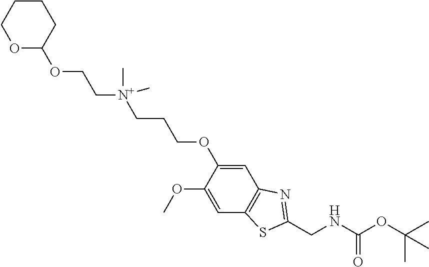 [SMILES: COc1cc2sc(CNC(=O)OC(C)(C)C)nc2cc1OCCC[N+](C)(C)CCOC1CCCCO1]

This was prepared from tert-butyl N-{[6-methoxy-5-(3-{methyl[2-(oxan-2-yloxy)ethyl]amino}propoxy)-1,3-benzothiazol-2-yl]methyl}carbamate by the same procedure as for Example (18b) affording a clear oil (201 mg, 74%). M/z 524.4 (M)⁺.

### c. (3-{[2-(Aminomethyl)-6-methoxy-1,3-benzothiazol-5-yl]oxy}propyl)(2-hydroxyethyl)dimethylazanium chloride hydrochloride

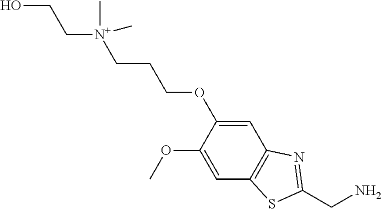 [SMILES: COc1cc2sc(CN)nc2cc1OCCC[N+](C)(C)CCO]

This was prepared from (3-{[2-({[(tert-butoxy)carbonyl]amino}methyl)-6-methoxy-1,3-benzothiazol-5-yl]oxy}propyl)dimethyl[2-(oxan-2-yloxy)ethyl]azanium iodide by the method of Example (18c) with the difference that the reaction time was lengthened to 2 h from 1.5 h, affording an oil in quantitative yield. M/z 340.3 (M)⁺.

### d. 3-[[2-[[[2-(2-tert-butoxy-2-oxo-ethyl)indane-2-carbonyl]amino]methyl]-6-methoxy-1,3-benzothiazol-5-yl]oxy]propyl-(2-hydroxyethyl)-dimethyl-ammonium chloride

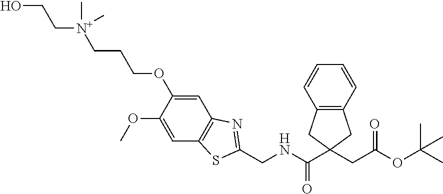 [SMILES: COc1cc2sc(CNC(=O)C3(CC(=O)OC(C)(C)C)Cc4ccccc4C3)nc2cc1OCCC[N+](C)(C)CCO]

This was prepared from (3-{[2-(aminomethyl)-6-methoxy-1,3-benzothiazol-5-yl]oxy}propyl)(2-hydroxyethyl)dimethylazanium chloride hydrochloride by the method of Example (18d) affording a white solid (68 mg, 69%). M/z 598.4 (M)⁺

### e. 2-[2-[[5-[3-[2-hydroxyethyl(dimethyl)ammonio]propoxy]-6-methoxy-1,3-benzothiazol-2-yl]methylcarbamoyl]indan-2-yl]acetate

This was prepared from 3-[[2-[[[2-(2-tert-butoxy-2-oxo-ethyl)indane-2-carbonyl]amino]methyl]-6-methoxy-1,3-benzothiazol-5-yl]oxy]propyl-(2-hydroxyethyl)-dimethyl-ammonium chloride by the method of Example (18e) affording the title compound as a white solid (40 mg, 68%). M/z 542.4 (M+H)⁺. ¹H NMR (400 MHz, d₆-DMSO) δ 13.10 (1H, bs), 7.65 (1H, s), 7.50 (1H, s), 7.15 (2H, m), 7.05 (2H, m), 5.50 (1H, bs), 4.60 (2H, d), 4.15 (2H, t), 3.90 (2H, m), 3.85 (3H, s), 3.55 (2H, m), 3.46 (2H, m), 3.35 (2H, d), 3.15 (6H, s), 2.87 (2H, d), 2.40 (2H, m), 2.30 (2H, m).

### Example 21 2-[5,6-difluoro-2-[[5-[3-[2-hydroxyethyl(dimethyl)ammonio]propoxy]-6-methoxy-1,3-benzothiazol-2-yl]methylcarbamoyl]indan-2-yl]acetate

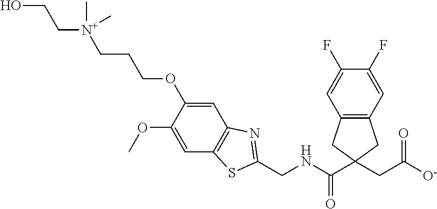 [SMILES: COc1cc2sc(CNC(=O)C3(CC(=O)[O-])Cc4cc(F)c(F)cc4C3)nc2cc1OCCC[N+](C)(C)CCO]

This was prepared in an analogous manner to Example 20 except that 2-[(tert-butoxy)carbonyl]-5,6-difluoro-2,3-dihydro-1H-indene-2-carboxylic acid was used in place of 2-[(tert-butoxy)carbonyl]-2,3-dihydro-1H-indene-2-carboxylic acid. The title compound was isolated as a white solid (35 mg). M/z 578.3 (M+H)⁺. ¹H NMR (400 MHz, d₆-DMSO) δ 13.00 (1H, bs), 7.65 (1H, s), 7.52 (1H, s), 7.20 (2H, t), 5.50 (1H, bs), 4.60 (2H, d), 4.15 (2H, t), 3.90 (2H, m), 3.85 (3H, s), 3.55 (2H, m), 3.45 (2H, m), 3.35 (2H, d), 3.15 (6H, s), 2.90 (2H, d), 2.40 (2H, m), 2.30 (2H, m).

### Example 22 2-[2-[[5-[3-[bis(2-hydroxyethyl)-methyl-ammonio]propoxy]-6-methoxy-1,3-benzothiazol-2-yl]methylcarbamoyl]indan-2-yl]acetate

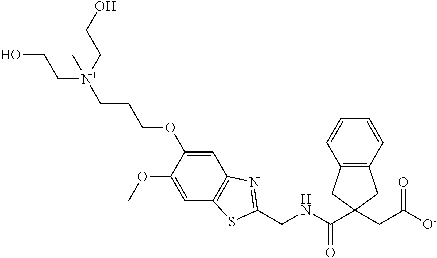 [SMILES: COc1cc2sc(CNC(=O)C3(CC(=O)[O-])Cc4ccccc4C3)nc2cc1OCCC[N+](C)(CCO)CCO]

This was prepared in an analogous manner to Example 20 except that 3-(bis(2-((tetrahydro-2H-pyran-2-yl)oxy)ethyl)amino)propan-1-ol was used in place of 3-(methyl(2-((tetrahydro-2H-pyran-2-yl)oxy)ethyl)amino)propan-1-ol. The title compound was isolated as a white solid (31 mg). M/z 572.4 (M+H)⁺. ¹H NMR (400 MHz, d₆-DMSO) δ 13.00 (1H, bs), 7.65 (1H, s), 7.50 (1H, s), 7.20 (2H, m), 7.10 (2H, m), 5.50 (2H, bs), 4.60 (2H, d), 4.20 (2H, t), 3.95 (4H, m), 3.85 (3H, s), 3.60 (2H, m), 3.50 (4H, m), 3.40 (2H, d), 3.15 (3H, s), 2.90 (2H, d), 2.20 (2H, m).

### Example 23 2-[2-[[5-[3-[bis(2-hydroxyethyl)-methyl-ammonio]propoxy]-6-methoxy-1,3-benzothiazol-2-yl]methylcarbamoyl]-5,6-difluoro-indan-2-yl]acetate

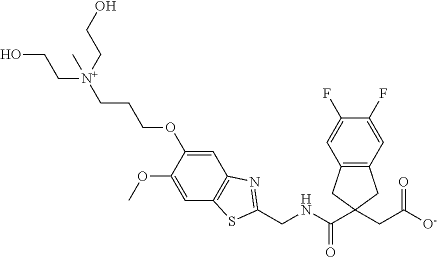 [SMILES: COc1cc2sc(CNC(=O)C3(CC(=O)[O-])Cc4cc(F)c(F)cc4C3)nc2cc1OCCC[N+](C)(CCO)CCO]

This was prepared in an analogous manner to Example 22 except that 2-[(tert-butoxy)carbonyl]-5,6-difluoro-2,3-dihydro-1H-indene-2-carboxylic acid was used in place of 2-[(tert-butoxy)carbonyl]-2,3-dihydro-1H-indene-2-carboxylic acid. The title compound was isolated as a white solid (10 mg). M/z 608.4 (M+H)⁺. ¹H NMR (400 MHz, d₆-DMSO) δ 13.00 (1H, bs), 7.65 (1H, s), 7.50 (1H, s), 7.20 (2H, t), 5.40 (2H, bs), 4.60 (2H, d), 4.10 (2H, t), 3.95 (4H, m), 3.85 (3H, s), 3.60 (2H, m), 3.50 (4H, m), 3.40 (2H, d), 3.15 (3H, s), 2.90 (2H, d), 2.20 (2H, m).

### Example 24 2-[2-[[5-[2-(4-methylpiperazin-1-yl)-2-oxo-ethoxy]-1,3-benzothiazol-2-yl]methylcarbamoyl]indan-2-yl]acetic acid

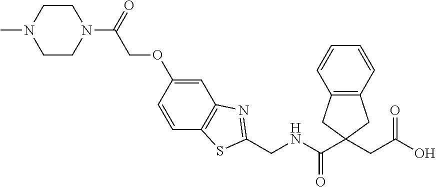 [SMILES: CN1CCN(C(=O)COc2ccc3sc(CNC(=O)C4(CC(=O)O)Cc5ccccc5C4)nc3c2)CC1]

### a. 2-hydroxy-1-(4-methylpiperazin-1-yl)ethan-1-one

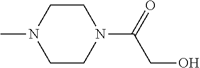

To a stirred solution of 1-methylpiperazine (500 mg, 4.99 mmol) in dioxane (5 mL) was added ethyl 2-hydroxyacetate (520 mg, 4.99 mmol) at room temperature. The reaction mixture was heated at 120° C. for 12 h and evaporated the solvent affording a pale yellow thick mass (200 mg, crude). M/z 159.1 (M+H)⁺.

### b. Tert-butyl N-[(5-bromo-1,3-benzothiazol-2-yl)methyl]carbamate

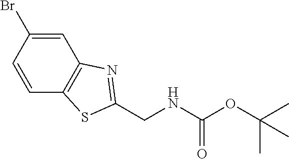 [SMILES: CC(C)(C)OC(=O)NCc1nc2cc(Br)ccc2s1]

CuO (1 g, 12.6 mmol) was added to a stirred solution of 5-bromo-2-iodo-aniline (2.5 g, 8.30 mmol) and tert-butyl N-(2-amino-2-thioxo-ethyl)carbamate (2 g, 10.9 mmol) in DMF (15 mL) at RT and the reaction mixture was purged with argon for 15 min. Then dppf (929 mg, 1.60 mmol) and Pd₂(dba)₃ (768 mg, 0.8 mmol) were added to the reaction mixture and degassed with argon for further 5 min. The reaction mixture was stirred in a sealed tube at 70° C. for 4 h and filtered through celite pad which was washed with EtOAc (50 mL). The filtrate was washed with water (2×30 mL) and concentrated under reduced pressure. The crude compound was purified by flash chromatography eluting with 20% EtOAc in petroleum ether affording an off white solid (2 g, 71%). M/z 343.0 (M+H)⁺.

### c. (5-bromo-1,3-benzothiazol-2-yl)methanamine hydrochloride

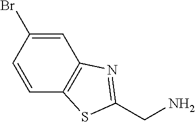 [SMILES: NCc1nc2cc(Br)ccc2s1]

4N HCl in dioxane (30 mL) was added to a solution of tert-butyl N-[(5-bromo-1,3-benzothiazol-2-yl)methyl]carbamate (3 g, 8.7 mmol) in dioxane (50 mL) at 0° C. The reaction mixture was stirred at RT for 4 h and concentrated under reduced pressure. The crude compound was triturated with n-pentane (20 mL) and Et₂O (20 mL) affording a pale yellow solid (2.2 g, 90%). M/z 243.0 (M)⁺.

### d. tert-butyl 2-[2-[(5-bromo-1,3-benzothiazol-2-yl)methylcarbamoyl]indan-2-yl]acetate

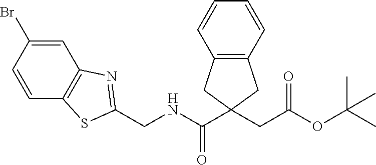 [SMILES: CC(C)(C)OC(=O)CC1(C(=O)NCc2nc3cc(Br)ccc3s2)Cc2ccccc2C1]

Et₃N (1.5 mL, 10.8 mmol) was added to a stirred solution of (5-bromo-1,3-benzothiazol-2-yl)methanamine hydrochloride (800 mg, 2.8 mmol) in DMF (10 mL) at RT and stirred for 15 min. Then 2-(2-tert-butoxy-2-oxo-ethyl)indane-2-carboxylic acid (1 g, 3.6 mmol), EDC·HCl (833 mg, 4.3 mmol) and HOBt (684 mg, 5.0 mmol) were added. The reaction mixture was stirred at RT for 16 h, diluted with ice cold water (100 mL) and extracted with EtOAc (2×50 mL). The organic layer was washed with brine solution, dried over Na₂SO₄ and concentrated under reduced pressure. The crude compound was purified by flash chromatography eluting with 25% EtOAc in petroleum ether affording an off white solid (1 g, 56%). M/z 501.1 (M+H)⁺.

### e. Tert-butyl 2-[2-[[5-(4,4,5,5-tetramethyl-1,3,2-dioxaborolan-2-yl)-1,3-benzothiazol-2-yl]methylcarbamoyl]indan-2-yl]acetate

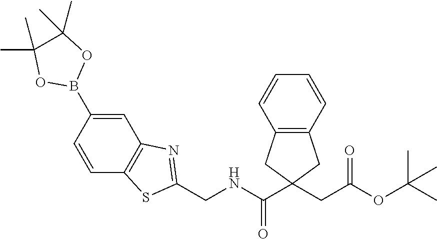 [SMILES: CC(C)(C)OC(=O)CC1(C(=O)NCc2nc3cc(B4OC(C)(C)C(C)(C)O4)ccc3s2)Cc2ccccc2C1]

To a solution of tert-butyl 2-[2-[(5-bromo-1,3-benzothiazol-2-yl)methylcarbamoyl]indan-2-yl]acetate (1.5 g, 3.0 mmol) in dioxane (20 mL) was added potassium acetate (588 mg, 6.0 mmol) and Bpin (838 mg, 3.3 mmol) at RT and the reaction mixture was purged with argon for 15 min. Then PdCl₂(dppf)·DCM (171 mg, 0.21 mmol) was added to the reaction mixture and purged with argon for further 5 min. The reaction mixture was stirred in sealed tube at 90° C. for 4 h. The reaction mixture was filtered through celite pad and washed the pad with EtOAc (50 mL). The organic extracts were washed with water (2×50 mL) and brine, then dried over sodium sulphate, filtered and the solvent removed to give crude product (1.6 g, crude) as a brown semi solid. Mixture of boronic acid M/z 467.2 (M+H)⁺ and boronate ester M/z 549.2 (M+H)⁺.

### f. [2-[[[2-(2-tert-butoxy-2-oxo-ethyl)indane-2-carbonyl]amino]methyl]-1,3-benzothiazol-5-yl]boronic acid

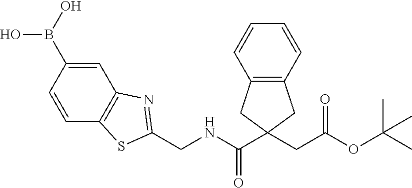 [SMILES: CC(C)(C)OC(=O)CC1(C(=O)NCc2nc3cc(B(O)O)ccc3s2)Cc2ccccc2C1]

To a solution of tert-butyl 2-[2-[[5-(4,4,5,5-tetramethyl-1,3,2-dioxaborolan-2-yl)-1,3-benzothiazol-2-yl]methylcarbamoyl]indan-2-yl]acetate (1.6 g, 2.9 mmol) in THF:H₂O (4:1, 20 mL) was added sodium periodate (1.9 g, 8.7 mmol) at RT and stirred for 30 min. Then 1N HCl (2 mL, 2.0 mmol) was added to the reaction mixture at RT and stirred at RT for 16 h. The reaction mixture was diluted with water and extracted with EtOAc. The organic extracts were washed with water and brine, then dried over sodium sulphate, filtered and the solvent removed. The crude compound was purified by column chromatography (100-200 silica gel, gradient 10% MeOH/DCM) to yield the product (900 mg, 66%) as a yellow solid. M/z 467.2 (M+H)⁺.

### g. Tert-butyl 2-(2-(((5-(2-(4-methylpiperazin-1-yl)-2-oxoethoxy)benzo[d]thiazol-2-yl)methyl)carbamoyl)-2,3-dihydro-1H-inden-2-yl)acetate

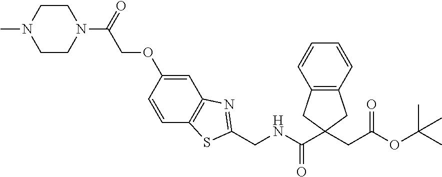 [SMILES: CN1CCN(C(=O)COc2ccc3sc(CNC(=O)C4(CC(=O)OC(C)(C)C)Cc5ccccc5C4)nc3c2)CC1]

To a solution of [2-[[[2-(2-tert-butoxy-2-oxo-ethyl)indane-2-carbonyl]amino]methyl]-1,3-benzothiazol-5-yl]boronic acid (300 mg, 0.64 mmol) in DCM (10 mL) was added Cu(OAc)₂(175 mg, 0.96 mmol), triethylamine (0.2 mL, 1.28 mmol) and molecular sieves (0.5 g) at room temperature. The reaction mixture was stirred for 10 minutes, then added 2-hydroxy-1-(4-methylpiperazin-1-yl)ethan-1-one (153 mg, 0.96 mmol) and stirred at room temperature under air for 16 h. The reaction mixture was filtered through celite pad, washed the pad with dichloromethane and filtrate was evaporated to get the crude compound. The crude was chromatographed on silica eluting with 3% MeOH in DCM affording tert-butyl 2-(2-(((5-(2-(4-methylpiperazin-1-yl)-2-oxoethoxy)benzo[d]thiazol-2-yl)methyl)carbamoyl)-2,3-dihydro-1H-inden-2-yl)acetate as a pale brown solid (60 mg, 16%). M/z 579.3 (M+H)⁺.

### h. 2-[2-[[5-[2-(4-methylpiperazin-1-yl)-2-oxo-ethoxy]-1,3-benzothiazol-2-yl]methylcarbamoyl]indan-2-yl]acetic acid

A solution of tert-butyl 2-(2-(((5-(2-(4-methylpiperazin-1-yl)-2-oxoethoxy)benzo[d]thiazol-2-yl)methyl)carbamoyl)-2,3-dihydro-1H-inden-2-yl)acetate (50 mg, 0.086 mmol) in DCM (4 mL) was treated with TFA (0.5 mL) at room temperature for 2 h. The mixture was evaporated and the residue was triturated with diethyl ether (6 mL). The crude compound was purified by preparative HPLC [SYMMETRY-C₈ (3000*19), 7 u, Mobile phase: A: 0.1% Formic Acid in H₂O, B: MeCN] and freeze dried affording an off white solid (15 mg, 34%). M/z 523.2 (M+H)⁺. ¹H NMR (500 MHz, MeOD): δ 7.79 (1H, d, J=9 Hz), 7.44 (1H, s), 7.23-7.21 (2H, m), 7.16-7.15 (2H, m), 7.13 (1H, d, J=9 Hz), 4.94 (2H, s), 4.75 (2H, s), 3.85-3.75 (4H, bs), 3.52 (2H, d, J=16.5 Hz), 3.16-3.07 (4H, bs), 3.09 (2H, d, J=16.5 Hz), 2.82 (2H, s), 2.78 (3H, s).

### Example 25 2-[2-[[5-[2-(4-methylpiperazin-1-yl)ethoxy]-1,3-benzothiazol-2-yl]methylcarbamoyl]indan-2-yl]acetic acid

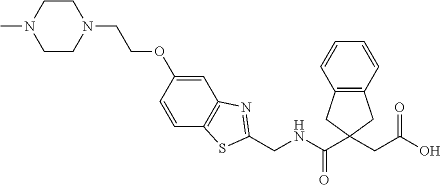 [SMILES: CN1CCN(CCOc2ccc3sc(CNC(=O)C4(CC(=O)O)Cc5ccccc5C4)nc3c2)CC1]

This was prepared in an analogous manner to Example 24 using 2-(4-methylpiperazin-1-yl)ethanol in step-d. The title compound was isolated as a white solid. M/z 509.2 (M+H)⁺. ¹H ¹H NMR (500 MHz, DMSO-d₆): δ 12.16 (1H, bs), 8.72 (1H, t, J=6 Hz), 7.88 (1H, d, J=9 Hz), 7.48 (1H, s), 7.22-7.21 (2H, m), 7.17-7.14 (2H, m), 7.04 (1H, d, J=9 Hz), 4.62 (2H, d, J=6 Hz), 4.19 (2H, bs), 3.55-3.30 (4H, bs), 3.22-3.10 (4H, m), 3.10-3.00 (2H, m), 2.99 (2H, d, J=16.5 Hz), 2.98-2.85 (2H, m), 2.80-2.70 (2H, m), 2.74 (3H, m).

### Example 26 2-[2-[[6-[3-(dimethylamino)azetidine-1-carbonyl]-1,3-benzothiazol-2-yl]methylcarbamoyl]indan-2-yl]acetic acid

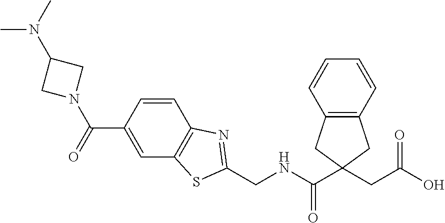 [SMILES: CN(C)C1CN(C(=O)c2ccc3nc(CNC(=O)C4(CC(=O)O)Cc5ccccc5C4)sc3c2)C1]

### a. Methyl 2-[(tert-butoxycarbonylamino)methyl]-1,3-benzothiazole-6-carboxylate

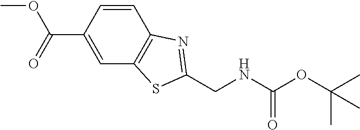 [SMILES: COC(=O)c1ccc2nc(CNC(=O)OC(C)(C)C)sc2c1]

To a stirred solution of methyl 4-amino-3-iodobenzoate (6 g, 21.6 mmol) in acetonitrile (60 mL) was added tert-butyl (2-amino-2-thioxoethyl)carbamate (4.9 g, 25.9 mmol) and CuO (2.6 g, 32.4 mmol) at room temperature. The reaction mixture was purged with argon for 15 minutes, then dppf (2.4 g, 4.33 mmol) and Pd₂(dba)₃ (2 g, 2.16 mmol) was added to the reaction mixture. The reaction mixture was purged with argon for further 5 minutes and heated in a sealed tube at 80° C. for 16 h. The reaction mixture was filtered through celite pad, washed the pad with DCM (60 mL) and filtrate was evaporated. The crude was chromatographed on silica eluting with 20-30% EtOAc in petroleum ether affording a yellow solid (4 g, 57%). M/z 323.1 (M+H)⁺.

### b. 2-[(tert-butoxycarbonylamino)methyl]-1,3-benzothiazole-6-carboxylic acid

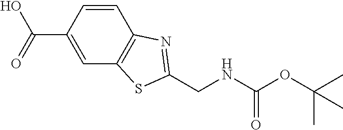 [SMILES: CC(C)(C)OC(=O)NCc1nc2ccc(C(=O)O)cc2s1]

A solution of methyl 2-(((tert-butoxycarbonyl)amino)methyl)benzo[d]thiazole-6-carboxylate (2.5 g, 7.76 mmol) in THF/water (1:1, 100 mL) was added LiOH·H₂O (652 mg, 15.5 mmol) at room temperature and stirred for 6 h. The reaction mixture was evaporated, resulting residue was diluted with water (10 mL) and adjusted the pH to -7 with saturated citric acid. The product was extracted with 10% MeOH in DCM (2×50 mL) and evaporated affording a yellow solid (2 g, 83%). M/z 307.0 (M−H)⁺.

### c. Tert-butyl N-[[6-[3-(dimethylamino)azetidine-1-carbonyl]-1,3-benzothiazol-2-yl]methyl]carbamate

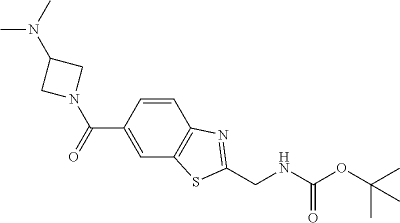 [SMILES: CN(C)C1CN(C(=O)c2ccc3nc(CNC(=O)OC(C)(C)C)sc3c2)C1]

A solution of 2-(((tert-butoxycarbonyl)amino)methyl)benzo[d]thiazole-6-carboxylic acid (500 mg, 1.62 mmol) in DMF (5 mL) was added N,N-dimethylazetidin-3-amine hydrochloride (281 mg, 1.62 mmol) and Et₃N (0.7 mL, 4.87 mmol) at room temperature and stirred for 10 minutes. Then T3P (750 mg, 2.43 mmol) was added and stirred for 12 h. The reaction mixture was diluted with cold water (10 mL), extracted with EtOAc (2×25 mL) and the organic layer was evaporated affording a pale yellow solid (525 mg, crude). M/z 391.2 (M+H)⁺.

### d. (2-(aminomethyl)benzo[d]thiazol-6-yl)(3-(dimethylamino)azetidin-1-yl)methanone hydrochloride

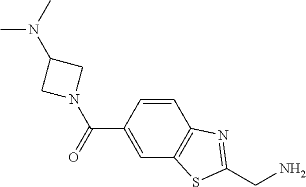 [SMILES: CN(C)C1CN(C(=O)c2ccc3nc(CN)sc3c2)C1]

A solution of tert-butyl ((6-(3-(dimethylamino)azetidine-1-carbonyl)benzo[d]thiazol-2-yl)methyl)carbamate (520 mg, 1.33 mmol) in dioxane (5 mL) was added 4M HCl in dioxane (4 mL) at room temperature and stirred for 3 h. The reaction mixture evaporated and resulting residue was triturated with diethyl ether (15 mL) affording a yellow solid (500 mg, crude). M/z 291.0 (M+H)⁺.

### e. Tert-butyl 2-[2-[[6-[3-(dimethylamino)azetidine-1-carbonyl]-1,3-benzothiazol-2-yl]methylcarbamoyl]indan-2-yl]acetate

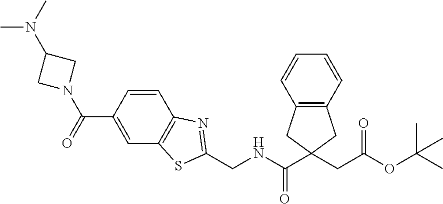 [SMILES: CN(C)C1CN(C(=O)c2ccc3nc(CNC(=O)C4(CC(=O)OC(C)(C)C)Cc5ccccc5C4)sc3c2)C1]

A solution of (2-(aminomethyl)benzo[d]thiazol-6-yl)(3-(dimethylamino)azetidin-1-yl)methanone hydrochloride (220 mg, 0.67 mmol) in DMF (4 mL) was added Et₃N (0.5 mL, 3.37 mmol) at room temperature and stirred for 10 minutes. Then 2-(2-(tert-butoxy)-2-oxoethyl)-2,3-dihydro-1H-indene-2-carboxylic acid (208 mg, 0.74 mmol) and T3P (660 mg, 1.03 mmol) was added and stirred for 12 h. The reaction mixture was diluted with cold water (10 mL), extracted with EtOAc (2×30 mL) and the organic layer was evaporated to give crude compound. The crude was chromatographed on silica eluting with 2% MeOH in DCM affording a brown solid (120 mg, 33%). M/z 549.3 (M+H)⁺.

### f. 2-[2-[[6-[3-(dimethylamino)azetidine-1-carbonyl]-1,3-benzothiazol-2-yl]methylcarbamoyl]indan-2-yl]acetic acid

A solution of tert-butyl 2-(2-(((6-(3-(dimethylamino)azetidine-1-carbonyl)benzo[d]thiazol-2-yl)methyl)carbamoyl)-2,3-dihydro-1H-inden-2-yl)acetate (110 mg, 0.20 mmol) in DCM (5 mL) was treated with TFA (2 mL) at room temperature for 4 h. The mixture was evaporated and the residue was triturated with diethyl ether (10 mL). The crude compound was purified by preparative HPLC [YMC-TRIART-C18 (150*25), 10 u, Mobile phase: A: 0.1% Formic Acid in H₂O, B: MeCN] and freeze dried affording the title product as an off white solid (53 mg, 54%). M/z 493.2 (M+H). ¹H NMR (500 MHz, DMSO-d₆): δ 12.16 (1H, bs), 8.79 (1H, t, J=6 Hz), 8.35 (1H, d, J=1.5 Hz), 8.00 (1H, d, J=8.5 Hz), 7.75 (1H, dd, J=8.5 Hz, J=1.5 Hz), 7.23-7.21 (2H, m), 7.16-7.14 (2H, m), 4.69 (2H, d, J=6 Hz), 4.62-4.58 (1H, m), 4.48-4.42 (1H, m), 4.31-4.19 (2H, m), 4.15-4.05 (1H, m), 3.46 (2H, d, J=16.5 Hz), 3.01 (2H, d, J=16.5 Hz), 2.85-2.65 (8H, bs).

### Example 27 2-[2-[[5-(4-methylpiperazine-1-carbonyl)-1,3-benzothiazol-2-yl]methylcarbamoyl]indan-2-yl]acetic acid

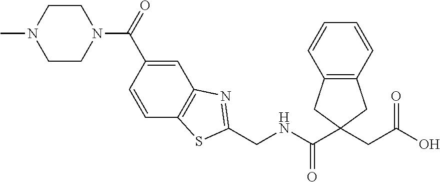 [SMILES: CN1CCN(C(=O)c2ccc3sc(CNC(=O)C4(CC(=O)O)Cc5ccccc5C4)nc3c2)CC1]

This was prepared in an analogous manner to Example 26 starting from methyl 3-amino-4-iodobenzoate in step-a and using 1-methylpiperazine in step-c. The title compound was isolated as a white solid. M/z 493.2 (M+H)⁺. ¹H NMR (500 MHz, DMSO-d₆): δ 12.15 (1H, bs), 9.80 (1H, bs), 8.79 (1H, t, J=5.5 Hz), 8.13 (1H, d, J=8.5 Hz), 8.00 (1H, s), 7.46 (1H, d, J=8.5 Hz), 7.23-7.21 (2H, m), 7.15-7.13 (2H, m), 4.67 (2H, d, J=5.5 Hz), 3.47 (2H, d, J=16 Hz), 3.18-3.01 (8H, m), 2.99 (2H, d, J=16 Hz), 2.79 (2H, s), 2.75 (3H, s).

### Example 28 2-[2-[[5-[2-(dimethylamino)ethylcarbamoyl]-1,3-benzothiazol-2-yl]methylcarbamoyl]indan-2-yl]acetic acid

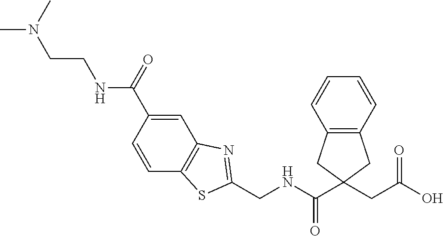 [SMILES: CN(C)CCNC(=O)c1ccc2sc(CNC(=O)C3(CC(=O)O)Cc4ccccc4C3)nc2c1]

This was prepared in an analogous manner to Example 26 starting from methyl 3-amino-4-iodobenzoate in step-a and using N′,N′-dimethylethane-1,2-diamine in step-c. The title compound was isolated as a white solid. M/z 481.2 (M+H)⁺. ¹H NMR (500 MHz, DMSO-d₆): δ 11.42 (1H, bs), 8.79 (1H, t, J=5.5 Hz), 8.68 (1H, t, J=5 Hz), 8.40 (1H, s), 8.11 (1H, d, J=8.5 Hz), 7.88 (1H, dd, J=8.5 Hz, J=1.5 Hz), 7.23-7.21 (2H, m), 7.15-7.13 (2H, m), 4.68 (2H, d, J=6.0 Hz), 3.51-3.48 (2H, m), 3.46 (2H, d, J=16.5 Hz), 3.00 (2H, d, J=16.5 Hz), 2.88-2.82 (2H, bs), 2.75 (2H, s), 2.55-2.50 (6H, bs).

### Example 29 2-[2-[[6-(4-methylpiperazine-1-carbonyl)-1,3-benzothiazol-2-yl]methylcarbamoyl]indan-2-yl]acetic acid

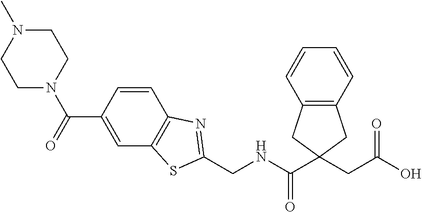 [SMILES: CN1CCN(C(=O)c2ccc3nc(CNC(=O)C4(CC(=O)O)Cc5ccccc5C4)sc3c2)CC1]

This was prepared in an analogous manner to Example 26 starting from methyl 4-amino-3-iodobenzoate in step-a and using 1-methylpiperazine in step-c. The title compound was isolated as a white solid. M/z 493.2 (M+H)⁺. ¹H NMR (500 MHz, MeOD): δ 8.07 (1H, d, J=1.0 Hz), 7.99 (1H, d, J=8.5 Hz), 7.57 (1H, dd, J=8.5 Hz, J=1 Hz), 7.23-7.21 (2H, m), 7.16-7.15 (2H, m), 4.79 (2H, s), 3.88-3.75 (4H, m), 3.54 (2H, d, J=16 Hz), 3.20-3.15 (4H, m), 3.10 (2H, d, J=16 Hz), 2.83 (2H, s), 2.81 (3H, s).

### Example 30 2-[2-[[6-[2-(dimethylamino)ethylcarbamoyl]-1,3-benzothiazol-2-yl]methylcarbamoyl]indan-2-yl]acetic acid

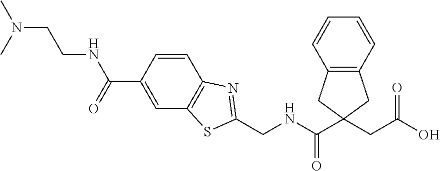 [SMILES: CN(C)CCNC(=O)c1ccc2nc(CNC(=O)C3(CC(=O)O)Cc4ccccc4C3)sc2c1]

This was prepared in an analogous manner to Example 26 starting from methyl 4-amino-3-iodobenzoate in step-a and using N′,N′-dimethylethane-1,2-diamine in step-c. The title compound was isolated as a white solid. M/z 481.2 (M+H)⁺. ¹H NMR (500 MHz, DMSO-d₆): δ 12.15 (1H, bs), 8.77 (2H, t, J=5.5 Hz), 8.51 (1H, s), 8.02 (1H, d, J=8.5 Hz), 7.96 (1H, dd, J=8.5 Hz, J=1.5 Hz), 7.23-7.21 (2H, m), 7.15-7.13 (2H, m), 4.68 (2H, d, J=6.0 Hz), 3.63-3.60 (2H, m), 3.48 (2H, d, J=16.5 Hz), 3.26-3.20 (2H, m), 3.00 (2H, d, J=16.5 Hz), 2.83 (6H, s), 2.76 (2H, s).

### Example 31 2-[2-[[5-[4-[3-(dimethylamino)propyl]piperazine-1-carbonyl]-1,3-benzothiazol-2-yl]methylcarbamoyl]indan-2-yl]acetic acid

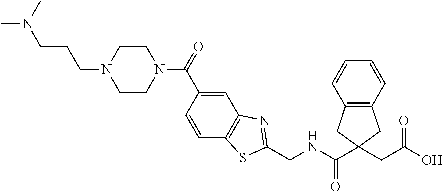 [SMILES: CN(C)CCCN1CCN(C(=O)c2ccc3sc(CNC(=O)C4(CC(=O)O)Cc5ccccc5C4)nc3c2)CC1]

This was prepared in an analogous manner to Example 26 starting from methyl 3-amino-4-iodobenzoate in step-a and using N,N-dimethyl-3-piperazin-1-yl-propan-1-amine in step-c. The title compound was isolated as a white solid. M/z 564.2 (M+H)⁺. ¹H NMR (500 MHz, DMSO-d₆): δ 9.07 (1H, bs), 8.10 (1H, d, J=8.5 Hz), 7.89 (1H, s), 7.39 (1H, d, J=8.5 Hz), 7.22-7.20 (2H, m), 7.14-7.13 (2H, m), 4.67 (2H, d, J=5.5 Hz), 3.64-3.62 (2H, m), 3.46 (2H, d, J=16 Hz), 3.25-3.15 (2H, bs), 3.01 (2H, d, J=16 Hz), 2.72 (2H, s), 2.48-2.25 (8H, bs) 2.18 (6H, s), 1.58-1.55 (2H, m).

### Example 32 2-[2-[[5-[3-(dimethylamino)azetidine-1-carbonyl]-1,3-benzothiazol-2-yl]methylcarbamoyl]indan-2-yl]acetic acid

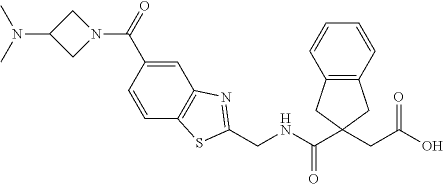 [SMILES: CN(C)C1CN(C(=O)c2ccc3sc(CNC(=O)C4(CC(=O)O)Cc5ccccc5C4)nc3c2)C1]

This was prepared in an analogous manner to Example 26 starting from methyl 3-amino-4-iodobenzoate in step-a. The title compound was isolated as a white solid. M/z 493.2 (M+H)⁺. ¹H NMR (500 MHz, DMSO-d₆): δ 12.05 (1H, bs), 8.94 (1H, bs), 8.11 (1H, s), 8.10 (1H, d, J=8.0 Hz), 7.65 (1H, dd, J=8 Hz, J=1 Hz), 7.22-7.20 (2H, m), 7.14-7.13 (2H, m), 4.67 (2H, d, J=5.5 Hz), 4.34-4.31 (1H, m), 4.13-4.06 (2H, m), 3.86-3.83 (1H, m), 3.46 (2H, d, J=16 Hz), 3.10-3.05 (1H, m), 2.99 (2H, d, J=16 Hz), 2.73 (2H, s), 2.08 (6H, s).

### Example 33 2-[2-[[6-methoxy-5-(4-methylpiperazine-1-carbonyl)-1,3-benzothiazol-2-yl]methylcarbamoyl]indan-2-yl]acetic acid

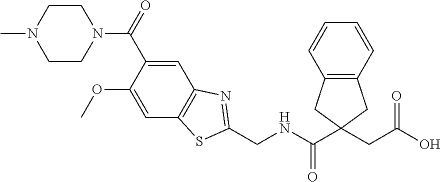 [SMILES: COc1cc2sc(CNC(=O)C3(CC(=O)O)Cc4ccccc4C3)nc2cc1C(=O)N1CCN(C)CC1]

### a. Methyl 4-iodo-2-methoxy-5-nitrobenzoate

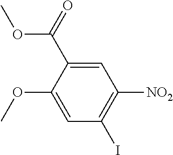 [SMILES: COC(=O)c1cc([N+](=O)[O-])c(I)cc1OC]

A solution of commercially-available methyl 4-iodo-2-methoxybenzoate (1.05 g, 3.6 mmol) in concentrated sulfuric acid (1.6 ml) was treated at 0° C. with a mixture of concentrated nitric acid/concentrated sulfuric acid (0.6 mL/1 mL). The mixture was stirred at room temperature for 5 h then added to ice/water and extracted twice with ethyl acetate. The combined ethyl acetate extracts were washed with saturated aqueous sodium chloride solution. The organic phase was dried (MgSO₄) and evaporated to afford a yellow solid (1.03 g, 85%) that was used without purification. M/z 338.4 (M+H)⁺.

### b. Methyl 5-amino-4-iodo-2-methoxybenzoate

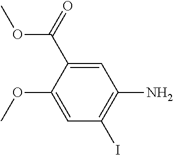

A mixture of methyl 4-iodo-2-methoxy-5-nitrobenzoate (600 mg, 1.8 mmol), iron powder (840 mg, 15 mmol) and methanol (9 mL) was treated with aqueous hydrochloric acid (0.2M; 9 mL, 1.8 mmol) then heated to reflux for 3 h. The mixture was allowed to cool to room temperature then filtered through celite and evaporated. The residue was dissolved in ethyl acetate and washed with saturated aqueous sodium bicarbonate solution, dried (MgSO₄) and evaporated to afford a yellow solid. This was triturated with methanol and filtered, affording recovered starting material (183 mg). The filtrate was evaporated and the residue purified by chromatography on an SCX cartridge eluting with 2M ammonia in methanol affording a yellow oil (132 mg, 24%). M/z 308.0 (M+H)⁺.

### c. Methyl 2-({[(tert-butoxy)carbonyl]amino}methyl)-6-methoxy-1,3-benzothiazole-5-carboxylate

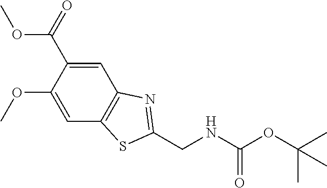 [SMILES: COC(=O)c1cc2nc(CNC(=O)OC(C)(C)C)sc2cc1OC]

A solution of methyl 5-amino-4-iodo-2-methoxybenzoate (132 mg, 0.43 mmol) in in ACN (3 mL) was treated with tert-butyl (2-amino-2-thioxoethyl)carbamate (100 mg, 0.52 mmol), calcium oxide (50 mg, 0.52 mmol), tris(dibenzylideneacetone)dipalladium(0) (38 mg, 0.41 mmol) and dppf (90 mg, 0.16 mmol) then degassed and flushed with argon. The mixture was heated at 65° C. in a sealed vial for 5h then cooled, diluted with ethyl acetate and washed with 10% aqueous citric acid solution then saturated aqueous sodium chloride solution. The organic extract was dried (MgSO₄) and evaporated. The residue was chromatographed on silica eluting with 0-100% ethyl acetate in hexane affording an oil (97 mg, 64%). M/z 353.3 (M+H)⁺.

### d. 2-({[(Tert-butoxy)carbonyl]amino}methyl)-6-methoxy-1,3-benzothiazole-5-carboxylic acid

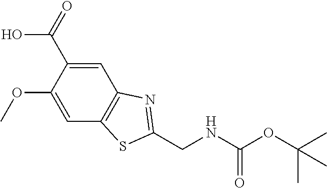 [SMILES: COc1cc2sc(CNC(=O)OC(C)(C)C)nc2cc1C(=O)O]

A solution of methyl 2-({[(tert-butoxy)carbonyl]amino}methyl)-6-methoxy-1,3-benzothiazole-5-carboxylate (96 mg, 0.27 mmol) in THF (3 mL) was treated with aqueous lithium hydroxide solution (1M; 0.5 mL, 0.5 mmol). After 16 h the mixture was reduced in volume by evaporation and acidified to pH 4 with aqueous hydrochloric acid (1M) then extracted with DCM. The DCM extract was washed with water, saturated aqueous sodium chloride solution, dried (MgSO₄) and evaporated to give a brown foam (55 mg, 60%). M/z 283.3 (M−t-Bu)⁺.

### e. Tert-butyl N-{[6-methoxy-5-(4-methylpiperazine-1-carbonyl)-1,3-benzothiazol-2-yl]methyl}carbamate

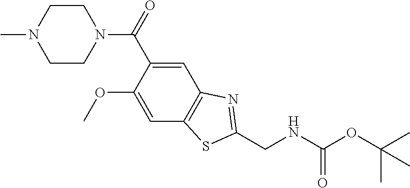 [SMILES: COc1cc2sc(CNC(=O)OC(C)(C)C)nc2cc1C(=O)N1CCN(C)CC1]

A solution of 2-({[(tert-butoxy)carbonyl]amino}methyl)-6-methoxy-1,3-benzothiazole-5-carboxylic acid (55 mg, 0.16 mmol), 1-methylpiperazine (20 mg, 0.2 mmol), DIPEA (63 mg, 0.5 mmol) in DCM (4 mL) was treated with HATU (74 mg, 0.2 mmol). After 4 h the mixture was diluted with DCM and washed with water then saturated aqueous sodium chloride solution, dried (MgSO₄) and evaporated to give a brown foam (135 mg). This was chromatographed on silica eluting with 0-10% methanol in DCM affording an oil (41 mg, 60%). M/z 421.2 (M+H)⁺.

### f. [6-Methoxy-5-(4-methylpiperazine-1-carbonyl)-1,3-benzothiazol-2-yl]methanamine

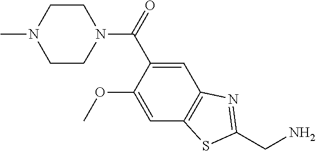 [SMILES: COc1cc2sc(CN)nc2cc1C(=O)N1CCN(C)CC1]

A solution of tert-butyl N-{[6-methoxy-5-(4-methylpiperazine-1-carbonyl)-1,3-benzothiazol-2-yl]methyl}carbamate (41 mg, 0.01 mmol) in DCM (1 mL) was treated with TFA (0.5 mL). After 3 h the mixture was evaporated and the residue purified by chromatography on an SCX cartridge eluting with 2M ammonia in methanol affording a yellow oil (21 mg, 66%). M/z 321.2 (M+H)⁺.

### g. Tert-butyl 2-[2-[[6-methoxy-5-(4-methylpiperazine-1-carbonyl)-1,3-benzothiazol-2-yl]methylcarbamoyl]indan-2-yl]acetate

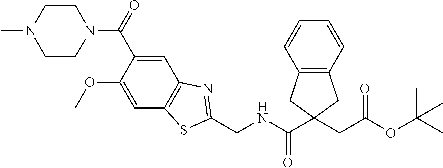 [SMILES: COc1cc2sc(CNC(=O)C3(CC(=O)OC(C)(C)C)Cc4ccccc4C3)nc2cc1C(=O)N1CCN(C)CC1]

A solution of [6-methoxy-5-(4-methylpiperazine-1-carbonyl)-1,3-benzothiazol-2-yl]methanamine (21 mg, 0.07 mmol), 2-(2-(tert-butoxy)-2-oxoethyl)-2,3-dihydro-1H-indene-2-carboxylic acid (22 mg, 0.08 mmol), DIPEA (26 mg, 0.2 mmol) in DCM (3 mL) was treated with HATU (26 mg, 0.07 mmol). After 16 h the mixture was diluted with DCM and washed with water then saturated aqueous sodium chloride solution, dried (MgSO₄) and evaporated to give a yellow oil. This was chromatographed on silica eluting with 0-10% methanol in DCM affording an oil (32 mg, 84%). M/z 579.4 (M+H)⁺.

### h. 2-[2-[[6-methoxy-5-(4-methylpiperazine-1-carbonyl)-1,3-benzothiazol-2-yl]methylcarbamoyl]indan-2-yl]acetic acid

A solution of tert-butyl 2-[2-[[6-methoxy-5-(4-methylpiperazine-1-carbonyl)-1,3-benzothiazol-2-yl]methylcarbamoyl]indan-2-yl]acetate (32 mg, 0.05 mmol) in DCM (2 mL) was treated with TFA (0.25 mL). After 2 h the mixture was evaporated. The residue as purified by MDAP and product-containing fractions were freeze-dried to afford the title compound as white solid (20 mg, 69%). M/z 523.1 (M+H)⁺. ¹H NMR (400 MHz, d₆-DMSO) δ 12.20 (1H, bs), 8.80 (1H, bs), 7.95 (1H, s), 7.75 (1H, s), 7.22 (1H, m), 7.15 (2H, m), 5.40 (2H, bs), 4.60 (2H, m), 3.85 (3H, s), 3.50 (2H, d), 3.45-3.20 (8H, m), 3.15 (3H, s), 2.90 (2H, d).

### Example 34 2-[5,6-difluoro-2-[[6-methoxy-5-(4-methylpiperazine-1-carbonyl)-1,3-benzothiazol-2-yl]methylcarbamoyl]indan-2-yl]acetic acid

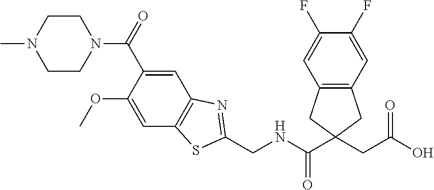 [SMILES: COc1cc2sc(CNC(=O)C3(CC(=O)O)Cc4cc(F)c(F)cc4C3)nc2cc1C(=O)N1CCN(C)CC1]

This was prepared in an analogous manner to Example 33 except that 2-[(tert-butoxy)carbonyl]-5,6-difluoro-2,3-dihydro-1H-indene-2-carboxylic acid was used in place of 2-[(tert-butoxy)carbonyl]-2,3-dihydro-1H-indene-2-carboxylic acid. The title compound was isolated as a white solid (35 mg). M/z 573.2 (M+H)⁺. ¹H NMR (400 MHz, d₆-DMSO) δ 12.50 (1H, bs), 8.80 (1H, bs), 7.75 (1H, s), 7.70 (1H, s), 7.25 (2H, t), 4.60 (2H, m), 3.85 (3H, s), 3.45 (2H, d), 3.15 (3H, s), 3.00 (2H, d).

### Example 35 2-[2-[[5-(4,4-dimethylpiperazin-4-ium-1-carbonyl)-6-methoxy-1,3-benzothiazol-2-yl]methylcarbamoyl]-5,6-difluoro-indan-2-yl]acetate

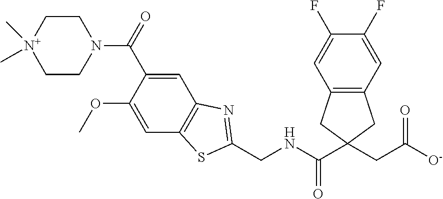 [SMILES: COc1cc2sc(CNC(=O)C3(CC(=O)[O-])Cc4cc(F)c(F)cc4C3)nc2cc1C(=O)N1CC[N+](C)(C)CC1]

### a. Tert-butyl 2-[2-[[5-(4,4-dimethylpiperazin-4-ium-1-carbonyl)-6-methoxy-1,3-benzothiazol-2-yl]methylcarbamoyl]-5,6-difluoro-indan-2-yl]acetate iodide

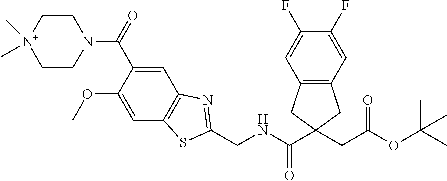 [SMILES: COc1cc2sc(CNC(=O)C3(CC(=O)OC(C)(C)C)Cc4cc(F)c(F)cc4C3)nc2cc1C(=O)N1CC[N+](C)(C)CC1]

A solution of tert-butyl 5,6-difluoro-2-({[6-methoxy-5-(4-methylpiperazine-1-carbonyl)-1,3-benzothiazol-2-yl]methyl}carbamoyl)-2,3-dihydro-1H-indene-2-carboxylate, the final intermediate of Example 34, (140 mg, 0.23 mmol) in THF (5 mL) was treated with iodomethane (365 mg, 2.6 mmol). After 2 h the mixture was evaporated affording a white solid (170 mg, 98%). M/z 629.3 (M)⁺.

### b. 2- [2-[[5-(4,4-dimethylpiperazin-4-ium-1-carbonyl)-6-methoxy-1,3-benzothiazol-2-yl]methylcarbamoyl]-5,6-difluoro-indan-2-yl]acetate

A solution of Tert-butyl 2-[2-[[5-(4,4-dimethylpiperazin-4-ium-1-carbonyl)-6-methoxy-1,3-benzothiazol-2-yl]methylcarbamoyl]-5,6-difluoro-indan-2-yl]acetate iodide (170 mg, 0.27 mmol) in DCM (4 mL) was treated with TFA (0.5 mL). After 2 h the mixture was evaporated. The residue as purified by MDAP and product-containing fractions were freeze-dried to afford the title compound as white solid (20 mg, 69%). M/z 574.2 (M+H)⁺. ¹H NMR (400 MHz, d₆-DMSO) δ 9.80 (1H, bs), 7.80 (2H, m), 7.25 (2H, t), 4.60 (2H, m), 4.10 (1H, m), 3.85 (3H, s), 3.80 (1H, m), 3.60-3.30 (12H, m), 3.20 (2H, d), 3.00 (2H, d).

### Example 36 2-[2-[[5-[3-(dimethylamino)azetidine-1-carbonyl]-6-methoxy-1,3-benzothiazol-2-yl]methylcarbamoyl]indan-2-yl]acetic acid

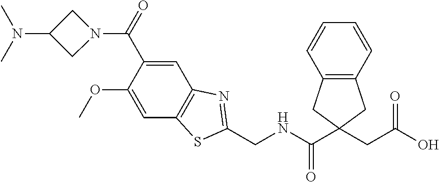 [SMILES: COc1cc2sc(CNC(=O)C3(CC(=O)O)Cc4ccccc4C3)nc2cc1C(=O)N1CC(N(C)C)C1]

This was prepared using the same procedures as Example 33 with the difference that 3-dimethylaminoazetidine was used in place of 1-methylpiperazine, affording the title compounds as a white solid (62 mg). M/z 523.2 (M+H)⁺. ¹H NMR (400 MHz, d₆-DMSO) δ 12.30 (1H, bs), 8.80 (1H, s), 7.78 (1H, s), 7.72 (1H, s), 7.50 (1H, s), 7.20 (1H, m), 7.10 (1H, m), 5.40 (2H, bs), 4.62 (2H, m), 3.90 (3H, s), 4.20-3.80 (5H, m), 3.50 (2H, d), 3.30 (6H, s), 2.90 (2H, d).

### Example 37 2-[2-[[5-[3-(dimethylamino)azetidine-1-carbonyl]-6-methoxy-1,3-benzothiazol-2-yl]methylcarbamoyl]-5,6-difluoro-indan-2-yl]acetic acid

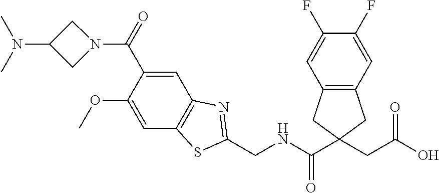 [SMILES: COc1cc2sc(CNC(=O)C3(CC(=O)O)Cc4cc(F)c(F)cc4C3)nc2cc1C(=O)N1CC(N(C)C)C1]

This was prepared using the same procedures as Example 36 with the difference that 2-[(tert-butoxy)carbonyl]-5,6-difluoro-2,3-dihydro-1H-indene-2-carboxylic acid was used in place of 2-[(tert-butoxy)carbonyl]-2,3-dihydro-1H-indene-2-carboxylic acid, affording the title compounds as a white solid (48 mg). M/z 559.2 (M+H)⁺. ¹H NMR (400 MHz, d₆-DMSO) δ 12.30 (1H, bs), 8.85 (1H, bs), 7.78 (1H, s), 7.72 (1H, s), 7.50 (1H, s), 7.25 (2H, t), 4.60 (2H, d), 3.85 (3H, s), 4.10-3.70 (5H, m), 3.50 (4H, m), 3.30 (6H, s), 2.95 (2H, d).

### Example 38 2-[2-[[5-[4-(dimethylamino)piperidine-1-carbonyl]-6-methoxy-1,3-benzothiazol-2-yl]methylcarbamoyl]indan-2-yl]acetic acid

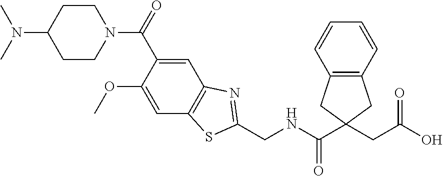 [SMILES: COc1cc2sc(CNC(=O)C3(CC(=O)O)Cc4ccccc4C3)nc2cc1C(=O)N1CCC(N(C)C)CC1]

This was prepared using the same procedures as Example 34 with the difference that 3-dimethylaminoazetidine was used in place of 1-methylpiperazine, affording the title compounds as a white solid (75 mg). M/z 551.7 (M+H)⁺. ¹H NMR (400 MHz, d₆-DMSO) δ 9.00 (1H, bs), 7.80 (2H, m), 7.25 (2H, m), 7.15 (2H, m), 4.65 (2H, m), 4.50 (1H, m), 3.85 (3H, s), 3.50 (2H, d), 3.00-2.60 (5H, m), 2.40 (1H, m), 2.20 (6H, s), 1.80 (1H, m), 1.60 (1H, m), 1.50 (2H, m).

### Example 39 2-[2-[[6-methoxy-5-[4-(trimethylammonio)piperidine-1-carbonyl]-1,3-benzothiazol-2-yl]methylcarbamoyl]indan-2-yl]acetate

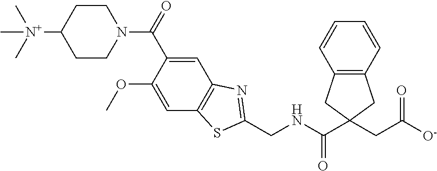 [SMILES: COc1cc2sc(CNC(=O)C3(CC(=O)[O-])Cc4ccccc4C3)nc2cc1C(=O)N1CCC([N+](C)(C)C)CC1]

This was prepared from tert-butyl 2-[({5-[4-(dimethylamino)piperidine-1-carbonyl]-6-methoxy-1,3-benzothiazol-2-yl}methyl)carbamoyl]-2,3-dihydro-1H-indene-2-carboxylate, the final intermediate of Example 38, by the quaternisation and TFA deprotection sequence as described in Example 35, affording the compound as a white solid (83 mg). M/z 565.7 (M+H)⁺. ¹H NMR (400 MHz, d-DMSO) δ 9.00 (1H, bs), 7.80 (2H, s), 7.20 (4H, m), 7.10 (2H, m), 4.70 (2H, m), 3.85 (3H, m), 3.60 (2H, m), 3.45 (2H, d), 3.05 (9H, s), 2.90 (3H, m), 2.20 (2H, m), 2.00 (2H, m), 1.50 (2H, m).

### Example 40 2-[2-[[5-[2-[(dimethylamino)methyl]morpholine-4-carbonyl]-6-methoxy-1,3-benzothiazol-2-yl]methylcarbamoyl]indan-2-yl]acetic acid

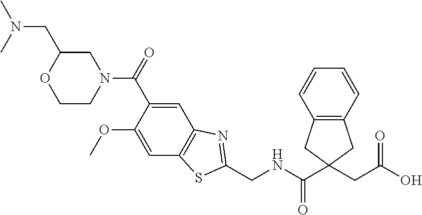 [SMILES: COc1cc2sc(CNC(=O)C3(CC(=O)O)Cc4ccccc4C3)nc2cc1C(=O)N1CCOC(CN(C)C)C1]

This was prepared using the same procedures as Example 33 with the difference that dimethyl[(morpholin-2-yl)methyl]amine was used in place of 1-methylpiperazine, affording the title compounds as a white solid (70 mg). M/z 567.7 (M+H)⁺. ¹H NMR (400 MHz, d-DMSO) δ 9.00 (1H, bs), 7.80 (1H, s), 7.70 (1H, s), 7.20 (2H, m), 7.10 (2H, m), 4.65 (2H, m), 4.20-4.00 (2H, m), 3.95 (2H, m), 3.85 (3H, s), 3.80-3.50 (3H, m), 3.45 (2H, d), 3.10 (6H, m), 3.05 (2H, d), 2.90 (2H, m).

### Example 41 2-[2-[[6-methoxy-5-[2-[(trimethylammonio)methyl]morpholine-4-carbonyl]-1,3-benzothiazol-2-yl]methylcarbamoyl]indan-2-yl]acetate

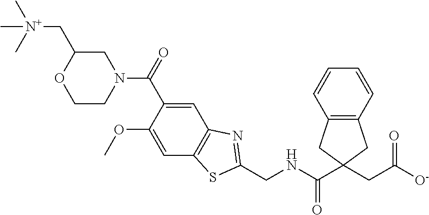 [SMILES: COc1cc2sc(CNC(=O)C3(CC(=O)[O-])Cc4ccccc4C3)nc2cc1C(=O)N1CCOC(C[N+](C)(C)C)C1]

This was prepared from tert-butyl 2-{[(5-{2-[(dimethylamino)methyl]morpholine-4-carbonyl}-6-methoxy-1,3-benzothiazol-2-yl)methyl]carbamoyl}-2,3-dihydro-1H-indene-2-carboxylate, the final intermediate of Example 40, by the quaternisation and TFA deprotection sequence as described in Example 35, affording the compound as a white solid (77 mg). M/z 581.6 (M+H)⁺. ¹H NMR (400 MHz, d-DMSO) δ 13.30 (1H, bs), 7.80 (1H, s), 7.70 (1H, s), 7.20 (2H, m), 7.10 (2H, m), 4.65 (2H, m), 4.20-4.00 (2H, m), 3.95 (2H, m), 3.85 (3H, s), 3.80-3.50 (3H, m), 3.45 (2H, d), 3.15 (9H, s), 3.05 (2H, d), 2.80 (2H, m).

### Example 42 2-[2-[[6-methoxy-5-[3-(trimethylammonio)azetidine-1-carbonyl]-1,3-benzothiazol-2-yl]methylcarbamoyl]indan-2-yl]acetate

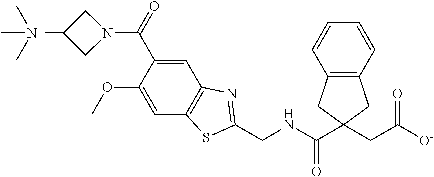 [SMILES: COc1cc2sc(CNC(=O)C3(CC(=O)[O-])Cc4ccccc4C3)nc2cc1C(=O)N1CC([N+](C)(C)C)C1]

This was prepared from tert-butyl 2-[({5-[3-(dimethylamino)azetidine-1-carbonyl]-6-methoxy-1,3-benzothiazol-2-yl}methyl)carbamoyl]-2,3-dihydro-1H-indene-2-carboxylate, the final intermediate of Example 36, by the quaternisation and TFA deprotection sequence as described in Example 35, affording the compound as a white solid (68 mg). M/z 537.6 (M+H)⁺. ¹H NMR (400 MHz, d₆-DMSO) δ 13.40 (1H, bs), 7.80 (2H, m), 7.20 (2H, m), 7.10 (2H, m), 4.65 (2H, d), 4.40 (2H, m), 4.30 (2H, m), 4.15 (1H, m), 3.90 (2H, m), 3.85 (3H, s), 3.55 (2H, m), 3.35 (2H, d), 3.15 (9H, s), 2.90 (2H, d).

### Example 43 2-[5,6-difluoro-2-[[6-methoxy-5-[3-(trimethylammonio)azetidine-1-carbonyl]-1,3-benzothiazol-2-yl]methylcarbamoyl]indan-2-yl]acetate

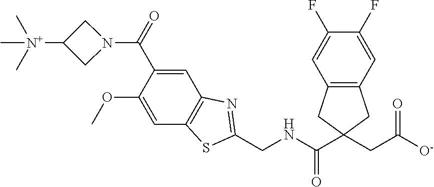 [SMILES: COc1cc2sc(CNC(=O)C3(CC(=O)[O-])Cc4cc(F)c(F)cc4C3)nc2cc1C(=O)N1CC([N+](C)(C)C)C1]

This was prepared from tert-butyl 2-[({5-[3-(dimethylamino)azetidine-1-carbonyl]-6-methoxy-1,3-benzothiazol-2-yl}methyl)carbamoyl]-5,6-difluoro-2,3-dihydro-1H-indene-2-carboxylate, the final intermediate of Example 37, by the quaternisation and TFA deprotection sequence as described in Example 35, affording the compound as a white solid (75 mg). M/z 573.6 (M+H)⁺. ¹H NMR (400 MHz, d₆-DMSO) δ 13.30 (1H, bs), 7.80 (2H, m), 7.20 (2H, t), 4.65 (2H, d), 4.40 (2H, m), 4.30 (2H, m), 4.15 (1H, m), 3.90 (2H, m), 3.85 (3H, s), 3.55 (2H, m), 3.35 (2H, d), 3.15 (9H, s), 2.90 (2H, d).

### Example 44 2-[2-[[5-[(1,1-dimethylpiperidin-1-ium-4-yl)methoxy]-6-methoxy-1,3-benzothiazol-2-yl]methylcarbamoyl]-5,6-difluoro-indan-2-yl]acetate

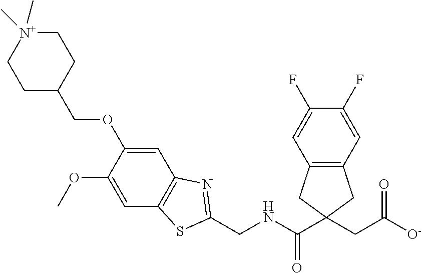 [SMILES: COc1cc2sc(CNC(=O)C3(CC(=O)[O-])Cc4cc(F)c(F)cc4C3)nc2cc1OCC1CC[N+](C)(C)CC1]

This was prepared from tert-butyl 5,6-difluoro-2-[({6-methoxy-5-[(1-methylpiperidin-4-yl)methoxy]-1,3-benzothiazol-2-yl}methyl)carbamoyl]-2,3-dihydro-1H-indene-2-carboxylate, the final intermediate in the synthesis of Example 10, by the quaternisation and TFA deprotection sequence as described in Example 35, affording the compound as a white solid (56 mg). M/z 574.4 (M+H)⁺. ¹H NMR (400 MHz, d₆-DMSO) (13.10 (1H, bs), 7.60 (1H, s), 7.45 (1H, s), 7.20 (2H, t), 4.65 (2H, d), 4.05 (2H, m), 3.85 (3H, s), 3.50 (2H, m), 3.45 (2H, d), 3.40 (2H, m), 3.10 (3H, s), 3.05 (3H, s), 2.90 (2H, d), 2.30 (2H, s), 2.05 (1H, m), 1.90 (2H, m), 1.80 (2H, m).

### Example 45 2-[2-[[6-methoxy-5-(4-methylpiperazin-1-yl)sulfonyl-1,3-benzothiazol-2-yl]methylcarbamoyl]indan-2-yl]acetic acid

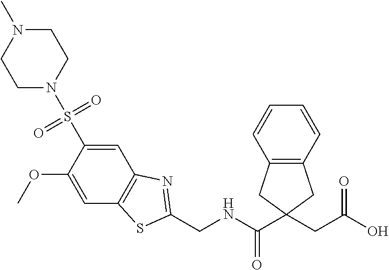 [SMILES: COc1cc2sc(CNC(=O)C3(CC(=O)O)Cc4ccccc4C3)nc2cc1S(=O)(=O)N1CCN(C)CC1]

### a. Tert-butyl N-{[5-(benzylsulfanyl)-6-methoxy-1,3-benzothiazol-2-yl]methyl}carbamate

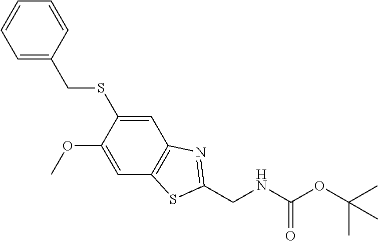 [SMILES: COc1cc2sc(CNC(=O)OC(C)(C)C)nc2cc1SCc1ccccc1]

A mixture of tert-butyl N-[(5-bromo-6-methoxy-1,3-benzothiazol-2-yl)methyl]carbamate (200 mg, 0.54 mmol), benzyl mercaptan (100 mg, 0.8 mmol), Xantphos (31 mg, 0.05 mmol), tris(dibenzylideneacetone)dipalladium(0) (25 mg, 0.027 mmol) and DIPEA (277 mg, 2.1 mmol) in 1,4-dioxane (5 mL) was heated at 115° C. in a sealed tube for 1.25 h then evaporated. The residue was treated with toluene and re-evaporated. The residue was chromatographed on silica eluting with 5-25% ethyl acetate in toluene affording a pale-yellow solid (220 mg, 99%). M/z 317.8 (M+H)⁺ for loss of BOC group.

### b. Tert-butyl N-{[5-(chlorosulfonyl)-6-methoxy-1,3-benzothiazol-2-yl]methyl}carbamate

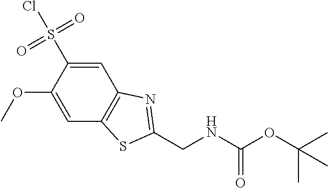 [SMILES: COc1cc2sc(CNC(=O)OC(C)(C)C)nc2cc1S(=O)(=O)Cl]

A solution of tert-butyl N-{[5-(benzylsulfanyl)-6-methoxy-1,3-benzothiazol-2-yl]methyl}carbamate (220 mg, 0.54 mmol) in acetic acid (3 mL) and water (0.4 mL) was treated with N-chlorosuccinimide (215 mg, 1.6 mmol). After 0.5 h the mixture was diluted with water and extracted twice with ethyl acetate. The combined extracts were washed with saturated aqueous sodium chloride solution, dried (Na₂SO₄) and evaporated. The residue was dissolved in toluene and re-evaporated to give a yellow oil that was used directly in the next step (210 mg, 100%). M/z 373.8 (M−H)⁻ for loss of a proton from the corresponding sulfonic acid.

### c. Tert-butyl N-({6-methoxy-5-[(4-methylpiperazin-1-yl)sulfonyl]-1,3-benzothiazol-2-yl}methyl)carbamate

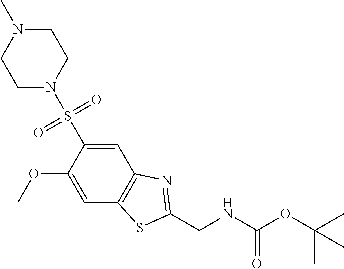 [SMILES: COc1cc2sc(CNC(=O)OC(C)(C)C)nc2cc1S(=O)(=O)N1CCN(C)CC1]

A solution of tert-butyl N-{[5-(chlorosulfonyl)-6-methoxy-1,3-benzothiazol-2-yl]methyl}carbamate (210 mg, 0.54 mmol) in DCM (5 mL) was treated at 0° C. with triethylamine (81 mg, 0.8 mmol) then 1-methylpiperazine (64 mg, 0.64 mmol). After 0.5 h the mixture was diluted with DCM and washed with dilute aqueous sodium bicarbonate solution, water, then dried (Na₂SO₄) and evaporated. The residue was chromatographed on silica eluting with 2-8% 2M ammonia/methanol in DCM affording a colourless oil (195 mg, 79%). M/z 457.6 (M+H)⁺.

### d. {6-Methoxy-5-[(4-methylpiperazin-1-yl)sulfonyl]-1,3-benzothiazol-2-yl}methanamine

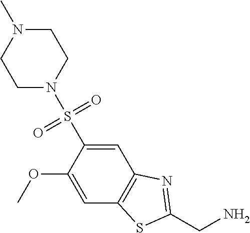 [SMILES: COc1cc2sc(CN)nc2cc1S(=O)(=O)N1CCN(C)CC1]

A solution of tert-butyl N-({6-methoxy-5-[(4-methylpiperazin-1-yl)sulfonyl]-1,3-benzothiazol-2-yl}methyl)carbamate (195 mg, 0.43 mmol) in DCM (3 mL) was treated with TFA (1 mL). After 1 h, toluene was added and the mixture evaporated. More toluene was added and the mixture re-evaporated. The residue was chromatographed on silica eluting with 2-12% 2M ammonia/methanol in DCM affording a colourless oil (133 mg, 87%). M/z 357.4 (M+H)⁺.

### e. Tert-butyl 2-[2-[[6-methoxy-5-(4-methylpiperazin-1-yl)sulfonyl-1,3-benzothiazol-2-yl]methylcarbamoyl]indan-2-yl]acetate

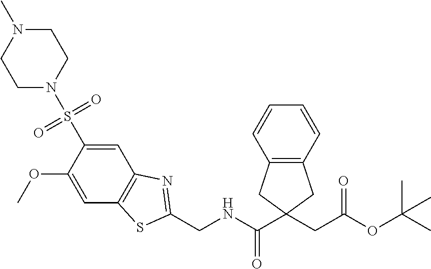 [SMILES: COc1cc2sc(CNC(=O)C3(CC(=O)OC(C)(C)C)Cc4ccccc4C3)nc2cc1S(=O)(=O)N1CCN(C)CC1]

A solution of {6-methoxy-5-[(4-methylpiperazin-1-yl)sulfonyl]-1,3-benzothiazol-2-yl}methanamine (133 mg, 0.37 mmol), 2-(2-(tert-butoxy)-2-oxoethyl)-2,3-dihydro-1H-indene-2-carboxylic acid (103 mg, 0.37 mmol) and DIPEA (145 mg, 1.1 mmol) in DMF (2 mL) was treated with HATU (213 mg, 0.56 mmol). After 0.33 h the mixture was partitioned between ethyl acetate and dilute aqueous sodium bicarbonate solution, then washed with water, dried (Na₂SO₄) and evaporated. The residue was chromatographed on silica eluting with 2-10% 2M ammonia/methanol in DCM affording a light brown foam (233 mg, 95%). M/z 615.4 (M+H)⁺

### f. 2-[2-[[6-methoxy-5-(4-methylpiperazin-1-yl)sulfonyl-1,3-benzothiazol-2-yl]methylcarbamoyl]indan-2-yl]acetic acid

A solution of tert-butyl 2-[2-[[6-methoxy-5-(4-methylpiperazin-1-yl)sulfonyl-1,3-benzothiazol-2-yl]methylcarbamoyl]indan-2-yl]acetate (92 mg, 0.15 mmol) in DCM (3 mL) was treated with TFA (1.5 mL). After 3 h the mixture was diluted with toluene and evaporated. Toluene was added and the mixture re-evaporated. The residue was purified by reverse phase chromatography (C18 cartridge) 5-20% ACN in 2M ammonia/methanol affording the title compound as a white solid (66 mg, 79%). M/z 559.2 (M+H)⁺. ¹H NMR (400 MHz, d-DMSO) δ 9.00 (1H, bs), 8.20 (1H, s), 7.95 (1H, s), 7.22 (1H, m), 7.15 (1H, m), 4.65 (2H, m), 3.95 (3H, s), 3.50 (2H, d), 3.15 (4H, m), 2.90 (2H, d), 2.30 (4H, m), 2.20 (3H, s).

### Example 46 2-[2-[[5-[[4-(dimethylamino)-1-piperidyl]sulfonyl]-6-methoxy-1,3-benzothiazol-2-yl]methylcarbamoyl]indan-2-yl]acetic acid

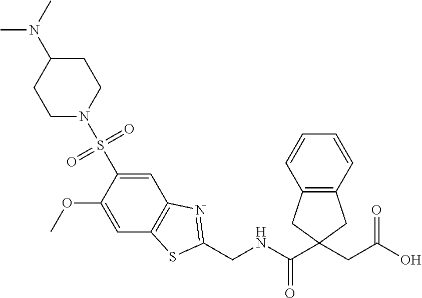 [SMILES: COc1cc2sc(CNC(=O)C3(CC(=O)O)Cc4ccccc4C3)nc2cc1S(=O)(=O)N1CCC(N(C)C)CC1]

This was prepared by the same procedures as for Example 45 with the difference that N,N-dimethylpiperidin-4-amine was used in place of 1-methylpiperazine, affording the title compound as a white solid (69 mg). M/z 587.2 (M+H)⁺. ¹H NMR (400 MHz, d-DMSO) δ 9.00 (1H, bs), 8.20 (1H, s), 7.90 (1H, s), 7.20 (2H, m), 7.10 (2H, m), 4.65 (2H, d), 3.90 (3H, s), 3.70 (2H, m), 3.40 (2H, d), 2.90 (2H, d), 2.60 (1H, t), 2.30 (2H, m), 2.20 (6H, s), 1.80 (2H, m), 1.40 (2H, m).

### Example 47 2-[2-[[6-methoxy-5-[[4-(trimethylammonio)-1-piperidyl]sulfonyl]-1,3-benzothiazol-2-yl]methylcarbamoyl]indan-2-yl]acetate

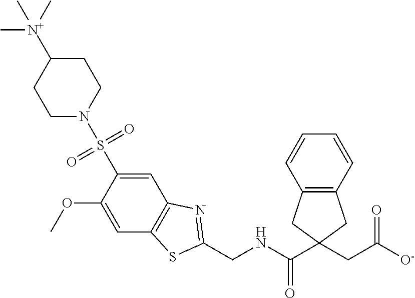 [SMILES: COc1cc2sc(CNC(=O)C3(CC(=O)[O-])Cc4ccccc4C3)nc2cc1S(=O)(=O)N1CCC([N+](C)(C)C)CC1]

This compound was prepared from tert-butyl 2-{[(5-{[4-(dimethylamino)piperidin-1-yl]sulfonyl}-6-methoxy-1,3-benzothiazol-2-yl)methyl]carbamoyl}-2,3-dihydro-1H-indene-2-carboxylate, the final intermediate in the synthesis of Example 46, by the quaternisation and TFA deprotection sequence as described in Example 35, affording the compound as a white solid (91 mg). M/z 601.3 (M+H)⁺. ¹H NMR (400 MHz, d-DMSO) δ 8.20 (1H, s), 8.00 (1H, s), 7.15 (2H, m), 7.10 (2H, m), 4.65 (2H, d), 3.95 (3H, s), 3.90 (2H, m), 3.40 (2H, d), 3.00 (9H, s), 2.90 (2H, d), 2.60 (1H, m), 2.30 (1H, m), 2.10 (2H, m), 1.60 (2H, m).

### Example 48 2-[2-[(6-cyano-1,3-benzothiazol-2-yl)methylcarbamoyl]indan-2-yl]acetic acid

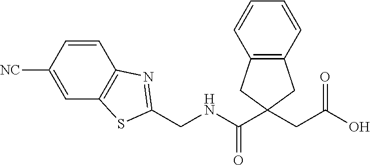 [SMILES: N#Cc1ccc2nc(CNC(=O)C3(CC(=O)O)Cc4ccccc4C3)sc2c1]

### a. Tert-butyl 2-[2-[(6-bromo-1,3-benzothiazol-2-yl)methylcarbamoyl]indan-2-yl]acetate

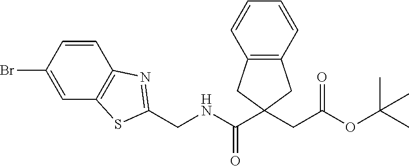 [SMILES: CC(C)(C)OC(=O)CC1(C(=O)NCc2nc3ccc(Br)cc3s2)Cc2ccccc2C1]

This was prepared by the same procedures as for Example 24 step-b to step-d starting with 4-bromo-2-iodo-aniline, affording the title compound as a white solid. M/z 501.1 (M+H)⁺.

### b. Tert-butyl 2-[2-[(6-cyano-1,3-benzothiazol-2-yl)methylcarbamoyl]indan-2-yl]acetate

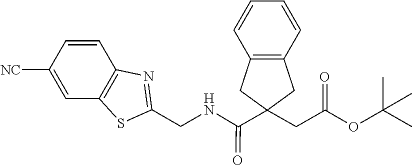 [SMILES: CC(C)(C)OC(=O)CC1(C(=O)NCc2nc3ccc(C#N)cc3s2)Cc2ccccc2C1]

To a stirred solution of tert-butyl 2-[2-[(6-bromo-1,3-benzothiazol-2-yl)methylcarbamoyl]indan-2-yl]acetate (300 mg, 0.59 mmol) in DMF (6 mL) was added Zn(CN)₂ (140 mg, 1.19 mmol) and purged with argon for 10 minutes. Then Pd₂(dba)₃ (55 mg, 0.05 mmol) and Xantphos (70 mg, 0.11 mmol) were added and purged with argon for further 5 minutes. The reaction mixture was heated in sealed tube at 90° C. for 4 h, and then filtered through celite pad, washed the pad with EtOAc (50 mL) and the filtrate was evaporated. The crude was purified by silica gel chromatography eluting with 20-30% EtOAc in petroleum ether affording an off white solid (180 mg, 68%). M/z 448.2 (M+H)⁺.

### c. 2-[2-[(6-cyano-1,3-benzothiazol-2-yl)methylcarbamoyl]indan-2-yl]acetic acid

A solution of tert-butyl 2-[2-[(6-cyano-1,3-benzothiazol-2-yl)methylcarbamoyl]indan-2-yl]acetate (160 mg, 0.35 mmol) in DCM (5 mL) was treated with TFA (2.5 mL) at room temperature for 4 h. The mixture was evaporated and the residue was triturated with diethyl ether (10 mL). The crude compound was purified by preparative HPLC [X-BRIDGE-C18 (150*30), 5 u, Mobile phase: A: 0.1% Formic Acid in H₂O, B: MeCN] affording the title product as a white solid (26 mg, 19%). M/z 392.1 (M+H). ¹H NMR (500 MHz, DMSO-d₆): δ 9.31 (1H, bs), 8.65 (1H, s), 8.09 (1H, d, J=8.5 Hz), 7.90 (1H, dd, J=8.5 Hz, J=1.5 Hz), 7.22-7.20 (2H, m), 7.14-7.13 (2H, m), 4.71 (2H, d, J=5.5 Hz), 3.45 (2H, d, J=16.5 Hz), 2.99 (2H, d, J=16.5 Hz), 2.70 (2H, s).

### Example 49 2-[2-[(5-cyano-1,3-benzothiazol-2-yl)methylcarbamoyl]indan-2-yl]acetic acid

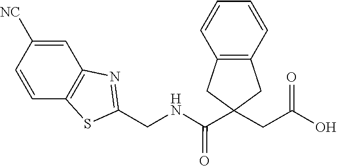 [SMILES: N#Cc1ccc2sc(CNC(=O)C3(CC(=O)O)Cc4ccccc4C3)nc2c1]

This compound was prepared from tert-butyl 2-[2-[(5-bromo-1,3-benzothiazol-2-yl)methylcarbamoyl]indan-2-yl]acetate using the conditions described in example 48 affording the compound as a white solid. M/z 392.1 (M+H)⁺. ¹H NMR (500 MHz, DMSO-d₆): δ 10.7 (1H, bs), 8.46 (1H, s), 8.28 (1H, d, J=8.5 Hz), 7.79 (1H, d, J=8.5 Hz), 7.19-7.18 (2H, m), 7.13-7.11 (2H, m), 4.70 (2H, d, J=5 Hz), 3.42 (2H, d, J=16 Hz), 2.95 (2H, d, J=16 Hz), 2.57 (2H, s).

The following compounds were synthesized according to Methods A-C described above. Subsequently:

### Method D. Final Stages to Synthesise the Examples

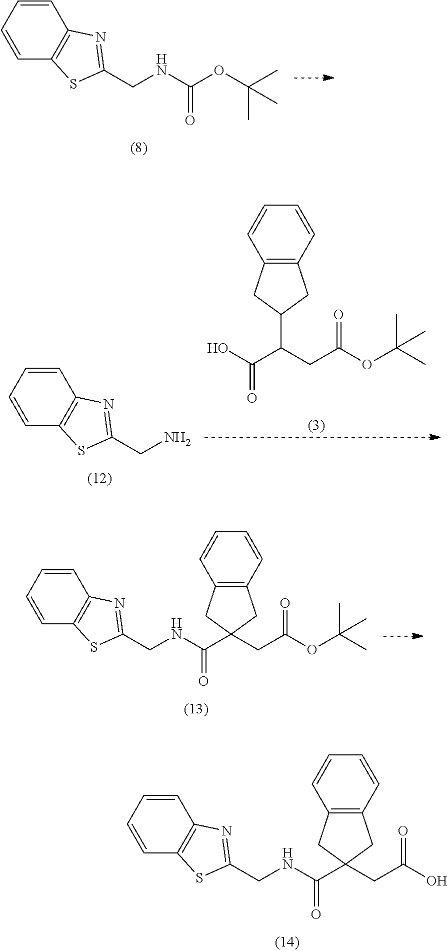 [SMILES: CC(C)(C)OC(=O)NCc1nc2ccccc2s1 ; NCc1nc2ccccc2s1 ; CC(C)(C)OC(=O)CC(C(=O)O)C1Cc2ccccc2C1 ; CC(C)(C)OC(=O)CC1(C(=O)NCc2nc3ccccc3s2)Cc2ccccc2C1 ; O=C(O)CC1(C(=O)NCc2nc3ccccc3s2)Cc2ccccc2C1]

The final stages of the syntheses generally involve acid-catalysed removal of the BOC group from (8) to reveal the free amines (12) followed by coupling with acids of type (3), usually with the standard peptide coupling reagent HATU (for a comprehensive review of the myriad available peptide coupling reagents, see Valeur, E. and Bradley, M, Chem. Soc. Rev., 2008, 28, 606-631). Finally further acid treatment with TFA removes the t-butyl ester to afford the Examples of the invention.

### Method E. Functional Group Manipulation after Amide Coupling of Aminomethylbenzothiazole and Indanyl Moieties

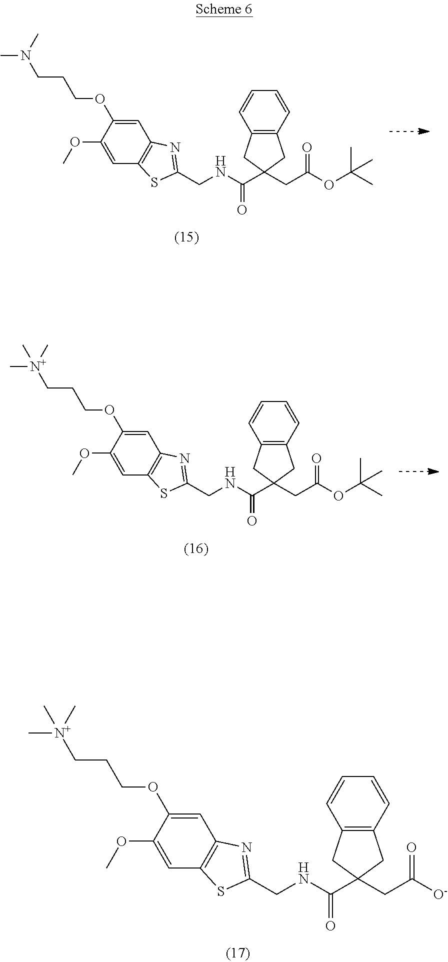

As an example of this approach, alkylation of tert-butyl N-[(5-hydroxy-6-methoxy-1,3-benzothiazol-2-yl)methyl]carbamate with 3-chloro-N,N-dimethylpropan-1-amine, removal of the tert-butoxycarbonyl protecting group and coupling with acid (3) can generate the N,N-dimethylaminopropyloxy intermediate (15). Reaction with an alkylating agent such as iodomethane then generates the corresponding quaternary ammonium salt (16) and finally removal of the tert-butyl ester reveals the carboxylate acid, generating zwitterionic (17) containing both a positive and a negative charge.

### Example 50 2-[2-[[6-(3-hydroxypropoxy)-1,3-benzothiazol-2-yl]methylcarbamoyl]indan-2-yl]acetic acid

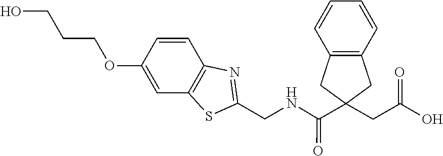 [SMILES: O=C(O)CC1(C(=O)NCc2nc3ccc(OCCCO)cc3s2)Cc2ccccc2C1]

### a. Tert-butyl N-[(6-bromo-1,3-benzothiazol-2-yl)methyl]carbamate

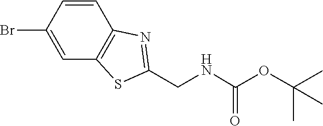 [SMILES: CC(C)(C)OC(=O)NCc1nc2ccc(Br)cc2s1]

To a stirred solution of 4-bromo-2-iodo-aniline (3 g, 10.13 mmol) and tert-butyl (2-amino-2-thioxoethyl) carbamate (1.92 g, 10.13 mmol) in DMF (30 mL) was added CuO (0.8 g, 10.13 mmol) at room temperature and the reaction mixture was degassed with argon for 15 minutes. Then Dppf (280 mg, 0.50 mmol) and Pd₂(dba)₃ (185.4 mg, 0.20) were added and the resulting reaction mixture was degassed with argon for further 5 minutes. The reaction mixture was stirred in sealed tube at 60° C. for 3h, and then filtered through celite pad and washed the pad with EtOAc (50 mL). The filtrate was washed with water (2×30 mL) and concentrated under reduced pressure. The crude compound was purified by silica gel chromatography eluting with 22% EtOAc in petroleum ether affording as a yellow solid (5 g, 72%). M/z 343 (M+H)⁺.

### b. Tert-butyl N-[[6-(4,4,5,5-tetramethyl-1,3,2-dioxaborolan-2-yl)-1,3-benzothiazol-2-yl]methyl]carbamate

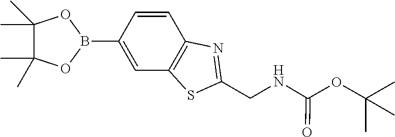 [SMILES: CC(C)(C)OC(=O)NCc1nc2ccc(B3OC(C)(C)C(C)(C)O3)cc2s1]

To a stirred solution of tert-butyl N-[(6-bromo-1,3-benzothiazol-2-yl)methyl]carbamate (1.3 g, 3.80 mmol), and bis(pinacolato)diboron (1.44 g, 5.70 mmol) in 1,4-dioxane (15 mL) was added KOAc (745 mg, 7.60 mmol) at room temperature and the reaction mixture was purged with argon for 15 minutes. Then PdCl₂(dppf)·DCM (155 mg, 0.190 mmol) was added and the reaction mixture purged with argon for further 5 minutes. The reaction mixture was stirred to reflux in sealed tube for 12 h, and then filtered through celite pad and washed with EtOAc (50 mL). The filtrate was washed with water (2×30 mL), the organic layer was dried with sodium sulphate, filtered and concentrated under reduced pressure to get a brown solid (1.5 g, crude). M/z 391.2 (M+H)⁺.

### c. Tert-butyl N-[(6-hydroxy-1,3-benzothiazol-2-yl)methyl]carbamate

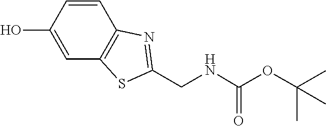 [SMILES: CC(C)(C)OC(=O)NCc1nc2ccc(O)cc2s1]

To a stirred solution of tert-butyl N-[[6-(4,4,5,5-tetramethyl-1,3,2-dioxaborolan-2-yl)-1,3-benzothiazol-2-yl]methyl]carbamate (1.5 g, 3.84 mmol) in THF (15 mL) was added 1N NaOH (3.84 mL g, 3.84 mmol) at 0° C. and stirred for 10 minutes. Then H₂O₂ (30% in H₂O, 0.21 mL, 8.84 mmol) was added at 0° C. and the reaction mixture stirred to room temperature for 1 h. The reaction mixture was partitioned between ethyl acetate (100 mL) and water (70 mL). The aqueous phase was extracted with ethyl acetate (2×100 mL) and the combined organic extracts were washed with brine, dried with Na₂SO₄, filtered and evaporated. The crude product was purified by silica gel chromatography eluting with 40% EtOAc in petroleum ether affording a white solid (1.0 g, 93.4%). M/z 281.1 (M+H)⁺.

### d. Tert-butyl N-[[6-(3-hydroxypropoxy)-1,3-benzothiazol-2-yl]methyl]carbamate

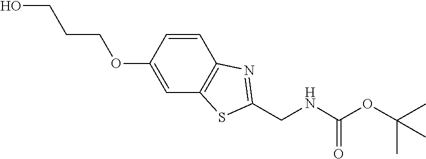 [SMILES: CC(C)(C)OC(=O)NCc1nc2ccc(OCCCO)cc2s1]

To a solution of tert-butyl N-[(6-hydroxy-1,3-benzothiazol-2-yl)methyl]carbamate (300 mg, 1.07 mmol) in DMF (5 mL) was added K₂CO₃ (222 mg, 1.60 mmol), 3-bromopropan-1-ol (224 mg, 1.60 mmol) at room temperature and heated at 80° C. for 2 h. The reaction mixture was diluted with water (20 mL) and extracted with EtOAc (2×30 mL). The combined organic extract was dried, filtered and evaporated. The crude was purified by silica gel chromatography eluting with 45-60% EtOAc in petroleum ether affording a yellow solid (210 m, 58%). M/z=338.9 (M+H)⁺.

### e. 3-[[2-(aminomethyl)-1,3-benzothiazol-6-yl]oxy]propan-1-ol hydrochloride

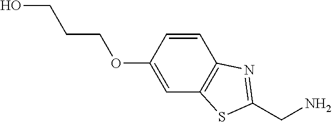 [SMILES: NCc1nc2ccc(OCCCO)cc2s1]

To a solution of tert-butyl N-[[6-(3-hydroxypropoxy)-1,3-benzothiazol-2-yl]methyl]carbamate (210 mg, 0.62 mmol) in dioxane (5 mL) was added 4M HCl in dioxane (2 mL) at room temperature and stirred for 3 h. The reaction mixture was evaporated and the resulting residue was triturated with diethyl ether (20 mL) affording an off white solid (165 mg, crude). M/z=238.9 (M+H)⁺.

### f. Methyl indane-2-carboxylate

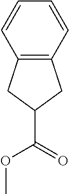

To a stirred solution of 2,3-dihydro-1H-indene-2-carboxylic acid (20 g, 123 mmol) in methanol (200 mL) was added conc. H₂SO₄ (10 mL, 185 mmol) drop wise at room temperature and stirred at 80° C. for 16 h. The reaction mixture was evaporated to get residue. The residue was dissolved in water (100 mL) and extracted with EtOAc (2×100 mL). The organic layer was washed with sat. sodium bicarbonate, brine and evaporated affording a light brown liquid (20 g, 92%). M/z 177.1 (M+H)⁺.

### g. Methyl 2-(2-tert-butoxy-2-oxo-ethyl)indane-2-carboxylate

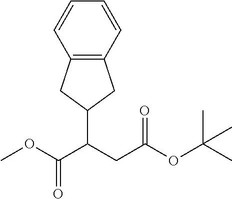 [SMILES: COC(=O)C(CC(=O)OC(C)(C)C)C1Cc2ccccc2C1]

To a solution of methyl 2,3-dihydro-1H-indene-2-carboxylate (5 g, 28.3 mmol) in THF (100 mL) was added NaHMDS (21 mL, 42.5 mmol, 2M in THF) at −78° C. under argon and stirred at −78° C. for 1 h. Then tert-butyl 2-bromoacetate solution (6.4 mL, 42.5 mmol) in THF (30 mL) was added drop wise for 15 minutes at −78° C. and stirred at same temperature for 2 h. The reaction mixture was quenched with sat. ammonium chloride solution (50 mL) at −78° C. and allowed to stir at room temperature for 30 minutes. The organic layer was separated, aqueous layer was extracted with EtOAc (2×100 mL), and the combined organic layer was evaporated to get crude compound. The crude compound was triturated with n-pentane (50 mL) at −78° C. and stirred at same temperature for 15 minutes. The resulting solid was filtered and dried under vacuum affording an off white solid (3.7 g, 45%). M/z=313.0 (M+Na)⁺.

### h. 2-(2-tert-butoxy-2-oxo-ethyl)indane-2-carboxylic acid

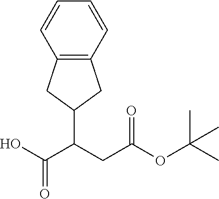 [SMILES: CC(C)(C)OC(=O)CC(C(=O)O)C1Cc2ccccc2C1]

To a stirred solution of methyl 2-(2-(tert-butoxy)-2-oxoethyl)-2,3-dihydro-1H-indene-2-carboxylate (430 g, 1.48 mol) in THF (2.15 L) and ethanol (2.15 L) was added 0.5 M LiOH·H₂O (6.8 L, 2.96 mol) drop wise at room temperature and stirred at same temperature for 2 h. The reaction mixture was evaporated to get the residue and the residue was diluted with H₂O (1 L) and extracted with diethyl ether. The aqueous layer was acidified with 1N HCl to pH 3-4. The resulting precipitate was filtered, washed with water, n-pentane and dried under vacuum affording a white solid (254.5 g, 62%). M/z 275.2 (M−H)⁻. ¹H NMR (300 MHz, DMSO-d₆): δ 12.4 (1H, bs), 7.18-7.10 (4H, m), 3.39 (2H, d, J=16.2 Hz), 2.92 (2H, d, J=16.2 Hz), 2.64 (2H, s), 1.37 (9H, s).

### i. Tert-butyl 2-[2-[[6-(3-hydroxypropoxy)-1,3-benzothiazol-2-yl]methylcarbamoyl]indan-2-yl]acetate

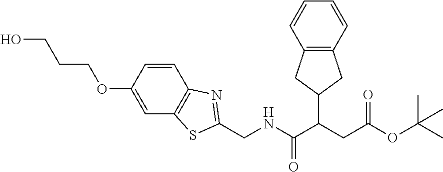 [SMILES: CC(C)(C)OC(=O)CC(C(=O)NCc1nc2ccc(OCCCO)cc2s1)C1Cc2ccccc2C1]

To a solution of 2-(2-(tert-butoxy)-2-oxoethyl)-2,3-dihydro-1H-indene-2-carboxylic acid (150 mg, 0.54 mmol) in DMF (6 mL) was added Et₃N (0.2 mL, 1.62 mmol), EDC. HCl (125 mg, 0.65 mmol), HOBt (74 mg, 0.54 mmol) and 3-[[2-(aminomethyl)-1,3-benzothiazol-6-yl]oxy]propan-1-ol hydrochloride (164 mg, 0.59 mmol) at room temperature and stirred for 12 h. The reaction mixture was diluted with cold water (20 mL) and extracted with EtOAc (2×30 mL) and evaporated. The crude was purified by silica gel chromatography with 3-5% MeOH in DCM affording a yellow solid (125 mg, 46%). M/z=497.2 (M+H)⁺.

### j. 2-[2-[[6-(3-hydroxypropoxy)-1,3-benzothiazol-2-yl]methylcarbamoyl]indan-2-yl]acetic acid

To a solution of tert-butyl 2-[2-[[6-(3-hydroxypropoxy)-1,3-benzothiazol-2-yl]methylcarbamoyl]indan-2-yl]acetate (110 mg, 0.22 mmol) in DCM (5 mL) was added TFA (2 mL) at 0° C. and stirred at room temperature for 2 h. The mixture was evaporated and the residue was triturated with diethyl ether (15 mL). The crude compound was purified by preparative HPLC [HPLC [SYMMETRY-C8 (300*19 mm), 7 u, Mobile phase: A: 0.1% Formic Acid in H₂O, B: MeCN] affording the title compound as an off white solid (20 mg, 20%). M/z 441.1 (M+H)⁺. ¹H NMR (500 MHz, DMSO-d₆): δ 12.12 (1H, bs), 8.69 (1H, t, J=6 Hz), 7.79 (1H, d, J=9 Hz), 7.57 (1H, d, J=2.5 Hz), 7.22-7.19 (2H, m), 7.15-7.13 (2H, m), 7.06 (1H, dd, J=9 Hz, J=2.5 Hz), 4.60 (2H, d, J=6 Hz), 4.55 (1H, t, J=5 Hz), 4.08 (2H, t, J=6.5 Hz), 3.57 (2H, td, J=6 Hz, J=5 Hz), 3.44 (2H, d, J=16 Hz), 3.00 (2H, d, J=16 Hz), 2.73 (2H, s), 1.89-1.86 (2H, m).

### Example 51 2-[2-[(6-propoxy-1,3-benzothiazol-2-yl)methylcarbamoyl]indan-2-yl]acetic acid

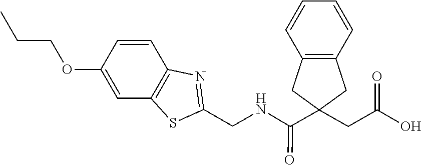 [SMILES: CCCOc1ccc2nc(CNC(=O)C3(CC(=O)O)Cc4ccccc4C3)sc2c1]

This was prepared in an analogous manner to Example 50 using 1-bromopropane in step-d. The title compound was isolated as white solid (37 mg, 38%). M/z 425.1 (M+H)⁺. ¹H NMR (500 MHz, DMSO-d₆): δ 12.14 (1H, bs), 8.97 (1H, bs), 7.78 (1H, d, J=9 Hz), 7.56 (1H, d, J=2.5 Hz), 7.21-7.20 (2H, m), 7.14-7.12 (2H, m), 7.06 (1H, dd, J=9.0 Hz, J=2.5 Hz), 4.60 (2H, d, J=5.5 Hz), 3.98 (2H, t, J=6.5 Hz), 3.47 (2H, d, J=16.5 Hz), 3.00 (2H, d, J=16 Hz), 2.70 (2H, s), 1.77-1.72 (2H, m), 1.00 (3H, t, J=7.5 Hz).

### Example 52 2-[2-[[5-[3-(dimethylamino)propoxy]-6-methoxy-1,3-benzothiazol-2-yl]methylcarbamoyl]indan-2-yl]acetic acid

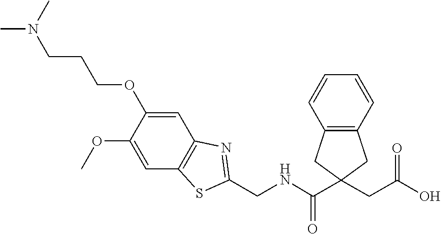 [SMILES: COc1cc2sc(CNC(=O)C3(CC(=O)O)Cc4ccccc4C3)nc2cc1OCCCN(C)C]

### a. 4-bromo-5-methoxy-2-nitroaniline

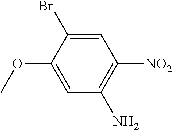

To a stirred solution of 5-methoxy-2-nitroaniline (100 g, 595 mmol) in acetonitrile (2.5 L) was added NBS (106 g, 595 mmol) portion wise at room temperature. The mixture was cooled to 0° C. and added TFA (46 mL, 595 mmol) drop wise for 30 minutes and allowed to stir at room temperature for 16 h. The reaction mixture was diluted with water (1 L) and adjusted the pH to ˜ 8 with 1N NaOH. The resulting precipitate was filtered, washed with water (500 mL) and dried under vacuum affording a yellow solid. (105 g, 72%). M/z 247 (M+H)⁺.

### b. 1-Bromo-4-iodo-2-methoxy-5-nitrobenzene

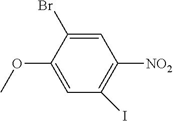

To a stirred solution of 4-bromo-5-methoxy-2-nitroaniline (50 g, 203 mmol) in acetonitrile (750 mL) was added concentrated H₂SO₄ (24 mL, 457 mmol) drop wise at −10° C. Then NaNO₂ (28 g, 406 mmol) in water (175 mL) was added drop wise at −10° C. for 15 minutes and stirred at same temperature for 30 min. After that KI solution (135 g, 813 mmol) in water (175 mL) was added drop wise at −10° C. for 20 minutes and stirred at same temperature for 30 min. The reaction mixture was quenched with sodium metabisulphite solution (309 g, 1.62 mmol) in water (1.6 L) at -10° C. to 0° C. for 1 h. Then water (1 L) was added and allowed to stir at room temperature for 30 minutes. The resulting precipitate was filtered, washed with water (1 L) and dried under vacuum affording a yellow solid. (60 g, 82%). M/z 357.8 (M+H)⁺.

### c. 5-Bromo-2-iodo-4-methoxyaniline

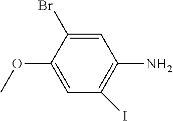

To a stirred solution of 1-bromo-4-iodo-2-methoxy-5-nitrobenzene (106 g, 296 mmol) in EtOH: H₂O (800 mL: 200 mL) was added Fe (49.7 g, 890 mmol), NH₄Cl (80 g, 1.48 mmol) at room temperature and stirred at 90° C. for 2 h. Then the reaction mixture was cooled to 60° C., added additional amount of Fe (33 g, 593 mmol), NH₄Cl (80 g 1.48 mmol) and stirred at 90° C. for 30 minutes. The reaction mixture was filtered through ciliate pad, washed the pad with methanol (1 L) and filtrate was concentrated to give residue. The residue was diluted with cold water (1 L) and adjusted the pH to ˜8 with 1N NaOH. The resulting precipitate was filtered and dried under vacuum affording a light brown solid (90 g, 92%). M/z 327.8 (M+H)⁺.

### d. Tert-butyl N-[(5-bromo-6-methoxy-1,3-benzothiazol-2-yl)methyl]carbamate

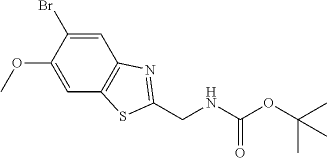 [SMILES: COc1cc2sc(CNC(=O)OC(C)(C)C)nc2cc1Br]

To a stirred solution of 5-bromo-2-iodo-4-methoxyaniline (50 g, 152 mmol) in acetonitrile (560 mL) was added tert-butyl(2-amino-2-thioxoethyl)carbamate (35 g, 183 mmol), CaO (17 g, 305 mmol) and degassed with argon for 20 minutes. Then Pd₂(dba)₃ (14 g, 15.2 mmol), dppf (25.4 g, 15.8 mmol) was added and purged with argon for further 5 minutes and the reaction mixture was stirred at 80° C. for 4 hour. The reaction mixture was filtered through celite pad and washed the pad with EtOAc (300 mL). The filtrate was washed with water and evaporated to get crude compound. The crude compound was dissolved in acetonitrile (200 mL), on standing for 1 hour solid was precipitated out. The resulting solid was filtered, washed with acetonitrile (50 mL) and dried under vacuum affording an off white solid (34 g, 60%). M/z 372.9 (M+H)⁺.

### e. Tert-butyl N-[[6-methoxy-5-(4,4,5,5-tetramethyl-1,3,2-dioxaborolan-2-yl)-1,3-benzothiazol-2-yl]methyl]carbamate

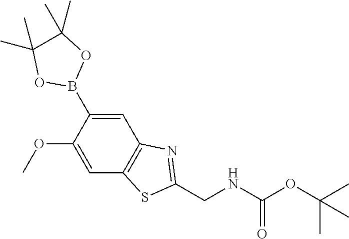 [SMILES: COc1cc2sc(CNC(=O)OC(C)(C)C)nc2cc1B1OC(C)(C)C(C)(C)O1]

To a stirred solution of tert-butyl ((5-bromo-6-methoxybenzo[d]thiazol-2-yl)methyl)carbamate (5 g, 13.44 mmol) in dioxane (100 mL) was added BPin (6.8 g, 26.8 mmol), KOAc (4.6 g, 47.0 mmol) and purged with argon for 15 minutes. Then Pd₂Cl₂(dppf). DCM (1.1 g, 1.34 mmol) was added and purged with argon for further 5 minutes. The reaction mixture was heated at 100° C. for 16 h. The reaction mixture was filtered through celite pad and washed the pad with EtOAc (50 mL). The filtrate was washed with water, brine and evaporated affording a white solid (12 g, crude). M/z 339 (M+H)⁺.

### f. Tert-butyl N-[(5-hydroxy-6-methoxy-1,3-benzothiazol-2-yl)methyl]carbamate

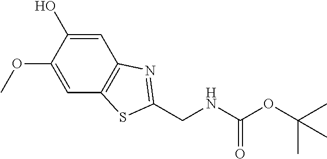 [SMILES: COc1cc2sc(CNC(=O)OC(C)(C)C)nc2cc1O]

To a stirred solution of tert-butyl N-[[6-methoxy-5-(4,4,5,5-tetramethyl-1,3,2-dioxaborolan-2-yl)-1,3-benzothiazol-2-yl]methyl]carbamate (12 g, 35.5 mmol) in THF (180 mL) was added 1N NaOH (35 mL, 35.5 mmol), 30% H₂O₂ (6.2 mL 81.6 mmol) at 0° C. and stirred at same temperature for 30 minutes. The reaction mixture was partitioned between water and EtOAc. The organic layer was separated washed with water, brine and evaporated to get crude compound. The crude compound was chromatographed on silica eluting with 30% EtOAc in pet ether affording an off white solid. (2.5 g 54%). M/z 311.0 (M+H)⁺. ¹H NMR (500 MHz, CDCl₃): δ 7.50 (1H, s), 7.25 (1H, s), 5.76 (1H, s), 5.30 (1H, s), 4.68 (2H, d, J=5.5 Hz), 3.97 (3H, s), 1.54 (9H, s). M/z 311.0 (M+H)⁺.

### g. Tert-butyl N-[[5-[3-(dimethylamino)propoxy]-6-methoxy-1,3-benzothiazol-2-yl]methyl]carbamate

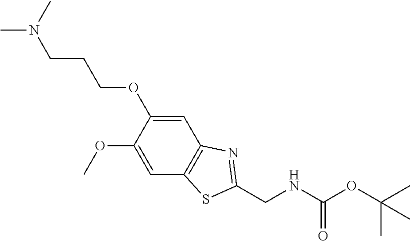 [SMILES: COc1cc2sc(CNC(=O)OC(C)(C)C)nc2cc1OCCCN(C)C]

To a solution of tert-butyl N-[(5-hydroxy-6-methoxy-1,3-benzothiazol-2-yl)methyl]carbamate (750 mg, 2.41 mmol) in DMF (5 mL) was added K₂CO₃ (1 g, 7.25 mmol), 3-chloro-N,N-dimethylpropan-1-amine (355 mg, 2.90 mmol) at room temperature and heated at 80° C. for 4 h. The reaction mixture was diluted with water (25 mL) and extracted with EtOAc (2×30 mL). The combined organic layer was dried, filtered and evaporated affording a pale brown liquid (1 g, crude). M/z 395.8 (M+H)⁺.

### h. 3-[[2-(aminomethyl)-6-methoxy-1,3-benzothiazol-5-yl]oxy]-N,N-dimethyl-propan-1-amine hydrochloride

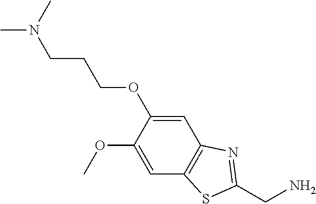 [SMILES: COc1cc2sc(CN)nc2cc1OCCCN(C)C]

To a solution of tert-butyl N-[[5-[3-(dimethylamino)propoxy]-6-methoxy-1,3-benzothiazol-2-yl]methyl]carbamate (1 g, 2.53 mmol) in dioxane (5 mL) was added 4M HCl in dioxane (6 mL) at room temperature and stirred for 6 h. The reaction mixture was evaporated and the resulting residue was triturated with diethyl ether (25 mL) affording a pale yellow solid (0.92 g, crude). M/z 296.2 (M+H)⁺.

### i. Tert-butyl 2-[2-[[5-[3-(dimethylamino)propoxy]-6-methoxy-1,3-benzothiazol-2-yl]methylcarbamoyl]indan-2-yl]acetate

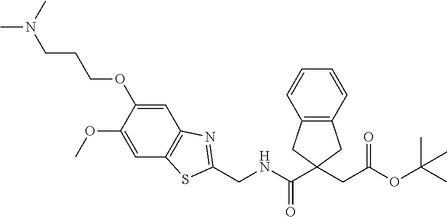 [SMILES: COc1cc2sc(CNC(=O)C3(CC(=O)OC(C)(C)C)Cc4ccccc4C3)nc2cc1OCCCN(C)C]

To a solution of 3-[[2-(aminomethyl)-6-methoxy-1,3-benzothiazol-5-yl]oxy]-N,N-dimethyl-propan-1-amine hydrochloride (450 mg, 1.52 mmol) in DMF (6 mL) was added Et₃N (1.1 mL, 7.62 mmol) and stirred for 10 minutes. Then 2-(2-(tert-butoxy)-2-oxoethyl)-2,3-dihydro-1H-indene-2-carboxylic acid (463 mg, 1.67 mmol), EDC. HCl (440 mg, 2.28 mmol) and HOBt (210 mg, 1.52 mmol) was added at room temperature and stirred for 16 h. The reaction mixture was diluted with cold water (30 mL) and extracted with EtOAc (2×40 mL) and evaporated to get crude compound. The crude was chromatographed on silica eluting with 10-12% MeOH in DCM affording a yellow solid (310 mg, 56%). M/z 554.2 (M+H)⁺.

### j. 2-[2-[[5-[3-(dimethylamino)propoxy]-6-methoxy-1,3-benzothiazol-2-yl]methylcarbamoyl]indan-2-yl]acetic acid

To a solution of tert-butyl 2-[2-[[5-[3-(dimethylamino)propoxy]-6-methoxy-1,3-benzothiazol-2-yl]methylcarbamoyl]indan-2-yl]acetate (120 mg, 0.21 mmol) in DCM (5 mL) was added TFA (2 mL) at 0° C. and stirred at room temperature for 2 h. The mixture was evaporated and the residue was triturated with diethyl ether (15 mL). The crude compound was purified by preparative HPLC [YMC-TRIART(150×25 mm), 10 u, Mobile phase: A: 0.1% Formic Acid in H₂O, B: MeCN]affording the title compound as an off white solid (32 mg, 30%). M/z 498.1 (M+H)⁺. ¹H NMR (500 MHz, DMSO-d₆): δ 9.00 (1H, bs), 7.55 (1H, s), 7.43 (1H, s), 7.21-7.20 (2H, m), 7.14-7.12 (2H, m), 4.60 (2H, d, J=6 Hz), 4.04 (2H, t, J=6.5 Hz), 3.81 (3H, s), 3.45 (2H, d, J=16 Hz), 2.98 (2H, d, J=16 Hz), 2.69 (2H, s), 2.40 (2H, t, J=7 Hz), 2.17 (6H, s), 1.91-1.85 (2H, m).

### Example 53 2-[2-[[6-methoxy-5-[3-(trimethylammonio)propoxy]-1,3-benzothiazol-2-yl]methylcarbamoyl]indan-2-yl]acetate

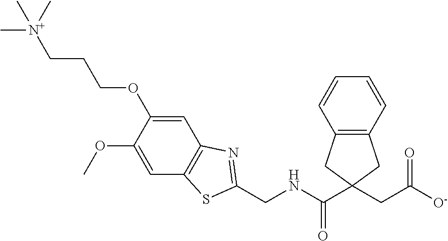 [SMILES: COc1cc2sc(CNC(=O)C3(CC(=O)[O-])Cc4ccccc4C3)nc2cc1OCCC[N+](C)(C)C]

### a. 3-[[2-[[[2-(2-tert-butoxy-2-oxo-ethyl)indane-2-carbonyl]amino]methyl]-6-methoxy-1,3-benzothiazol-5-yl]oxy]propyl-trimethyl-ammonium iodide

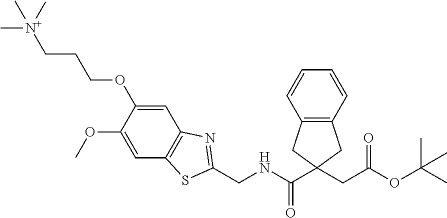 [SMILES: COc1cc2sc(CNC(=O)C3(CC(=O)OC(C)(C)C)Cc4ccccc4C3)nc2cc1OCCC[N+](C)(C)C]

To a solution of tert-butyl 2-[2-[[5-[3-(dimethylamino)propoxy]-6-methoxy-1,3-benzothiazol-2-yl]methylcarbamoyl]indan-2-yl]acetate (200 mg, 0.36 mmol) in acetonitrile (5 mL) was added Mel (1 mL) at 0° C. and stirred at room temperature for 16 h. The mixture was evaporated and resulting residue was purified by silica gel chromatography eluting with 15-20% 7M NH₃/MeOH in DCM affording a pale yellow solid (100 mg, 49%). M/z 568.3 (M)⁺.

### b. 2-[2-[[6-methoxy-5-[3-(trimethylammonio)propoxy]-1,3-benzothiazol-2-yl]methylcarbamoyl]indan-2-yl]acetate

To a solution of 3-[[2-[[[2-(2-tert-butoxy-2-oxo-ethyl)indane-2-carbonyl]amino]methyl]-6-methoxy-1,3-benzothiazol-5-yl]oxy]propyl-trimethyl-ammonium iodide (90 mg, 0.15 mmol) in DCM (5 mL) was treated with TFA (1.5 mL) at 0° C. and stirred at room temperature for 4 h. The mixture was evaporated and the residue was triturated with diethyl ether (10 mL). The crude compound was purified by preparative HPLC [X-BRIDGE-C18 (150*30), 5 u, Mobile phase: A: 0.1% Formic Acid in H₂O, B: MeCN] affording the title compound as an off white solid (8.2 mg, 10%). M/z 512.3 (M+H)⁺. ¹H NMR (500 MHz, DMSO-d₆): 12.27 (1H, bs), 7.64 (1H, s), 7.53 (1H, s), 7.17-7.15 (2H, m), 7.11-7.09 (2H, m), 4.61 (2H, d, J=5.5 Hz), 4.12 (2H, t, J=6 Hz), 3.82 (3H, s), 3.51-3.49 (2H, m), 3.40 (2H, d, J=16 Hz), 3.10 (9H, s), 2.90 (2H, d, J=16 Hz), 2.40 (2H, s), 2.24-2.21 (2H, m).

### Example 54 2-[2-[[6-methoxy-5-[2-[2-(trimethylammonio)ethoxy]ethoxy]-1,3-benzothiazol-2-yl]methylcarbamoyl]indan-2-yl]acetate

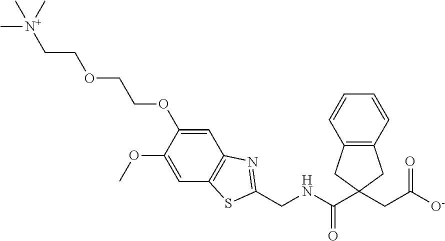 [SMILES: COc1cc2sc(CNC(=O)C3(CC(=O)[O-])Cc4ccccc4C3)nc2cc1OCCOCC[N+](C)(C)C]

### c. Tert-butyl N-[[5-[2-(2-chloroethoxy)ethoxy]-6-methoxy-1,3-benzothiazol-2-yl]methyl]carbamate

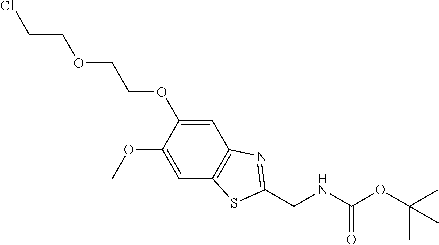 [SMILES: COc1cc2sc(CNC(=O)OC(C)(C)C)nc2cc1OCCOCCCl]

A solution of tert-butyl N-[(5-hydroxy-6-methoxy-1,3-benzothiazol-2-yl)methyl]carbamate (600 mg, 1.93 mmol) in acetonitrile (10 mL) was added Cs₂CO₃ (692 mg, 2.12 mmol) and 1-chloro-2-(2-chloroethoxy)ethane (304 mg, 2.12 mmol) at room temperature. The mixture was heated at 70° C. for 16 h, then filtered through celite pad and washed the pad with EtOAc (15 mL). The filtrate was concentrated and the residue was chromatographed on silica eluting with 25% EtOAc in petroleum ether affording an off white solid (250 mg, 32%). M/z 417.1 (M+H)⁺

### d. Tert-butyl N-[[5-[2-[2-(dimethylamino)ethoxy]ethoxy]-6-methoxy-1,3-benzothiazol-2-yl]methyl]carbamate

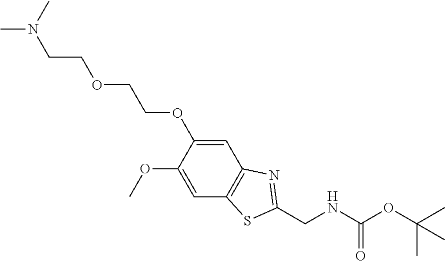 [SMILES: COc1cc2sc(CNC(=O)OC(C)(C)C)nc2cc1OCCOCCN(C)C]

A solution of tert-butyl ((5-(2-(2-chloroethoxy)ethoxy)-6-methoxybenzo[d]thiazol-2-yl)methyl)carbamate (250 mg, 0.60 mmol) in acetone (5 mL) was added Cs₂CO₃ (293 mg, 0.90 mmol) and dimethylamine (2 mL, 2M in THF) at 0° C. The mixture was heated at 90° C. in a sealed tube for 20 h. The reaction mixture was filtered through celite pad and washed the pad with EtOAc (15 mL). The filtrate was concentrated and the residue was chromatographed on silica eluting with 10-20% MeOH in DCM affording a pale yellow solid (210 mg, 84%). M/z 426.2 (M+H)⁺

### e. 2-(2-((2-(aminomethyl)-6-methoxybenzo[d]thiazol-5-yl)oxy)ethoxy)-N,N-dimethylethan-1-amine hydrochloride

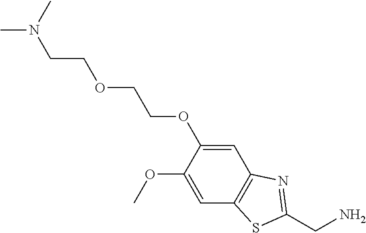 [SMILES: COc1cc2sc(CN)nc2cc1OCCOCCN(C)C]

A solution of tert-butyl ((5-(2-(2-(dimethylamino)ethoxy)ethoxy)-6-methoxybenzo[d]thiazol-2-yl)methyl)carbamate (200 mg, 0.47 mmol) in dioxane (4 mL) was added 4M HCl in dioxane (5 mL) at room temperature. The mixture was stirred at room temperature for 4 h and concentrated under reduced pressure. The residue was triturated with diethyl ether (10 mL) affording an off white solid (180 mg, crude). M/z 326.1 (M+H)⁺.

### f. Tert-butyl 2-[2-[[5-[2-[2-(dimethylamino)ethoxy]ethoxy]-6-methoxy-1,3-benzothiazol-2-yl]methylcarbamoyl]indan-2-yl]acetate

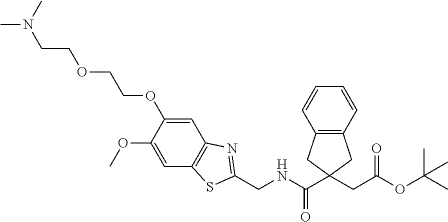 [SMILES: COc1cc2sc(CNC(=O)C3(CC(=O)OC(C)(C)C)Cc4ccccc4C3)nc2cc1OCCOCCN(C)C]

A solution of 2-(2-((2-(aminomethyl)-6-methoxybenzo[d]thiazol-5-yl)oxy)ethoxy)-N,N-dimethylethan-1-amine hydrochloride (300 mg, 0.92 mmol) in DMF (8 mL) was added Et₃N (0.4 mL, 2.76 mmol) and stirred for 10 minutes. Then 2-(2-(tert-butoxy)-2-oxoethyl)-2,3-dihydro-1H-indene-2-carboxylic acid (280 mg, 1.01 mmol) and T3P (0.9 mL, 1.38 mmol) was added at room temperature and stirred for 16 h. The reaction mixture was partitioned between water (15 mL) and EtOAc (30 mL). The organic layer was evaporated and resulting crude compound was chromatographed on silica eluting with 10%-20% MeOH in DCM affording an off white solid (200 mg, 38%). M/z 584.2 (M+H)⁺.

### g. 2-[2-[[2-[[[2-(2-tert-butoxy-2-oxo-ethyl)indane-2-carbonyl]amino]methyl]-6-methoxy-1,3-benzothiazol-5-yl]oxy]ethoxy]ethyl-trimethyl-ammonium

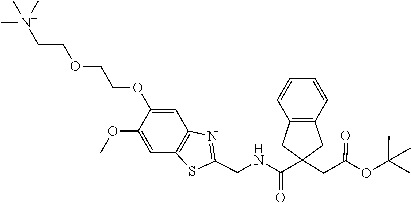 [SMILES: COc1cc2sc(CNC(=O)C3(CC(=O)OC(C)(C)C)Cc4ccccc4C3)nc2cc1OCCOCC[N+](C)(C)C]

A solution of tert-butyl 2-(2-(((5-(2-(2-(dimethylamino)ethoxy)ethoxy)-6-methoxybenzo[d]thiazol-2-yl)methyl)carbamoyl)-2,3-dihydro-1H-inden-2-yl)acetate (200 mg, 0.34 mmol) in acetonitrile (5 mL) was added Mel (1 mL) at 0° C. and stirred at room temperature for 8 h. The mixture was evaporated and the residue was purified by preparative TLC eluting with 10% MeOH in DCM affording an off white solid (60 mg, 30%). M/z 598.1 (M)⁺.

### h. 2-[2-[[6-methoxy-5-[2-[2-(trimethylammonio)ethoxy]ethoxy]-1,3-benzothiazol-2-yl]methylcarbamoyl]indan-2-yl]acetate

A solution of 2-(2-((2-((2-(2-(tert-butoxy)-2-oxoethyl)-2,3-dihydro-1H-indene-2-carboxamido)methyl)-6-methoxybenzo[d]thiazol-5-yl)oxy)ethoxy)-N,N,N-trimethylethan-1-aminium (120 mg, 0.20 mmol) in DCM (5 mL) was treated with TFA (1 mL) at 0° C. and stirred at room temperature for 4 h. The mixture was evaporated and the residue was triturated with diethyl ether (10 mL). The crude compound was purified by preparative HPLC [X-BRIDGE-C18 (150*30), 5 u, Mobile phase: A: 0.1% Formic Acid in H₂O, B: MeCN] and freeze dried affording the title product as an off white solid (46 mg, 43%). M/z 542.2 (M+H)⁺. ¹H NMR (500 MHz, DMSO-d₆): δ 9.85 (1H, bs), 7.58 (1H, s), 7.49 (1H, s), 7.20-7.18 (2H, m), 7.13-7.11 (2H, m), 4.60 (2H, d, J=5.0 Hz), 4.20 (2H, t, J=4 Hz), 3.94 (2H, bs), 3.85 (2H, t, J=4 Hz), 3.82 (3H, s), 3.53 (2H, t, J=4.5 Hz), 3.43 (2H, d, J=16 Hz), 3.10 (9H, s), 2.96 (2H, d, J=16 Hz), 2.62 (2H, s).

### Example 55 2-[5,6-difluoro-2-[[6-methoxy-5-[2-[2-(trimethylammonio)ethoxy]ethoxy]-1,3-benzothiazol-2-yl]methylcarbamoyl]indan-2-yl]acetate

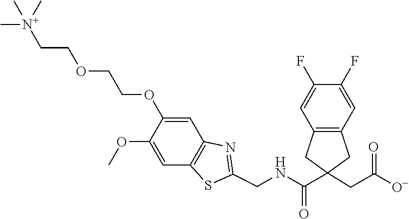 [SMILES: COc1cc2sc(CNC(=O)C3(CC(=O)[O-])Cc4cc(F)c(F)cc4C3)nc2cc1OCCOCC[N+](C)(C)C]

### a. Dimethyl 4,5-difluorophthalate

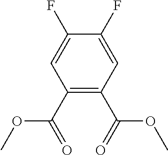

To an ice-cooled solution of 4,5-difluorophthalic acid (11.9 g, 58.9 mmol) in MeOH (250 mL) was added concentrated H₂SO₄ (40 mL, 0.75 mol) keeping the temperature <20° C. The mixture was stirred at 65° C. for 4 h. The cooled reaction mixture was concentrated in vacuo, then the residue was cautiously added to EtOAc and aq. NaHCO₃. The aq. phase was extracted with EtOAc and the combined organic extracts were washed with aq. NaHCO₃, then brine, dried (Na₂SO₄), filtered and concentrated in vacuo to yield the title compound as a colourless oil (12.98 g, 96%). ¹H NMR (CDCl₃) δ 7.56 (2H, t, J=8.7 Hz), 3.91 (6H, s).

### b. (4,5-Difluoro-1,2-phenylene)dimethanol

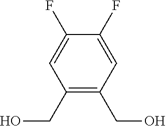 [SMILES: OCc1cc(F)c(F)cc1CO]

To an ice-cooled solution of lithium aluminium hydride (1M in THF, 226 mL, 0.226 mol) was added a solution of dimethyl 4,5-difluorophthalate (12.98 g, 56.4 mmol) in THF (100 mL) over 30 min keeping the temperature below 12° C. The mixture was stirred in the ice bath for 30 min, then at RT for 1 h. The reaction mixture was cooled to 0° C. then, cautiously, water (8.5 mL), 15% aq. NaOH (8.5 mL) and water (26 mL) were added successively, keeping the temperature below 15° C. Celite was added and the mixture stirred at RT for 1 h, then filtered through a celite pad, washing through with more THF. The filtrate was concentrated in vacuo to yield the title compound as a white solid (9.52 g, 97%). ¹H NMR (d6-DMSO) δ 7.36 (2H, t, J=10.1 Hz), 5.29 (2H, t, J=5.5 Hz), 4.47 (4H, d, J=5.4 Hz).

### c. 1,2-Bis(bromomethyl)-4,5-difluorobenzene

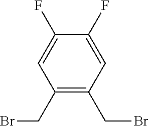 [SMILES: Fc1cc(CBr)c(CBr)cc1F]

A mixture of (4,5-difluoro-1,2-phenylene)dimethanol (9.52 g, 54.7 mmol) and 48% hydrobromic acid (68.5 mL) was stirred at 110° C. for 1 h. The cooled reaction mixture was diluted with water and then extracted with Et₂O. The aq. phase was extracted with Et₂O and the combined organic extracts were washed with water, then brine, dried (Na₂SO₄), filtered and concentrated in vacuo to leave a residue. FCC (1-10% EtOAc in hexane) to yield the title compound as a colourless oil (15.2 g, 93%). ¹H NMR (CDCl₃) δ 7.20 (2H, t, J=9.1 Hz), 4.55 (4H, s).

### d. Diethyl 5,6-difluoro-1,3-dihydro-2H-indene-2,2-dicarboxylate

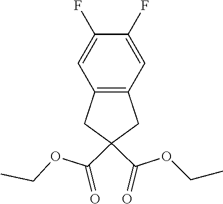 [SMILES: CCOC(=O)C1(C(=O)OCC)Cc2cc(F)c(F)cc2C1]

Sodium hydride (60% in oil, 4.46 g, 112 mmol) was added over 15 min to a mixture of 1,2-bis(bromomethyl)-4,5-difluorobenzene (15.2 g, 50.7 mmol) and diethyl malonate (9.74 g, 60.8 mmol) in THF (200 mL) keeping the temperature below 20° C. The mixture was stirred at RT for 4 h, then saturated ammonium chloride was added. The mixture was concentrated in vacuo and then extracted twice with EtOAc. The combined organic extracts were washed with brine, dried (Na₂SO₄), filtered and concentrated in vacuo to leave a residue. FCC (5-25% EtOAc in hexane) yielded the title compound as a colourless oil (9.95 g, 66%). ¹H NMR (CDCl₃) δ 6.97 (2H, t, J=8.7 Hz), 4.21 (4H, q, J=7.1 Hz), 3.52 (4H, s), 1.26 (6H, t, J=7.1 Hz).

### e. 5,6-Difluoro-2,3-dihydro-1H-indene-2-carboxylic acid

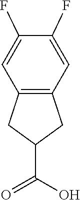 [SMILES: O=C(O)C1Cc2cc(F)c(F)cc2C1]

To a solution of diethyl 5,6-difluoro-1,3-dihydro-2H-indene-2,2-dicarboxylate (9.94 g, 33.3 mmol) in dioxane (130 mL) was added water (130 mL) and concentrated HCl (140 mL). The mixture was refluxed for 23 h. The cooled reaction mixture was diluted with water and extracted with Et₂O (×3). The combined organic extracts were washed with water, then brine, dried (Na₂SO₄), filtered and concentrated in vacuo to yield the title compound as a colourless solid (6.6 g, quant.). M/z 197 (M−H)⁻.

### f. Methyl 5,6-difluoro-2,3-dihydro-1H-indene-2-carboxylate

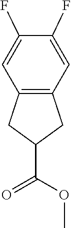

To an ice-cooled solution of 5,6-difluoro-2,3-dihydro-1H-indene-2-carboxylic acid (6.6 g, 33.3 mmol) in MeOH (200 mL) was added concentrated H₂SO₄ (40 mL, 0.75 mol) keeping the temperature <20° C. The mixture was stirred at 65° C. for 1 h. The cooled reaction mixture was concentrated in vacuo, then the residue was cautiously added to EtOAc and aq. NaHCO₃. The aq. phase was extracted with more EtOAc and the combined organic extracts were washed with brine, dried (Na₂SO₄), filtered and concentrated in vacuo to leave a residue. FCC (5-25% EtOAc in hexane) yielded the title compound as a pale yellow solid (5.97 g, 84%). ¹H NMR (CDCl₃) δ 6.98 (2H, t, J=8.8 Hz), 3.73 (3H, s), 3.39 (1H, m), 3.24-3.12 (4H, m).

### g. Methyl 2-(2-(tert-butoxy)-2-oxoethyl)-5,6-difluoro-2,3-dihydro-1H-indene-2-carboxylate

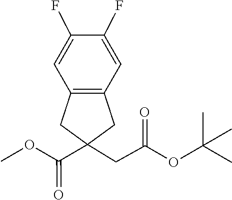 [SMILES: COC(=O)C1(CC(=O)OC(C)(C)C)Cc2cc(F)c(F)cc2C1]

To a solution of methyl 5,6-difluoro-2,3-dihydro-1H-indene-2-carboxylate (5.97 g, 28.2 mmol) in THF (120 mL), cooled to −78° C., was added sodium bis(trimethylsilyl)amide (1M in THF, 42.2 mL, 42.2 mol) over 15 min. The mixture was stirred at −78° C. for 45 min then a solution of tert-butyl bromoacetate (8.24 g, 42.2 mmol) in THF (15 mL) was added over 10 min. The reaction mixture was allowed to warm to −10° C. over 1 h. Saturated ammonium chloride was added and the mixture was concentrated under reduced pressure. The residue was extracted twice with EtOAc and the combined organic extracts were washed with brine, dried (Na₂SO₄), filtered and concentrated in vacuo to leave a residue. FCC (5-20% EtOAc in hexane) yielded the title compound as a yellow gum (8.78 g, 96%). ¹H NMR (CDCl₃) δ 6.96 (2H, t, J=8.9 Hz), 3.72 (3H, s), 3.47 (2H, d, J=16.2 Hz), 2.90 (2H, d, J=16.2 Hz), 2.71 (2H, s), 1.42 (9H, s).

### h. 2-[(tert-butoxy)carbonyl]-5,6-difluoro-2,3-dihydro-1H-indene-2-carboxylic acid

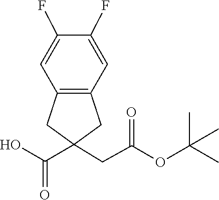 [SMILES: CC(C)(C)OC(=O)CC1(C(=O)O)Cc2cc(F)c(F)cc2C1]

To a solution of methyl 2-(2-(tert-butoxy)-2-oxoethyl)-5,6-difluoro-2,3-dihydro-1H-indene-2-carboxylate (0.834 g, 2.56 mmol) in THF (25 mL) and MeOH (10 mL) was added lithium hydroxide (0.5M in water, 10.2 mL, 5.1 mmol). The mixture was stirred at RT for 2.5 h, then concentrated in vacuo. The residual solution was layered with EtOAc and acidified by addition of 6M HCl. The aq. phase was extracted with more EtOAc and the combined organic extracts were washed with brine, dried (Na₂SO₄), filtered and concentrated in vacuo to leave a residue. FCC (2-6% MeOH in DCM) yielded the title compound as a cream solid (0.59 g, 74%). ¹H NMR (d6-DMSO) δ 12.47 (1H, bs), 7.26 (2H, t, J=9.2 Hz), 3.33 (2H, d, J=16.4 Hz), 2.91 (2H, d, J=16.4 Hz), 2.67 (2H, s), 1.37 (9H, s). M/z 311 (M−H)⁻.

### i. 2-[5,6-difluoro-2-[[6-methoxy-5-[2-[2-(trimethylammonio)ethoxy]ethoxy]-1,3-benzothiazol-2-yl]methylcarbamoyl]indan-2-yl]acetate

This was prepared in an analogous manner to Example 54 using 2-[(tert-butoxy)carbonyl]-5,6-difluoro-2,3-dihydro-1H-indene-2-carboxylic acid in step-d. The title compound was isolated as an orange solid (58.5 mg). M/z 578.5 (M+H)⁺. ¹H NMR (500 MHz, DMSO-d₆): δ 13.05 (1H, bs), 7.60 (1H, s), 7.61 (1H, s), 7.22-7.21 (1H, m), 7.17-7.14 (1H, m), 4.62 (2H, d, J=6 Hz), 4.21 (2H, m), 3.93 (2H, m), 3.87 (2H, s), 3.82 (3H, s), 3.58 (2H, m), 3.36 (2H, m), 3.10 (9H, s), 2.89-2.81 (2H, m), 2.32 (2H, m).

### Example 56 2-[5,6-difluoro-2-[[6-methoxy-5-[2-(trimethylammonio)ethoxy]-1,3-benzothiazol-2-yl]methylcarbamoyl]indan-2-yl]acetate

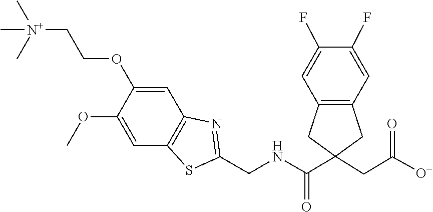 [SMILES: COc1cc2sc(CNC(=O)C3(CC(=O)[O-])Cc4cc(F)c(F)cc4C3)nc2cc1OCC[N+](C)(C)C]

This was prepared in an analogous manner to Example 54 using in 2-Chloro-N,N-dimethylethylamine hydrochloride in step-a and 2-[(tert-butoxy)carbonyl]-5,6-difluoro-2,3-dihydro-1H-indene-2-carboxylic acid in step-d. The title compound was isolated as a white solid (13 mg, 12%). M/z 534.3 (M+H)⁺. ¹H NMR (500 MHz, DMSO-d₆): δ 13.16 (1H, bs), 7.69 (1H, s), 7.63 (1H, s), 7.22-7.21 (1H, m), 7.17-7.14 (1H, m), 4.61 (2H, m), 4.53 (2H, m), 3.82 (5H, m), 3.32 (2H, m), 3.21 (9H, s), 2.89-2.81 (2H, m), 2.32 (2H, m).

### Example 57: LasB Inhibitory Activity Measurements

The relevance of LasB to PA infection has been shown in experiments measuring lung burden in a rat model of chronic lung infection following infection with WT PA (which expresses LasB) and a mutant form of PA (ΔlasB PA) in which LasB is not expressed. It could be clearly seen in that following infection, whereas a wild type strain is able to persist at least for 14 days, a LasB deficient strain was not able to persist beyond day 5. The relevance of LasB to PA biofilm development was also shown. Biofilms formed after 3 days by both PA26 wt and PA26 lasB deletion strains were investigated by confocal imaging and subsequent analysis (with Comstat software). This study demonstrated that biofilms formed by the PA26 lasB deletion strain were highly reduced in thickness and biomass compared to the wt strain, demonstrating the essential role of LasB in PA biofilm development.

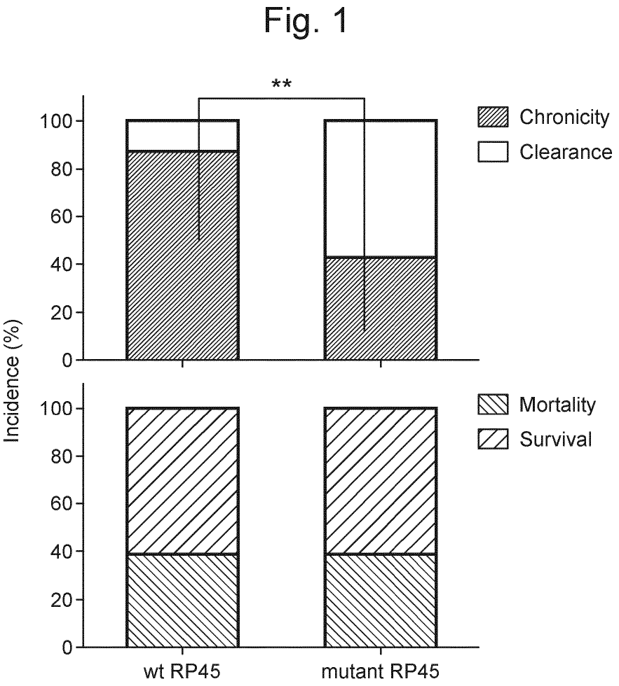

The relevance of LasB to Pseudomonas aeruginosa (PA) infection is illustrated in FIG. 1, which shows incidence of mortality versus survival, and chronic colonisation versus bacterial clearance, in a mouse model of lung infection. Chronicity of the infection is defined by PA lung burden higher than 10′CFU seven days after infection. In this infection model, both wild type strain (expressing LasB; “wt RP45”) and the isogenic lasB deleted strain (which does not express LasB; “mutant RP45”) cause similar mortality (in around 40% of infected mice); however the incidence of chronic colonization was significantly lower for the mutant strain in comparison to the wt counterpart (87% for the wt vs 43% for the lasB deleted strain; Fisher exact test p<0.01). This finding shows the role of LasB in establishment of chronic colonization.

Experiments were therefore conducted (1) to measure the potency of inhibition of compounds of the invention against purified Pseudomonas aeruginosa LasB enzyme and also experiments were conducted (2) to measure the ability of compounds of the invention to inhibit LasB-catalysed elastin degradation. The first assay uses a commercial fluorescent synthetic peptide and purified LasB enzyme. The LasB hydrolysis kinetics are measured allowing the determination of the IC50 and Ki of the inhibitors; the second is a more physiological assay using dialysed Pseudomonas aeruginosa supernatant as source of enzyme, plus its natural substrate Elastin. It is an “end point assay” that determines the percentage of LasB inhibition by each compound for one particular time point and inhibitor concentration. Technical details are described below:

### Fluorometric Assay to Determine Ki

This assay uses commercially available substrate (Abz-Ala-Gly-Leu-Ala-p-Nitro-Benzyl-Amide (Ex: 340 nm, Em: 415 nm) from Peptide International) and purified LasB protein from P. aeruginosa (provided by Merck or Charles River Laboratories). It is performed to determine LasB elastase activity and assess compound inhibition in 96-well plate format. All compounds of Formula (I) were assessed using the method described below.

Method: 10 to 140 ng/ml purified LasB is incubated with 250 μM Abz-Ala-Gly-Leu-Ala-p-Nitro-Benzyl-Amide in 50 mM Tris-HCl pH 7.4, 2.5 mM CaCl₂), 0.01% of Triton X100 at 37° C. LasB activity (corresponding to fluorescence emission induced by substrate hydrolysis) is measured over 30 min at 37° C. with a fluorescence plate reader such as the Perkin Elmer Envision or similar. Different range of inhibitor concentrations are routinely assessed depending of inhibitor potency from 0.0016 to 200 μM (2-fold dilutions series) in order to determine IC50.

The equation used to calculate the Ki from IC50 is: Ki=IC50/(1+([S]/Km)) where [S]=250 μM and Km=214 μM.

### Elastin Assay to Determine % Inhibition

The Elastin assay uses as source of enzyme dialysed supernatant from P. aeruginosa PAO1 and the Elastin Congo-Red as substrate. The natural LasB substrate, elastin, is complexed with the congo-red dye (Elastin Congo-Red, ECR). The elastolysis activity from the culture supernatant will degrade elastin and release the congo-red dye into the supernatant. This red dye release can be measured with a spectrophotometer.

All compounds of Formula (I) were assessed using the method described below.

Method: To determine LasB elastase activity and assess compound inhibition, an overnight culture of P. aeruginosa strain PAO1 is diluted in LB medium. After reaching an OD₆₀₀ₙₘ of 0.6, this culture is diluted and incubated for additional 18-24 hours in a shaking incubator. Culture supernatants are recovered by centrifugation and filtrated through a 0.22 μM filter. These supernatants are dialysed (filtration molecules <20 kDa) into a 50 mM Tris-HCl pH 7.4, 2.5 mM CaCl₂ solution at 4° C. under agitation for 24 hours. Supernatant dialysed is then mixed volume/volume with the ECR suspension (20 mg/mL of ECR in 100 mM Tris-HCl pH 7.4 buffer supplemented with 1 mM CaCl2)) supplemented with Triton X100 (final concentration of 0.01%) in presence of DMSO (positive control) and/or different concentrations of compound (routinely 50 to 1.56 μM). As a negative control, the dialysed supernatant is replaced by Tris-HCl solution (50 mM Tris-HCl pH 7.4, 2.5 mM CaCl₂). The mixed reaction is then incubated overnight in a 37° C. shaking incubator. The reaction supernatant is recovered by centrifugation and the release of congo-red is measured by its absorbance at 495 nm (OD₄₉₅ₙₘ).

Percentage inhibition is determined using the following equation:

((OD₄₉₅ nm value of positive control−OD₄₉₅ₙₘ value of negative control)−(OD₄₉₅ₙₘ value of treated supernatant−OD₄₉₅ₙₘ value of negative control))/(OD₄₉₅ₙₘ value of positive control−OD₄₉₅ₙₘ value of negative control)×100.

Results are shown in the Table below and categorised into A, B and C for both assays. The Ki values are grouped as A (Ki=0.00 to 0.05 μM), B (Ki=0.05 to 0.2 μM) and C (Ki=0.2 to 10.00 μM). Similarly, for the elastase hydrolysis assay, values are grouped into A (>80% inhibition), B (60 to 80% inhibition) and C (10 to 60% inhibition) all at 25 μM inhibitor concentration. (n.d. not determined).

| | | Elastin hydrolysis |
|---|---|---|
| | | % inhibition @ 25 μM |
| Example | Ki (μM) | inhibitor concentration |
| 1 | C | C |
| 2 | C | ND |
| 3 | B | B |
| 4 | C | ND |
| 5 | C | ND |
| 6 | B | B |
| 7 | C | ND |
| 8 | B | B |
| 9 | A | A |
| 10 | A | A |
| 11 | A | A |
| 12 | B | B |
| 13 | B | B |
| 14 | B | B |
| 15 | B | B |
| 16 | A | B |
| 17 | B | B |
| 18 | A | B |
| 19 | A | A |
| 20 | A | B |
| 21 | A | A |
| 22 | A | B |
| 23 | A | A |
| 24 | C | B |
| 25 | C | B |
| 26 | C | ND |
| 27 | B | B |
| 28 | C | ND |
| 29 | C | ND |
| 30 | C | ND |
| 31 | C | ND |
| 32 | B | B |
| 33 | A | B |
| 34 | A | A |
| 35 | B | B |
| 36 | A | A |
| 37 | A | A |
| 38 | B | B |
| 39 | C | ND |
| 40 | B | B |
| 41 | B | B |
| 42 | C | B |
| 43 | B | B |
| 44 | A | A |
| 45 | B | B |
| 46 | B | B |
| 47 | B | B |
| 48 | B | B |
| 49 | B | B |

For the following compounds the Ki values are grouped as A (Ki=0.00 to 0.050 μM), B (Ki=0.05 to 0.1 μM) and C (Ki=0.1 to 10.00 μM). Similarly, for the elastase hydrolysis assay, values are grouped into A (>75% inhibition), B (60 to 75% inhibition) and C (10 to 60% inhibition) all at 25 μM inhibitor concentration. (n.d. not determined).

| | | Elastin hydrolysis |
|---|---|---|
| | | % inhibition @ 50/25 μM |
| Example | Ki (μM) | inhibitor concentration |
| 50 | B | A |
| 51 | B | A |
| 52 | B | A |
| 53 | A | A |
| 54 | B | B |
| 55 | A | A |
| 56 | B | B |

### Example 58: Inhibition of LasB-Mediated IL-1β Activation

The activity of compounds of the invention to inhibit LasB-mediated hydrolysis of pro-IL-1β to IL-1β was demonstrated using an enzymatic in vitro assay, using purified LasB and a reporter substrate (a FRET peptide mimicking the LasB IL-1β cleavage site). Hydrolysis of this FRET peptide was continuously monitored using a Victor multimode plate reader (Perkin Elmer) with excitation 355 nm and emission at 450 nm in the presence of varying concentrations of compounds of the invention. Inhibitory constants (Ki) were determined for certain compounds of the invention (at least 2 independent replicates) using a competitive inhibitor model. Results are shown in the table below.

| | Ki (LasB-mediated hydrolysis |
|---|---|
| Example | of pro-IL-1β to IL-1β)/μM |
| 9 | 0.16 |
| 11 | 0.22 |
| 19 | 0.27 |
| 20 | 0.34 |
| 22 | 0.40 |
| 23 | 0.50 |
| 44 | 0.30 |
| 53 | 0.70 |
| 55 | 0.42 |

### Example 59: In Vivo Efficacy of Compounds of the Invention

Experiments were conducted to demonstrate the efficacy of compounds of the invention in treating a mouse model of Pseudomonas aeruginosa lung infection.

Mice were dosed by intranasal inoculation of PA (PAO1), then sacrificed after 24 hours. The extent of infection in the lung was quantified by bacterial load (CFU determination, colony forming units) and the levels of proinflammatory IL-1β. Statistical analysis on both readouts were performed by ANOVA with a Dunnett post-test.

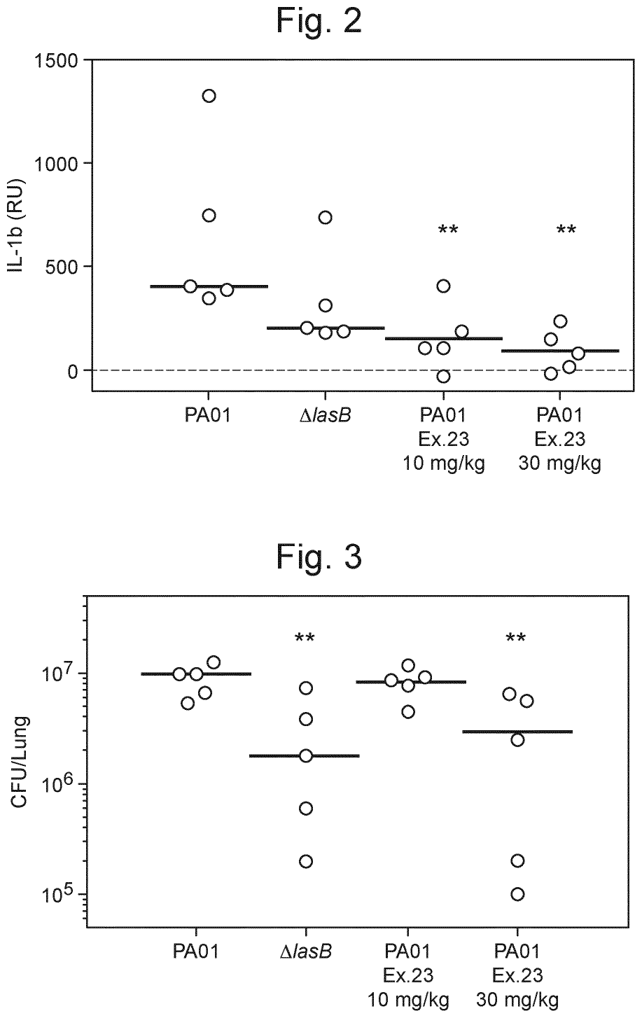

Compounds were administered intravenously in a two-dose regimen (1 hour and 2 hours post infection) at two different doses (10 and 30 mg/kg). As shown in FIG. 2, the compound of Example 23 inhibited the production and activation of IL-1β in mice infected by wild-type PA (PA01) at a similar level than the lasB deleted mutant (ΔlasB), which cannot produce LasB. As shown in FIG. 3, the compound of Example 23 reduced the extent of infection in the lung to the level of the LasB deleted mutant (ΔlasB), as determined by the CFU levels.

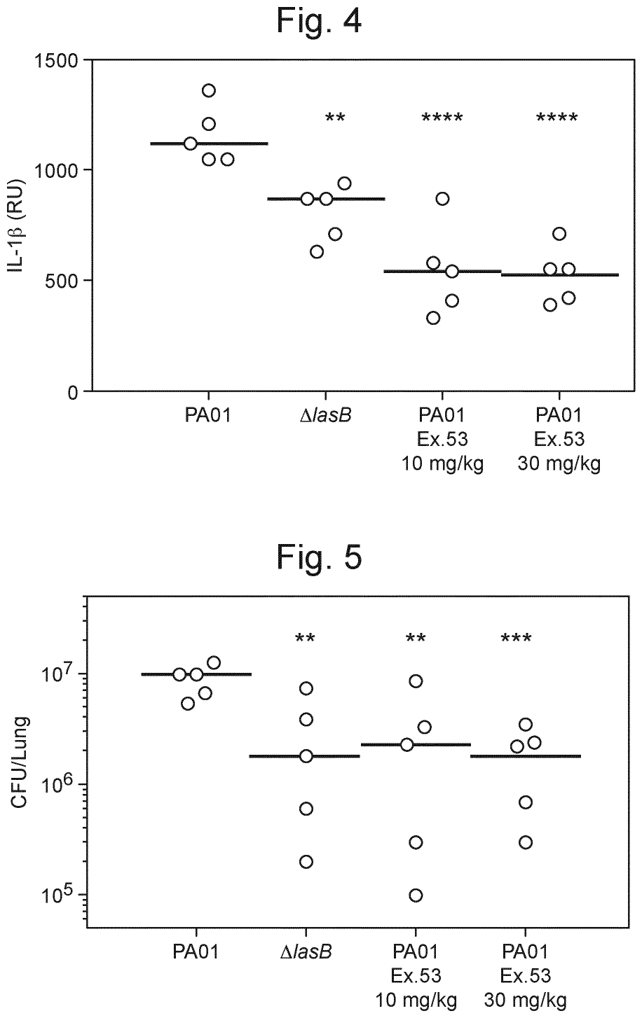

As shown in FIG. 4, the compound of Example 53 inhibited the production and activation of IL-1β in mice infected by wild-type PA (PA01) at a similar level than the lasB deleted mutant (ΔlasB), which cannot produce LasB. As shown in FIG. 5, the compound of Example 53 reduced the extent of infection in the lung to the level of the LasB deleted mutant (ΔlasB), as determined by the CFU levels.

### Example 60: Improvement in CFTR Function in CF-Mutant Human Bronchial Epithelial Cells Treated with CFTR Modulators and LasB Inhibitors

This example describes experiments showing that LasB impacted CFTR function in CF mutant cells can be mitigated by treatment with CFTR modulators and LasB inhibitors.

Wild Type human bronchial epithelial (HBE) cells and CF-mutant human bronchial epithelial cells (F508del-CFTR) are grown at 37° C. for 5 days and loaded onto 96-well plate cultures. The cells are loaded with blue membrane potential dye dissolved in chloride-free buffer. The plate is read in a fluorescence plate reader at 37° C. (excitation: 530 nM; emission; 560 nM). CFTR is stimulated with forskolin.

A control signal defined as 100% transmission is obtained from WT human bronchial epithelial cells alone. A minor suppression of forskolin-activated CFTR response is expected for such WT cells in the presence of LasB.

Moderate suppression in the forskolin-activated CFTR response will be observed for CF-mutant cells. This moderate suppression will be reduced by addition of a CFTR modulator such as VX-809 (lumacaftor).

Substantial suppression in the forskolin-activated CFTR response is expected for CF-mutant cells in the presence of LasB, even following the addition of a CFTR modulator, due to the deleterious effect of LasB on expressed CFTR protein. Addition of a LasB inhibitor such as the compound of Example 53 will therefore mitigate this deleterious effect and result in forskolin-activated CFTR response being substantially restored.

These effects are summarized in the table below:

| | Maximum forskolin- |
|---|---|
| | activated response of CFTR |
| | channels compared to WT |
| | human bronchial epithelial cells |
| WT HBE cells | 100% activation |
| WT HBE cells + 1 μM LasB | Minor suppression of CFTR |
| | activation |
| F508del-CFTR cells | moderate suppression of CFTR |
| | activation |
| F508del-CFTR cells + 10 μM | Minor suppression of CFTR |
| VX-809 | activation |
| F508del-CFTR cells + 10 μM | substantial suppression of CFTR |
| VX-809 + 0.01 μM LasB | activation |
| F508del-CFTR cells + 10 μM | Minor suppression of CFTR |
| VX-809 + 0.01 μM LasB + 10 | activation |
| μM Example 53 | |

Thus, a detrimental effect on CFTR function of LasB on the beneficial effects of CFTR modulators may be reversed by the addition of a LasB inhibitor such as the compound of Example 53.

### Example 61: Inhibition of LasB-Mediated Reduction of CFTR Levels on Airway Cells

These experiments measured the amount of CFTR protein expressed in airway cells under normal conditions and also exposed to 100 nM LasB. A reduction in CFTR protein expression was observed in LasB exposed cells compared to those grown under normal conditions. The effect of adding increasing concentrations of a LasB inhibitor (here the compound of Example 23) on this reduction of CFTR by LasB was then examined.

### Cell Culture:

Normal Human Bronchial Epithelial (NHBE) airway cells were expanded in Pneumacult™ EX medium on plastic PureCol™ coated dishes. Cells were plated on Millicell inserts (200K/insert) and differentiated at air-liquid interface for 26 days in modified Lonza Medium. Prior to treatment, cells were washed in 1×DPBS buffer.

### Treatment Procedures:

Treatments were 100 nM LasB from Pseudomonas aeruginosa and 0, 1, 10, or 100 μM Example 23 for 24 hrs. A stock solution of LasB (200 nM) was prepared in buffer containing 50 mM Tris HCL pH 7.4, 2.5 mM CaCl₂ and 0.01% TritonX-100. A 20 mM stock solution of Example 23 was made in DMSO and sonicated for 10 minutes. The DMSO stock was diluted, 10 μL into 990 μL buffer, to give a 200 μM solution. This was further diluted 1 in 10 in buffer to give a 20 μM solution and then again diluted 1 in 10 to give a 2 μM solution. The solutions were mixed 1:1 with LasB (200 nM) to give the final concentrations of LasB and Example 23 as indicated above for addition to the cell cultures. 20 μl were applied apically to cell cultures for 24 hours before processing and analysis.

Analysis—CFTR Immunoprecipitation (IP) and Western Blotting (WB):

The cells were lysed with 1% NP-40 lysis buffer containing protease inhibitors (1 μg/ml leupeptin, 2 μg/ml aprotinin, 50 g/ml pefabloc, 121 μg/ml benzamidine, 3.5 μg/ml E64) and stored at −80° C. until IP/WB analysis. CFTR was immunoprecipitated with rabbit polyclonal antibody 155 against CFTR and bound to immobilized Protein A/Protein G agarose beads (Santa Cruz Biotechnology, Inc.) and then eluted in 20 μl sample loading buffer. 15 μl were loaded on 4-20% gradient gels (BioRad Laboratories, Inc.) and transferred to nitrocellulose membranes. Transferred membranes were blocked in blocking buffer (Odyssey) and then probed with CFTR antibodies 596 and 217 (CFTR Antibody Distribution Program, from the Cystic Fibrosis Foundation) and then with IRDye 680-goat anti-mouse immunoglobulin G (Molecular Probes, Inc.). Anti-actin (Cell Signaling) was used as a loading control for a standard “house-keeping” protein. Protein bands were visualized using a Sapphire Biomolecular Imager (Azure Biosystems) and quantitated with AzureSpot Analysis Software.

### Results:

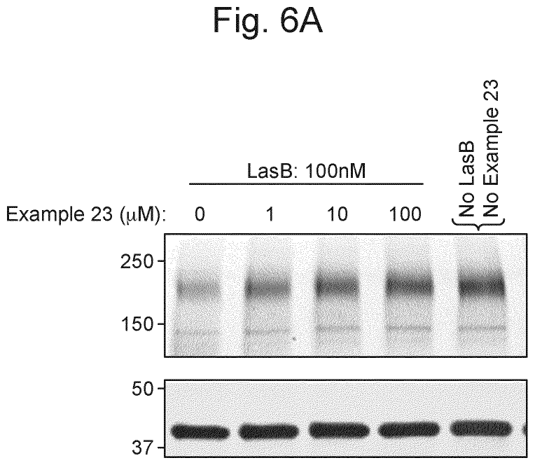

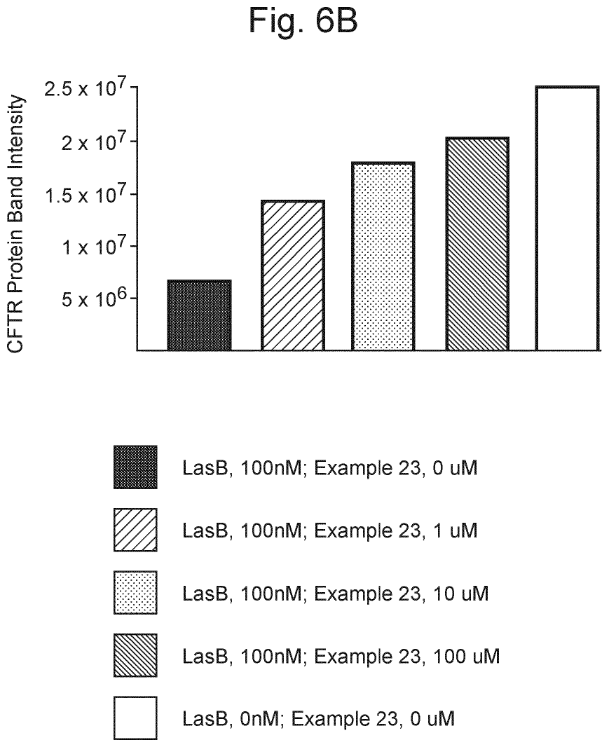

FIG. 6A is a photographic representation of the plate, showing CFTR protein (varying intensities; upper spot) plus a standard “house-keeping” protein (actin) as a control (lower spot). Lane 5 shows the “natural” level of CFTR expression, with no added LasB or Example 23. Lane 1 shows the effect of LasB alone, showing a significant reduction in CFTR levels which is ameliorated by increasing concentrations of Example 23 (lanes 2 to 4). FIG. 6B is the same experiment but here the data is shown in a quantitative form, again showing the significant reduction in CFTR levels by LasB and the amelioration of this reduction by increasing levels of Example 23.

### Conclusion:

Inhibitors of LasB such as the compound of Example 23 are able to counteract the reduction in CFTR expression caused by LasB in a dose-related manner, returning the CFTR level to a similar level as observed in the non-LasB exposed cells.

1. A combination comprising (i) a compound which is an indane according to Formula (I) or a pharmaceutically acceptable salt thereof; and (ii) one or more CFTR modulator; 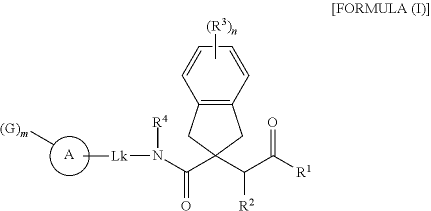
wherein:
R¹ is selected from:
—NHOH, —OH, —OR^{1a} and —OCH₂OC(O)R^{1a}, wherein R^{1a} is selected from an unsubstituted C₁ to C₄ alkyl group and phenyl; and
wherein when the compound of Formula (I) contains a positively charged nitrogen atom, R¹ may be O⁻, such that the compound forms a zwitterion;
R² is selected from H and unsubstituted C₁ to C₂ alkyl;
each R³ group is independently selected from halogen, —OH, —NH₂, methyl and —CF₃;
n is an integer from 0 to 4;
R⁴ is selected from H and unsubstituted C₁ to C₂ alkyl;
Lk is a linking group;
\textcircled{A} is a cyclic group selected from C₆ to C₁₀ aryl, 5- to 14-membered heteroaryl, and 4- to 14-membered carbocyclic and heterocyclic groups; wherein when \textcircled{A} is a heterocyclic or heteroaryl group comprising at least one nitrogen atom, said nitrogen atom(s) are independently selected from secondary, tertiary and quaternary nitrogen atom(s);
m is an integer from 0 to 3; and
each G group is selected from:
a 4- to 10-membered nitrogen-containing heterocyclic group which is unsubstituted or is substituted; wherein the nitrogen atom(s) in said heterocyclic group are independently selected from secondary, tertiary and quaternary nitrogen atom(s);
C₂ to C₄ alkoxy; C₁ to C₄ alkyl; C₂ to C₄ alkenyl; C₂ to C₄ alkynyl; and —NR^{Y}—C₁ to C₄ alkyl each of which is unsubstituted or is substituted; wherein R¹ is H or unsubstituted C₁ to C₃ alkyl;
methoxy which is unsubstituted or is substituted by one, two or three halogen substituents; halogen; —OH; —NR²⁰R²¹ and —N⁺R²⁰R²¹R²², wherein R²⁰ and R²¹ are each independently selected from H and C₁ to C₃ alkyl which is unsubstituted or substituted;
C₃ to C₆ carbocyclyl; —O—C₃ to C₆ carbocyclyl; and —NR^{Y}—C₃ to C₆ carbocyclyl; wherein R¹ is H or unsubstituted C₁ to C₃ alkyl; and
R⁶, wherein each R⁶ group is independently selected from:
—R^{6a}R^{A}, —O—R^{6a}R^{A}, —NR²⁰—R^{6a}R^{A}, —R^{6b}R^{B}, —O—R^{6b}R^{B}, and —NR²⁰—R^{6b}R^{B};
—R^{X}R^{R}, —O—R^{X}R^{R}, —O—R^{X}—C(O)—R^{R}, —R^{X}—C(O)—R^{R}, —NR²⁰—R^{X}R^{R}, and —NR²⁰—R^{X}—C(O)—R^{R}; and
CN; —C(O)NR²⁰R²¹; —C(O)NR²¹—R^{X}R^{B}; —C(O)NR⁴⁰R⁴¹; —SO₂R²⁰; —SO₂—R^{X}R^{B}; —SO₂NR²⁰R²¹; —SO₂—NR²⁰—R^{X}R^{B}; and —SO₂NR⁴⁰R⁴¹;
wherein:
each R^{X} is independently selected from R^{6a} and R^{6b};
each R^{6a} is independently selected from C₁ to C₄ alkylene, C₂ to C₄ alkenylene and C₂ to C₄ alkynylene; and each R^{6a} is independently unsubstituted or is substituted;
each R^{6b} is independently selected from [C₁ to C₃ alkylene]-[5-6-membered carbocyclyl or heterocyclyl], [C₂ to C₃ alkenylene]-[5-6-membered carbocyclyl or heterocyclyl] and [C₂ to C₃ alkynylene]-[5-6-membered carbocyclyl or heterocyclyl];
R^{A} is selected from —NR²⁰R³⁰; —N⁺R²⁰R²¹R³⁰; —NR²⁰NR²¹R²²; —NR²⁰N⁺R²¹R²²R²³; —N⁺R²⁰R²¹NR²²R²³; —NR²⁰C(NR²¹)NR²²R³⁰; —NR²⁰C(N⁺R²¹R²²)NR²³R³⁰; —C(NR²⁰)NR²¹R²²; and —C(N⁺R²⁰R²¹)NR²²R²³;
R^{B} is selected from —NR²⁰R²¹; —N⁺R²⁰R²¹R²²; —NR²⁰NR²¹R²²; —NR²⁰N⁺R²¹R²²R²³; —N⁺R²⁰R²¹NR²²R²³; —NR²⁰C(NR²¹)NR²²R²³; —NR²⁰C(N⁺R²¹R²²)NR²³R²⁴; —C(NR²⁰)NR²¹R²²; and —C(N⁺R²⁰R²¹)NR²²R²³;
R⁴⁰ and R⁴¹, together with the nitrogen atom to which they are attached, form a 4- to 6-membered heterocyclic group which is unsubstituted or substituted, wherein any nitrogen atom in the ring is independently selected from secondary, tertiary and quaternary nitrogen atoms;
each R^{R} is independently substituted 4- to 10-membered heteroaryl or heterocyclic group which is unsubstituted or substituted and which comprises at least one nitrogen atom, and said nitrogen atom(s) are independently selected from secondary, tertiary and quaternary nitrogen atom(s);
R²⁰, R²¹, R²², R²³ and R²⁴ are each independently selected from H and C₁ to C₃ alkyl which is unsubstituted or substituted; and
each R³⁰ is independently selected from C₂ to C₃ alkyl which is unsubstituted or substituted.

2-29. (canceled)

30. The combination of claim 1, wherein Lk is selected from -L- and —(CH₂)_{d}-L′—(CH₂)ₑ—; wherein:
i) L is selected from a bond and a C₁ to C₃ alkylene group which is unsubstituted or is substituted by one group selected from halogen, —OH, —OMe, —NR²⁰R²¹; —N⁺R²⁰R²¹R²², and —CF₃; wherein R²⁰, R²¹ and R²² are each independently selected from H and C₁ to C₃ alkyl which is unsubstituted or is substituted with one —OH or —OMe group or with one, two or three halogen groups;
and
ii) d is 0 or 1; e is 0 or 1; and L′ is selected from the moieties: 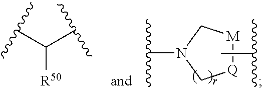
wherein
R⁵⁰ is selected from —R⁶⁰, —C(O)OR^{6c}; —C(O)NR¹⁰R⁶⁰; and —C(O)R⁶⁰;
R⁶⁰ is selected from
i) H;
ii) a C₁ to C₄ alkyl group which is unsubstituted or is substituted with one, two or three groups independently selected from —OH; —NR¹⁰R¹¹; —N⁺R¹⁰R¹¹R¹²; and halogen; and
iii) a cyclic group selected from 3- to 10-membered carbocyclic and heterocyclic groups, 5- to 10-membered heteroaromatic groups and 6- to 10-membered aromatic groups; which cyclic group is unsubstituted or is substituted by one or two substituents independently selected from —OH; —NR¹⁰R¹¹; —N⁺R¹⁰R¹¹R¹²; halogen; and C₁ to C₄ alkyl groups which are themselves each independently unsubstituted or substituted with one, two or three groups independently selected from —OH; —NR¹⁰R¹¹; —N⁺R¹⁰R¹¹R¹²; and halogen;
wherein when said cyclic group is a heterocyclic group comprising at least one nitrogen atom, said nitrogen atom(s) are independently selected from secondary, tertiary and quaternary nitrogen atom(s);
the moiety -M-Q- is selected from —CH₂—CH₂—; —CH₂—NH—; and —CH₂—O—;
wherein a hydrogen atom from one of M and Q is replaced with the bond to the moiety —(CH₂)ₑ—NR⁴—; with the proviso that when e is 0, the moiety -M-Q- is bonded to the —NR⁴— moiety of Formula (I) via a ring carbon atom;
r is 1 or 2; and
each R¹⁰, R¹¹ and R¹² is independently H or methyl.

31. The combination of claim 1, wherein Lk is —CH₂—.

32. The combination of claim 1, wherein \textcircled{A} is selected from benzothiazole, thiazole, pyrazole, benzene, benzofuran, benzimidazole, benzothiophene, benzoxazole, indole, isoquinoline, 2,3-dihydrobenzofuran, 2,3-dihydrobenzo[b][1,4]dioxine, and 4,5,6,7-tetrahydrothiazolo[5,4-c]pyridine.

33. The combination of claim 1, wherein \textcircled{A} is benzothiazole.

34. The combination of claim 1, wherein m is 1 or 2.

35. The combination of claim 1, wherein:
R¹ is selected from —OH and —NHOH, or where the compound of Formula (I) contains a positively charged nitrogen atom, R¹ may be O⁻, such that the compound forms a zwitterion;
R² is H;
R⁴ is H;
n is 0; or n is 2 and each R³ group is halogen;
Lk is —CH₂—;
\textcircled{A} is benzothiazole; and
m is 2.

36. The combination of claim 1, wherein each G group is independently selected from:
i) a 4- to 10-membered nitrogen-containing heterocyclic group which is unsubstituted or is substituted by one or two substituents independently selected from —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴;
—NR¹⁰C(NR¹¹)R¹²; —C(NR¹¹)R¹²; halogen, —OH; and C₁ to C₄ alkoxy; C₁ to C₄ alkyl; C₂ to C₄ alkenyl; C₂ to C₄ alkynyl; and —NR^{Y}—C₁ to C₄ alkyl; wherein each alkyl, alkenyl, alkoxy and alkynyl group is independently unsubstituted or is substituted with one, two or three groups independently selected from —OH, halogen; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²;
wherein the nitrogen atom(s) in said heterocyclic group are independently selected from secondary, tertiary and quaternary nitrogen atom(s);
ii) C₂ to C₄ alkoxy; C₁ to C₄ alkyl; C₂ to C₄ alkenyl; C₂ to C₄ alkynyl; and —NR^{Y}—C₁ to C₄ alkyl each of which is unsubstituted or is substituted with one, two or three groups independently selected from —OH, halogen; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²; and methoxy which is substituted by one, two or three halogen substituents;
iii) halogen, —OH and unsubstituted methoxy; and
iv) C₃ to C₆ carbocyclyl; —O—C₃ to C₆ carbocyclyl; and —NR^{Y}—C₃ to C₆ carbocyclyl; wherein each carbocyclyl group is unsubstituted or is substituted with one or two groups independently selected from —OH, halogen; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; —C(NR¹¹)R¹²; C₁ to C₄ alkoxy; C₁ to C₄ alkyl; C₂ to C₄ alkenyl; C₂ to C₄ alkynyl; and —NR^{Y}—C₁ to C₄ alkyl; wherein each alkyl, alkenyl, alkoxy and alkynyl group is independently unsubstituted or is substituted with one, two or three groups independently selected from —OH, halogen; methoxy; —NR¹⁰R¹¹; —N⁺R¹⁰R¹¹R¹²; —NR¹⁰C(NR¹¹)NR¹²R¹³; —NR¹⁰C(N⁺R¹¹R¹²)NR¹³R¹⁴; —NR¹⁰C(NR¹¹)R¹²; and —C(NR¹¹)R¹²;
v) each R¹⁰, R¹¹, R¹², R¹³ and R¹⁴ is independently H or methyl; and
vi) R^{Y} is H or unsubstituted C₁ to C₃ alkyl.

37. The combination of claim 1, wherein each G is independently selected from:
a 4- to 6-membered nitrogen-containing heterocyclic group which is unsubstituted or is substituted by one or two substituents selected from C₁ to C₂ alkyl; —NR¹⁰R¹¹; and —N⁺R¹⁰R¹¹R¹²;
C₂ to C₄ alkoxy; C₁ to C₄ alkyl; C₂ to C₄ alkenyl; C₂ to C₄ alkynyl; and —NR^{Y}—C₁ to C₄ alkyl; each of which is unsubstituted or is substituted with one or two groups independently selected from —NR¹⁰R¹¹ and —N⁺R¹⁰R¹¹R¹²; and
chlorine, bromine, —OH and methoxy.

38. The combination of claim 1, wherein the moiety \textcircled{A}-(G)ₘ is 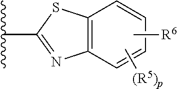
wherein:
p is 0 or 1;
R⁵ is selected from —OMe, —OH, halogen, —NR²⁰R²¹; —N⁺R²⁰R²¹R²², —CF₃, and R⁶; and
each R⁶ is independently selected from:
C₂ to C₄ alkoxy which is unsubstituted or is substituted with a group selected from —OH; —NR¹⁰R¹¹; —N⁺R¹⁰R¹¹R¹²; —OR^{6c} and —NR¹⁰R^{6c}, wherein R^{6c} is a C₁ to C₃ alkyl group which is unsubstituted or substituted with a group selected from OH; —NR¹⁰R¹¹; —N⁺R¹⁰R¹¹R¹²; —NR¹⁰NR¹¹R¹²; —NR¹⁰N⁺R¹¹R¹²R¹³; —N⁺R¹⁰R¹¹NR¹²R¹³; —NR¹⁰C(NR¹¹)NR¹²R¹³; —NR¹⁰C(N⁺R¹¹R¹²)NR¹³R¹⁴; —C(NR¹⁰)NR¹¹R¹²; and —C(NR¹⁰R¹¹)NR¹²R¹³; and each R¹⁰, R¹¹, R¹², R¹³ and R¹⁴ is independently H or methyl;
—R^{6a}R^{A}, —O—R^{6a}R^{A}, —NR²⁰—R^{6a}R^{A}, —R^{6b}R^{B}, —O—R^{6b}R^{B}, and —NR²⁰—R^{6b}R^{B};
—R^{X}R^{R}, —O—R^{X}R^{R}, —O—R^{X}—C(O)—R^{R}, —R^{X}—C(O)—R^{R}, —NR²⁰—R^{X}R^{R}, and —NR²⁰—R^{X}—C(O)—R^{R}; and
—CN; —C(O)NR²⁰R²¹; —C(O)NR²¹—R^{X}R^{B}; —C(O)NR⁴⁰R⁴¹; —SO₂R²⁰; —SO₂—R^{X}R^{B}; —SO₂NR²⁰R²¹; —SO₂—NR²⁰—R^{X}R^{B}; and —SO₂NR⁴⁰R⁴¹;
wherein:
each R^{X} is independently selected from R^{6a} and R^{6b};
each R^{6a} is independently selected from C₁ to C₄ alkylene, C₂ to C₄ alkenylene and C₂ to C₄ alkynylene; and each R^{6a} is independently unsubstituted or is substituted by one group selected from —OH, halogen; —NR²⁰R²¹; —N⁺R²⁰R²¹R²²; —NR²⁰C(NR²¹)NR²²R²³; —NR²⁰C(N⁺R²¹R²²)NR²³R²⁴; —NR²⁰C(NR²¹)R²²; NR²⁰C(N⁺R²¹R²²)R²³; —C(NR²⁰)NR²¹R²²; —C(N⁺R²⁰R²¹)NR²²R²³; —C(NR²⁰)R²¹; and —C(N⁺R²⁰R²¹)R²²; —C(O)NR²⁰R²¹; —C(O)N⁺R²⁰R²¹R²²; —C(O)—R²⁰, and methoxy which is unsubstituted or is substituted by one, two or three halogen substituents;
each R^{6b} is independently selected from [C₁ to C₃ alkylene]-C(R^{z})₂, [C₂ to C₃ alkenylene]—C(R^{z})₂ and [C₂ to C₃ alkynylene]—C(R^{z})₂; wherein the two R^{Z} groups are attached together to form, together with the atom to which they are attached, a 5- or 6-membered carbocyclic or heterocyclic group;
R^{A} is selected from —NR²⁰R³⁰; —N⁺R²⁰R²¹R³⁰; —NR²⁰NR²¹R²²; —NR²⁰N⁺R²¹R²²R²³; —N⁺R²⁰R²¹NR²²R²³; —NR²⁰C(NR²¹)NR²²R³⁰; —NR²⁰C(N⁺R²¹R²²)NR²³R³⁰; —C(NR²⁰)NR²¹R²²; and —C(N⁺R²⁰R²¹)NR²²R²³;
R^{B} is selected from —NR²⁰R²¹; —N⁺R²⁰R²¹R²²; —NR²⁰NR²¹R²²; —NR²⁰N⁺R²¹R²²R²³; —N⁺R²⁰R²¹NR²²R²³; —NR²⁰C(NR²¹)NR²²R²³; —NR²⁰C(NR²¹R²²)NR²³R²⁴; —C(NR²⁰)NR²¹R²²; and —C(N⁺R²⁰R²¹)NR²²R²³;
R⁴⁰ and R⁴¹, together with the nitrogen atom to which they are attached, form a 4- to 6-membered heterocyclic group, wherein any nitrogen atom in the ring is independently selected from secondary, tertiary and quaternary nitrogen atoms;
each R^{R} is independently a 4- to 10-membered heteroaryl or heterocyclic group comprising at least one nitrogen atom, and said nitrogen atom(s) are independently selected from secondary, tertiary and quaternary nitrogen atom(s);
wherein each R^{R}, and each ring formed by —NR⁴⁰R⁴¹, is independently unsubstituted or is substituted with one, two or three groups independently selected from
i) halogen, —CN;
ii) oxo, providing that said R^{R} group is a heterocyclic group;
iii) —R²⁰, —R⁷—OR²⁰; —R⁷—NR²⁰R²¹; —R⁷—N⁺R²⁰R²¹R²²; —R⁷—NR²⁰C(NR²¹)NR²²R²³; —R⁷—NR²⁰C(N⁺R²¹R²²)NR²³R²⁴; —R⁷—NR²⁰C(NR²¹)R²²; —R⁷—NR²⁰C(N⁺R²¹R²²)R²³;
—R⁷—C(NR²⁰)NR²¹R²²; —R⁷—C(N⁺R²⁰R²¹)NR²²R²³; —R⁷—C(NR²⁰)R²¹; and —R⁷—C(N⁺R²⁰R²¹)R²²;
each R⁷ is independently selected from a bond and unsubstituted C₁ to C₃ alkylene;
R²⁰, R²¹, R²², R²³ and R²⁴ are each independently selected from H and C₁ to C₃ alkyl which is unsubstituted or is substituted with one —OH or —OMe group or with one, two or three halogen groups;
each R³⁰ is independently selected from C₂ to C₃ alkyl which is unsubstituted or is substituted with one —OH or —OMe group or with one, two or three halogen groups;
with the proviso that the compound is other than:
2-(2-(((4-ethoxybenzo[d]thiazol-2-yl)methyl)carbamoyl)-2,3-dihydro-1H-inden-2-yl)acetic acid;
2-[2-[(6-ethoxy-1,3-benzothiazol-2-yl)methylcarbamoyl]indan-2-yl]acetic acid;
2-[2-[[6-(2-hydroxyethoxy)-1,3-benzothiazol-2-yl]methylcarbamoyl]indan-2-yl]acetic acid;
2-[2-[[6-[2-(dimethylamino)ethoxy]-1,3-benzothiazol-2-yl]methylcarbamoyl]indan-2-yl]acetic acid;
2-[2-[[6-[2-(trimethylammonio)ethoxy]-1,3-benzothiazol-2-yl]methylcarbamoyl]indan-2-yl]acetate;
2-[2-[[5-[2-(dimethylamino)ethoxy]-1,3-benzothiazol-2-yl]methylcarbamoyl]indan-2-yl]acetic acid;
2-[2-[[5-[2-(trimethylammonio)ethoxy]-1,3-benzothiazol-2-yl]methylcarbamoyl]indan-2-yl]acetate;
2-(2-(((5-(3-(dimethylamino)propoxy)-6-methoxybenzo[d]thiazol-2-yl)methyl)carbamoyl)-5,6-difluoro-2,3-dihydro-1H-inden-2-yl)acetic acid;
2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate; and
2-(2-(((5-(2-(dimethylamino)ethoxy)-6-methoxybenzo[d]thiazol-2-yl)methyl)carbamoyl)-2,3-dihydro-1H-inden-2-yl)acetic acid.

39. The combination of claim 38, wherein
Lk is —CH₂—;
R⁵ is selected from —OMe, —OH, halogen, —NR¹⁰R¹¹; —N⁺R¹⁰R¹¹R¹², and —CF₃;
R⁶ is C₂ to C₄ alkoxy which is unsubstituted or is substituted with a group selected from —OH; —NR¹⁰R¹¹; —N⁺R¹⁰R¹¹R¹²; —OR^{6c} and —NR¹⁰R^{6c}, wherein R^{6c} is a C₁ to C₃ alkyl group which is unsubstituted or substituted with a group selected from OH;
—NR¹⁰R¹¹; —N⁺R¹⁰R¹¹R¹²; —NR¹⁰N⁺R¹¹R¹²; —NR¹⁰N⁺R¹¹R¹²R¹³; —N⁺R¹⁰R¹¹NR¹²R¹³; —NR¹⁰C(NR¹¹)NR¹²R¹³; —NR¹⁰C(N⁺R¹¹R¹²)NR¹³R¹⁴; —C(NR¹⁰)NR¹¹R¹²; and —C(N⁺R¹⁰R¹¹)NR¹²R¹³; and
each R¹⁰, R¹¹, R¹², R¹³ and R¹⁴ is independently H or methyl;
with the proviso that the compound is other than:
2-(2-(((4-ethoxybenzo[d]thiazol-2-yl)methyl)carbamoyl)-2,3-dihydro-1H-inden-2-yl)acetic acid;
2-[2-[(6-ethoxy-1,3-benzothiazol-2-yl)methylcarbamoyl]indan-2-yl]acetic acid;
2-[2-[[6-(2-hydroxyethoxy)-1,3-benzothiazol-2-yl]methylcarbamoyl]indan-2-yl]acetic acid;
2-[2-[[6-[2-(dimethylamino)ethoxy]-1,3-benzothiazol-2-yl]methylcarbamoyl]indan-2-yl]acetic acid;
2-[2-[[6-[2-(trimethylammonio)ethoxy]-1,3-benzothiazol-2-yl]methylcarbamoyl]indan-2-yl]acetate;
2-[2-[[5-[2-(dimethylamino)ethoxy]-1,3-benzothiazol-2-yl]methylcarbamoyl]indan-2-yl]acetic acid;
2-[2-[[5-[2-(trimethylammonio)ethoxy]-1,3-benzothiazol-2-yl]methylcarbamoyl]indan-2-yl]acetate;
2-(2-(((5-(3-(dimethylamino)propoxy)-6-methoxybenzo[d]thiazol-2-yl)methyl)carbamoyl)-5,6-difluoro-2,3-dihydro-1H-inden-2-yl)acetic acid;
2-(5,6-difluoro-2-(((6-methoxy-5-(3-(trimethylammonio)propoxy)benzo[d]thiazol-2-yl)methyl)carbamoyl)-2,3-dihydro-1H-inden-2-yl)acetate; and
2-(2-(((5-(2-(dimethylamino)ethoxy)-6-methoxybenzo[d]thiazol-2-yl)methyl)carbamoyl)-2,3-dihydro-1H-inden-2-yl)acetic acid.

40. The combination of claim 39, wherein:
Lk is —CH₂—;
R⁵ is selected from —OMe and —OH; and
R⁶ is C₂ to C₄ alkoxy which is substituted with a group selected from —NR¹⁰R¹¹; —N⁺R¹⁰R¹¹R¹²; and —OR^{6c}, wherein R^{6c} is a C₁ to C₃ alkyl group which is unsubstituted or substituted with a group selected from —NR¹⁰R¹¹; and —N⁺R¹⁰R¹¹R¹².

41. The combination of claim 38, wherein
Lk is selected from a bond and a C₁ to C₃ alkylene group which is unsubstituted or is substituted by one group selected from halogen, —OH, —OMe, —NR²⁰R²¹; —N⁺R²⁰R²¹R²², and —CF₃; wherein R²⁰, R²¹ and R²² are each independently selected from H and C₁ to C₃ alkyl which is unsubstituted or is substituted with one —OH or —OMe group or with one, two or three halogen groups;
R⁵ is selected from —OMe, —OH, halogen, —NR²⁰R²¹; —N⁺R²⁰R²¹R²², —CF₃, and R⁶;
each R⁶ is independently selected from:
—R^{6a}R^{A}, —O—R^{6a}R^{A}, —NR²⁰—R^{6a}R^{A}, —R^{6b}R^{B}, —O—R^{6b}R^{B}, and —NR²⁰—R^{6b}R^{B};
—R^{X}R^{R}, —O—R^{X}R^{R}, —O—R^{X}—C(O)—R^{R}, —R^{X}—C(O)—R^{R}, —NR²⁰—R^{X}R^{R}, and —NR²⁰—R^{X}—C(O)—R^{R}; and
—CN; —C(O)NR²⁰R²¹; —C(O)NR²¹—R^{X}R^{B}; —C(O)NR⁴⁰R⁴¹; —SO₂R²⁰; —SO₂—R^{X}R^{B}; —SO₂NR²⁰R²¹; —SO₂—NR²⁰—R^{X}R^{B}; and —SO₂NR⁴⁰R⁴¹;
wherein:
each R^{X} is independently selected from R^{6a} and R^{6b};
each R^{6a} is independently selected from C₁ to C₄ alkylene, C₂ to C₄ alkenylene and C₂ to C₄ alkynylene; and each R^{6a} is independently unsubstituted or is substituted by one group selected from —OH, halogen; —NR²⁰R²¹; —N⁺R²⁰R²¹R²²; —NR²⁰C(NR²¹)NR²²R²³; —NR²⁰C(N⁺R²¹R²²)NR²³R²⁴; —NR²⁰C(NR¹)R²²; —NR²⁰C(N⁺R²¹R²²)R²³; —C(NR²⁰)NR²¹R²²; —C(NR²⁰R²¹)NR²²R²³; —C(NR²⁰)R²¹; and —C(N⁺R²⁰R²¹)R²²; —C(O)NR²⁰R²¹; —C(O)N⁺R²⁰R²¹R²²; —C(O)—R²⁰, and methoxy which is unsubstituted or is substituted by one, two or three halogen substituents;
each R^{6b} is independently selected from [C₁ to C₃ alkylene]-C(R^{z})₂, [C₂ to C₃ alkenylene]—C(R^{z})₂ and [C₂ to C₃ alkynylene]—C(R^{z})₂; wherein the two R^{z} groups are attached together to form, together with the atom to which they are attached, a 5- or 6-membered carbocyclic or heterocyclic group;
R^{A} is selected from —NR²⁰R³⁰; —N⁺R²⁰R²¹R³⁰; —NR²⁰NR²¹R²²; —NR²⁰N⁺R²¹R²²R²³; —N⁺R²⁰R²¹NR²²R²³; —NR²⁰C(NR²¹)NR²²R³⁰; —NR²⁰C(NR²¹R²²)NR²³R³⁰; —C(NR²⁰)NR²¹R²²; and —C(N⁺R²⁰R²¹)NR²²R²³;
R^{B} is selected from —NR²⁰R²¹; —N⁺R²⁰R²¹R²²; —NR²⁰NR²¹R²²; —NR²⁰N⁺R²¹R²²R²³; —N⁺R²⁰R²¹NR²²R²³; —NR²⁰C(NR²¹)NR²²R²³; —NR²⁰C(NR²¹R²²)NR²³R²⁴; —C(NR²⁰)N²¹R²²; and —C(N⁺R²⁰R²¹)NR²²R²³;
R⁴⁰ and R⁴¹, together with the nitrogen atom to which they are attached, form a 4- to 6-membered heterocyclic group, wherein any nitrogen atom in the ring is independently selected from secondary, tertiary and quaternary nitrogen atoms;
each R^{R} is independently a 4- to 10-membered heteroaryl or heterocyclic group comprising at least one nitrogen atom, and said nitrogen atom(s) are independently selected from secondary, tertiary and quaternary nitrogen atom(s);
wherein each R^{R}, and each ring formed by —NR⁴⁰R⁴¹, is independently unsubstituted or is substituted with one, two or three groups independently selected from
i) halogen, —CN;
ii) oxo, providing that said R^{R} group is a heterocyclic group;
iii) —R²⁰, —R⁷—OR²⁰; —R⁷—NR²⁰R²¹; —R⁷—N⁺R²⁰R²¹R²²; —R⁷—NR²⁰C(NR²¹)NR²²R²³; —R⁷—NR²⁰C(N⁺R²¹R²²)NR²³R²⁴; —R⁷—NR²⁰C(NR²¹)R²²; —R⁷—NR²⁰C(N⁺R²¹R²²)R²³;
—R⁷—C(NR²⁰)NR²¹R²²; —R⁷—C(N⁺R²⁰R²¹)NR²²R²³; —R⁷—C(NR²⁰)R²¹; and
—R⁷—C(N⁺R²⁰R²¹)R²²;
each R⁷ is independently selected from a bond and unsubstituted C₁ to C₃ alkylene;
R²⁰, R²¹, R²², R²³ and R²⁴ are each independently selected from H and C₁ to C₃ alkyl which is unsubstituted or is substituted with one —OH or —OMe group or with one, two or three halogen groups;
each R³⁰ is independently selected from C₂ to C₃ alkyl which is unsubstituted or is substituted with one —OH or —OMe group or with one, two or three halogen groups.

42. The combination of claim 41, wherein:
Lk is —CH₂—;
R⁵ is selected from —OMe and —OH; and
R⁶ is selected from: —O—R^{6a}R^{A}, —O—R^{6b}R^{B}, —O—R^{X}R^{R}, and —O—R^{X}—C(O)—R^{R}, wherein:
each R^{X} is an R^{6a} group;
each R^{6a} is independently an unsubstituted C₁ to C₄ alkylene group;
each R^{6b} is independently a [C₁ to C₃ alkylene]-C(R^{z})₂ group; wherein the two R^{z} groups are attached together to form, together with the atom to which they are attached, a 5- or 6-membered heterocyclic group;
R^{A} is selected from —NR²⁰R³⁰; —N⁺R²⁰R²¹R³⁰; —NR²⁰NR²¹R²²; and —NR²⁰N⁺R²¹R²²R²³;
R^{B} is selected from —NR²⁰R²¹; —N⁺R²⁰R²¹R²²; —NR²⁰NR²¹R²²; and —NR²⁰N⁺R²¹R²²R²³;
each R^{R} is independently a 5- to 6-membered heteroaryl or 4- to 6-membered heterocyclic group comprising at least one nitrogen atom, and said nitrogen atom(s) are independently selected from secondary, tertiary and quaternary nitrogen atom(s); and
wherein each R^{R} is independently unsubstituted or is substituted with one or two groups independently selected from —R²⁰; —R⁷—NR²⁰R²¹; and —R⁷—N⁺R²⁰R²¹R²².

43. The combination of claim 1, wherein:
R¹ is selected from —OH and —NHOH, or where the compound of Formula (I) contains a positively charged nitrogen atom, R¹ may be O⁻, such that the compound forms a zwitterion;
R² is H;
R⁴ is H;
n is 0; or n is 2 and each R³ group is fluorine;
Lk is —CH₂—;
The moiety \textcircled{A}-(G)ₘ is 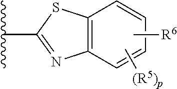
p is 1;
R⁵ is —OMe; and
R⁶ is selected from:
—O—R^{6a}R^{A} and —O—R^{X}R^{R}; and
C₂ to C₄ alkoxy which is substituted with a group selected from —NR¹⁰R¹¹; —N⁺R¹⁰R¹¹R¹²; and —OR^{6c}, wherein R^{6c} is a C₁ to C₃ alkyl group which is unsubstituted or substituted with a group selected from —NR¹⁰R¹¹; and —N⁺R¹⁰R¹¹R¹²;
R^{X} is R^{6a};
R^{6a} is an unsubstituted C₁ to C₄ alkylene group;
R^{A} is selected from —NR²⁰R³⁰; —N⁺R²⁰R²¹R³⁰;
R^{R} is a 5- to 6-membered heterocyclic group comprising at least one nitrogen atom, and said nitrogen atom(s) are independently selected from secondary, tertiary and quaternary nitrogen atom(s); and R^{R} is unsubstituted or is substituted with one or two R²⁰ groups;
R¹⁰, R¹¹ and R¹² are each independently H or methyl;
R²⁰ and R²¹ are each independently selected from H and C₁ to C₃ alkyl which is unsubstituted or is substituted with one —OH or —OMe group;
each R³⁰ is independently selected from C₂ to C₃ alkyl which is unsubstituted or is substituted with one —OH or —OMe group.

44. The combination of claim 1, wherein said CFTR modulator is selected from CFTR potentiators, CFTR correctors and CFTR amplifiers.

45. The combination of claim 1, wherein said CFTR modulator is selected from ivacaftor, lumacaftor, tezacaftor, elexacaftor, VX659, VX152 and VX-440 and combinations thereof.

46. The combination of claim 1, further comprising an antibiotic agent.

47. A pharmaceutical composition, comprising:
(i) a compound which is an indane according to Formula (I) as defined in claim 1 or a pharmaceutically acceptable salt thereof;
(ii) one or more CFTR modulator; and
(iii) one or more pharmaceutically acceptable excipient, carrier or diluent.

48. The pharmaceutical composition of claim 47, wherein said CFTR modulator is selected from CFTR potentiators, CFTR correctors and CFTR amplifiers.

49. The pharmaceutical composition of claim 47, wherein said CFTR modulator is selected from ivacaftor, lumacaftor, tezacaftor, elexacaftor, VX659, VX152 and VX-440 and combinations thereof.

50. The pharmaceutical composition of claim 47, further comprising an antibiotic agent.

51. The pharmaceutical composition of claim 50, wherein the antibiotic agent is selected from tobramycin, neomycin, streptomycin, gentamycin, ceftazidime, ticarcillin, piperacillin, tazobactam, imipenem, meropenem, rifampicin, ciprofloxacin, amikacin, colistin, aztreonam, azithromycin and levofloxacin.

52. A method of treating a disease associated with CFTR downregulation or decreased CFTR function in a subject, comprising administering to said subject an effective amount of a combination according to claim 1.

53. The method of claim 52, wherein said disease is cystic fibrosis (CF) or chronic obstructive pulmonary disease.

54. The method of claim 52, wherein said subject has a CFTR mutation selected from F508del; G178R, G1244E, S549R, G551D, G1349D, S1251N, G551S, S549N, S1255P, A455E, E193K, R117C, A1067T, F1052V, R347H, D110E, F1074L, R352Q, D110H, G1069R, R1070Q, D579G, K1060T, R1070W, D1152H, L206W, S945L, D1270N, P67L, S977F, E56K, R74W, E831X and R117H.

55. The method of claim 52, wherein said combination further treats bacterial infection in said subject.

56. A method of treating a disease associated with CFTR downregulation or decreased CFTR function in a subject, comprising administering to said subject an effective amount of a pharmaceutical composition according to claim 47.

57. The method of claim 56, wherein said disease is cystic fibrosis (CF) or chronic obstructive pulmonary disease.

58. The method of claim 56, wherein said subject has a CFTR mutation selected from F508del; G178R, G1244E, S549R, G551D, G1349D, S1251N, G551S, S549N, S1255P, A455E, E193K, R117C, A1067T, F1052V, R347H, D110E, F1074L, R352Q, D110H, G1069R, R1070Q, D579G, K1060T, R1070W, D1152H, L206W, S945L, D1270N, P67L, S977F, E56K, R74W, E831X and R117H.

59. The method of claim 56, wherein said pharmaceutical composition further treats bacterial infection in said subject.
